(19) United States
(12) Patent Application Publication
(10) Pub. No.: US 2024/0261237 A1
(43) Pub. Date: Aug. 8, 2024

(54) REDUCING SIDE EFFECTS OF NMDA RECEPTOR ANTAGONISTS

(71) Applicants: Raj MEHAR, New York, NY (US); Timothy WHITAKER, New York, NY (US); Seelos Therapeutics, Inc., New York, NY (US)

(72) Inventors: Raj Mehra, New York, NY (US); Timothy Whitaker, New York, NY (US)

(21) Appl. No.: 18/289,466

(22) PCT Filed: May 16, 2022
(86) PCT No.: PCT/US2022/029453

§ 371 Date: Nov. 3, 2023

Related U.S. Application Data
(60) Provisional application No. 63/188,738, filed on May 14, 2021.

(51) Int. Cl. A61K 31/135; A61K 9/00; A61P 25/24

(52) U.S. Cl. CPC A61K 31/135; A61K 9/0043; A61P 25/24

(57) ABSTRACT

The present disclosure relates to compositions comprising racemic ketamine, or a pharmaceutically acceptable salt thereof, for use in treating psychiatric disorders.

### TECHNICAL FIELD

The present disclosure relates to compositions and methods for treating psychiatric disorders.

### BACKGROUND

Psychiatric disorders are among the most disabling of all medical disorders and are a major public health problem. Many psychiatric disorders appear early in life, can occur chronically throughout life, and can adversely affect the prognosis of other medical illnesses such as cardiovascular and neurological conditions.

While medication and cognitive behavioral therapy can be effective for some individuals with psychiatric disorders such as depression, up to 20% do not respond to these interventions, and many of those who do respond, eventually relapse. For example, an estimated 50% of depressed individuals are only partially (inadequately) treated by available clinical interventions. See Al Harbi, Patient Prefer. Adherence, Vol. 6, pp. 369-388 (2012). In the NIMH Sequenced Treatment Alternatives to Relieve Depression (STAR*D) study, approximately half of patients treated with a first-line antidepressant therapy had reduction of symptoms to at least half of the original intensity and only approximately one-third of patients achieved remission (Chan 2013). While these patients may eventually recover, many require a trial and error approach to therapy, and many will ultimately develop treatment resistant depression over time. See, e.g., Sackheim, J. Clin. Psychiatry, Vol. 62, Suppl. 16, pp. 10-17 (2001).

While medications such as tricyclic antidepressants and monoamine oxidase inhibitors revolutionized the treatment of depression, many other psychiatric disorders have not been amenable to treatment with existing therapies. See, e.g., Smith B L, Amer. Psychol. Assoc. Monitor, Vol. 43, No. 6, p. 36 (2012). In many instances, medications for treating psychiatric disorders take weeks to months to achieve their full effects and may not be efficacious for all subjects at any safe dose. For example, most antidepressants require an average of 6 weeks to begin to have an effect on depressive symptoms. Some patients do not respond to antidepressant medications at all, and for others, only some types seem to work to ameliorate symptoms. In the meantime, individuals continue to suffer from depression, have a risk of self-harm, and experience a negative impact in their personal and professional lives. See, e.g., Burcusa and Iacono, Clin. Psychol. Rev. Vol. 27, No. 8, pp. 959-985 (2007). Providing rapid-onset and sustained treatment of psychiatric disorders would have a substantial impact on public health.

### SUMMARY

Some embodiments provide a method for treating major depressive disorder (MDD) in a subject in need thereof, comprising intranasally administering to the subject a therapeutically effective amount of racemic ketamine, or a pharmaceutically acceptable salt thereof.

Some embodiments provide a method for treating post-traumatic stress disorder (PTSD) in a subject in need thereof, comprising intranasally administering to the subject a therapeutically effective amount of racemic ketamine, or a pharmaceutically acceptable salt thereof.

Some embodiments provide a method for treating treatment-resistant depression (TRD) in a subject in need thereof, comprising intranasally administering to the subject a therapeutically effective amount of racemic ketamine, or a pharmaceutically acceptable salt thereof. In some embodiments, the TRD is treatment-resistant unipolar depression. In some embodiments, the TRD is treatment-resistant bipolar depression.

Some embodiments provide a method for treating bipolar depression in a subject in need thereof, comprising intranasally administering to the subject a therapeutically effective amount of racemic ketamine, or a pharmaceutically acceptable salt thereof.

Some embodiments provide a method for treating post-partum depression in a subject in need thereof, comprising intranasally administering to the subject a therapeutically effective amount of racemic ketamine, or a pharmaceutically acceptable salt thereof.

Some embodiments provide a method for treating chronic pain in a subject in need thereof, comprising intranasally administering to the subject a therapeutically effective amount of racemic ketamine, or a pharmaceutically acceptable salt thereof.

Some embodiments provide a method for treating neuropathic pain in a subject in need thereof, comprising intranasally administering to the subject a therapeutically effective amount of racemic ketamine, or a pharmaceutically acceptable salt thereof.

Some embodiments provide a method for treating Rett syndrome in a subject in need thereof, comprising intranasally administering to the subject a therapeutically effective amount of racemic ketamine, or a pharmaceutically acceptable salt thereof.

Some embodiments provide a method for treating epilepsy in a subject in need thereof, comprising intranasally administering to the subject a therapeutically effective amount of racemic ketamine, or a pharmaceutically acceptable salt thereof.

Some embodiments provide a method for treating agitation associated with dementia in a subject in need thereof, comprising intranasally administering to the subject a therapeutically effective amount of racemic ketamine, or a pharmaceutically acceptable salt thereof.

Some embodiments provide a method for treating agitation associated with schizophrenia in a subject in need thereof, comprising intranasally administering to the subject a therapeutically effective amount of racemic ketamine, or a pharmaceutically acceptable salt thereof.

Some embodiments provide a method for treating agitation associated with bipolar disorder in a subject in need thereof, comprising intranasally administering to the subject a therapeutically effective amount of racemic ketamine, or a pharmaceutically acceptable salt thereof.

Some embodiments provide a method for reducing one or more side effects of ketamine in a subject in need thereof, the method comprising intranasally administering a therapeutically effective amount of racemic ketamine, or a pharmaceutically acceptable salt thereof, to the subject.

In some embodiments, the subject has been previously diagnosed with and/or is currently suffering from, post-traumatic stress disorder. In some embodiments, the subject has been previously diagnosed with and/or is currently suffering from, major depressive disorder. In some embodiments, the subject has been previously diagnosed with and/or is currently suffering from, treatment-resistant depression. In some embodiments, the subject has been previously diagnosed with and/or is currently suffering from, bipolar depression. In some embodiments, the subject has been previously diagnosed with and/or is currently suffering from, post-partum depression. In some embodiments, the subject has been previously diagnosed with and/or is currently suffering from, post-partum depression and is not currently breastfeeding. In some embodiments, the subject has been previously diagnosed with and/or is currently suffering from, chronic pain. In some embodiments, the subject has been previously diagnosed with and/or is currently suffering from, neuropathic pain. In some embodiments, the subject has been previously diagnosed with and/or is currently suffering from, Rett syndrome. In some embodiments, the subject has been previously diagnosed with and/or is currently suffering from, epilepsy. In some embodiments, the subject has been previously diagnosed with and/or is currently suffering from, agitation associated with dementia. In some embodiments, the subject has been previously diagnosed with and/or is currently suffering from, agitation associated with schizophrenia. In some embodiments, the subject has been previously diagnosed with and/or is currently suffering from, agitation associated with bipolar disorder.

### DESCRIPTION OF THE FIGURES

### Abbreviations

N: Number of observations; SD: Standard Deviation; Min: Minimum; Max: Maximum; IN: Intranasal.

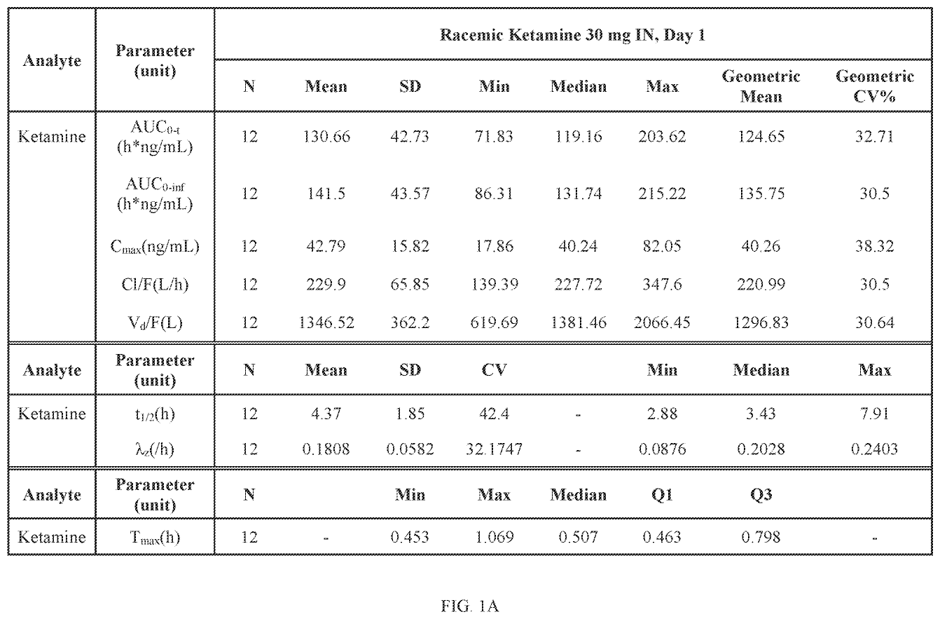

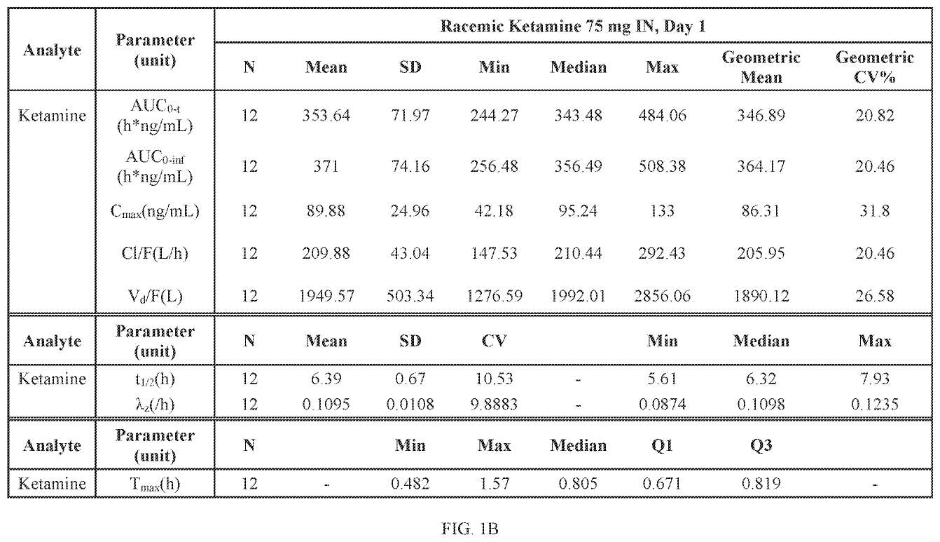

FIGS. 1A-1C are tables describing pharmacokinetic parameters for ketamine on Day 1 in Part A of the study described in Example 1 for 30 mg racemic ketamine (FIG. 1A), 75 mg racemic ketamine (FIG. 1B), or 90 mg racemic ketamine (FIG. 1C).

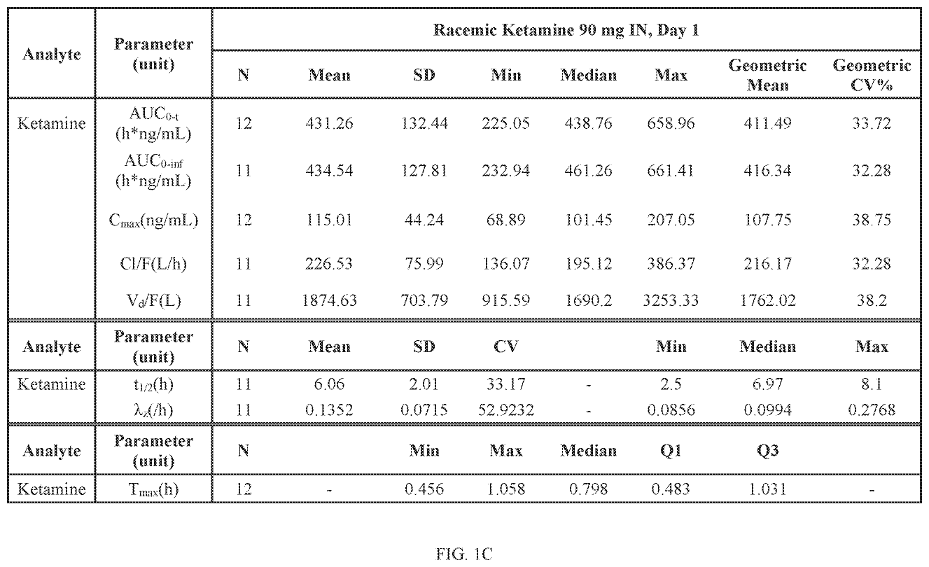

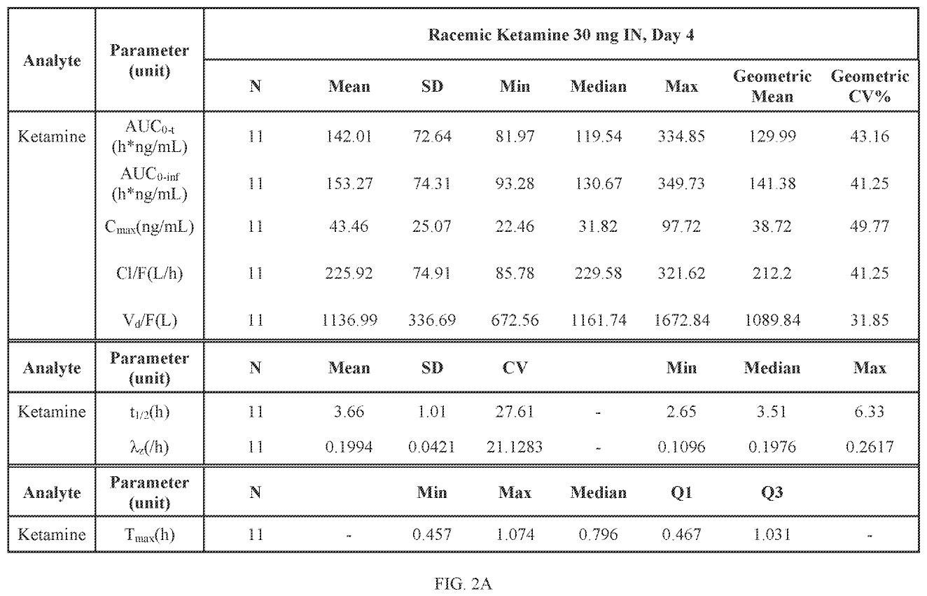

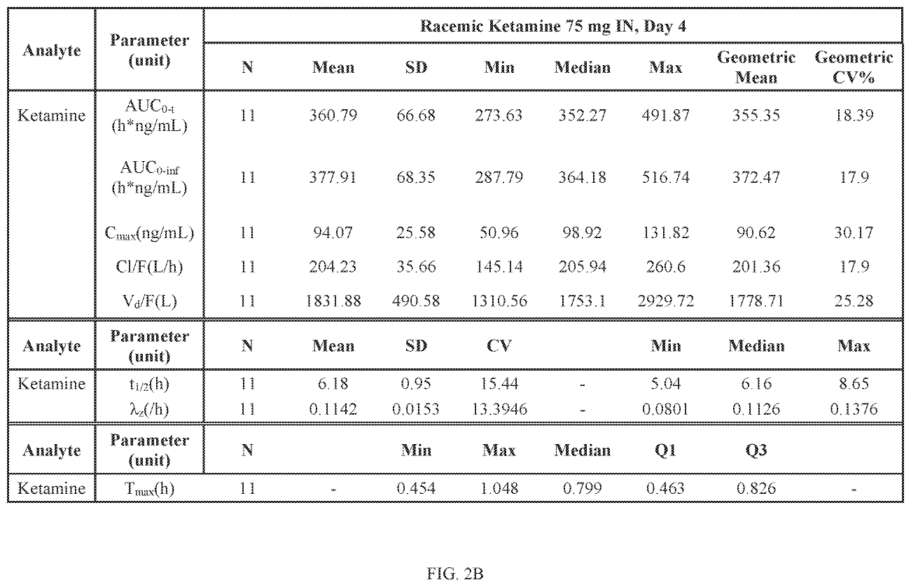

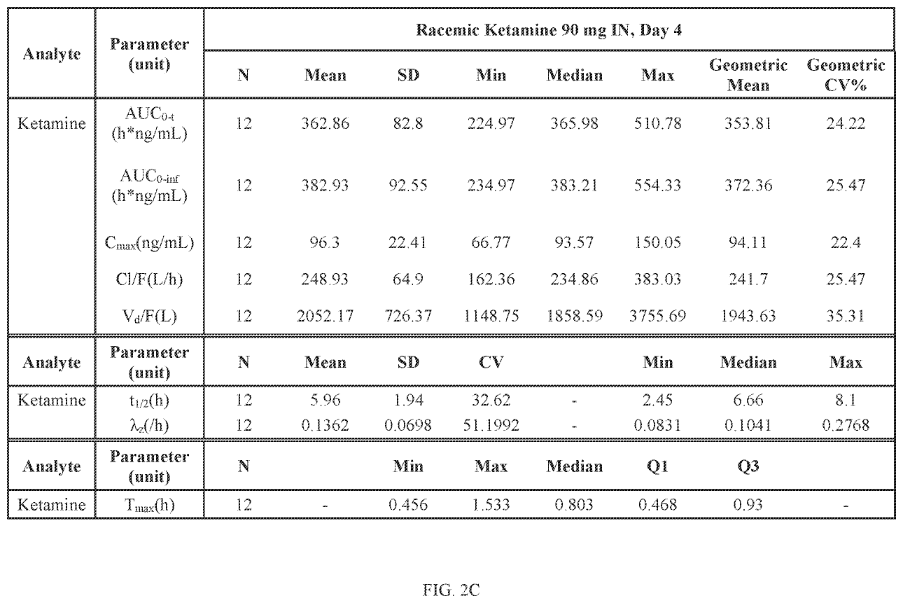

FIGS. 2A-2C are tables describing pharmacokinetic parameters for ketamine on Day 4 in Part A of the study described in Example 1 for 30 mg racemic ketamine (FIG. 2A), 75 mg racemic ketamine (FIG. 2B), or 90 mg racemic ketamine (FIG. 2C).

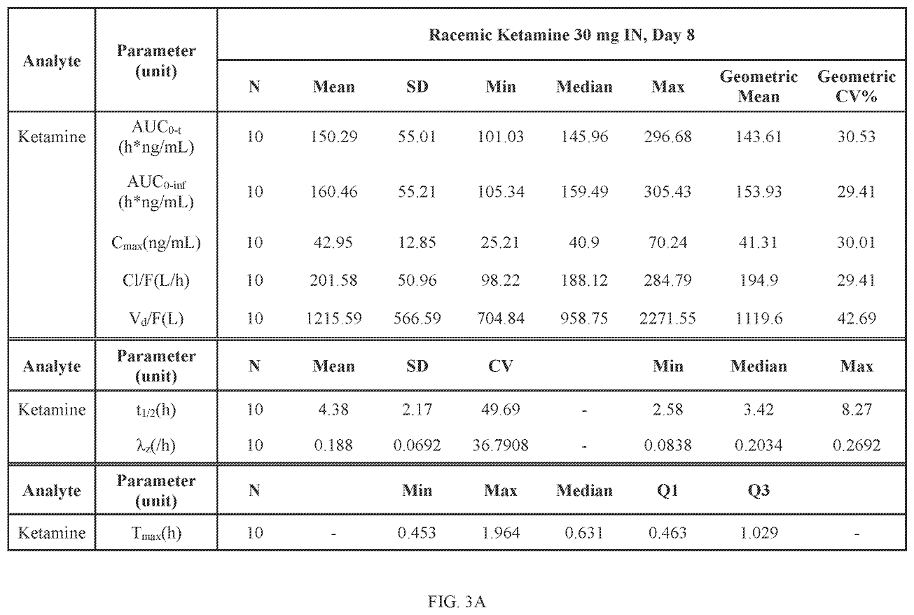

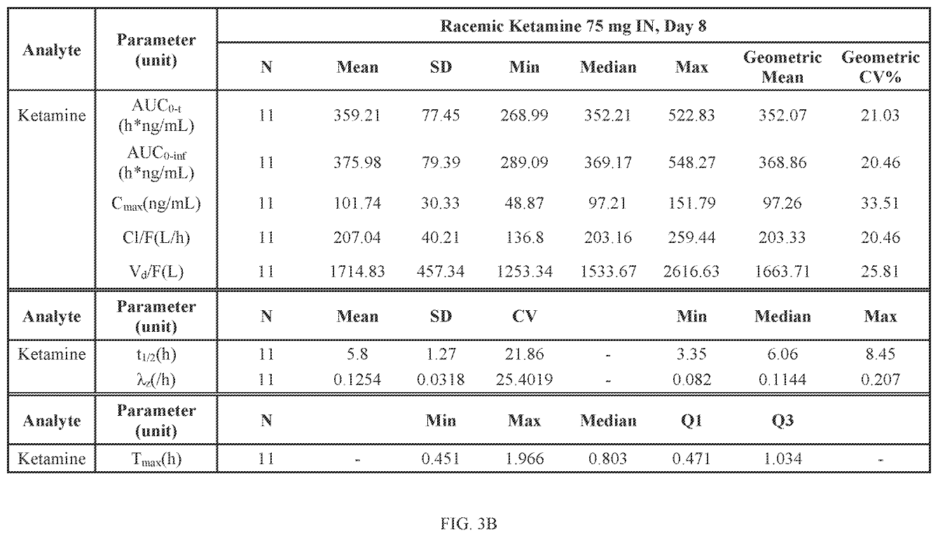

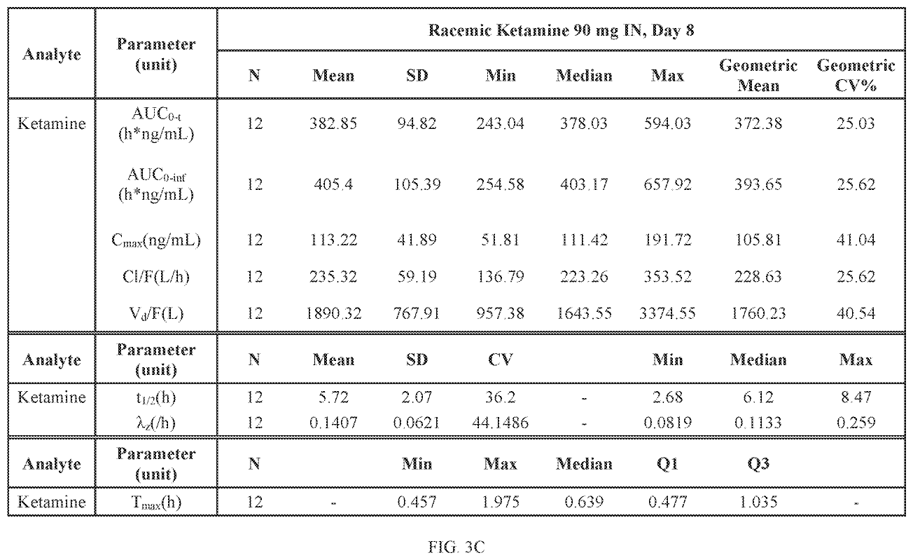

FIGS. 3A-3C are tables describing pharmacokinetic parameters for ketamine on Day 8 in Part A of the study described in Example 1 for 30 mg racemic ketamine (FIG. 3A), 75 mg racemic ketamine (FIG. 3B), or 90 mg racemic ketamine (FIG. 3C).

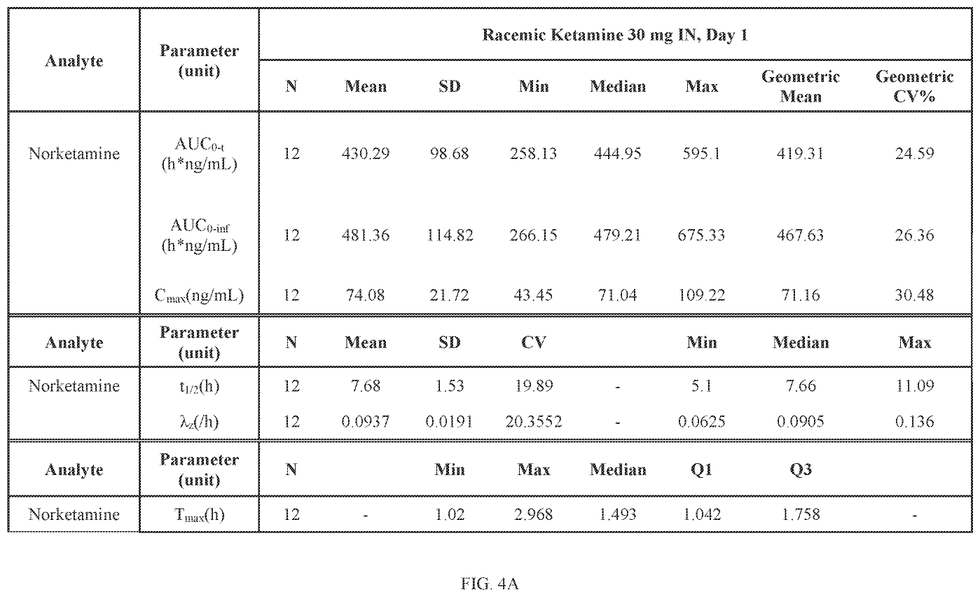

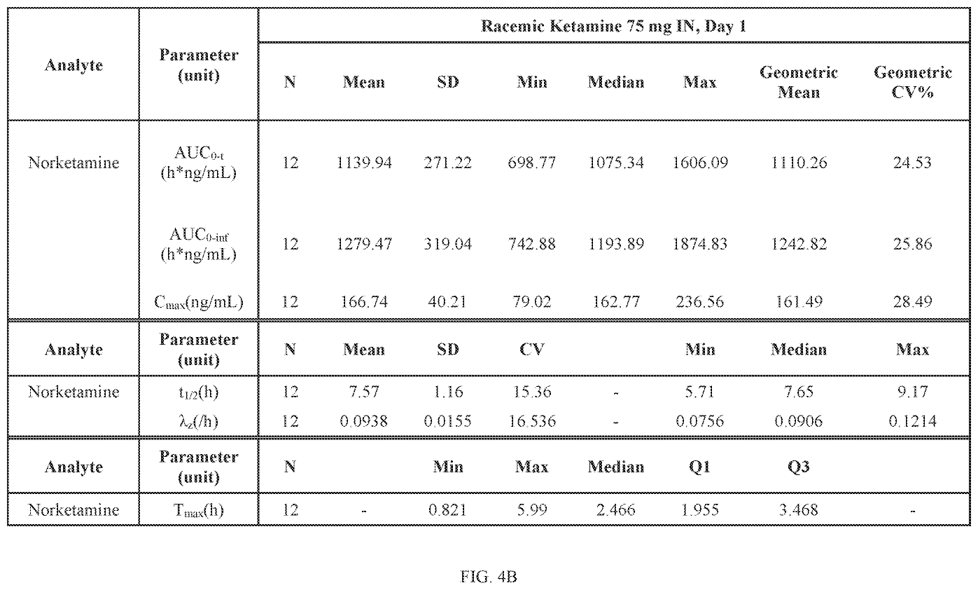

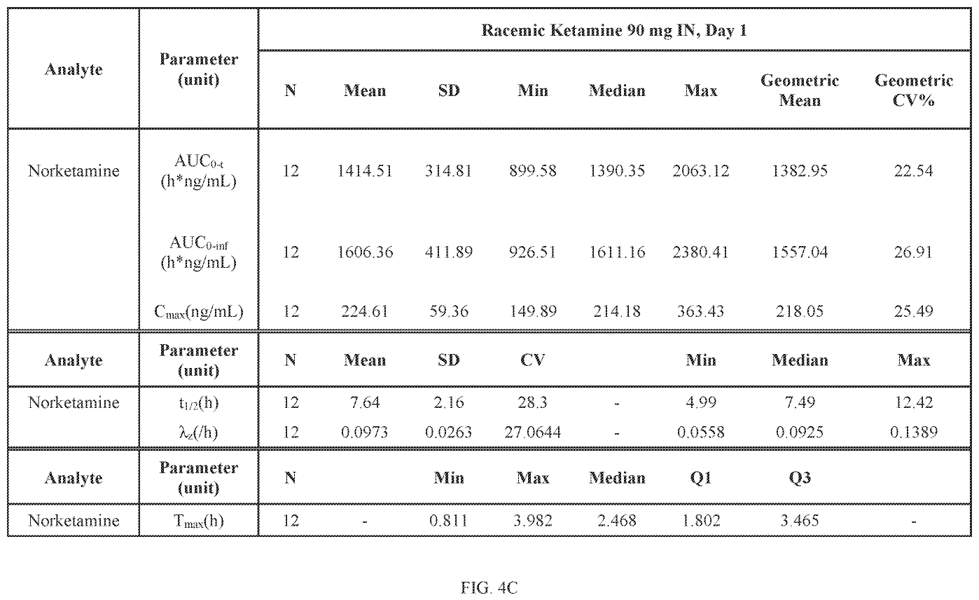

FIGS. 4A-4C are tables describing pharmacokinetic parameters for norketamine on Day 1 in Part A of the study described in Example 1 for 30 mg racemic ketamine (FIG. 4A), 75 mg racemic ketamine (FIG. 4B), or 90 mg racemic ketamine (FIG. 4C).

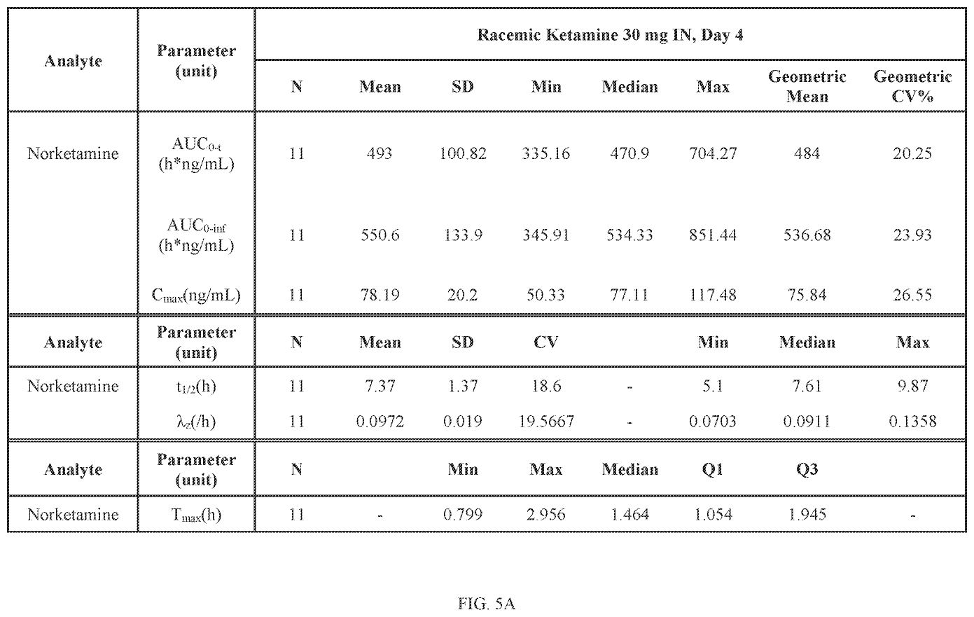

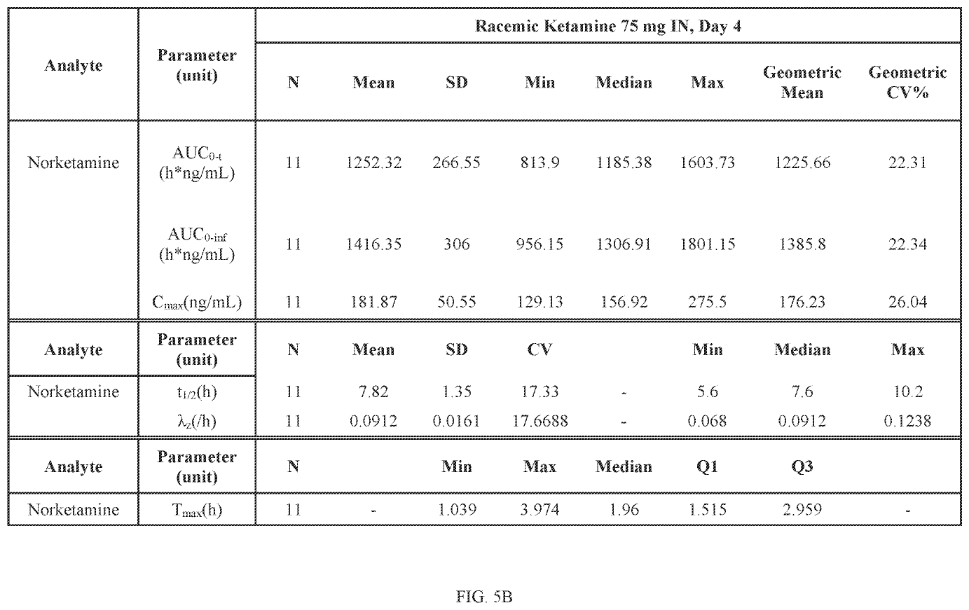

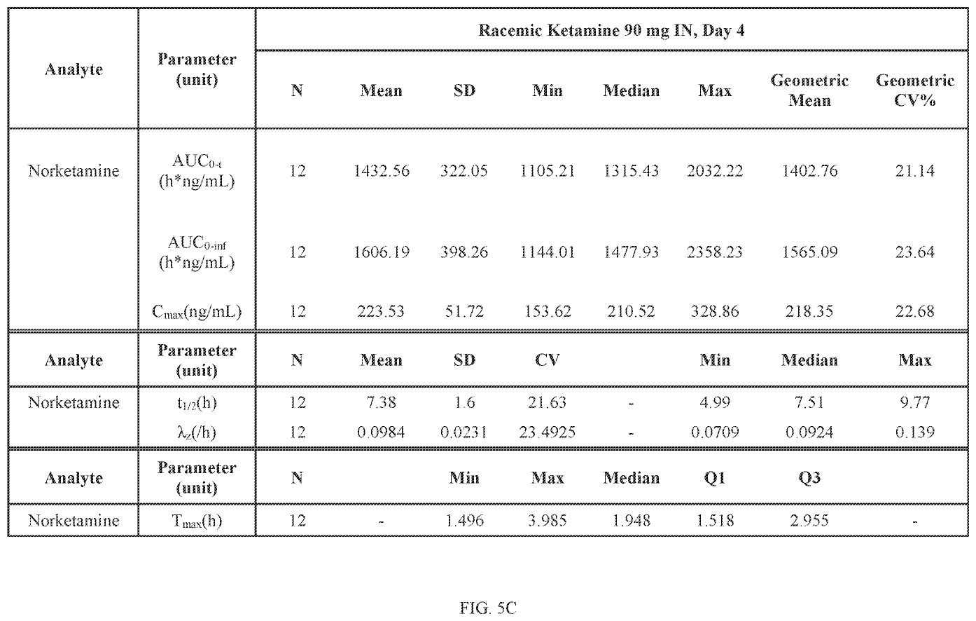

FIGS. 5A-5C are tables describing pharmacokinetic parameters for norketamine on Day 4 in Part A of the study described in Example 1 for 30 mg racemic ketamine (FIG. 5A), 75 mg racemic ketamine (FIG. 5B), or 90 mg racemic ketamine (FIG. 5C).

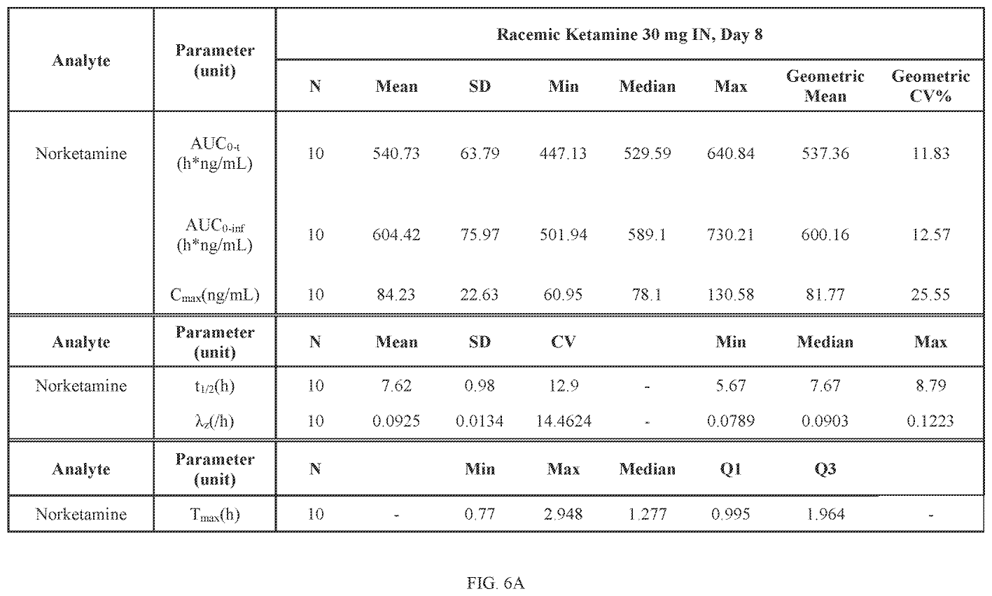

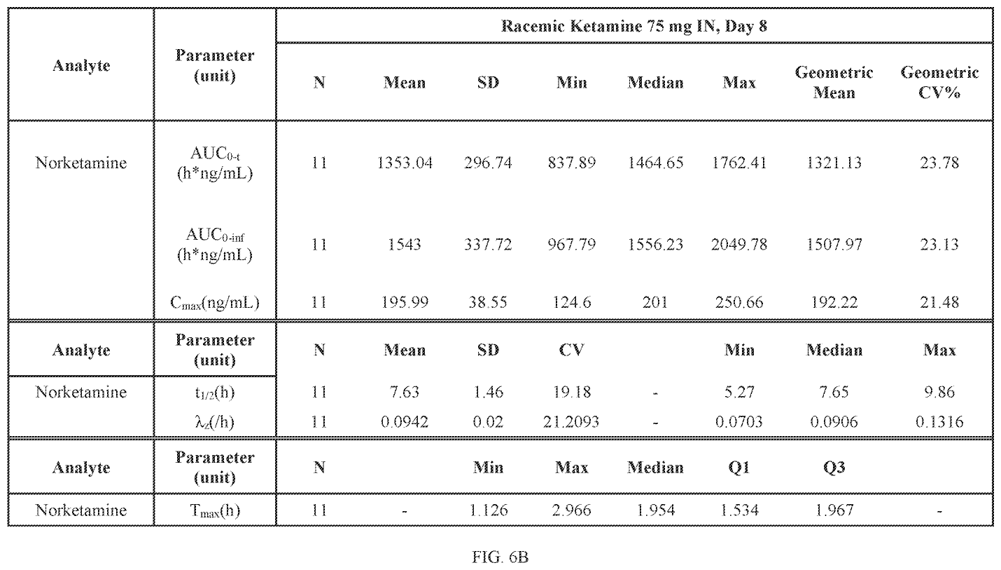

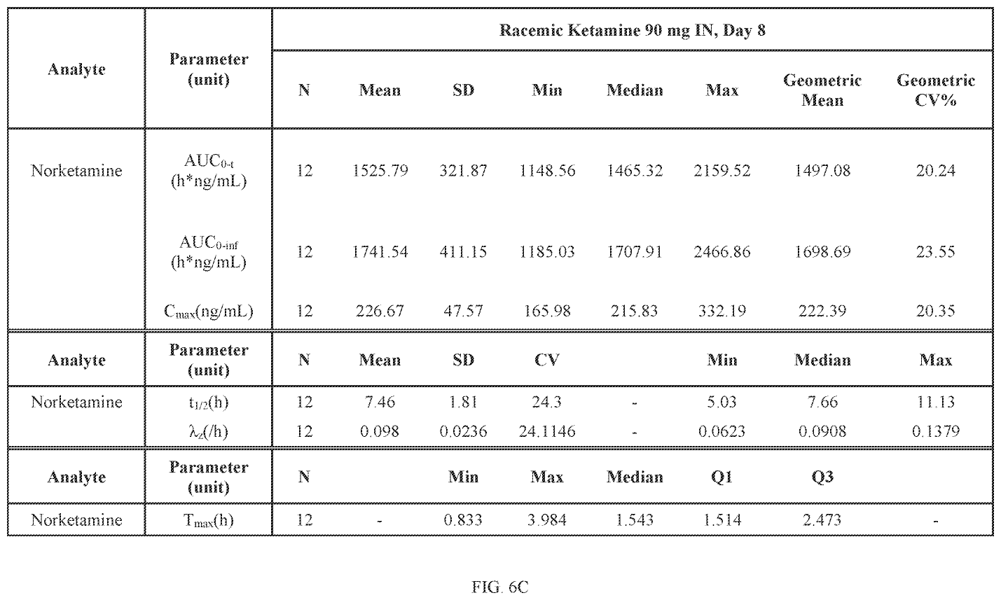

FIGS. 6A-6C are tables describing pharmacokinetic parameters for norketamine on Day 8 in Part A of the study described in Example 1 for 30 mg racemic ketamine (FIG. 6A), 75 mg racemic ketamine (FIG. 6B), or 90 mg racemic ketamine (FIG. 6C).

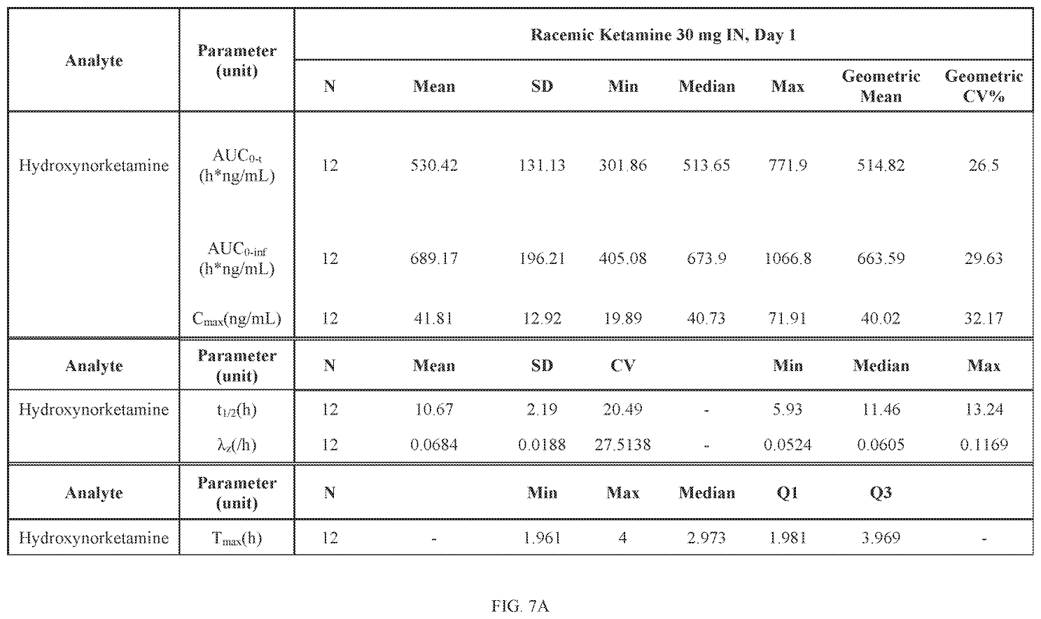

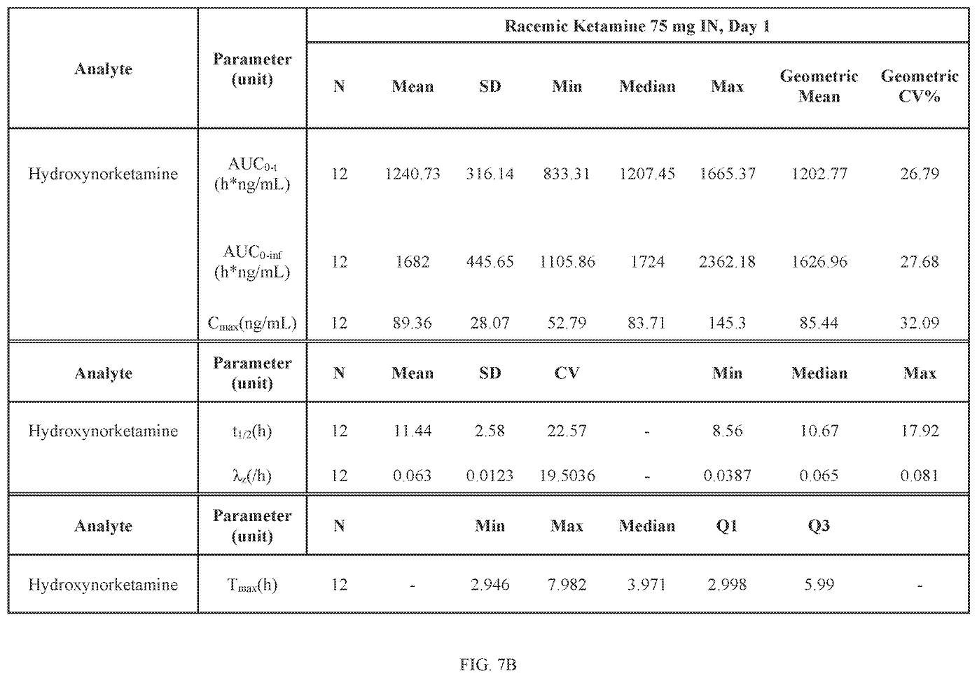

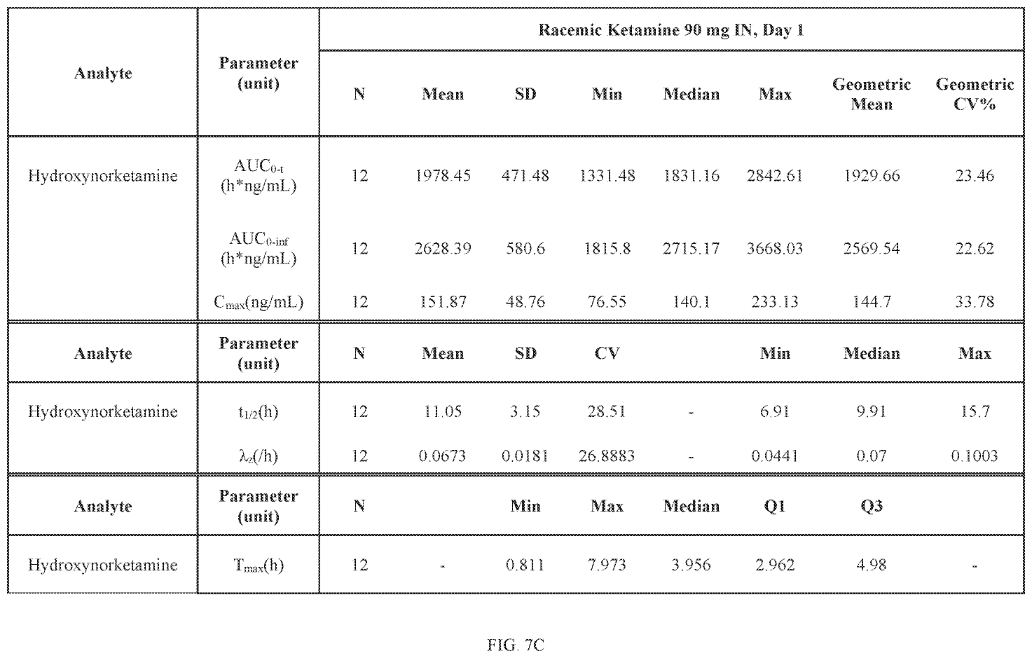

FIGS. 7A-7C are tables describing pharmacokinetic parameters for hydroxynorketamine on Day 1 in Part A of the study described in Example 1 for 30 mg racemic ketamine (FIG. 7A), 75 mg racemic ketamine (FIG. 7B), or 90 mg racemic ketamine (FIG. 7C).

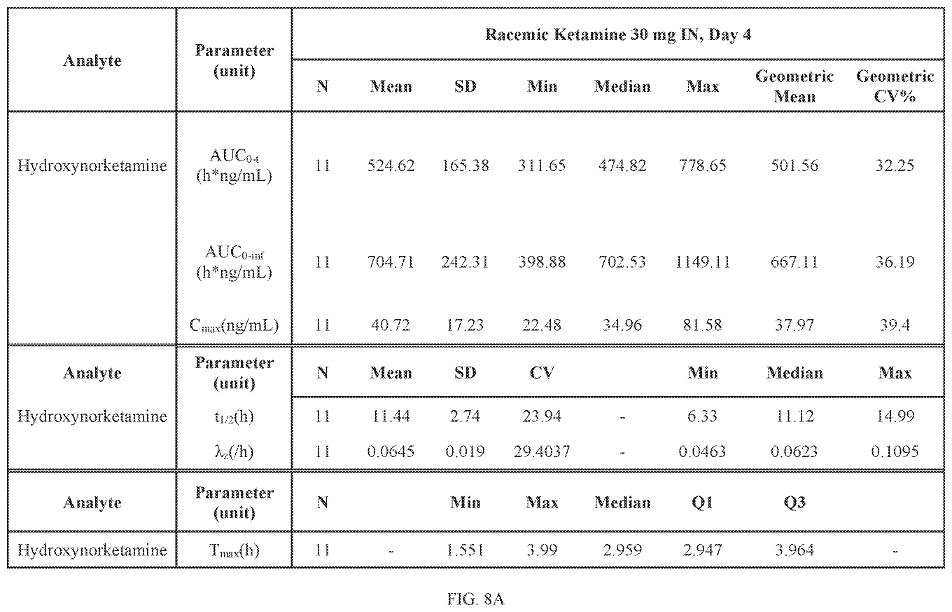

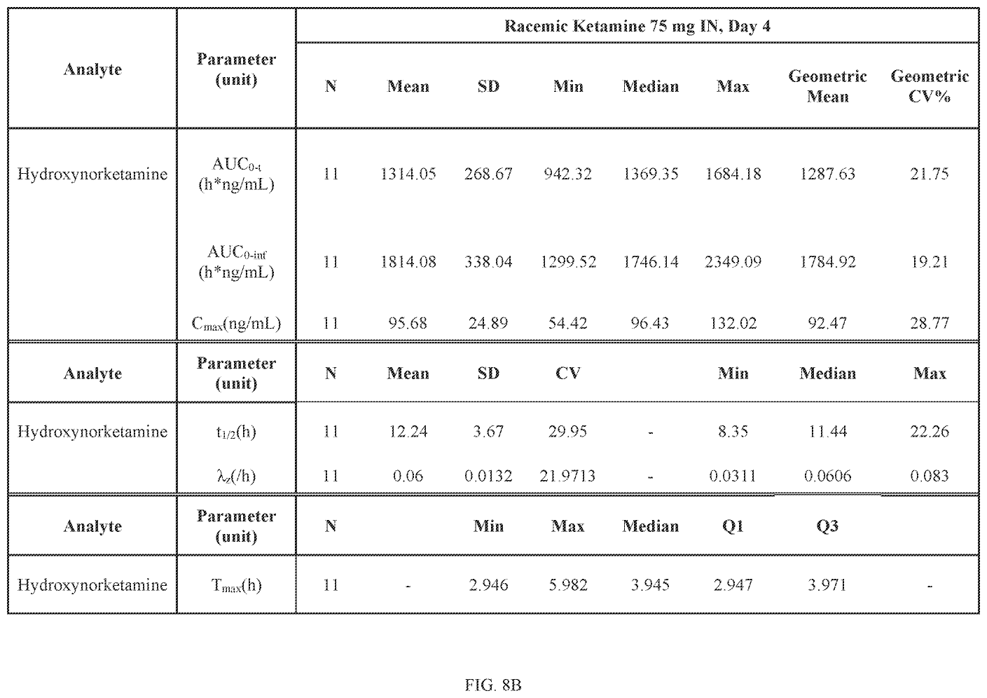

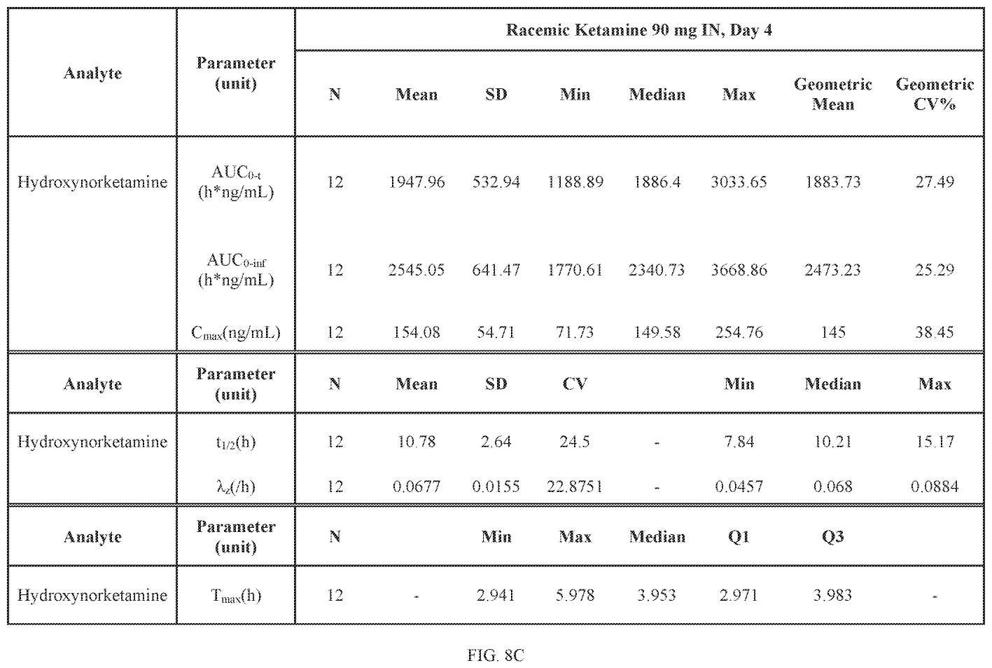

FIGS. 8A-8C are tables describing pharmacokinetic parameters for hydroxynorketamine on Day 4 in Part A of the study described in Example 1 for 30 mg racemic ketamine (FIG. 8A), 75 mg racemic ketamine (FIG. 8B), or 90 mg racemic ketamine (FIG. 8C).

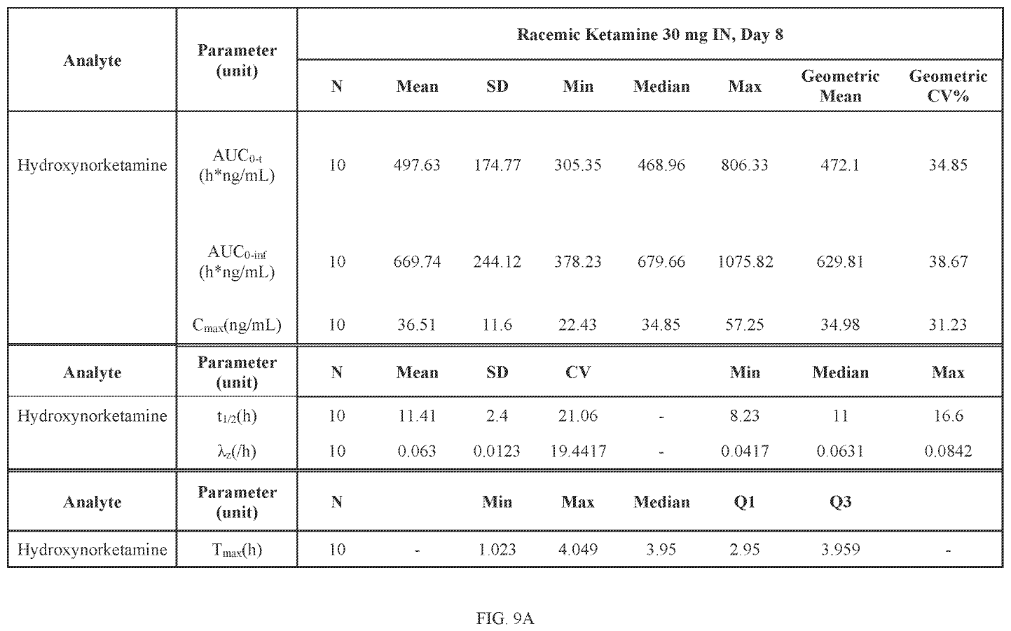

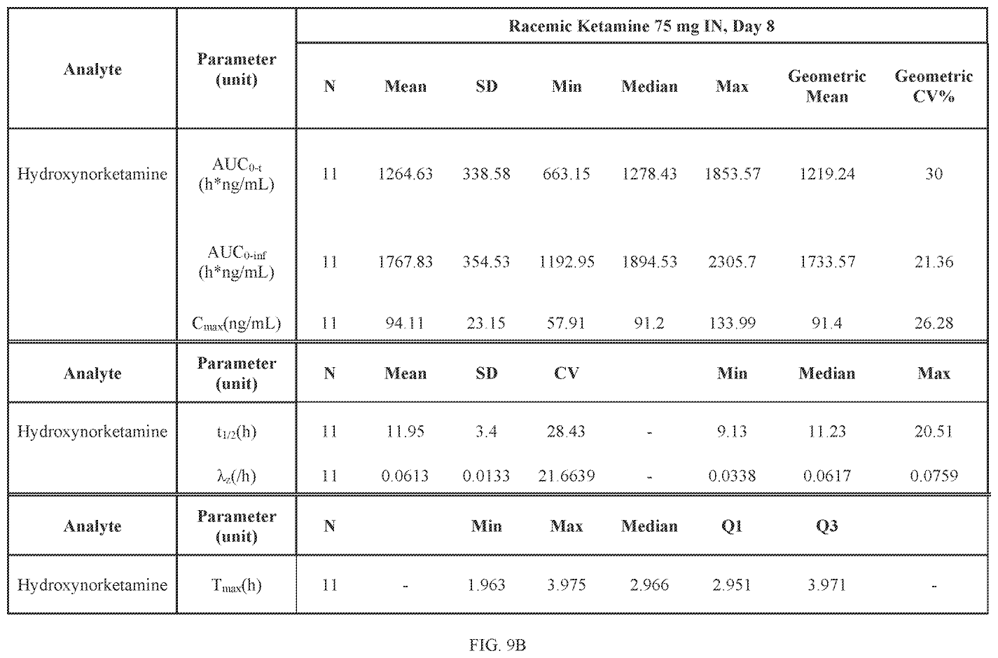

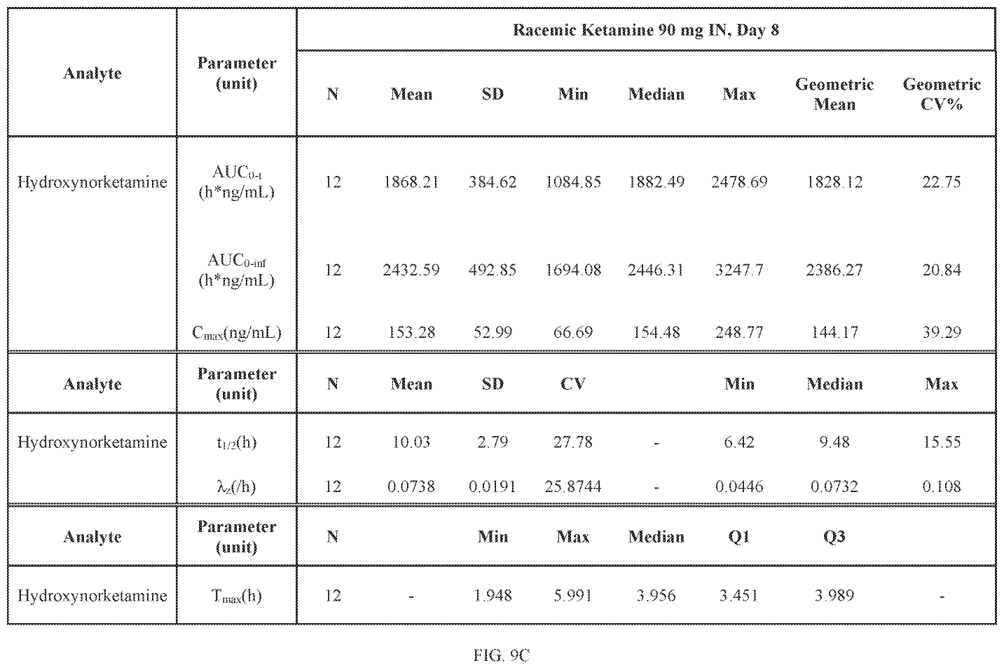

FIGS. 9A-9C are tables describing pharmacokinetic parameters for hydroxynorketamine on Day 8 in Part A of the study described in Example 1 for 30 mg racemic ketamine (FIG. 9A), 75 mg racemic ketamine (FIG. 9B), or 90 mg racemic ketamine (FIG. 9C).

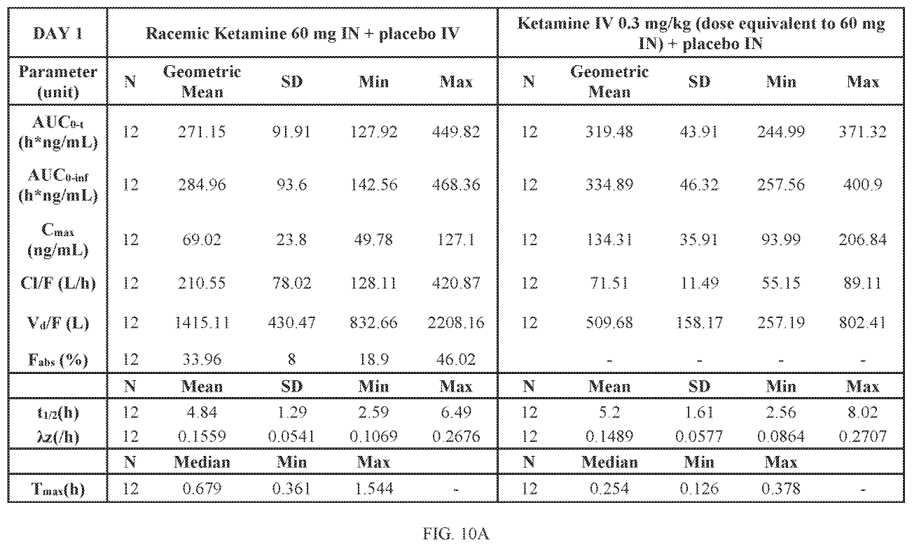

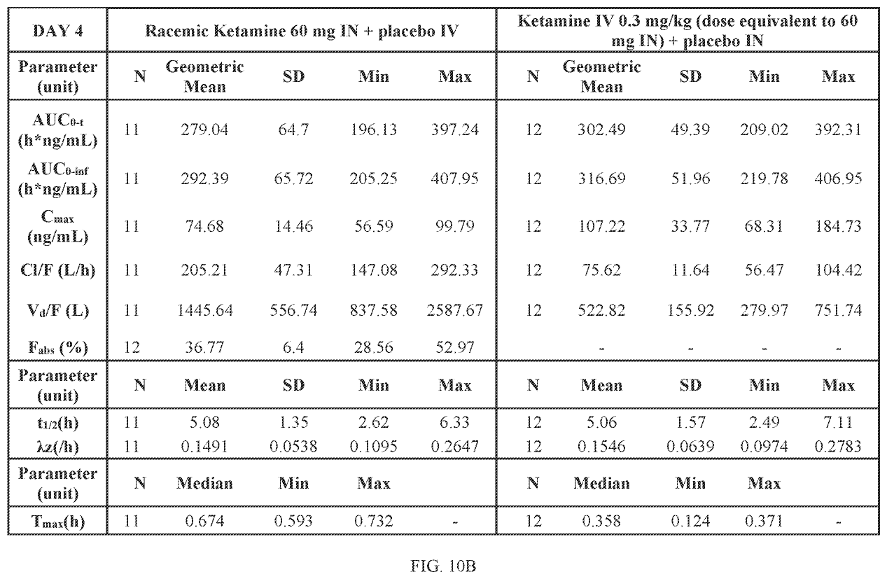

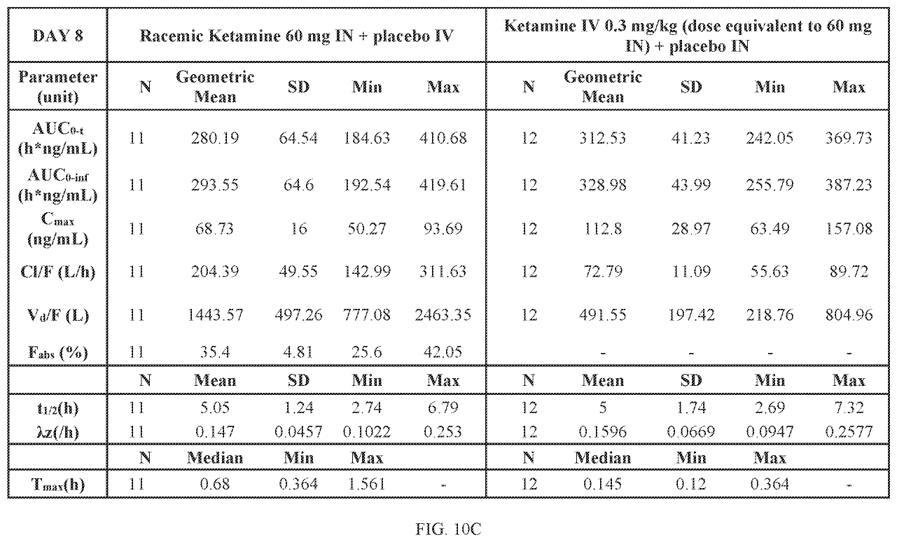

FIGS. 10A-10C are tables describing pharmacokinetic parameters for ketamine on Day 1 (FIG. 10A), Day 4 (FIG. 10B), or Day 8 (FIG. 10C) in Part B of the study described in Example 1 for racemic ketamine 60 mg IN+placebo IV and ketamine IV 0.3 mg/kg racemic ketamine (dose equivalent to 60 mg IN)+placebo IN.

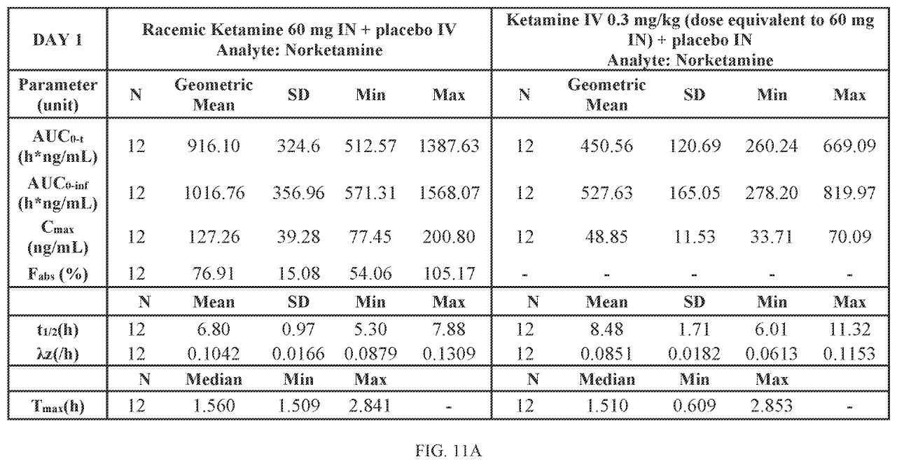

FIGS. 11A-11C are tables describing pharmacokinetic parameters for norketamine on Day 1 (FIG. 11A), Day 4 (FIG. 11B), or Day 8 (FIG. 11C) in Part B of the study described in Example 1 for racemic ketamine 60 mg IN+placebo IV and ketamine IV 0.3 mg/kg racemic ketamine (dose equivalent to 60 mg IN)+placebo IN.

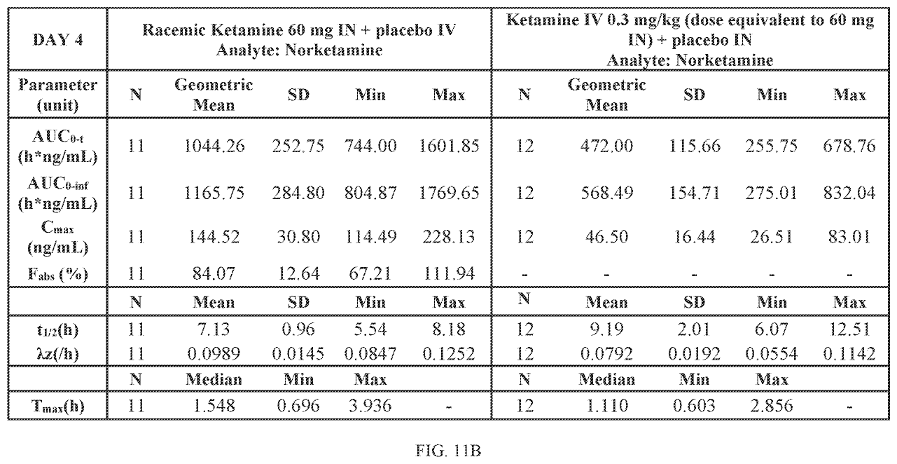

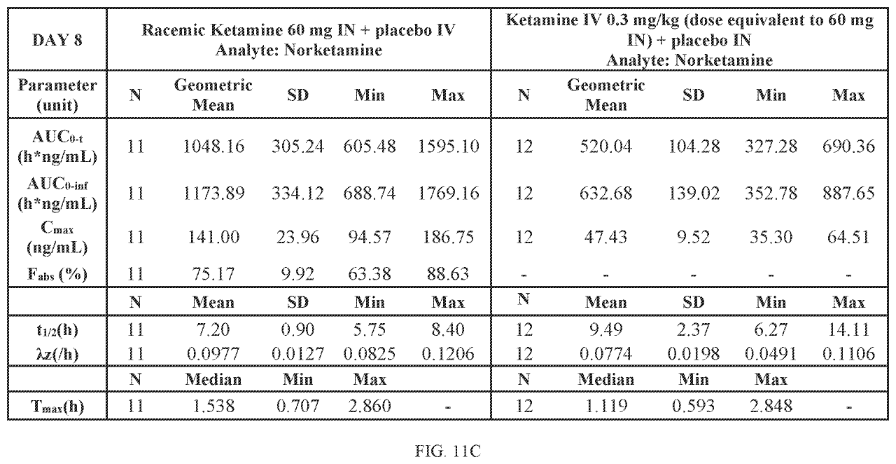

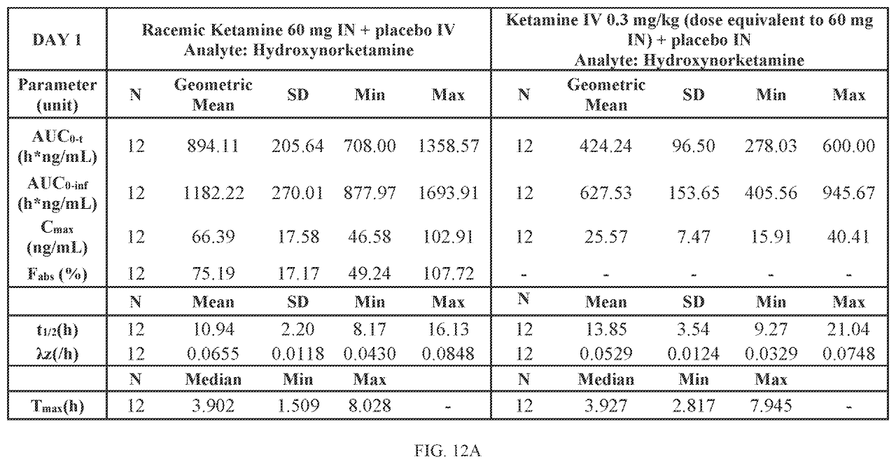

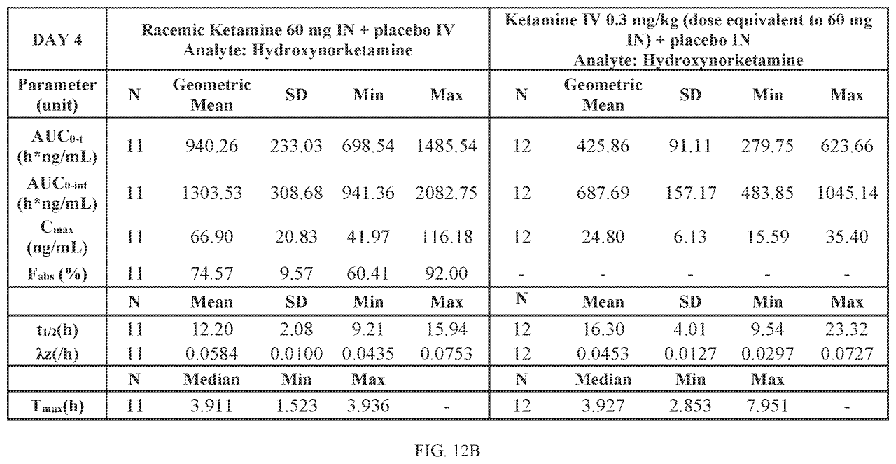

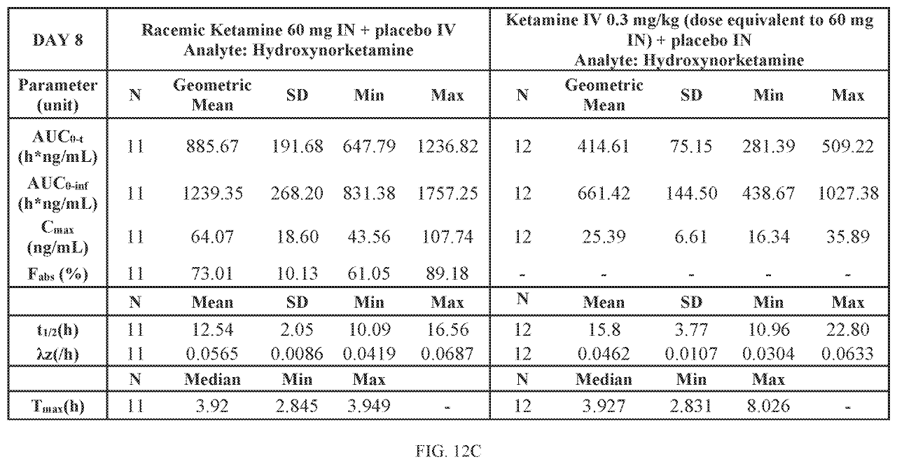

FIGS. 12A-12C are tables describing pharmacokinetic parameters for hydroxynorketamine on Day 1 (FIG. 12A), Day 4 (FIG. 12B), or Day 8 (FIG. 12C) in Part B of the study described in Example 1 for racemic ketamine 60 mg IN+placebo IV and ketamine IV 0.3 mg/kg racemic ketamine (dose equivalent to 60 mg IN)+placebo IN.

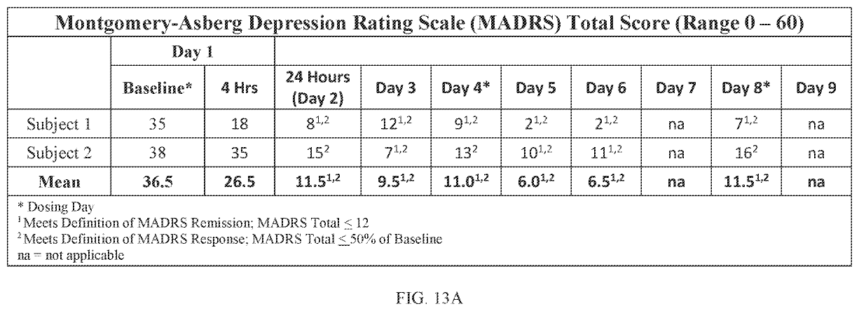

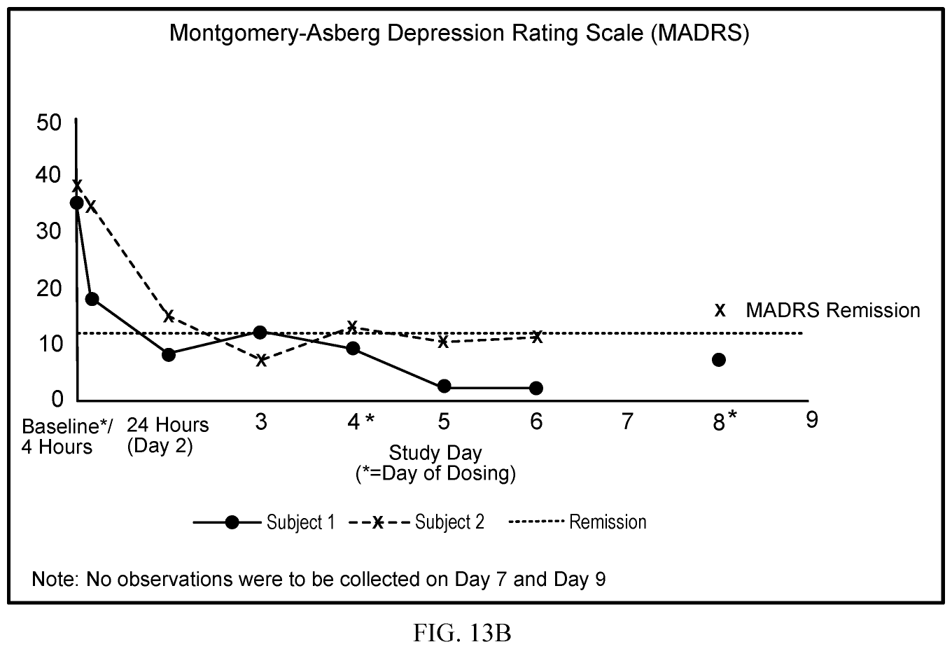

FIGS. 13A-13B are a table (FIG. 13A) and a graph (FIG. 13B) describing the MADRS depressions scores for Subject 1 and Subject 2 from Day 1 to Day 9 described in Example 3 of the study.

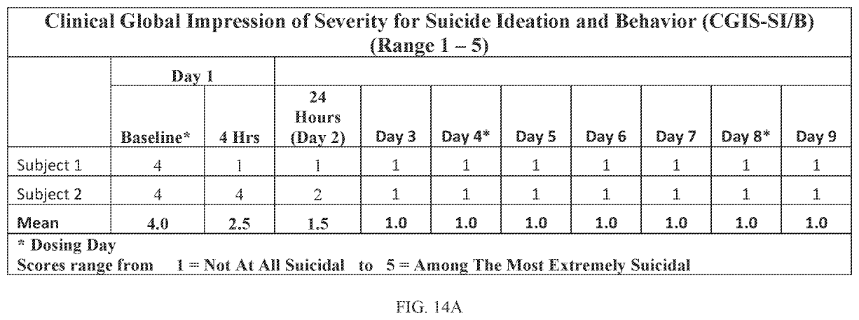

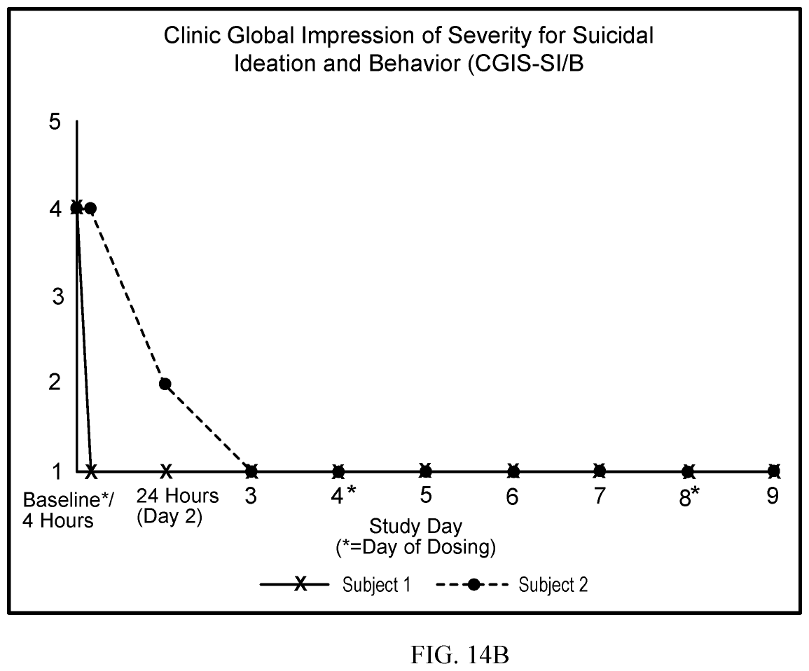

FIGS. 14A-14B are a table (FIG. 14A) and a graph (FIG. 14B) describing the CGIS-SI/B suicide ideation scores for Subject 1 and Subject 2 from Day 1 to Day 9 described in Example 4 of the study.

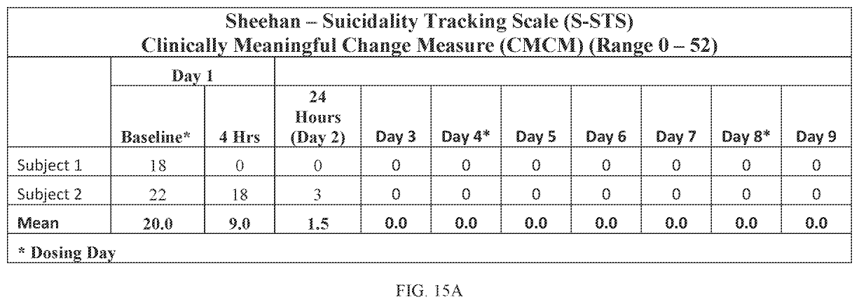

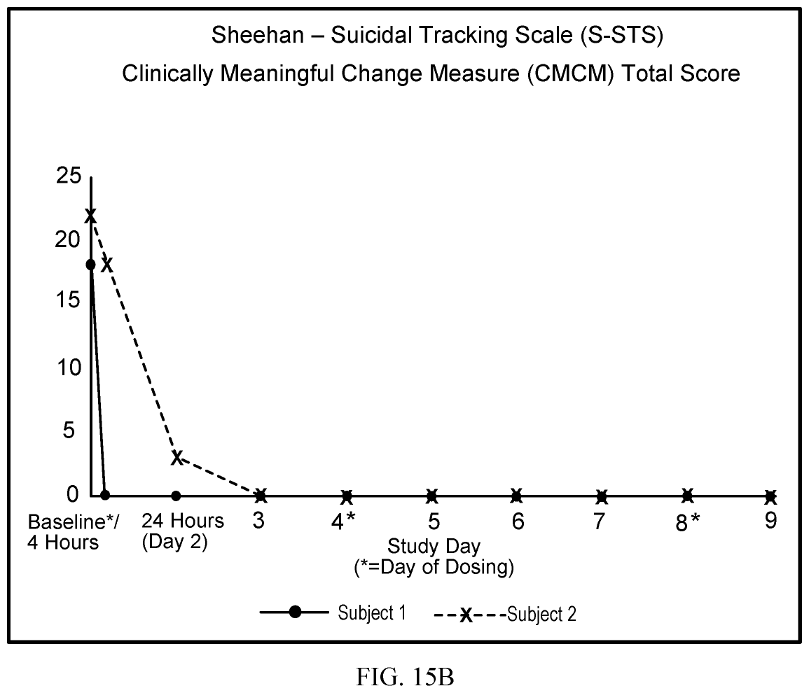

FIGS. 15A-15B are a table (FIG. 15A) and a graph (FIG. 15B) describing the STS suicidality scores for Subject 1 and Subject 2 from Day 1 to Day 9 described in Example 4 of the study.

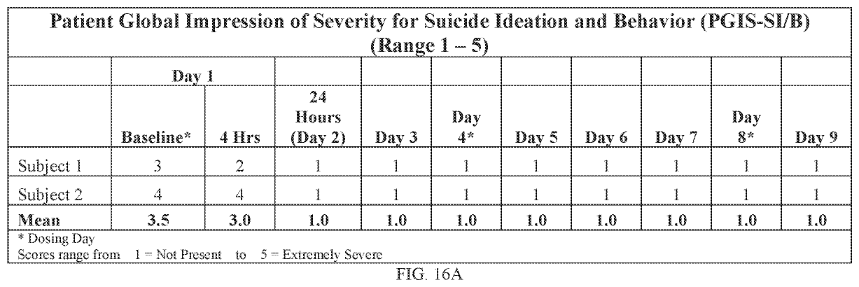

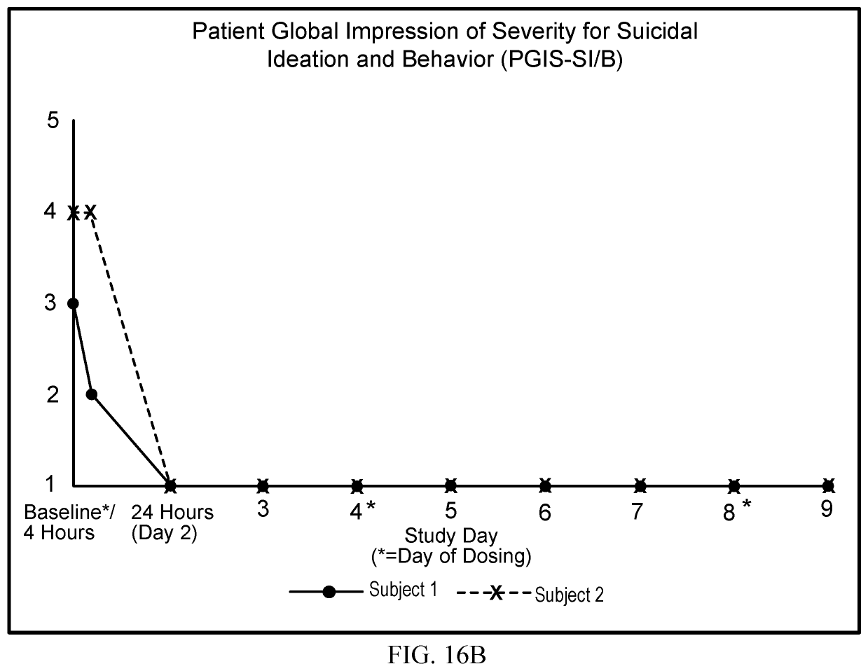

FIGS. 16A-16B are a table (FIG. 16A) and a graph (FIG. 16B) describing the PGIS-SI/B suicide ideation scores for Subject 1 and Subject 2 from Day 1 to Day 9 described in Example 4 of the study.

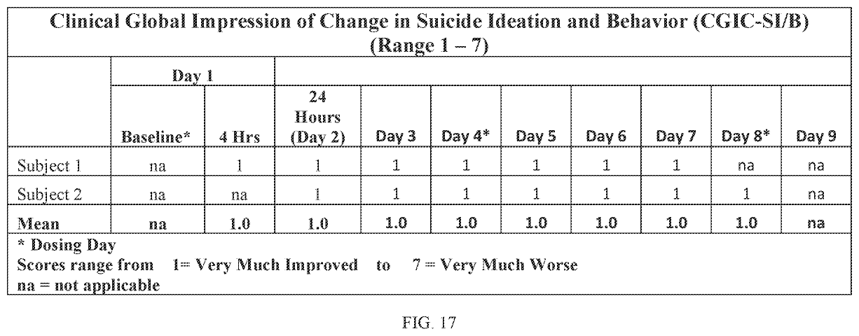

FIG. 17 is a table describing the CGIC-SI/B suicide ideation scores for Subject 1 and Subject 2 from Day 1 to Day 8 described in Example 4 of the study.

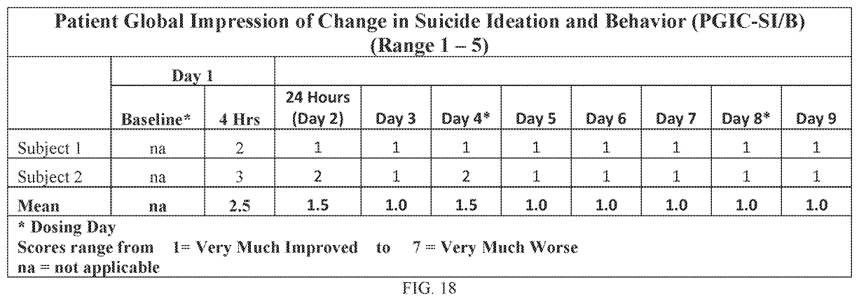

FIG. 18 is a table describing the PGIC-SI/B scores for Subject 1 and Subject 2 from Day 1 to Day 9 described in Example 3 of the study.

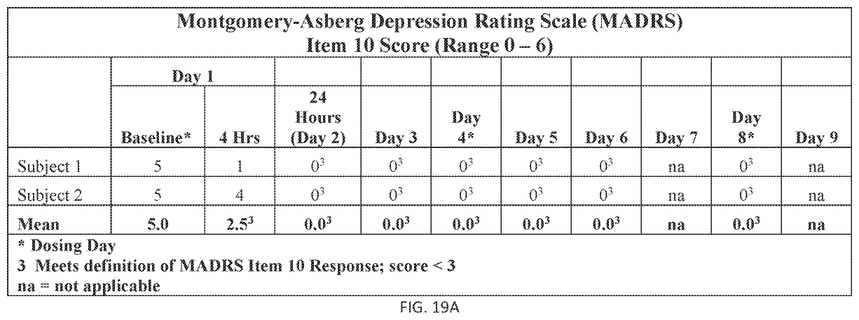

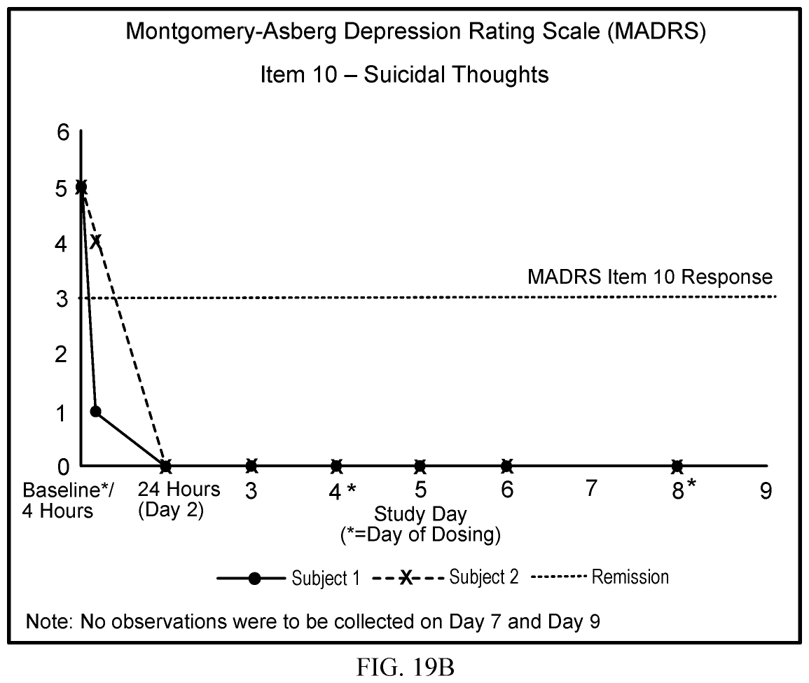

FIGS. 19A-19B are a table (FIG. 19A) and a graph (FIG. 19B) describing the MADRS item 10 scores for Subject 1 and Subject 2 from Day 1 to Day 9 described in Example 4 of the study.

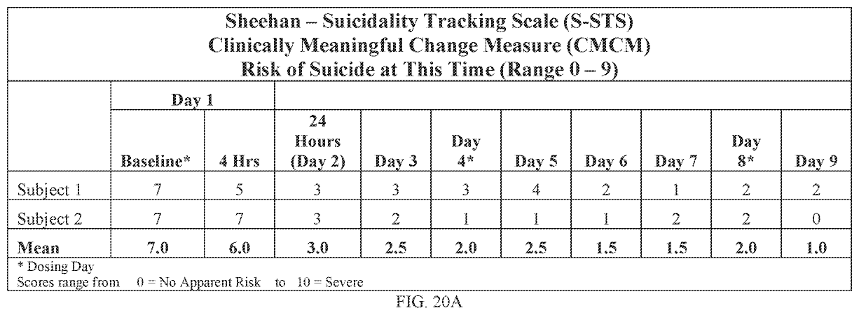

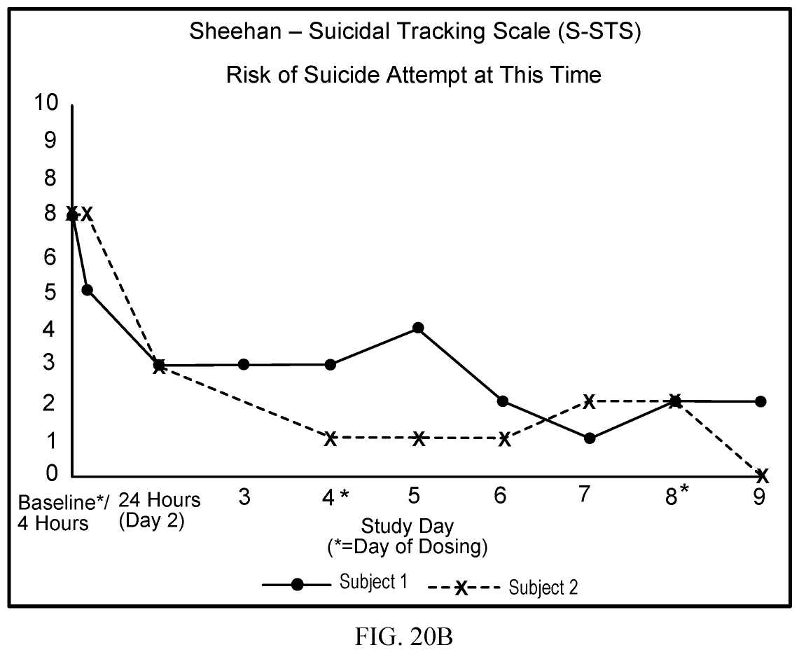

FIGS. 20A-20B are a table (FIG. 20A) and a graph (FIG. 20B) describing the STS-CMCM (“risk of suicide at this time”) scores for Subject 1 and Subject 2 from Day 1 to Day 9 described in Example 4 of the study.

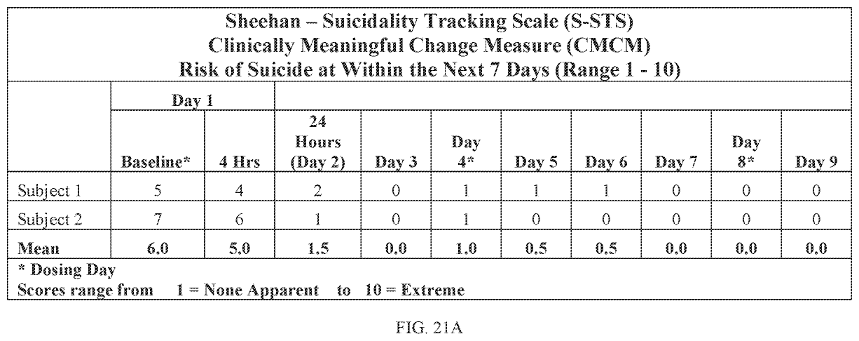

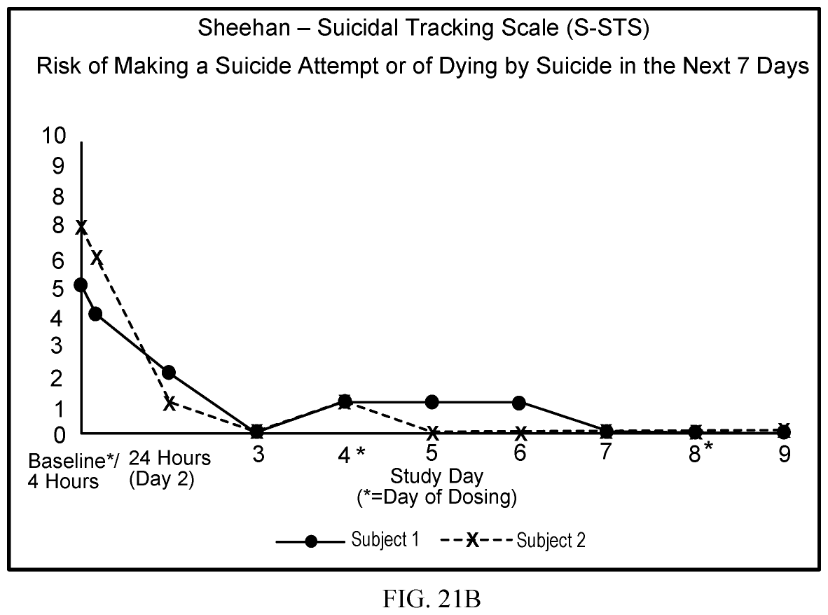

FIGS. 21A-21B are a table (FIG. 21A) and a graph (FIG. 21B) describing the STS-CMCM (“risk of suicide at within the next 7 day”) scores for Subject 1 and Subject 2 from Day 1 to Day 9 described in Example 4 of the study.

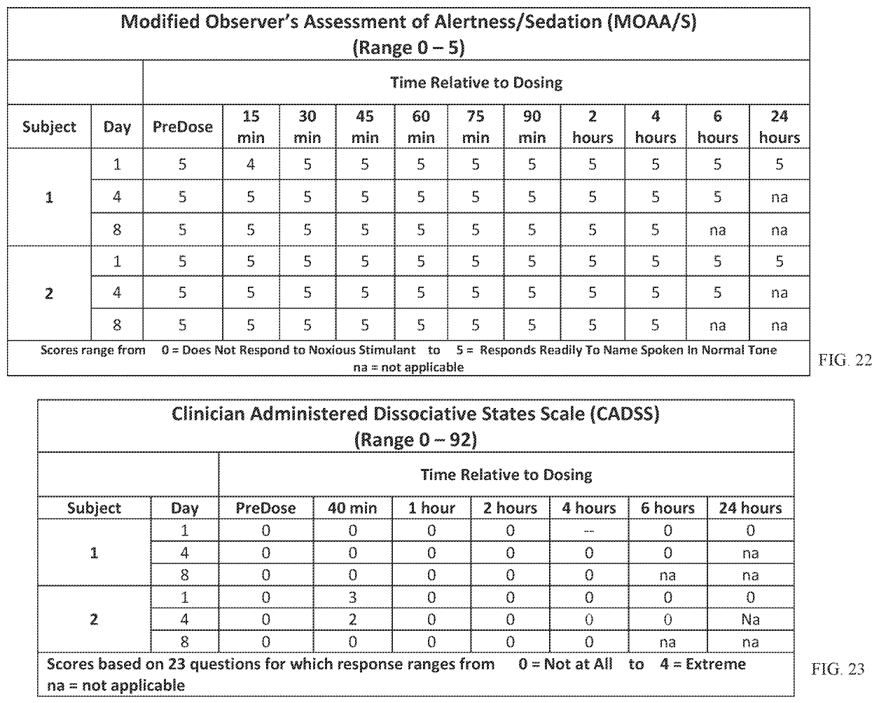

FIG. 22 is a table describing the MOAA/S alertness/sedation scores for Subject 1 and Subject 2 from pre-dose to 24 hours on Day 1 and Day 4 described in Example 4 of the study.

FIG. 23 is a table describing the CADSS dissociation scores from Subject 1 and Subject 2 from pre-dose to 24 hours on Day 1 and Day 4 described in Example 4 of the study.

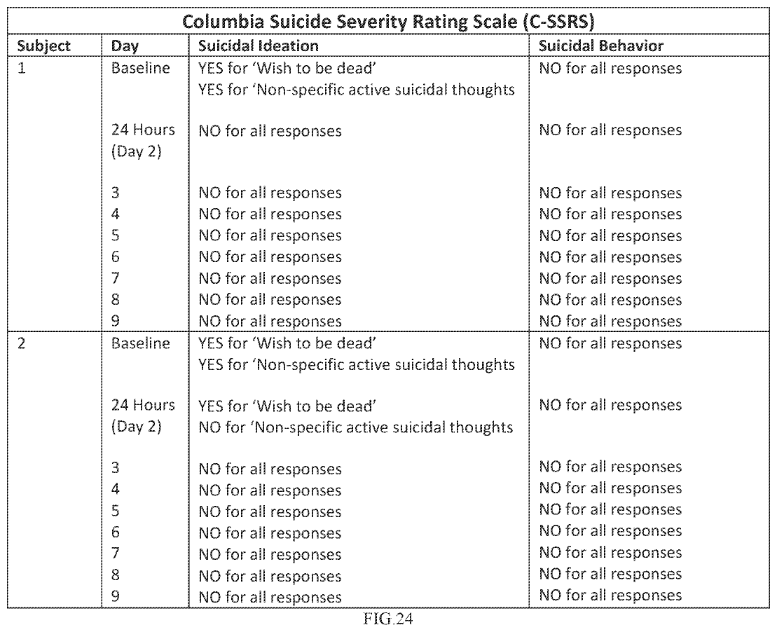

FIG. 24 is a table describing the C-SSRS scores for Subject 1 from Day 1 to Day 9 for Subject 1 and Day 1 to Day 4 for Subject 2 described in Example 4 of the study.

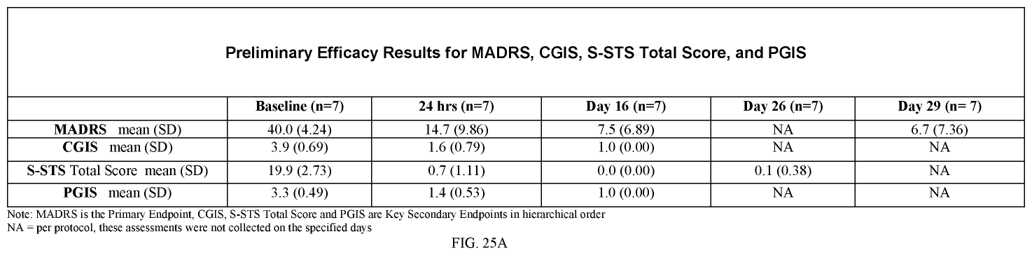

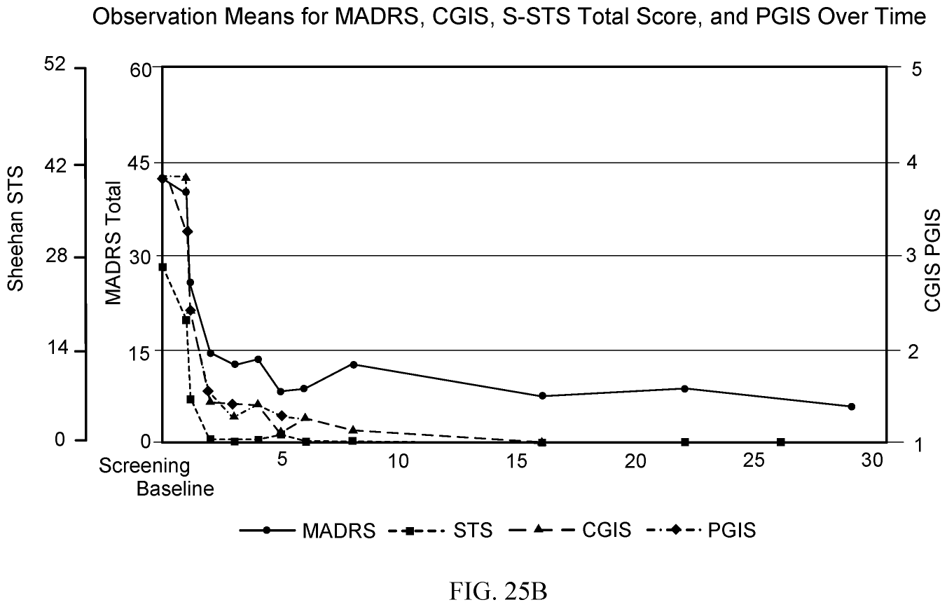

FIGS. 25A-25B are a table (FIG. 25A) and a graph (FIG. 25B) describing the preliminary efficacy results for MADRS, CGIS, STS total score, and PGIS for 7 subjects from Day 1 to Day 30 as described in Example 4 of the study.

### DETAILED DESCRIPTION

### Definitions

In order that the present disclosure can be more readily understood, certain terms are first defined. As used in this application, except as otherwise expressly provided herein, each of the following terms shall have the meaning set forth below. Additional definitions are set forth throughout the application.

Unless defined otherwise, all technical and scientific terms used herein have the same meaning as commonly understood by one of ordinary skill in the art to which this disclosure is related. For purposes of the present disclosure, the following terms are defined.

Units, prefixes, and symbols are denoted in their Système International de Unites (SI) accepted form. Numeric ranges are inclusive of the numbers defining the range. The headings provided herein are not limitations of the various aspects of the disclosure, which can be had by reference to the specification as a whole. Accordingly, the terms defined immediately below are more fully defined by reference to the specification in its entirety.

The terms “a,” “an,” or “the” as used herein not only include aspects with one member, but also include aspects with more than one member. For instance, the singular forms “a,” “an,” and “the” include plural referents unless the context clearly dictates otherwise. Thus, for example, reference to “a cell” includes a plurality of such cells and reference to “the agent” includes reference to one or more agents known to those skilled in the art, and so forth.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The terms “about” and “approximately” as used herein shall generally mean an acceptable degree of error for the quantity measured given the nature or precision of the measurements. Typical, exemplary degrees of error are within 10% or within 5% of a given value or range of values. Any reference to “about X” specifically indicates at least the values X, 0.95×, 0.96×, 0.97×, 0.98×, 0.99×, 1.01×, 1.02×, 1.03×, 1.04×, and 1.05×. Thus, “about X” is intended to provide written description support for a claim limitation of, e.g., “0.98×.” The terms “about” and “approximately,” particularly in reference to a given quantity, encompass and describe the given quantity itself.

When “about” or “approximately” is applied to the beginning of a numerical range, it applies to both ends of the range. Thus, “from about 5 to 20%” is equivalent to “from about 5% to about 20%.” When “about” is applied to the first value of a set of values, it applies to all values in that set. Thus, “about 5, 10, or 15 mg” is equivalent to “about 5, about 10, or about 15 mg.”

“Racemic ketamine” refers to a 1:1 mixture of the two enantiomers of ketamine: (R)-2-(2-chlorophenyl)-2-(methylamino)cyclohexanone and (S)-2-(2-chlorophenyl)-2-(methylamino)cyclohexanone.

An “equivalent dose” refers to an equivalent dose of an active agent based on bioavailability. An equivalent dose based on bioavailability can be determined by comparing the extent and rate of drug absorption of two or more dosages (e.g., dosages formulated as an intranasal formulation and as an intravenous formulation, respectively) of an active agent, for example, by determining the area under the blood or plasma concentration-time curve (AUC) and/or the maximum concentration (Cₘₐₓ), respectively. Accordingly, as used herein, an equivalent dose based on bioavailability is present when the two dosages each exhibit an AUC and/or Cₘₐₓ within about 80% to about 125% of one another.

The psychiatric disorders described herein would be understood by one skilled in the art, for example, as described in the Diagnostic and Statistical Manual of Mental Disorders 5th edition (DSM 5), which is hereby incorporated by reference in its entirety.

“Treatment” or “therapy” of a subject refers to any type of intervention or process performed on, or the administration of an active agent to, the subject with the objective of reversing, alleviating, ameliorating, inhibiting, or slowing down, the onset, progression, development, severity, or recurrence of a symptom, complication, condition, or biochemical indicia associated with a disease. In some embodiments, “treatment” includes resolution of a particular disorder, including a reduction in one or more symptoms of the disorder and/or a reduction in in the severity of one or more symptoms associated with the disorder.

“Administering” or “administration” refer to the physical introduction of a therapeutic agent to a subject, using any of the various methods and delivery systems known to those skilled in the art. Routes of administration can include oral, intranasal, intravenous, intranasal, intramuscular, subcutaneous, intraperitoneal, spinal or other parenteral routes of administration, for example by injection or infusion (e.g., intravenous infusion). Administration can also be performed, for example, once, a plurality of times, and/or over one or more extended periods. In some embodiments, the administration is intranasal.

A “subject” includes any human or non-human animal. The term “non-human animal” includes, but is not limited to, vertebrates such as non-human primates, sheep, dogs, and rodents such as mice, rats, and guinea pigs. In some embodiments, the subject is a human.

An “effective amount” or “therapeutically effective amount” of a therapeutic agent is any amount of the drug that, when used alone or in combination with one or more additional therapies, slowing down the onset of a psychiatric disorder or promotes regression of the disorder evidenced by a decrease in severity of disorder symptoms, an increase in frequency and duration of disorder symptom-free periods, or a ameliorating an impairment or disability due to the disorder affliction. The ability of one or more additional therapies to promote disorder regression can be evaluated using a variety of methods known to the skilled practitioner, such as in human subjects during clinical trials, in animal model systems predictive of efficacy in humans, or by assaying the activity of the agent in in vitro assays.

As used herein, a measure of a treatment effect is “clinically meaningful” based on the practical importance of a treatment effect. For example, whether the treatment effect has a real genuine, palpable, and/or noticeable effect on the subject (e.g., a lack of clinically meaningful effect occurs when the difference in the subject is small enough that it may be considered similar, such as prior to and after administration of a treatment as provided herein). One skilled in the art would recognize whether a particular effect is “clinically meaningful.” For example, a subject having a baseline score indicating severe depression (using any of the scales described herein) and a post-treatment score indicating remission of the severe depression would be a clinically meaningful effect.

As used herein, “AUC₀₋₁” refers to the area under the plasma concentration-time curve from time=0 to the time at which the last measurable concentration occurs. In some embodiments, the last measurable concentration occurs at time=32 hours (e.g., AUC₀₋₁=AUC₀₋₃₂).

A subject that is “not responsive” refers to a subject that has been, or is currently being, treated with one or more therapies that are not providing a clinically meaningful change towards the desired outcome (e.g., subjects that are not responsive includes patients that are refractory to a particular treatment). For example, a subject may exhibit no measurable change in response to therapy. A non-responsive subject could also, for example, exhibit a positive change in a depression scale score, but the change is not clinically meaningful.

As used herein, a psychiatric evaluation or side effect profile test score that is “substantially similar” or “substantially the same” as a reference score, corresponds to the same score, with a skilled artisan understanding that particular test scores may vary to a reasonable extent (such as #10%) while still describing a given value, due to, for example, experimental error, routine subject-to-subject evaluation, and routine statistical analysis.

The phrase “pharmaceutically acceptable” indicates that the substance or composition must be compatible chemically and/or toxicologically, with the other ingredients comprising a formulation, and/or the mammal being treated therewith.

As used herein, the term “pharmaceutically acceptable carrier” refers to a substance that aids the administration of an active agent to a cell, an organism, or a subject. “Pharmaceutically acceptable carrier” refers to a carrier or excipient that can be included in the compositions of the disclosure and that causes no significant adverse toxicological effect on the subject. Non-limiting examples of pharmaceutically acceptable carriers include water, NaCl, normal saline solutions, lactated Ringer's, normal sucrose, normal glucose, binders, fillers, disintegrants, lubricants, coatings, sweeteners, flavors and colors, liposomes, dispersion media, microcapsules, cationic lipid carriers, isotonic and absorption delaying agents, and the like. The carrier may also be substances for providing the formulation with stability, sterility and isotonicity (e.g., antimicrobial preservatives, antioxidants, chelating agents and buffers), for preventing the action of microorganisms (e.g. antimicrobial and antifungal agents, such as parabens, chlorobutanol, phenol, sorbic acid and the like) or for providing the formulation with an edible flavor etc. In some instances, the carrier is an agent that facilitates the delivery of a small molecule drug or antibody to a target cell or tissue. One of skill in the art will recognize that other pharmaceutical carriers are useful in the present disclosure.

As used in the methods described herein, the term “reducing” refers to a reduction in the indicated parameter relative to the baseline measurement (or measurements) of the same parameter in the subject taken prior to the initiation of administration of racemic ketamine, or a pharmaceutically acceptable salt thereof, or a reduction in the indicated parameter relative to the baseline measurement (or measurements) of the same parameter. In some embodiments, the same parameter is measured in a healthy subject (for example, a subject that does not have a psychiatric disorder as described herein). In some embodiments, the same parameter is measured relative to another treatment modality (for example, the standard of care treatment for a psychiatric disorder as described herein).

Similarly, the term “increasing,” as used herein, refers to an increase in the indicated parameter relative to the baseline measurement (or measurements) of the same parameter in the subject taken prior to the initiation of administration of racemic ketamine, or a pharmaceutically acceptable salt thereof, or an increase in the indicated parameter relative to the baseline measurement (or measurements) of the same parameter. In some embodiments, the same parameter is measured in a healthy subject (for example, a subject that does not have a psychiatric disorder as described herein). In some embodiments, the same parameter is measured relative to another treatment modality (for example, the standard of care treatment for a psychiatric disorder as described herein).

The term “synergy” or “synergistic” is used herein to mean that the effect of the combination of the two therapeutic agents of the combination therapy is greater than the sum of the effect of each agent when administered alone. A “synergistic amount” or “synergistically effective amount” is an amount of the combination of the two combination partners that results in a synergistic effect, as “synergistic” is defined herein. Determining a synergistic interaction between two combination partners, the optimum range for the effect and absolute dose ranges of each component for the effect may be definitively measured by administration of the combination partners over different w/w (weight per weight) ratio ranges and doses to subjects in need of treatment. However, the observation of synergy in in vitro models or in vivo models can be predictive of the effect in humans and other species and in vitro models or in vivo models exist, as described herein, to measure a synergistic effect. Exemplary synergistic effects includes, but are not limited to, enhanced therapeutic efficacy, decreased dose at equal or increased level of efficacy, reduced or delayed development of drug resistance, and simultaneous enhancement or equal therapeutic actions (e.g., the same therapeutic effect as at least one of the therapeutic agents) and reduction of unwanted drug effects (e.g., side effects and adverse events) of at least one of the therapeutic agents.

For example, a synergistic ratio of two therapeutic agents can be identified by determining a synergistic effect in, for example, an art-accepted in vivo model (e.g., an animal model) of depression (e.g., despair-based, reward-based, or anxiety-based mouse models).

As described herein, any concentration range, percentage range, ratio range, or integer range is to be understood to include the value of any integer within the recited range and, when appropriate, fractions thereof (such as one tenth and one hundredth of an integer), unless otherwise indicated.

Unless otherwise stated, any reference to an amount of ketamine in this disclosure is based on the free equivalent weight of ketamine. For example, 30 mg of ketamine refers to 30 mg of ketamine in the free form or an equivalent amount of a salt form of ketamine (e.g., ketamine hydrochloride).

Various aspects of the disclosure are described in further detail herein.

### Introduction

Psychiatric disorders are a growing public health concern, accounting for about 14% (and growing) to the global burden of diseases with at least 450 million people affected by psychiatric disorders worldwide. See, e.g., World Health Organization. WHO. Mental disorders fact sheet. Geneva 27, Switzerland; 2016. Psychiatric disorders are the main cause of the years lived with long term disabilities and dependency, including unipolar depression, schizophrenia, and bipolar disorder. See, e.g., Mathers, PLOS Med. 2006; 3:e442. A person with major psychiatric disorders has a 40-60% greater chance of dying prematurely than the general population. See, e.g., Semahegn, et al., System. Rev., Vol. 7, No. 10 (2018). Similarly, individuals with psychiatric disorders often report a lower quality of life. For example, over half of patients suffering from PTSD had severely impaired overall quality of life (see Rapaport et al., Am. J. Psychiatry, Vol. 162, pp. 1171-1178 (2005)) and nearly 40% had missed at least one of work day in the last month due to emotional problems (compared to only 5.4% of people not suffering from a psychiatric disorder). See, e.g., Stein, et al., Gen. Hosp. Psychiatry, Vol. 22, pp. 261-269 (2000).

In subjects with psychiatric disorders, maintaining adherence to treatment regimens with currently approved pharmacological interventions can be challenging. See, e.g., Farooq et. al., Neuropsychiatr Dis Treat., Vol. 10, pp. 1069-77 (2014). This non-adherence can be due to, for example, impairment of cognitive function due to the psychiatric disorder or to adversity to the sometime significant side effects that occur with many current therapies for psychiatric disorders. Many current pharmacological interventions can take weeks to months to achieve their full therapeutic effect, and many subjects are, or will become, resistant to these therapies. See, e.g, Kupfer, Dialogues Clin. Neurosci., Vol. 7, No. 3, pp. 191-205 (2005).

Ketamine has been used as an intravenous, short-acting anesthetic in both humans and animals. In addition to analgesia, ketamine produces a state of “dissociative anesthesia,” and is also used recreationally to induce these effects. See, e.g., Li and Vlisides, Front. Hum. Neurosci., Vol. 10, Article 612, pp. 1-15 (2016); and the Spravato® ((S)-ketamine) Package Insert dated Feb. 11, 2020; www.accessdata.fda.gov/drugsatfda_docs/label/2020/21124380031bl.pdf, which is hereby incorporated by reference in its entirety.

At low doses, ketamine produces mild sedation and euphoria, while at higher doses, individuals experience dissociative effective similar to phencyclidine hydrochloride (PCP). Other somatic effects of ketamine include vertigo, difficulties with balance, nausea, vomiting, sweating, tremor, dystonic movements, respiratory depression, and sleep apnea. See Zanos, et al., Pharmacol. Rev., Vol. 70, No. 3, pp. 621-660 (2018). The most frequently observed adverse events following administration of ketamine are manifested in psychic emergence phenomena, as such floating sensations, vivid dreams, hallucinations, hypertonus, and delirium. These effects can continue for up to 24 hours after administration. See Perumal, et al., J. Res. Pharm. Pract., Vol. 4, No. 2, pp. 89-93 (2015).

After administration, ketamine is demethylated to form norketamine, and both ketamine and norketamine can be hydroxylated, forming hydroxyphenylketamine, 6-hydroxyketamine, hydroxyphenylnorketamine, and 6-hydroxynorketamine (also referred to herein as hydroxynorketamine). The structures of these (racemic) compounds are shown below.

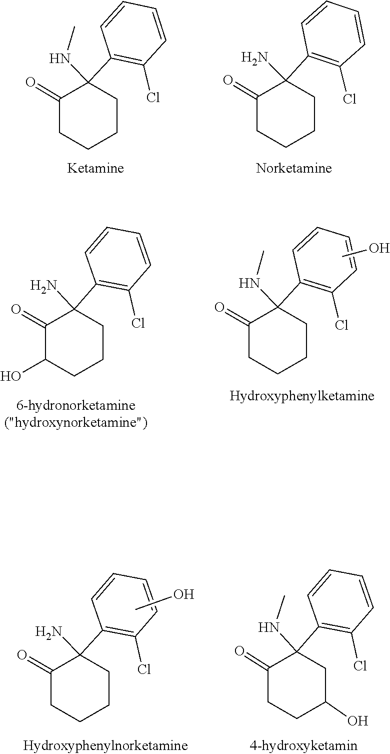

Each of these metabolites has a unique receptor binding profile and pharmacological activity. See, e.g., Zanos, et al., Pharmacol. Rev., Vol. 70, No. 3, pp. 621-660 (2018). For example, racemic ketamine binds to the NMDA receptor with a Ki of about 1.06 μM, (S)-norketamine and (R)-norketamine have K_{iS} of about 2.25 M and 26.46 μM respectively, and (2S,6S)-hydroxynorketamine and (2R,6R)-hydroxynorketamine have K_{iS} of about 21.19 μM and over 100 μM, respectively. See Moaddel, et al., Eur. J. Pharmacol., Vol. 698, pp. 228-234 (2013). Ketamine and norketamine both have anesthetic activity, and subjects administered ketamine or norketamine exhibit increased movement during the anesthetic recovery phase. In contrast, the same dose of 6-hydroxynorketamine provides no anesthetic or locomotor activity. See Leung and Baillie, J. Med. Chem., Vol. 29, pp. 2396-2399 (1986). However, like ketamine, 6-hydroxynorketamine does exhibit antidepressant properties. See Pham, et al., Biol. Psychiatry, Vol. 84, No. 1, pp. e3-e6 (2018).

The present application is based, in part, on the surprising discovery that intranasal administration of racemic ketamine provides advantageous properties relative to intravenous administration of racemic ketamine or intranasal administration of (R)- or (S)-ketamine (e.g., at least about 95% (R)-ketamine, or at least about 95% (S)-ketamine). Moreover, leveraging the different physiological and psychological effects of each enantiomer of ketamine, and the corresponding metabolites may provide beneficial treatments, including treatments with improved onset time and reduced negative side effects, for various psychiatric disorders as described herein.

### Formulations

Some embodiments provide a pharmaceutical composition comprising about 5% (w/v) to about 20% (w/v) of racemic ketamine, or a pharmaceutically acceptable salt thereof, and a pharmaceutically acceptable carrier; wherein the composition is formulated for intranasal administration.

In some embodiments, the pharmaceutical composition comprises an aqueous solution of about 7.5% (w/v) to about 15% (w/v) of racemic ketamine, or a pharmaceutically acceptable salt thereof, for example, about 7.5%, about 8%, about 8.5%, about 9%, about 9.5%, about 10%, about 10.5%, about 11%, about 11.5%, about 12%, about 12.5%, about 13%, about 13.5%, about 14%, about 14.5%, about 15%, or any value in between. In some embodiments, the pharmaceutical composition comprises an aqueous solution of about 7.5% (w/v) of racemic ketamine, or a pharmaceutically acceptable salt thereof. In some embodiments, the pharmaceutical composition comprises an aqueous solution of about 15% (w/v) of racemic ketamine, or a pharmaceutically acceptable salt thereof.

In some embodiments, the formulation provides about 30 mg to about 90 mg of racemic ketamine, or a pharmaceutically acceptable salt thereof, per dose. For example, about 30 mg, about 35 mg, about 40 mg, about 45 mg, about 50 mg, about 55 mg, about 60 mg, about 65 mg. about 70 mg, about 75 mg, about 80 mg, about 85 mg, about 90 mg, or any value in between. In some embodiments, the formulation provides about 45 mg to about 75 mg of racemic ketamine, or a pharmaceutically acceptable salt thereof, per dose. In some embodiments, the formulation provides about 60 mg to about 90 mg of racemic ketamine, or a pharmaceutically acceptable salt thereof, per dose. In some embodiments, the formulation provides about 30 mg, about 60 mg, about 75 mg, or about 90 mg of racemic ketamine, or a pharmaceutically acceptable salt thereof, per dose. In some embodiments, the formulation provides about 30 mg of racemic ketamine, or a pharmaceutically acceptable salt thereof, per dose. In some embodiments, the formulation provides about 60 mg of racemic ketamine, or a pharmaceutically acceptable salt thereof, per dose. In some embodiments, the formulation provides about 75 mg of racemic ketamine, or a pharmaceutically acceptable salt thereof, per dose. In some embodiments, the formulation provides about 90 mg of racemic ketamine, or a pharmaceutically acceptable salt thereof, per dose.

In some embodiments, the formulation provides a total amount of about 30 mg to about 90 mg of racemic ketamine, or a pharmaceutically acceptable salt thereof, over two doses (e.g., two spray discharges of an intranasal delivery device). For example, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, or any value in between. In some embodiments, the formulation provides a total amount of about 45 mg to about 75 mg of racemic ketamine, or a pharmaceutically acceptable salt thereof, over two doses. In some embodiments, the formulation provides a total amount of about 60 mg to about 90 mg of racemic ketamine, or a pharmaceutically acceptable salt thereof, per dose. In some embodiments, the formulation provides a total amount of about 30 mg, about 60 mg, about 75 mg, or about 90 mg of racemic ketamine, or a pharmaceutically acceptable salt thereof, over two doses. In some embodiments, the formulation provides about 60 mg of racemic ketamine, or a pharmaceutically acceptable salt thereof, over two doses. In some embodiments, the formulation provides about 75 mg of racemic ketamine, or a pharmaceutically acceptable salt thereof, over two doses. In some embodiments, the formulation provides about 90 mg of racemic ketamine, or a pharmaceutically acceptable salt thereof, over two doses.

In some embodiments, the composition further comprises a preservative. Exemplary preservatives include, but are not limited to parabens (e.g., alkyl parabens), benzyl alcohol, chlorobutanol, benzoic acid, sorbic acid, propylene glycol, quaternary ammonium salts (e.g., benzalkonium chloride and benzethonium chloride). In some embodiments, the preservative is benzalkonium chloride. In some embodiments, the racemic ketamine is in the form of a pharmaceutically acceptable salt, such as the hydrochloride salt. In some embodiments, the composition further comprises about 0.01 mg/mL to about 0.04 mg/ml benzalkonium chloride. In some embodiments, the composition further comprises about 0.02 mg/mL benzalkonium chloride.

In some embodiments, the composition further comprises one or more excipients selected from the group consisting of surfactants, antioxidants, buffers, and absorption enhancing agents.

Exemplary surfactants include, but are not limited to ionic, nonionic, and amphoteric surface active agents. For example, Tweens, PEGs, sorbitan esters, and ethoxylated fatty acids. In some embodiments, the composition further comprises a surfactant in an amount of about 1% to about 10% surfactant (w/v).

Exemplary antioxidants include, but are not limited to tocopherols, butyl hydroxytoluene, sodium metabisulfite, potassium metabisulfite, and ascorbyl palmitate. In some embodiments, the composition further comprises an antioxidant in an amount of about 0.001% to about 5% (w/w).

Exemplary absorption enhancing agents include, but are not limited to chitosan, caproic acid salts, and cyclopentadecalactone. In some embodiments, the composition further comprises an absorption enhancing agent in an amount of about 1% to about 10% (w/w).

Exemplary buffers include, but are not limited to citrate, phosphate, acetate, lactate, fumarate, tartrate, malate, and amino acid-based buffers. In some embodiments, the composition further comprises a buffer in an amount of about 0.1% to about 5% (w/w).

In some embodiments, the pharmaceutically acceptable carrier is water or saline.

In some embodiments, the formulation is as described in Table 1.

TABLE 1
Exemplary Formulation

| | | Product | Single |
|---|---|---|---|
| | | Concentration | Spray |
| Component | Function | (per mL) | (per 0.1 mL) |
| Ketamine | Active | 150 mg/mL | 15 mg |
| hydrochloride* | Ingredient | | |
| Benzalkonium | Antimicrobial | 0.02 mg/mL | 0.002 mg |
| chloride | preservative | | |
| Sodium hydroxide | Adjust pH | q.s | q.s |
| or HCl acid | | | |
| solution | | | |
| Purified Water | Aqueous Medium | q.s. to 1.0 mL | q.s. to 0.1 mL |

*1 mg of Ketamine = 1.15 mg Ketamine hydrochloride

At 15 mg per single spray, the formulation described in Table 1 provides a dose of 30 mg at 2 sprays, a dose of 60 mg at 4 sprays, and a dose of 90 mg at 6 sprays.

In some embodiments, the formulation is as described in Table 2.

TABLE 2
Exemplary Formulation

| | | Product | Single |
|---|---|---|---|
| | | Concentration | Spray |
| Component | Function | (per mL) | (per 0.1 mL) |
| Ketamine | Active | 75 mg/mL | 7.5 mg |
| hydrochloride* | Ingredient | | |
| Benzalkonium | Antimicrobial | 0.02 mg/mL | 0.002 mg |
| chloride | preservative | | |
| Sodium hydroxide | Adjust pH | q.s | q.s |
| or HCl acid | | | |
| solution | | | |
| Purified Water | Aqueous Medium | q.s. to 1.0 mL | q.s. to 0.1 mL |

*1 mg of Ketamine = 1.15 mg Ketamine hydrochloride

At 7.5 mg per single spray, the formulation described in Table 2 provides a dose of 30 mg at 4 sprays, a dose of 60 mg at 8 sprays, and a dose of 90 mg at 12 sprays.

### Methods of Treatment

Some embodiments provide a method for a treating psychiatric disorder in a subject in need thereof, comprising intranasally administering a therapeutically effective amount of racemic ketamine, or a pharmaceutically acceptable salt thereof, to the subject. In some embodiments, the psychiatric disorder is major depressive disorder, post-traumatic stress disorder, treatment-resistant depression, bipolar depression, post-partum depression, chronic pain, neuropathic pain, Rett syndrome, epilepsy, agitation associated with dementia, agitation associated with schizophrenia, or agitation associated with bipolar disorder.

In some embodiments, the psychiatric disorder is post-traumatic stress disorder, chronic pain, neuropathic pain, Rett syndrome, epilepsy, agitation associated with dementia, agitation associated with schizophrenia, or agitation associated with bipolar disorder.

In some embodiments, the subject is not currently suffering from depression. In some embodiments, the subject has not been determined to have depression. In some embodiments, the subject has been determined not to have depression. In some embodiments, the subject has not been diagnosed with depression. In some embodiments, the subject has been diagnosed as not having depression.

In some embodiments, the subject is not currently suffering from suicidality. In some embodiments, the subject has not been determined to have suicidality. In some embodiments, the subject has been determined not to have suicidality. In some embodiments, the subject has not been diagnosed with suicidality. In some embodiments, the subject has been diagnosed as not having suicidality.

In some embodiments, the subject is not currently suffering from suicidal ideation. In some embodiments, the subject has not been determined to have suicidal ideation. In some embodiments, the subject has been determined not to have suicidal ideation. In some embodiments, the subject has not been diagnosed with suicidal ideation. In some embodiments, the subject has been diagnosed as not having suicidal ideation.

### Major Depressive Disorder

Depression is characterized by depressed mood and a markedly diminished interest or pleasure in activities. Other symptoms may include significant weight loss or weight gain, decrease or increase in appetite, insomnia or hypersomnia, psychomotor agitation or retardation, fatigue or loss of energy, feelings of worthlessness or excessive or inappropriate guilt, and diminished ability to think or concentrate or indecisiveness. See Kennedy, Dialogues Clin. Neurosci., Vol. 10, No. 3, pp. 271-277 (2008). A variety of somatic symptoms may also be present. Though depressive feelings are common, depressive disorder is diagnosed only when the symptoms reach a threshold and last at least two weeks. Depression can vary in severity from mild to very severe. It is most often episodic but can be recurrent or chronic. More than 50% of those who initially suffer a single major depressive episode eventually develop another.

Some embodiments provide a method for treating major depressive disorder in a subject in need thereof, comprising intranasally administering to the subject a therapeutically effective amount of racemic ketamine, or a pharmaceutically acceptable salt thereof.

In some embodiments, the subject meets Diagnostic and Statistical Manual of Mental Disorders, Fifth Edition (DSM-5) criteria for a diagnosis of current MDD (unipolar without psychotic features), with symptoms present for at least 4 weeks, based on psychiatric intake and confirmed by the Mini International Psychiatric Interview Version 7.02 (MINI).

### Post-Traumatic Stress Disorder

Post-traumatic stress disorder (PTSD) is a mental health condition that develops following a traumatic event characterized by intrusive thoughts about the incident, recurrent distress/anxiety, flashback and avoidance of similar situations. Symptoms usually begin early, within 3 months of the traumatic event, but sometimes they begin years afterward. Symptoms must last more than a month and be severe enough to interfere with relationships or work to be considered PTSD. The course of the illness varies. Some people recover within 6 months, while others have symptoms that last much longer. In some people, the condition becomes chronic, potentially resulting in lifelong disability.

Some embodiments provide a method for treating post-traumatic stress disorder in a subject in need thereof, comprising intranasally administering to the subject a therapeutically effective amount of racemic ketamine, or a pharmaceutically acceptable salt thereof.

In some embodiments, the subject meets Diagnostic and Statistical Manual of Mental Disorders, Fifth Edition (DSM-5) criteria for a diagnosis of current PTSD (i.e. past 6 months). In some embodiments, if the subject's primary index trauma occurred during childhood and/or more than 15 years prior to Screening, current symptoms of PTSD are the result of a more recent trauma which reactivated an earlier traumatic response to the original event. In some embodiments, the subject meets criteria for current PTSD Suicidality Disorder as confirmed by the MINI. In some embodiments, the subject meets Diagnostic and Statistical Manual of Mental Disorders, Fifth Edition (DSM-5) criteria for a diagnosis of current PTSD based on psychiatric intake and confirmed by the Mini International Psychiatric Interview Version 7.02 for Suicidality Disorders (MINI). In some embodiments, the subject's diagnoses of PTSD and suicidality are considered primary (i.e., the primary sources of current symptoms and impairment.

### Treatment-Resistant Depression

Treatment-resistant Depression (TRD) applies to an individual with major depressive disorder (MDD) that has not responded to at least two types of medication, typically two antidepressants from two different drug classes. According to a 2021 study published in the Journal of Clinical Psychiatry, 30.9% of people in the United States who take medication for their MDD have treatment-resistant depression. The MDD can be unipolar, meaning that it does not oscillate between mania and depression or it can be bipolar, meaning that it oscillates between mania and depression.

Some embodiments provide a method for treating treatment-resistant depression in a subject in need thereof, comprising intranasally administering to the subject a therapeutically effective amount of racemic ketamine, or a pharmaceutically acceptable salt thereof. In some embodiments, the TRD is treatment-resistant unipolar depression. In some embodiments, the TRD is treatment-resistant bipolar depression.

### Bipolar Depression

Bipolar disorder, formerly called manic depression, is a mental health condition that causes extreme mood swings that include emotional highs (mania or hypomania) and lows (depression). Symptoms can cause unpredictable changes in mood and behavior, resulting in significant distress and difficulty in life. In some cases, mania may trigger a break from reality, i.e., psychosis.

Some embodiments provide a method for treating bipolar depression in a subject in need thereof, comprising intranasally administering to the subject a therapeutically effective amount of racemic ketamine, or a pharmaceutically acceptable salt thereof.

### Post-Partum Depression

Post-partum depression is a mental health condition suffered by new mothers characterized by mood swings, crying spells, anxiety, irritability, reduced concentration, sadness, and difficulty sleeping. Symptoms usually develop within the first few weeks after giving birth, but may begin earlier (sometimes during pregnancy) or up to a year after birth.

Some embodiments provide a method for treating post-partum depression in a subject in need thereof, comprising intranasally administering to the subject a therapeutically effective amount of racemic ketamine, or a pharmaceutically acceptable salt thereof.

### Chronic Pain

Chronic pain is defined as pain that lasts more than several months, i.e., longer than “normal healing”, and typically lasting at least 3-6 months. It is estimated that over 25 million U.S. adults had pain every day for the previous 3 months, and nearly 40 million adults had severe pain over the same period. Chronic pain interferes with daily life and can lead to depression, anxiety, and other psychiatric disorders.

Some embodiments provide a method for treating chronic pain in a subject in need thereof, comprising intranasally administering to the subject a therapeutically effective amount of racemic ketamine, or a pharmaceutically acceptable salt thereof.

### Neuropathic Pain

Neuropathic pain occurs when the nervous system is damaged or not working correctly. Damaged nerve fibers send the wrong signals to pain centers. Nerve function may change at the site of the nerve damage, as well as areas in the central nervous system (central sensitization). Neuropathic pain can manifest as spontaneous pain (pain that comes without stimulation), e.g., shooting, burning, stabbing, or electric shock-like pain; tingling, numbness, or a “pins and needles” feeling. Neuropathic pain can manifest as evoked pain, i.e., pain brought on by normally non-painful stimuli such as cold, gentle brushing against the skin, pressure, etc. (allodynia). Evoked pain also may mean the increase of pain by normally painful stimuli such as pinpricks and heat (hyperalgesia). This condition often results in emotional problems due to disturbed sleep and pain

Some embodiments provide a method for treating neuropathic pain in a subject in need thereof, comprising intranasally administering to the subject a therapeutically effective amount of racemic ketamine, or a pharmaceutically acceptable salt thereof.

### Rett Syndrome

Rett Syndrome is a neurodevelopmental disorder that almost exclusively affects girls. It is relatively rare, occurring one in every 10,000 to 15,000 female births and in all racial and ethnic groups worldwide. A congenital disease caused by MECP2 gene mutation, Rett Syndrome is characterized by normal early growth and development followed by a slowing of development, loss of purposeful use of the hands, distinctive hand movements, slowed brain and head growth, problems with walking, seizures, and intellectual disability. There is no cure for Rett syndrome. Treatment for the disorder focuses on the management of symptoms.

Some embodiments provide a method for treating Rett syndrome in a subject in need thereof, comprising intranasally administering to the subject a therapeutically effective amount of racemic ketamine, or a pharmaceutically acceptable salt thereof.

### Epilepsy

Epilepsy is a neurological disorder in which brain activity becomes abnormal, causing seizures or periods of unusual behavior, sensations and sometimes loss of awareness. Epilepsy affects both males and females of all races, ethnic backgrounds and ages. Epilepsy can manifest as mild symptoms, e.g., a blank stare for a few seconds during a seizure to more severe, e.g., repeated twitching of arms or legs. Epilepsy has no identifiable cause in about half the people with the condition. In the other half, the condition may be traced to various factors, including genetics, head injury, brain abnormalities, infections, prenatal injury or developmental disorders. Epileptic seizures can lead to circumstances that are dangerous depending upon when and where they happen, sometimes resulting in drowning, falling, and auto accidents. People with epilepsy are more likely to have psychological problems, especially depression, anxiety, and suicidal thoughts and behaviors.

Some embodiments provide a method for treating epilepsy in a subject in need thereof, comprising intranasally administering to the subject a therapeutically effective amount of racemic ketamine, or a pharmaceutically acceptable salt thereof.

Agitation Associated with Dementia

Agitation associated with dementia is one among a larger constellation of behavioral and psychological symptoms associated with all major forms of dementia. According to several observations, agitation prevalence ranges from 30 to 50% in Alzheimer's disease, 30% in dementia with Lewy bodies, 40% in frontotemporal dementia, and 40% in vascular dementia (VaD). With an overall prevalence of about 30%, agitation is the third most common neuropsychiatric symptoms (NPS) in dementia, after apathy and depression, and it is even more frequent (80%) in residents of nursing homes. Agitation adversely impacts cognitive performance, functional status, and patients' quality of life and enhances caregiver's distress. Moreover, agitation is associated with a higher admission rate to assisted living facilities, higher use of medications, long-term hospitalization, and higher mortality

Some embodiments provide a method for treating agitation associated with dementia in a subject in need thereof, comprising intranasally administering to the subject a therapeutically effective amount of racemic ketamine, or a pharmaceutically acceptable salt thereof.

Agitation Associated with Schizophrenia

Agitation, which is defined as excessive motor and verbal activity, is frequently observed in psychiatric patients and affects the treatment of schizophrenia. Individuals with schizophrenia are vulnerable to episodes of agitation, which can be defined as excessive verbal and motor behavior, especially during exacerbations of their disease. Agitation associated with psychosis is a frequent reason for emergency department (ED) visits by patients with psychiatric disorders, and requires immediate action to prevent escalation to a level that could put patients, staff, and others at risk.

Some embodiments provide a method for treating agitation associated with schizophrenia in a subject in need thereof, comprising intranasally administering to the subject a therapeutically effective amount of racemic ketamine, or a pharmaceutically acceptable salt thereof.

Agitation Associated with Bipolar Disorder

Agitation is a common manifestation of bipolar disorder, which includes symptoms ranging from inner tension and unease to violence and aggression. Agitation is often seen in bipolar patients during acute manic states, when increased energy levels and reduced need for sleep lead patients to collide with the limits of others. Agitation also occurs during mixed and depressive states, which are characterized by fluctuating energy levels and periods of irritability.

Some embodiments provide a method for treating agitation associated with bipolar disorder in a subject in need thereof, comprising intranasally administering to the subject a therapeutically effective amount of racemic ketamine, or a pharmaceutically acceptable salt thereof.

In some embodiments, the subject meets Diagnostic and Statistical Manual of Mental Disorders, Fifth Edition (DSM-5) criteria for a diagnosis of bipolar disorder I or II, current major depressive episode (without psychotic features and rapid cycling disease course, i.e., no less than 4 episodes of mood disturbance in the 12 months prior to screening), with symptoms present for at least 4 weeks, based on psychiatric intake and confirmed by the Mini International Psychiatric Interview Version 7.02 (MINI).

### Additional Methods and Embodiments

In some embodiments, the subject has been previously diagnosed with and/or is currently suffering from, post-traumatic stress disorder. In some embodiments, the subject has been previously diagnosed with and/or is currently suffering from, major depressive disorder. In some embodiments, the subject has been previously diagnosed with and/or is currently suffering from, treatment-resistant depression. In some embodiments, the subject has been previously diagnosed with and/or is currently suffering from, bipolar depression. In some embodiments, the subject has been previously diagnosed with and/or is currently suffering from, post-partum depression. In some embodiments, the subject has been previously diagnosed with and/or is currently suffering from, post-partum depression, and is not currently breastfeeding. In some embodiments, the subject has been previously diagnosed with and/or is currently suffering from, chronic pain. In some embodiments, the subject has been previously diagnosed with and/or is currently suffering from, neuropathic pain. In some embodiments, the subject has been previously diagnosed with and/or is currently suffering from, Rett syndrome. In some embodiments, the subject has been previously diagnosed with and/or is currently suffering from, epilepsy. In some embodiments, the subject has been previously diagnosed with and/or is currently suffering from, agitation associated with dementia. In some embodiments, the subject has been previously diagnosed with and/or is currently suffering from, agitation associated with schizophrenia. In some embodiments, the subject has been previously diagnosed with and/or is currently suffering from, agitation associated with bipolar disorder.

In some embodiments, the subject has not been previously diagnosed with suicidality. In some embodiments, the subject has not been previously diagnosed with suicidal ideation. In some embodiments, the subject has not previously exhibited suicidality. In some embodiments, the subject has not previously exhibited suicidal ideation. In some embodiments, the subject is not currently suffering from suicidality. In some embodiments, the subject is not currently suffering from suicidal ideation.

In some embodiments described herein, the psychiatric disorder resolves faster relative to the resolution observed after administration of an equivalent dose of intravenous racemic ketamine, or a pharmaceutically acceptable salt thereof. For example, in some embodiments, the MDD, PTSD, TRD, bipolar depression, post-partum depression, chronic pain, neuropathic pain, Rett syndrome, epilepsy, agitation associated with dementia, agitation associated with schizophrenia, or agitation associated with bipolar disorder, of the subject resolves faster.

In some embodiments, the psychiatric disorder resolves from about 1.2× faster to about 10× faster relative to the resolution observed after administration of an equivalent dose of intravenous racemic ketamine, or a pharmaceutically acceptable salt thereof, such as 1.2×, 1.4×, 1.6×, 1.8×, 2×, 2.5×, 3×, 3.5×, 4×, 4.5×, 5×, 5.5×, 6×, 6.5×, 7×, 7.5×, 8×, 8.5×, 9×, 9.5×, 10× faster, or any value in between.

In some embodiments described herein, the psychiatric disorder resolves faster relative to the resolution observed following administration of an equivalent dose of (S)-ketamine (e.g., intranasal (S)-ketamine), or a pharmaceutically acceptable salt thereof. For example, in some embodiments, the MDD, PTSD. TRD, bipolar depression, post-partum depression, chronic pain, neuropathic pain, Rett syndrome, epilepsy, agitation associated with dementia, agitation associated with schizophrenia, or agitation associated with bipolar disorder, of the subject resolves faster. In some embodiments, the psychiatric disorder resolves from about 1.2× faster to about 10× faster relative to the resolution observed following administration of an equivalent dose of (S)-ketamine, or a pharmaceutically acceptable salt thereof, such as 1.2×, 1.4×, 1.6×, 1.8×, 2×, 2.5×, 3×, 3.5×, 4×, 4.5×, 5×, 5.5×, 6×, 6.5×, 7×, 7.5×, 8×, 8.5×, 9×, 9.5×, 10× faster, or any value in between.

In some embodiments described herein, the subject compliance with treatment for a psychiatric disorder is improved relative to an equivalent dose of intravenous racemic ketamine, or a pharmaceutically acceptable salt thereof. In some embodiments described herein, the subject compliance with treatment for a psychiatric disorder is improved relative to an equivalent dose of (S)-ketamine, or a pharmaceutically acceptable salt thereof. For example, in some embodiments, the MDD, PTSD, TRD, bipolar depression, post-partum depression, chronic pain, neuropathic pain, Rett syndrome, epilepsy, agitation associated with dementia, agitation associated with schizophrenia, or agitation associated with bipolar disorder, is improved.

In some embodiments, the subject has a BMI of about 18.0 to about 40.0 kg/m².

In some embodiments, the subject has not undergone electroconvulsive therapy (ECT).

### Scales

Many methods can be used to assess and/or measure the psychiatric conditions described herein in a subject. Non-limiting examples include the Mini International Psychiatric Interview Version 7.02 for Suicidality Disorders (MINI); Clinical Global Impression (CGI); Profile of Mood States (POMS) (e.g., POMS 2^{nd} edition); CGIS; CGIC; Patient Global Impression (PGI); PGIS; PGIC; Choice Reaction Time (CRT) test; the Columbia-Suicide Severity Rating Scale (C-SSRS); the Montgomery-Åsberg Depression Rating Scale (MADRS); the Sheehan Suicide Tracking Scale Clinically Meaningful Change Measure (STS-CMCM also referred to as S-STS CMCM) (see, e.g., Ghasemi et al., Health Promot. Perspect., Vol. 5, No. 3, pp. 156-168 (2015), which is incorporated by reference herein in its entirety); Sheehan Disability Scale (SDS); QIDS-SR16 (Quick Inventory of Depressive Symptomatology-Self-report; Change in the total Hamilton Depression Rating Scale (HAM-D17); Change From Baseline in Snaith-Hamilton Pleasure Scale (SHAPS) Score up to Treatment Week 6; Patient Health Questionnaire 9-Item (PHQ-9) at Week 6; Quality of Life Scale (QOLS); Hamilton Depression Rating Scale-Suicidal Ideation (HDRS-SI); Sternberg short-term memory task (SSTM); CRPS Severity Score (CSS); HDRS-28 Total; Depression Score Using Goldberg Depression Screening Test; Change in Beck Scale for Suicidal Ideation (BSSI), Quick Inventory of Depressive Symptomatology (QIDS); Change from Baseline in Sleep Disturbance Using the Patient Reported Outcome Measurement Information System-Sleep Disturbance (PROMIS-SD); Patient-Reported Outcomes Measurement Information System 29 (PROMIS-29); Change from randomization to each study visit in the total score of the Inventory of Depressive Symptomatology-Self Rated (IDS-SR30); Pelvic Pain and Urgency/Frequency (PUF) patient symptom scale; Quality of life enjoyment and satisfaction survey (Q-LES-Q); Percent change in Hamilton Depression Scale; Richmond Agitation-Sedation Scale (RASS); Change From Baseline in Anxiety Symptoms Measured by Hamilton Anxiety 14 Item Scale (HAM-A); Patient's Global Impression of Change in Suicidal Ideation and Behavior (PGIC-SI/B); Change From Baseline in Sleep Quality as Measured by the Pittsburgh Sleep Quality Index; Inventory of Depressive Symptomatology Self-Report (IDS-SR) 10-life engagement; Inventory of Depressive Symptomatology Self-Report (IDS-SR) Total score; Pain Self-Efficacy Questionnaire (PSEQ); Change in Brief Psychotic Rating Scale (BPRS); Pain Catastrophizing Scale (PCS); Change in Hamilton Anxiety Rating Scale (HAM-A); Modified Observer's Assessment of Alertness/Sedation Scale (MOAA/S); Change From Augmentation Baseline for Non-Remitters in the Behavior Rating Inventory of Executive Function-Adult Version (BRIEF-A) Scale Total Score at Week 6; Clinician Administered Dissociative States Scale (CADSS); Change From Augmentation Baseline for Non-Remitters in the Multidimensional Assessment of Fatigue (MAF) Scale Total Score at Week 6; Patient's Global Impression of Severity in Suicidal Ideation and Behavior (PGIS-SI/B); Beck depression Inventory (BDI); Modified Bond-Lader Visual Analog Scale Score; Young Mania Rating Scale (YMRS); Systematic Assessment for Treatment Emergent Effects (SAFTEE); Edinburgh Postnatal Depression Scale (EPDS); Patient Global Impression Severity (PGIS); Beck Anxiety Inventory; Changes in Cortisol (CRT) during Trier Social Stress Test (TSST); Social Functioning Based on Postpartum Adjustment Questionnaire; Impact of Event Scale-Revised (IES-R); Chronic Pain Acceptance Questionnaire (CPAQ); Total pain relief (TOTPAR) at 8 hours; Average Daily Pain Score (ADPS); Percent change from baseline in “average pain for the past 24 hours” Numeric Pain Rating Scale (NPRS) score; Pain Intensity Score Using the Short-Form McGill Pain Questionnaire (SF-MPQ); Rett Syndrome Gross Motor Scale (RSGMS); Rett Syndrome Behaviour Questionnaire (RSBQ) total score; Gait Velocity as Measured by GAITRite System; Motor Behaviour Assessment Scale (MBA); Delay between the end of the seizure and recovery of oxygen saturation (SpO2); Rate of seizure-free patients; Time until the first seizure appears; Percentage of Participants With Seizure Freedom for Primary Generalized Tonic Clonic (PGTC) Seizures; Percentage reduction of the seizure frequency; Effect on Fatigue scores; Modified Fatigue Impact Scale (M-FIS) Effect anxiety-depression; Hospital anxiety depression scale (HAD); Quality of Life in Epilepsy: (QOLIE 31) including 7 subscores: global score calculated on the basis on the mean of each subscore; Clinician-Administered PTSD Scale for DSM-5 (CAPS-5); The Impact of Event Scale-Revised (IES-R); Number of Participants With Patient-Rated Inventory of Side Effects (PRISE); Davidson Trauma Scale (DTS); Connor-Davidson Resilience Scale (CD-RISC); 36-item Short Form Health Survey (SF-36); Pittsburgh Sleep Quality Index; Work Productivity and Activity Improvement Questionnaire (WPAI); Incidence of adverse events assessed by 4-items positive symptoms subscale of Brief Psychiatric Rating Scale (BPRS); Mean change from baseline in Cohen-Mansfield Agitation Inventory (CMAI) score at 14 weeks after dosing; Mini-Mental State Examination (MMSE) score; and PANSS-EC Change From Baseline. One of ordinary skill in the art will appreciate that various scales and combinations of scales can be used in the diagnosis, patient stratification, and monitoring of treatment of psychiatric disorders. This includes using scales, for example, for measuring depression (such as MADRS) to assess depression in a subject with another psychiatric disorder where depression may be a facet of that disorder (e.g., PTSD).

The Montgomery-Åsberg Depression Rating Scale (MADRS) is used to assess the severity of depression among subjects who have a diagnosis of depression. The MADRS includes 10 items and uses a 0 to 6 severity scale, scored following the interview. Higher scores indicate increasing depressive symptoms. Ratings can be added to form an overall score (range 0 to 60); no weights are used. Cut-off points include: 0 to 6—symptom absent, 7 to 19—mild depression, 20 to 34—moderate, 35 to 60—severe depression.

MADRS is a diagnostic questionnaire that can be used to measure the severity of a depressive episode in a subject. In some embodiments, the MADRS can be used to measure the severity of depressive symptoms in a subject, for example, a subject suffering from MDD, TRD, bipolar depression, and other psychiatric disorders described herein with a depression component. In some embodiments, the MADRS can be used to measure TRD. In some embodiments, the MADRS can be used to measure other psychiatric disorders, as described herein. For example, the MADRS includes 10 items directed to the following: 1) apparent sadness (e.g., representing despondency, gloom and despair that is more than just ordinary transient low spirits that is reflected in speech, facial expression, and posture); 2) reported sadness (e.g., representing reports of depressed mood, regardless of whether it is reflected in appearance or not and can include low spirits, despondency or the feeling of being beyond help and without hope); 3) inner tension (e.g., representing feelings of ill-defined discomfort, edginess, inner turmoil, mental tension mounting to either panic, dread or anguish); 4) reduced sleep (e.g., representing the experience of reduced duration or depth of sleep compared to the subject's own normal pattern when well); 5) reduced appetite (e.g., representing the feeling of a loss of appetite compared with when-well); 6) concentration difficulties (e.g., representing difficulties in collecting one's thoughts mounting to an incapacitating lack of concentration); 7) lassitude (e.g., representing difficulty in getting started or slowness in initiating and performing everyday activities); 8) inability to feel (e.g., representing the subjective experience of reduced interest in the surroundings, or activities that normally give pleasure, and the ability to react with adequate emotion to circumstances or people is reduced); 9) pessimistic thoughts (e.g., representing thoughts of guilt, inferiority, self-reproach, sinfulness, remorse and ruin); and 10) suicidal thoughts (e.g., representing the feeling that life is not worth living, that a natural death would be welcome, suicidal thoughts, and preparations for suicide). Each item is rated from 0 to 6, with 0 reflecting that the subject is not at all as described by the item and 6 reflecting that the subject is extremely like what is described by the item. For example, for apparent sadness, a score of 0 can indicate that the subject does not display any sadness, whereas a score of 6 can indicate that the subject looks miserable all the time, e.g., the subject is extremely despondent. As another example, for suicidal thoughts, a score of 0 can indicate that the subject enjoys life or takes it as it comes; a score of 2 can indicate that the subject is weary of life and may have fleeting suicidal thoughts; a score of 4 can indicate that the subject feels he or she would probably be better off dead (e.g., suicidal thoughts are common, and suicide is considered as a possible solution, but without specific plans or intention); and a score of 6 can indicate that the subject has explicit plans for suicide when there is an opportunity (e.g., the subject has made active preparations for suicide). Thus, the total score, after summation of each score for each item, is on a scale of 0 to 60. In some embodiments, a total score on the MADRS of 0 to 6 for the subject reflects the subject does not have symptoms related to depression; a score of 7 to 19 reflects that the subject has mild depression; a score of 20 to 34 reflects that the subject has moderate depression; and a score of 34 to 60 reflects that the subject has severe depression. The “MADRS Total Score” and the “total MADRS score” are used interchangeably herein.

In some embodiments, the MADRS Total Score of the subject is from 20-60 units prior to intranasal administration of the racemic ketamine, or a pharmaceutically acceptable salt thereof.

In some embodiments, the MADRS Total Score of the subject is from 30-60 units prior to intranasal administration of the racemic ketamine, or a pharmaceutically acceptable salt thereof. In some embodiments, the MADRS Total Score of the subject is reduced by at least 50% 24 hours after intranasal administration of the racemic ketamine, or a pharmaceutically acceptable salt thereof. In some embodiments, the MADRS Total Score of the subject is less than or equal to 15 units 24 hours after intranasal administration of the racemic ketamine, or a pharmaceutically acceptable salt thereof. In some embodiments, the MADRS Total Score of the subject is less than or equal to 12 units 48 hours after intranasal administration of the racemic ketamine, or a pharmaceutically acceptable salt thereof.

In some embodiments, the MADRS Total Score of the subject is 28 units to 35 units prior to intranasal administration of the racemic ketamine, or a pharmaceutically acceptable salt thereof. In some embodiments, the MADRS Total Score of the subject is reduced by at least 50% about 24 hours after intranasal administration of the racemic ketamine, or a pharmaceutically acceptable salt thereof. In some embodiments, the MADRS Total Score of the subject is less than or equal to 8 units about 24 hours after intranasal administration of the racemic ketamine, or a pharmaceutically acceptable salt thereof. In some embodiments, the MADRS Total Score of the subject is less than or equal to 6 units 48 hours after intranasal administration of the racemic ketamine, or a pharmaceutically acceptable salt thereof.

In some embodiments, the MADRS Total Score of the subject is 28 units to 35 units prior to intranasal administration of the racemic ketamine, or a pharmaceutically acceptable salt thereof, and decreases by 10 units to 20 units, e.g., by 10 units, 15, units, or 20 units after intranasal administration of the racemic ketamine, or a pharmaceutically acceptable salt thereof.

In some embodiments, the MADRS Total Score of the subject is 28 units to 35 units at about 5 minutes to about 24 hours prior to intranasal administration of the racemic ketamine, or a pharmaceutically acceptable salt thereof, and decreases by 10 units to 20 units, about 5 minutes to about 24 hours after intranasal administration of the racemic ketamine, or a pharmaceutically acceptable salt thereof.

In some embodiments, the MADRS Total Score of the subject is 28 units to 35 units at about 1 hour to about 4 hours, about 1.5 hours to about 5 hours, about 2 hours to about 6 hours, or about 5 hours to about hours, prior to intranasal administration of the racemic ketamine, or a pharmaceutically acceptable salt thereof, and decreases by 10 units to 20 units about 1 hour to about 4 hours, about 1.5 hours to about 5 hours, about 2 hours to about 6 hours, or about 5 hours to about 10 hours, after intranasal administration of the racemic ketamine, or a pharmaceutically acceptable salt thereof.

In some embodiments, the total MADRS score of the subject is 10 to 60 prior to intranasal administration of racemic ketamine, or a pharmaceutically acceptable salt thereof, as described herein. For example, 10 to 20, 10 to 30, 10 to 40, 10 to 50, 50 to 60, 40 to 60, 30 to 60, or 20 to 60 prior to intranasal administration of racemic ketamine as described herein. In some embodiments, the total MADRS score of the subject is 10, 15, 20, 25, 30, 35, 40, 45, 50, 55, or 60 prior to intranasal administration of racemic ketamine as described herein. In some embodiments, the total MADRS score of the subject is about 25 to about 35 prior to intranasal administration of racemic ketamine, or a pharmaceutically acceptable salt thereof, prior to the administration of intranasal racemic ketamine.

In some embodiments, the total MADRS score of the subject is measured about 5 minutes to about 24 hours prior to intranasal administration of the racemic ketamine, or a pharmaceutically acceptable salt thereof. For example, about 5 minutes to 1 hour, about 5 minutes to 6 hours, about 5 minutes to 12 hours, about 5 minutes to 18 hours, about 18 hours to 24 hours, about 12 hours to 24 hours, or about 6 hours to 24 hours prior to intranasal administration of the racemic ketamine, or a pharmaceutically acceptable salt thereof. In some embodiments, the total MADRS score of the subject is as described herein, about 1 hour to about 4 hours, about 1.5 hours to about 5 hours, about 2 hours to about 6 hours, about 5 hours to about 10 hours, or about 45 minutes to about 24 hours prior to intranasal administration of the racemic ketamine, or a pharmaceutically acceptable salt thereof.

In some embodiments, the total MADRS score of the subject is 0 to 6 after intranasal administration of racemic ketamine, or a pharmaceutically acceptable salt thereof, as described herein. For example, 0 to 2, 0 to 3, 0 to 4, 0 to 5, 5 to 6, 4 to 6, 3 to 6, 2 to 6, or 1 to 6 after intranasal administration of racemic ketamine as described herein. In some embodiments, the total MADRS score of the subject is 0, 1, 2, 3, 4, 5, or 6 after intranasal administration of racemic ketamine as described herein. In some embodiments, the total MADRS score of the subject is measured at about 5 minutes to about 24 hours after intranasal administration of the racemic ketamine, or a pharmaceutically acceptable salt thereof. For example, about 5 minutes to 1 hour, about 5 minutes to 6 hours, about 5 minutes to 12 hours, about 5 minutes to 18 hours, about 18 hours to 24 hours, about 12 hours to 24 hours, or about 6 hours to 24 hours after intranasal administration of the racemic ketamine, or a pharmaceutically acceptable salt thereof. In some embodiments, the total MADRS score of the subject is as described herein, about 1 hour to about 4 hours, about 1.5 hours to about 5 hours, about 2 hours to about 6 hours, about 5 hours to about 10 hours, or about 45 minutes to about 24 hours after intranasal administration of the racemic ketamine, or a pharmaceutically acceptable salt thereof.

In some embodiments, the total MADRS score of the subject is 10 to about 60 prior to intranasal administration of racemic ketamine, or a pharmaceutically acceptable salt thereof, and the total MADRS score of the subject is 0 to 6 after intranasal administration of racemic ketamine, or a pharmaceutically acceptable salt thereof. For example, 10 to 20, 10 to 30, 10 to 40, 10 to 50, 50 to 60, 40 to 60, 30 to 60, or 20 to 60 prior to intranasal administration of racemic ketamine as described herein and 0 to 2, 0 to 3, 0 to 4, 0 to 5, 5 to 6, 4 to 6, 3 to 6, 2 to 6, or 1 to 6 after intranasal administration of racemic ketamine as described herein. In some embodiments, the total MADRS score of the subject is 10, 15, 20, 25, 30, 35, 40, 45, 50, 55, or 60 prior to intranasal administration of racemic ketamine as described herein and the total MADRS score of the subject is 0, 1, 2, 3, 4, 5, or 6 after intranasal administration of racemic ketamine as described herein. In some embodiments, the total MADRS score of the subject is measured at about 5 minutes to about 24 hours prior to intranasal administration of the racemic ketamine, or a pharmaceutically acceptable salt thereof. For example, about 5 minutes to 1 hour, about 5 minutes to 6 hours, about 5 minutes to 12 hours, about 5 minutes to 18 hours, about 18 hours to 24 hours, about 12 hours to 24 hours, or about 6 hours to 24 hours prior to intranasal administration of the racemic ketamine, or a pharmaceutically acceptable salt thereof, and about 5 minutes to 1 hour, about 5 minutes to 6 hours, about 5 minutes to 12 hours, about 5 minutes to 18 hours, about 18 hours to 24 hours, about 12 hours to 24 hours, or about 6 hours to 24 hours after intranasal administration of the racemic ketamine, or a pharmaceutically acceptable salt thereof. In some embodiments, the total MADRS score of the subject is as described herein about 1 hour to about 4 hours, about 1.5 hours to about 5 hours, about 2 hours to about 6 hours, about 5 hours to about 10 hours, or about 45 minutes to about 24 hours prior to intranasal administration of the racemic ketamine, or a pharmaceutically acceptable salt thereof, and about 1 hour to about 4 hours, about 1.5 hours to about 5 hours, about 2 hours to about 6 hours, about 5 hours to about 10 hours, or about 45 minutes to about 24 hours after intranasal administration of the racemic ketamine, or a pharmaceutically acceptable salt thereof.

In some embodiments, the total MADRS score of the subject is reduced by about 1 to about 60 after intranasal administration of the racemic ketamine, or a pharmaceutically acceptable salt thereof. For example, the total MADRS score of the subject can be reduced by about 1 to about 60 after intranasal administration of the racemic ketamine, or a pharmaceutically acceptable salt thereof, relative to prior to intranasal administration of the racemic ketamine, or a pharmaceutically acceptable salt thereof. In some embodiments, the total MADRS score of the subject is reduced by about 1 to about 50, about 1 to about 40, about 1 to about 30, about 1 to about 20, about 1 to about 10, about 50 to about 60, about 40 to about 60, about 30 to about 60, about 20 to about 60, or about 10 to about 60 after intranasal administration of the racemic ketamine, or a pharmaceutically acceptable salt thereof. In some embodiments, the total MADRS score of the subject is reduced as described herein about 5 minutes to about 24 hours after intranasal administration of the racemic ketamine, or a pharmaceutically acceptable salt thereof. For example, about 5 minutes to 1 hour, about 5 minutes to 6 hours, about 5 minutes to 12 hours, about 5 minutes to 18 hours, about 18 hours to 24 hours, about 12 hours to 24 hours, or about 6 hours to 24 hours after intranasal administration of the racemic ketamine, or a pharmaceutically acceptable salt thereof. In some embodiments, the total MADRS score of the subject is reduced as described herein about 1 hour to about 4 hours, about 1.5 hours to about 5 hours, about 2 hours to about 6 hours, or about 5 hours to about 10 hours, after intranasal administration of the racemic ketamine, or a pharmaceutically acceptable salt thereof (e.g., relative to prior to intranasal administration of the racemic ketamine, or a pharmaceutically acceptable salt thereof). In some embodiments, the MADRS total score of the subject is decreased by about 20 points to about 30 points 24 hours after intranasal administration of racemic ketamine, or a pharmaceutically acceptable salt thereof, for example, about 20-25 points, about 22-27 points, or about 25-30 points.

In some embodiments, MADRS Item 10, e.g., suicidal thoughts, of the MADRS can be used to measure MDD. In some embodiments, MADRS Item 10, e.g., suicidal thoughts, of the MADRS can be used to measure TRD. In some embodiments, MADRS Item 10, e.g., suicidal thoughts, of the MADRS can be used to measure other psychiatric disorders, as described herein. In some embodiments, the score for MADRS Item 10 for the subject is 0 or 1 after intranasal administration of racemic ketamine, or a pharmaceutically acceptable salt thereof, as described herein. In some embodiments, the score for MADRS Item 10 for the subject is as described herein about 5 minutes to about 24 hours after intranasal administration of the racemic ketamine, or a pharmaceutically acceptable salt thereof. For example, about 5 minutes to 1 hour, about 5 minutes to 6 hours, about 5 minutes to 12 hours, about 5 minutes to 18 hours, about 18 hours to 24 hours, about 12 hours to 24 hours, or about 6 hours to 24 hours after intranasal administration of the racemic ketamine, or a pharmaceutically acceptable salt thereof. In some embodiments, the score for MADRS Item 10 for the subject is as described herein about 1 hour to about 4 hours, about 1.5 hours to about 5 hours, about 2 hours to about 6 hours, about 5 hours to about 10 hours, or about 45 minutes to about 24 hours after intranasal administration of the racemic ketamine, or a pharmaceutically acceptable salt thereof.

In some embodiments, the score for MADRS Item 10 for the subject is reduced by 1 to 6 after intranasal administration of the racemic ketamine, or a pharmaceutically acceptable salt thereof. For example, the score for MADRS Item 10 for the subject can be reduced by 1 to 6 after intranasal administration of the racemic ketamine, or a pharmaceutically acceptable salt thereof, relative to prior to intranasal administration of the racemic ketamine, or a pharmaceutically acceptable salt thereof. In some embodiments, the score for MADRS Item 10 for the subject is reduced by 1 to 5, 1 to 4, 1 to 3, 1 to 2, 5 to 6, 4 to 6, 3 to 6, or 2 to 6 after intranasal administration of the racemic ketamine, or a pharmaceutically acceptable salt thereof. In some embodiments, the score for MADRS Item 10 for the subject is reduced as described herein about 5 minutes to about 24 hours after intranasal administration of the racemic ketamine, or a pharmaceutically acceptable salt thereof. For example, about 5 minutes to 1 hour, about 5 minutes to 6 hours, about 5 minutes to 12 hours, about 5 minutes to 18 hours, about 18 hours to 24 hours, about 12 hours to 24 hours, or about 6 hours to 24 hours after intranasal administration of the racemic ketamine, or a pharmaceutically acceptable salt thereof. In some embodiments, the score for MADRS Item 10 for the subject is reduced as described herein about 1 hour to about 4 hours, about 1.5 hours to about 5 hours, about 2 hours to about 6 hours, or about 5 hours to about 10 hours, after intranasal administration of the racemic ketamine, or a pharmaceutically acceptable salt thereof (e.g., relative to prior to intranasal administration of the racemic ketamine, or a pharmaceutically acceptable salt thereof).

In some embodiments, the MADRS item 10 score of the subject is reduced by 4, 5, or 6 points 24 hours after intranasal administration of racemic ketamine. In some embodiments, the MADRS Item 10 score of the subject is reduced by 4 points. In some embodiments, the MADRS Item 10 score of the subject is reduced by 5 points. In some embodiments, the MADRS Item 10 score of the subject is reduced by 6 points.

In some embodiments, a subject is administered the MADRS prior to intranasal administration of racemic ketamine as described herein. For example, the MADRS can be administered to the subject about 1 hour to about 6 months prior to intranasal administration of racemic ketamine as described herein. In some embodiments, the MADRS is administered to the subject about 1 hour to about 6 hours, about 1 hour to about 1 day, about 1 hour to about 1 week, about 1 hour to about 1 month, about 1 hour about 3 months, about 3 months to about 6 months, about 1 month to about 6 months, about 1 week to about 5 months, or about 1 day to about 6 months prior to intranasal administration of racemic ketamine as described herein.

In some embodiments, the MADRS Item 10 Score of the subject is 4, 5, or 6 units prior to intranasal administration of the racemic ketamine, or a pharmaceutically acceptable salt thereof. In some embodiments, the MADRS Item 10 Score of the subject is 5 or 6 units prior to intranasal administration of the racemic ketamine, or a pharmaceutically acceptable salt thereof. In some embodiments, the MADRS Item 10 Score of the subject is reduced by at least 1 unit 4 hours administration of the racemic ketamine, or a pharmaceutically acceptable salt thereof.

In some embodiments, the MADRS Item 10 Score of the subject is 2 units or 3 units prior to intranasal administration of the racemic ketamine, or a pharmaceutically acceptable salt thereof. In some embodiments, the MADRS Item 10 Score of the subject is 1 unit or 2 units prior to intranasal administration of the racemic ketamine, or a pharmaceutically acceptable salt thereof. In some embodiments, the MADRS Item 10 Score of the subject is reduced by at least 1 unit about 4 hours administration of the racemic ketamine, or a pharmaceutically acceptable salt thereof.

In some embodiments, the MADRS Item 10 Score of the subject is 2 units or 3 units prior to intranasal administration of the racemic ketamine, or a pharmaceutically acceptable salt thereof, and decreases by 1 unit to 2 units after intranasal administration of the racemic ketamine, or a pharmaceutically acceptable salt thereof.

In some embodiments, the MADRS Item 10 Score of the subject is 2 units or 3 units at about 5 minutes to about 24 hours prior to intranasal administration of the racemic ketamine, or a pharmaceutically acceptable salt thereof, and decreases by 1 unit to 2 units, about 5 minutes to about 24 hours after intranasal administration of the racemic ketamine, or a pharmaceutically acceptable salt thereof.

In some embodiments, the MADRS Item 10 Score of the subject is 2 units or 3 units at about 1 hour to about 4 hours, about 1.5 hours to about 5 hours, about 2 hours to about 6 hours, or about 5 hours to about hours, prior to intranasal administration of the racemic ketamine, or a pharmaceutically acceptable salt thereof, and decreases by 1 unit or 2 units about 1 hour to about 4 hours, about 1.5 hours to about 5 hours, about 2 hours to about 6 hours, or about 5 hours to about 10 hours, after intranasal administration of the racemic ketamine, or a pharmaceutically acceptable salt thereof. 10 The Clinician-Administered PTSD Scale (CAPS, also referred to as CAPS-5 when in accordance with DSM-5) is a structured diagnostic interview that assesses PTSD diagnostic status and symptom severity. The CAPS includes (a) assessment of all PTSD criteria plus associated features such as dissociation; (b) global ratings of distress, impairment, response validity, symptom severity, and improvement since a previous assessment; (c) both dichotomous (present/absent) and continuous ratings for individual symptoms and overall disorder; (d) separate assessment of symptom frequency and intensity; (e) behaviorally anchored prompts and rating scales; and (f) assessment of trauma-relatedness for individual symptoms not inherently linked to the trauma (e.g., loss of interest, estrangement, difficulty concentrating). See Weathers, et al., Psychol. Assess. 2018; Vol. 30, No. 3, pp. 383-95.

The CAPS uses a 0-4 point scale to evaluate frequency and intensity of symptoms, where 0=absent, 1=mild/subthreshold, 2=moderate/threshold, 3=severe/markedly elevated, and 4=extreme/incapacitating.

The frequency and intensity thresholds for the two key severity ratings (2=moderate/threshold and 3=severe/markedly elevated) are provided in the interview form for each symptom so that interviewers can refer directly to them to make the appropriate severity rating. For example, a severity rating of 2 generally requires a minimum frequency of at least twice a month or some of the time (20% to 30%) and a minimum intensity of clearly present. Similarly, a severity rating of 3 generally requires a minimum frequency of twice a week and a minimum intensity of pronounced. Finally, a symptom is considered present and subsequently counted toward a PTSD diagnosis if its severity rating is 2 or higher.

In some embodiments, the subject has an average CAPS score of 3 or 4 prior to administration of intranasal racemic ketamine, or a pharmaceutically acceptable salt thereof. In some embodiments, the subject has an average CAPS score of 0 or 1 points at about 24 hours after the first administration of intranasal racemic ketamine, or a pharmaceutically acceptable salt thereof.

In some embodiments, the subject has CAPS-5 baseline severity of 3. In some embodiments, the subject has CAPS-5 baseline severity of 4.

The CGIS-SI/B scale is a 5-item clinician-rated measure of suicidality-specific symptom severity. Clinicians rate the most severe level of suicidality experienced by the subject during the specified recall period (e.g., at screening, at baseline, before intranasal administration of racemic ketamine, and/or after intranasal administration of racemic ketamine as described herein), with response reported on a 5-point Likert-type scale ranging from 1 (not at all suicidal) to 5 (among the most extremely suicidal). The clinician can subsequently rate how much a subject's suicidality changed compared with their condition at baseline using the CGIC-SI/B also on a 7 point Likert-type scale ranging from 1 (very much improved) to 7 (very much worse). See, e.g., Meltzer et al. Arch Gen Psychiatry. 2003; 60(1):82-91.

In some embodiments, the Clinical Global Impression of Severity for Suicide Ideation and Behavior (CGIS-SI/B) score of the subject is 4 or 5 units prior to intranasal administration of the racemic ketamine, or a pharmaceutically acceptable salt thereof. In some embodiments, the CGIS-SI/B score is 4 or greater, about 5 minutes to about 24 hours prior to intranasal administration of the racemic ketamine, or a pharmaceutically acceptable salt thereof. For example, about 5 minutes to 1 hour, about 5 minutes to 6 hours, about 5 minutes to 12 hours, about 5 minutes to 18 hours, about 18 hours to 24 hours, about 12 hours to 24 hours, or about 6 hours to 24 hours prior to intranasal administration of the racemic ketamine, or a pharmaceutically acceptable salt thereof. In some embodiments, the CGIS-SI/B score of the subject is 4 or 5 about 1 hour to about 4 hours, about 1.5 hours to about 5 hours, about 2 hours to about 6 hours, or about 5 hours to about 10 hours, prior to intranasal administration of the racemic ketamine, or a pharmaceutically acceptable salt thereof.

In some embodiments, the CGIS-SI/B score of the subject decreases by 1 to 4 (e.g., by 1 to 4 units) after intranasal administration of the racemic ketamine, or a pharmaceutically acceptable salt thereof. In some embodiments, the CGIS-SI/B score decreases by 1 to 3 (e.g., 1 to 3 units) after intranasal administration of the racemic ketamine, or a pharmaceutically acceptable salt thereof. In some embodiments, the CGIS-SI/B score decreases by 1 to 2 (e.g., 1 to 2 units) after intranasal administration of the racemic ketamine, or a pharmaceutically acceptable salt thereof. In some embodiments, the CGIS-SI/B score of the subject is decreased by 3 or 4 units about 24 hours after intranasal administration of racemic ketamine, or a pharmaceutically acceptable salt thereof. In some embodiments, the CGIS-SI/B score of the subject is decreased by 3 units. In some embodiments, the CGIS-SI/B score of the subject is decreased by 4 units.

In some embodiments, the CGIS-SI/B score of the subject is 1 or 2 units 24 hours after intranasal administration of the racemic ketamine, or a pharmaceutically acceptable salt thereof.

In some embodiments, the Clinical Global Impression of Severity for Suicide Ideation and Behavior (CGIS-SI/B) score of the subject is 2 or 3 units prior to intranasal administration of the racemic ketamine, or a pharmaceutically acceptable salt thereof. In some embodiments, the CGIS-SI/B score of the subject is 1 or 2 units about 24 hours after intranasal administration of the racemic ketamine, or a pharmaceutically acceptable salt thereof.

In some embodiments, the Clinical Global Impression of Severity for Suicide Ideation and Behavior (CGIS-SI/B) score of the subject is 2 or 3 units prior to intranasal administration of the racemic ketamine, or a pharmaceutically acceptable salt thereof, and decreases by 1 unit to 2 units after intranasal administration of the racemic ketamine, or a pharmaceutically acceptable salt thereof.

In some embodiments, the CGIS-SI/B score is 3 or greater (e.g., 3, 4, or 5) prior to intranasal administration of the racemic ketamine, or a pharmaceutically acceptable salt thereof, and decreases by 1 to 4 after intranasal administration of the racemic ketamine, or a pharmaceutically acceptable salt thereof. In some embodiments, the CGIS-SI/B score is 4 or greater (e.g., 4 or 5) prior to intranasal administration of the racemic ketamine, or a pharmaceutically acceptable salt thereof, and decreases by 1 to 4, e.g., by 1, 2, 3, or 4, after intranasal administration of the racemic ketamine, or a pharmaceutically acceptable salt thereof. In some embodiments, the CGIS-SI/B score is 2 to 5 (e.g., 2, 3, 4, or 5) prior to intranasal administration of the racemic ketamine, or a pharmaceutically acceptable salt thereof, and decreases by 1 to 4, e.g., by 1, 2, 3, or 4 after intranasal administration of the racemic ketamine, or a pharmaceutically acceptable salt thereof. In some embodiments, the CGIS-SI/B score of the subject is 2, 3, 4, or 5 at about 5 minutes to about 24 hours prior to intranasal administration of the racemic ketamine, or a pharmaceutically acceptable salt thereof, and decreases by 1, 2, 3, or 4, about 5 minutes to about 24 hours after intranasal administration of the racemic ketamine, or a pharmaceutically acceptable salt thereof. In some embodiments, the CGIS-SI/B score of the subject is 2, 3, 4, 5 at about 1 hour to about 4 hours, about 1.5 hours to about 5 hours, about 2 hours to about 6 hours, or about 5 hours to about 10 hours, prior to intranasal administration of the racemic ketamine, or a pharmaceutically acceptable salt thereof, and decreases by 1, 2, 3, or 4, about 1 hour to about 4 hours, about 1.5 hours to about 5 hours, about 2 hours to about 6 hours, or about 5 hours to about 10 hours, after intranasal administration of the racemic ketamine, or a pharmaceutically acceptable salt thereof.

In some embodiments, the Clinical Global Impression of Severity for Suicide Ideation and Behavior (CGIS-SI/B) score of the subject is 2 or 3 units at about 5 minutes to about 24 hours prior to intranasal administration of the racemic ketamine, or a pharmaceutically acceptable salt thereof, and decreases by 1 unit to 2 units, about 5 minutes to about 24 hours after intranasal administration of the racemic ketamine, or a pharmaceutically acceptable salt thereof.

In some embodiments, the CGIS-SI/B score is 2 or 3 prior to intranasal administration of the racemic ketamine, or a pharmaceutically acceptable salt thereof, and decreases by 1 to 2 after intranasal administration of the racemic ketamine, or a pharmaceutically acceptable salt thereof. In some embodiments, the Clinical Global Impression of Severity for Suicide Ideation and Behavior (CGIS-SI/B) score of the subject is 2 or 3 units at about 1 hour to about 4 hours, about 1.5 hours to about 5 hours, about 2 hours to about 6 hours, or about 5 hours to about 10 hours, prior to intranasal administration of the racemic ketamine, or a pharmaceutically acceptable salt thereof, and decreases by 1 unit or 2 units about 1 hour to about 4 hours, about 1.5 hours to about 5 hours, about 2 hours to about 6 hours, or about 5 hours to about 10 hours, after intranasal administration of the racemic ketamine, or a pharmaceutically acceptable salt thereof. In some embodiments, the CGIS-SI/B score of the subject is 2 or 3 at about 5 minutes to about 24 hours prior to intranasal administration of the racemic ketamine, or a pharmaceutically acceptable salt thereof, and decreases by 1 or 2, about 5 minutes to about 24 hours after intranasal administration of the racemic ketamine, or a pharmaceutically acceptable salt thereof.

In some embodiments, a first CGIS-SI/B score is determined about 1 hour to about 12 hours prior to intranasal administration of the racemic ketamine, or a pharmaceutically acceptable salt thereof, and a second CGIS-SI/B score is determined about 1 hour to about 24 hours after to intranasal administration of the racemic ketamine, or a pharmaceutically acceptable salt thereof. In some embodiments, the first CGIS-SI/B score from the subject is determined about 4 hours to about 8 hours prior to intranasal administration of the racemic ketamine, or a pharmaceutically acceptable salt thereof. In some embodiments, the second CGIS-SI/B score from the subject is determined about 4 hours to about 24 hours after intranasal administration of the racemic ketamine, or a pharmaceutically acceptable salt thereof. In some embodiments, the first CGIS-SI/B score and the second CGIS-SI/B score are determined at equivalent times before, and after, administration of the racemic ketamine, or a pharmaceutically acceptable salt thereof, such as 4 hours. In some embodiments, the first CGIS-SI/B score and the second CGIS-SI/B score are determined at different times before, and after, administration of the racemic ketamine, or a pharmaceutically acceptable salt thereof, such as 4 hours before, and 12 hours after, administration of the racemic ketamine, or a pharmaceutically acceptable salt thereof.

The Sheehan Suicidality Tracking Scale (Sheehan-STS, S-STS) is a prospective rating scale that tracks both treatment-emergent suicidal ideation and behaviors. The Sheehan-STS is a scale with 14 core items and nine additional questions contingent on the core responses that can be administered either by a clinician or patient through self-report. Each item in the Sheehan-STS is scored on a 5-point Likert scale (0=not at all, 1=a little, 2-moderately, 3=very, and 4-extremely).

The CMCM version of the S-STS (S-STS CMCM) has four parts, and can also provide a comprehensive description of suicidal ideation and behavior. See, e.g., Sheehan et al., Innov. Clin. Neurosci. Vol. 11, No. 9-10, pp. 93-140 (2014) The S-STS CMCM (version 01/01/19) is a clinician-rated outcome measure which assesses SI/B on multiple patient and clinician-rated items. It includes 13 suicidality items that are rated on a Likert-type scale ranging from “not at all” (0) to “extremely” (4), yielding a total score ranging from 0 to 52. The final 6 items are used only when the patient misses a visit and is unable to complete the scale; if that missed visit is due to attempted or completed suicide, the possible maximum score is 100. The CMCM also yields 5 different single-item global assessments: 1) subject-rated likelihood of a suicide attempt; 2) subject-rated treatment needed; 3) clinician global severity of suicidal impulses, thoughts, and behaviors; 4) clinician judgment of suicide risk at this time and level of management needed for suicidality; and 5) clinician judgment of subject's likelihood of making a suicide attempt or of dying by suicide in the next 7 days.

In some embodiments, the Sheehan-Suicidality Tracking Scale Clinically Meaningful Change Measure (STS-CMCM) score of the subject is from 15-52 units prior to intranasal administration of the racemic ketamine, or a pharmaceutically acceptable salt thereof. In some embodiments, the STS-CMCM score of the subject is from 20-52 units prior to intranasal administration of the racemic ketamine, or a pharmaceutically acceptable salt thereof. In some embodiments, the STS-CMCM score of the subject is reduced by at least 50% at about 24 hours after intranasal administration of the racemic ketamine, or a pharmaceutically acceptable salt thereof. In some embodiments, the STS-CMCM score of the subject is from 1-3 units about 24 hours after intranasal administration of the racemic ketamine, or a pharmaceutically acceptable salt thereof.

In some embodiments, the STS-CMCM score of the subject is reduced by at least 1 after intranasal administration of the racemic ketamine, or a pharmaceutically acceptable salt thereof. In some embodiments, the STS-CMCM score of the subject can be reduced by at least 2, at least 3, at least 4, or at least 5 after intranasal administration of the racemic ketamine, or a pharmaceutically acceptable salt thereof, relative to prior to intranasal administration of the racemic ketamine, or a pharmaceutically acceptable salt thereof. In some embodiments, the STS-CMCM score of the subject is reduced as described herein about 5 minutes to about 24 hours after intranasal administration of the racemic ketamine, or a pharmaceutically acceptable salt thereof. For example, about 5 minutes to 1 hour, about 5 minutes to 6 hours, about 5 minutes to 12 hours, about 5 minutes to 18 hours, about 18 hours to 24 hours, about 12 hours to 24 hours, or about 6 hours to 24 hours after intranasal administration of the racemic ketamine, or a pharmaceutically acceptable salt thereof. In some embodiments, the STS-CMCM score of the subject is reduced as described herein about 1 hour to about 4 hours, about 1.5 hours to about 5 hours, about 2 hours to about 6 hours, or about 5 hours to about 10 hours, after intranasal administration of the racemic ketamine, or a pharmaceutically acceptable salt thereof (e.g., relative to prior to intranasal administration of the racemic ketamine, or a pharmaceutically acceptable salt thereof). In some embodiments, a subject is administered the STS-CMCM prior to intranasal administration of racemic ketamine as described herein. For example, the STS-CMCM can be administered to the subject about 1 hour to about 6 months prior to intranasal administration of racemic ketamine as described herein. In some embodiments, the STS-CMCM is administered to the subject about 1 hour to about 6 hours, about 1 hour to about 1 day, about 1 hour to about 1 week, about 1 hour to about 1 month, about 1 hour about 3 months, about 3 months to about 6 months, about 1 month to about 6 months, about 1 week to about 5 months, or about 1 day to about 6 months prior to intranasal administration of racemic ketamine as described herein. In some embodiments, the STS-CMCM score of the subject is from 10 units to 20 units prior to intranasal administration of the racemic ketamine, or a pharmaceutically acceptable salt thereof. In some embodiments, the STS-CMCM score of the subject is from 5 units to 10 units prior to intranasal administration of the racemic ketamine, or a pharmaceutically acceptable salt thereof. In some embodiments, the STS-CMCM score of the subject is reduced by at least 50% at about 24 hours after intranasal administration of the racemic ketamine, or a pharmaceutically acceptable salt thereof. In some embodiments, the STS-CMCM score of the subject is 1 unit to 2 units about 24 hours after intranasal administration of the racemic ketamine, or a pharmaceutically acceptable salt thereof.

In some embodiments, the STS-CMCM score of the subject is 10 units to 20 units prior to intranasal administration of the racemic ketamine, or a pharmaceutically acceptable salt thereof, and decreases by 5 units to 10 units, e.g., by 5, 6, 7, 8, 9, or 10 units after intranasal administration of the racemic ketamine, or a pharmaceutically acceptable salt thereof.

In some embodiments, the STS-CMCM score of the subject is 10 units to 20 units at about 5 minutes to about 24 hours prior to intranasal administration of the racemic ketamine, or a pharmaceutically acceptable salt thereof, and decreases by 5, 6, 7, 8, or 10 units, about 5 minutes to about 24 hours after intranasal administration of the racemic ketamine, or a pharmaceutically acceptable salt thereof.

In some embodiments, the STS-CMCM score of the subject is 10 to 20 units at about 1 hour to about 4 hours, about 1.5 hours to about 5 hours, about 2 hours to about 6 hours, or about 5 hours to about 10 hours, prior to intranasal administration of the racemic ketamine, or a pharmaceutically acceptable salt thereof, and decreases by 5, 6, 7, 8, 9, or 10 units about 1 hour to about 4 hours, about 1.5 hours to about 5 hours, about 2 hours to about 6 hours, or about 5 hours to about 10 hours, after intranasal administration of the racemic ketamine, or a pharmaceutically acceptable salt thereof.

In some embodiments, the STS-CMCM total score of the subject is decreased by about 15 units to about 25 units about 24 hours after intranasal administration of racemic ketamine. In some embodiments, the STS-CMCM total score is decreased by about 15 units to about 20 units, about 17 units to about 22 units, or about 20 units to about 25 units.

In some embodiments, the STS-CMCM Risk of Suicide at Within the Next 7 Days score of the subject is from 5 to 10 units prior to intranasal administration of the racemic ketamine, or a pharmaceutically acceptable salt thereof. In some embodiments, the STS-CMCM Risk of Suicide at Within the Next 7 Days score of the subject is reduced by at least 50% 24 hours after intranasal administration of the racemic ketamine, or a pharmaceutically acceptable salt thereof. In some embodiments, the STS-CMCM Risk of Suicide at Within the Next 7 Days score of the subject is from 0-2 units 24 hours after intranasal administration of the racemic ketamine, or a pharmaceutically acceptable salt thereof. In some embodiments, the STS-CMCM Risk of Suicide at Within the Next 7 Days score of the subject is 0 or 1 unit 96 hours after intranasal administration of the racemic ketamine, or a pharmaceutically acceptable salt thereof.

In some embodiments, the STS-CMCM Risk of Suicide at Within the Next 7 Days score of the subject is 3 units to 5 units prior to intranasal administration of the racemic ketamine, or a pharmaceutically acceptable salt thereof. In some embodiments, the STS-CMCM Risk of Suicide at Within the Next 7 Days score of the subject is reduced by at least 50% 24 hours after intranasal administration of the racemic ketamine, or a pharmaceutically acceptable salt thereof. In some embodiments, the STS-CMCM Risk of Suicide at Within the Next 7 Days score of the subject is 0 units or 1 unit 24 hours after intranasal administration of the racemic ketamine, or a pharmaceutically acceptable salt thereof. In some embodiments, the STS-CMCM Risk of Suicide at Within the Next 7 Days score of the subject is 0 units 96 hours after intranasal administration of the racemic ketamine, or a pharmaceutically acceptable salt thereof.

In some embodiments, the STS-CMCM Risk of Suicide at Within the Next 7 Days score of the subject is 3 units to 5 units prior to intranasal administration of the racemic ketamine, or a pharmaceutically acceptable salt thereof, and decreases by 2 units to 4 units, e.g., by 2, 3, or 4 units after intranasal administration of the racemic ketamine, or a pharmaceutically acceptable salt thereof.

In some embodiments, the STS-CMCM Risk of Suicide at Within the Next 7 Days score of the subject is 3 units to 5 units at about 5 minutes to about 24 hours prior to intranasal administration of the racemic ketamine, or a pharmaceutically acceptable salt thereof, and decreases by 2, 3, or 4 units, about 5 minutes to about 24 hours after intranasal administration of the racemic ketamine, or a pharmaceutically acceptable salt thereof.

In some embodiments, the STS-CMCM Risk of Suicide at Within the Next 7 Days score of the subject is 3 units to 5 units at about 1 hour to about 4 hours, about 1.5 hours to about 5 hours, about 2 hours to about 6 hours, or about 5 hours to about 10 hours, prior to intranasal administration of the racemic ketamine, or a pharmaceutically acceptable salt thereof, and decreases by 2, 3, or 4 units about 1 hour to about 4 hours, about 1.5 hours to about 5 hours, about 2 hours to about 6 hours, or about 5 hours to about 10 hours, after intranasal administration of the racemic ketamine, or a pharmaceutically acceptable salt thereof.

The Columbia Suicide Severity Rating Scale (C-SSRS) is a suicidal ideation and behavior rating scale used to measure suicidality and/or suicidal ideation in a subject, in particular, to assesses severity and intensity of suicidal ideation, types of suicidal behaviors, and lethality of suicide attempts. For example, the C-SSRS can assess the lethality of suicide attempts and other features of suicidal ideation such as frequency, duration, controllability, reasons for ideation, and deterrents, all of which can be significantly predictive of completed suicide. See, e.g., https.//cssrs.columbia.edu/wp-content/uploads/C-SSRS-Baseline-Screening AU5.1_eng-USori.pdf and https./cssrs.columbia.edu/wp-content/uploads/C-SSRS-1-14.09.SinceLastVisit_AU5.1 eng-USori-1.pdf.

The C-SSRS provides several questions directed to suicidal ideation that the subject answers with a “yes” or a “no.” Such questions are directed to a wish to be dead; non-specific active suicidal thoughts; active suicidal ideation with any methods (not a plan) without intent to act; active suicidal ideation with some intent to act; without a specific plan; and active suicidal ideation with specific plan and intent. Additionally, the C-SSRS includes features that are rated by the subject to help assess the intensity of ideation. Such features include asking about the frequency (e.g., less than one a week, once a week, 2-5 times a week, daily or almost daily, and many times each day); duration (e.g., fleeting, less than an hour, 1-4 hours, 4-8 hours, more than 8 hours); controllability (e.g., easily able to control thoughts, can control thoughts with little difficulty, can control thoughts with some difficulty, can control thoughts with a lot of difficulty, unable to control thoughts, and does not attempt to control thoughts); deterrents (e.g., deterrents definitely stopped you from attempting suicide, deterrents probably stopped you, uncertain deterrent stopped you, deterrent most likely did not stop you, and deterrents definitely did not stop you); and reasons for ideation (e.g., completely to get attention, mostly to get attention, equally to get attention and to end/stop pain, mostly to end/stop pain, and completely to end/stop pain). The C-SSRS can also include questions directed to suicidal behavior and an actual suicide attempt such as asking about if an attempt was made; asking if anything was done to cause harm to one's self, and asking if the subject has done anything dangerous where he or she could have died.

In some embodiments, the C-SSRS score is 0-2 prior to intranasal administration of the racemic ketamine, or a pharmaceutically acceptable salt thereof. For example, the subject answers “yes” to 0, 1, or 2 questions prior to intranasal administration of the racemic ketamine, or a pharmaceutically acceptable salt thereof. In some embodiments, the subject answers “yes” to 0 questions on the C-SSRS as described herein, about 5 minutes to about 24 hours prior to intranasal administration of the racemic ketamine, or a pharmaceutically acceptable salt thereof. For example, about 5 minutes to 1 hour, about 5 minutes to 6 hours, about 5 minutes to 12 hours, about 5 minutes to 18 hours, about 18 hours to 24 hours, about 12 hours to 24 hours, or about 6 hours to 24 hours prior to intranasal administration of the racemic ketamine, or a pharmaceutically acceptable salt thereof. In some embodiments, the subject answers “yes” to 0 questions on the C-SSRS described herein, about 1 hour to about 4 hours, about 1.5 hours to about 5 hours, about 2 hours to about 6 hours, or about 5 hours to about 10 hours, prior to intranasal administration of the racemic ketamine, or a pharmaceutically acceptable salt thereof.

In some embodiments, the C-SSRS score of the subject is from 2 units to 9 units prior to intranasal administration of the racemic ketamine, or a pharmaceutically acceptable salt thereof. In some embodiments, the C-SSRS score of the subject is from 2 units to 5 units prior to intranasal administration of the racemic ketamine, or a pharmaceutically acceptable salt thereof. In some embodiments, the CSSR-S score of the subject is zero units 24 hours after intranasal administration of the racemic ketamine, or a pharmaceutically acceptable salt thereof.

In some embodiments, the C-SSRS score decreases by 1-2 (e.g., by 1 to 2 units) after intranasal administration of the racemic ketamine, or a pharmaceutically acceptable salt thereof.

In some embodiments, a first C-SSRS score is determined about 1 hour to about 12 hours prior to intranasal administration of the racemic ketamine, or a pharmaceutically acceptable salt thereof; and a second C-SSRS score is determined about 1 hour to about 24 hours after to intranasal administration of the racemic ketamine, or a pharmaceutically acceptable salt thereof. In some embodiments, the first C-SSRS score from the subject is determined about 4 hours to about 8 hours prior to intranasal administration of the racemic ketamine, or a pharmaceutically acceptable salt thereof. In some embodiments, the second C-SSRS score from the subject is determined about 4 hours to about 8 hours after intranasal administration of the racemic ketamine, or a pharmaceutically acceptable salt thereof. In some embodiments, the first C-SSRS score and the second C-SSRS score are determined at equivalent times before, and after, administration of the racemic ketamine, or a pharmaceutically acceptable salt thereof, such as 4 hours. In some embodiments, the first C-SSRS score and the second C-SSRS score are determined at different times before, and after, administration of the racemic ketamine, or a pharmaceutically acceptable salt thereof, such as 4 hours before, and 12 hours after, administration of the racemic ketamine, or a pharmaceutically acceptable salt thereof.

Two versions of the C-SSRS can be used: a Baseline version, which can assess lifetime, past month, and past twelve month suicidal ideation and behavior in a subject, and a “Since Last Visit” version, which can assess suicidal thoughts or behaviors the subject may have had since the last time the C-SSRS was administered to the subject. For example, a subject should only respond “yes” to being asked about making a suicide attempt in the “Since Last Visit” version, if the attempt was made after the Baseline version of the C-SSRS was administered. In some embodiments, a subject is administered the Baseline version of the C-SSRS prior to intranasal administration of racemic ketamine as described herein. For example, the Baseline version of the C-SSRS can be administered about 1 hour to about 6 months prior to intranasal administration of racemic ketamine as described herein. In some embodiments, the Baseline version of the C-SSRS is administered about 1 hour to about 6 hours, about 1 hour to about 1 day, about 1 hour to about 1 week, about 1 hour to about 1 month, about 1 hour about 3 months, about 3 months to about 6 months, about 1 month to about 6 months, about 1 week to about 5 months, or about 1 day to about 6 months prior to intranasal administration of racemic ketamine as described herein.

In some embodiments, occurrence of suicidal ideation after baseline is defined as having answered “yes” to at least 1 of the 5 suicidal ideation subcategories (i.e., wish to be dead; nonspecific active suicidal thoughts; active suicidal ideation with any methods without intent to act; active suicidal ideation with some intent to act; and active suicidal ideation with specific plan and intent) at a C-SSRS administration after the Baseline C-SSRS. In some embodiments, occurrence of suicidal behavior after baseline is defined as having answered “yes” to at least 1 of the 6 suicidal behavior sub-categories (i.e., actual attempt, interrupted attempt, aborted attempt, preparatory acts or behavior, suicide behavior, and suicide) at a C-SSRS administration after the Baseline C-SSRS. In some embodiments, a trained rater will complete the C-SSRS based on responses from the subject.

In some embodiments, the Since Last Visit version of the C-SSRS is administered to the subject after intranasal administration of the racemic ketamine, or a pharmaceutically acceptable salt thereof, as described herein. In some embodiments, the C-SSRS is administered to the subject, about 5 minutes to about 24 hours after intranasal administration of the racemic ketamine, or a pharmaceutically acceptable salt thereof, to the subject. For example, about 5 minutes to 1 hour, about 5 minutes to 6 hours, about 5 minutes to 12 hours, about 5 minutes to 18 hours, about 18 hours to 24 hours, about 12 hours to 24 hours, or about 6 hours to 24 hours after intranasal administration of the racemic ketamine, or a pharmaceutically acceptable salt thereof, to the subject. In some embodiments, the C-SSRS is administered to the subject, about 5 minutes to 1 hour, about 5 minutes to 6 hours, about 5 minutes to 12 hours, about 5 minutes to 18 hours, about 18 hours to 24 hours, about 12 hours to 24 hours, or about 6 hours to 24 hours after intranasal administration of the racemic ketamine, or a pharmaceutically acceptable salt thereof, to the subject.

In some embodiments, C-SSRS score is 0-2 after intranasal administration of racemic ketamine, or a pharmaceutically acceptable salt thereof, as described herein. For example, the subject answers “yes” to 0, 1, or 2 questions on the C-SSRS after intranasal administration of racemic ketamine as described herein. In some embodiments, the C-SSRS score of the subject is as described herein about 5 minutes to about 24 hours after intranasal administration of the racemic ketamine, or a pharmaceutically acceptable salt thereof. For example, about 5 minutes to 1 hour, about 5 minutes to 6 hours, about 5 minutes to 12 hours, about 5 minutes to 18 hours, about 18 hours to 24 hours, about 12 hours to 24 hours, or about 6 hours to 24 hours after intranasal administration of the racemic ketamine, or a pharmaceutically acceptable salt thereof. In some embodiments, the subject answers “yes” to 0, 1, or 2 questions on the C-SSRS, about 1 hour to about 4 hours, about 2 to about 12 hours, about 1.5 hours to about 5 hours, about 2 hours to about 6 hours, about 4 to about 8 hours, about 5 hours to about 10 hours, or about 45 minutes to about 24 hours after intranasal administration of the racemic ketamine, or a pharmaceutically acceptable salt thereof. In some embodiments, the C-SSRS is the Since Last Visit version of the C-SSRS.

In some embodiments, the subject has an intensity of ideation on the C-SSRS of 0 to 6 after intranasal administration of the racemic ketamine, or a pharmaceutically acceptable salt thereof. For example, the intensity of ideation of the subject can be 0, 1, 2, 3, 4, 5, or 6 after intranasal administration of the racemic ketamine, or a pharmaceutically acceptable salt thereof. In some embodiments, the intensity of ideation of the subject is as described herein about 5 minutes to about 24 hours after intranasal administration of the racemic ketamine, or a pharmaceutically acceptable salt thereof. For example, about 5 minutes to 1 hour, about 5 minutes to 6 hours, about 5 minutes to 12 hours, about 5 minutes to 18 hours, about 18 hours to 24 hours, about 12 hours to 24 hours, or about 6 hours to 24 hours after intranasal administration of the racemic ketamine, or a pharmaceutically acceptable salt thereof. In some embodiments, the intensity of ideation of the subject is as described herein, about 1 hour to about 4 hours, about 1.5 hours to about 5 hours, about 2 hours to about 6 hours, or about 5 hours to about 10 hours, after intranasal administration of the racemic ketamine, or a pharmaceutically acceptable salt thereof. In some embodiments, the Since Last Version of the C-SSRS is administered after intranasal administration of the racemic ketamine, or a pharmaceutically acceptable salt thereof.

In some embodiments, the C-SSRS score in a subject administered intranasal racemic ketamine, or a pharmaceutically acceptable salt thereof, is 1-2 points less than a subject administered an equivalent dose of intravenous racemic ketamine, or a pharmaceutically acceptable salt thereof, or (S)-ketamine, or a pharmaceutically acceptable salt thereof.

In some embodiments, the C-SSRS score in a subject administered intranasal racemic ketamine, or a pharmaceutically acceptable salt thereof, improves at a faster rate than a subject administered an equivalent dose of intravenous racemic ketamine, or a pharmaceutically acceptable salt thereof, or (S)-ketamine, or a pharmaceutically acceptable salt thereof. For example, the C-SSRS score improves at a rate of 0.25 points/hr, 0.5 points/hr, 0.75 points/hr, 1 point/hr, 1.25 points/hr, 1.5 points/hr, 1.75 points/hr, 2 points/hr, 2.25 points/hr, 2.5 points/hr, 2.75 points/hr, 3 points/hr, 3.25 points/hr, 3.5 points/hr, 3.75 points/hr, 4 points/hr, or any value in between, faster than a subject administered an equivalent dose of intravenous racemic ketamine, or a pharmaceutically acceptable salt thereof, or (S)-ketamine, or a pharmaceutically acceptable salt thereof.

Physician Withdrawal Checklist 20-item (PWC-20) is a clinical instrument designed to detect any potential benzodiazepine-like treatment discontinuation (withdrawal) symptoms caused by experimental anxiolytics of the non-SSRI type. It records 20 symptoms reported by a subject grouped into 5 different categories consisting of somatic, mood, cognitive, fatigue, and gastrointestinal issues. Each symptom is given a severity score according to 0=not present, 1=mild, 2=moderate, and 3=severe, for a total score that ranges from 0 to 60. A higher score is associated with more withdrawal symptoms with more severity; a lower score is associated with less withdrawal symptoms with less severity.

In some embodiments, the Physician Withdrawal Checklist 20-item (PWC-20) score of the subject is up to 60 units prior to intranasal administration of the racemic ketamine, or a pharmaceutically acceptable salt thereof. In some embodiments, PWC-20 score of the subject is from 5 to 50 units prior to intranasal administration of the racemic ketamine, or a pharmaceutically acceptable salt thereof. In some embodiments, PWC-20 score of the subject is from 10 to 40 units prior to intranasal administration of the racemic ketamine, or a pharmaceutically acceptable salt thereof. In some embodiments, PWC-20 score of the subject is from 15 to 30 units prior to intranasal administration of the racemic ketamine, or a pharmaceutically acceptable salt thereof. In some embodiments, PWC-20 score of the subject is from 20 to 30 units prior to intranasal administration of the racemic ketamine, or a pharmaceutically acceptable salt thereof. In some embodiments, PWC-20 score of the subject is from 0 to 15 units prior to intranasal administration of the racemic ketamine, or a pharmaceutically acceptable salt thereof. In some embodiments, PWC-20 score of the subject is 15 to 30 units prior to intranasal administration of the racemic ketamine, or a pharmaceutically acceptable salt thereof. In some embodiments, PWC-20 score of the subject is 30 to 60 units prior to intranasal administration of the racemic ketamine, or a pharmaceutically acceptable salt thereof.

In some embodiments, PWC-20 score of the subject is 60 to 40 units, 40 to 20, 20 to 10, or 10 to 0 units prior to intranasal administration of the racemic ketamine, or a pharmaceutically acceptable salt thereof, and is reduced by at least 80% at about 24 hours after intranasal administration of the racemic ketamine, or a pharmaceutically acceptable salt thereof. In some embodiments, PWC-20 score of the subject is at 60 to 40 units, 40 to 20, 20 to 10, or 10 to 0 units prior to intranasal administration of the racemic ketamine, or a pharmaceutically acceptable salt thereof, and reduced by at least 50% at about 24 hours after intranasal administration of the racemic ketamine, or a pharmaceutically acceptable salt thereof. In some embodiments, PWC-20 score of the subject is 60 to 40 units, 40 to 20, 20 to 10, or 10 to 0 units prior to intranasal administration of the racemic ketamine, or a pharmaceutically acceptable salt thereof, and reduced by at least 25% at about 24 hours after intranasal administration of the racemic ketamine, or a pharmaceutically acceptable salt thereof. In some embodiments, PWC-20 score of the subject is 60 to 40 units, 40 to 20, 20 to 10, 10 to 0 units prior to intranasal administration of the racemic ketamine, or a pharmaceutically acceptable salt thereof and reduced by at least 10% at about 24 hours after intranasal administration of the racemic ketamine, or a pharmaceutically acceptable salt thereof. In some embodiments, PWC-20 score of the subject is 60 to 40 units, 40 to 20, 20 to 10, 10 to 0 units prior to intranasal administration of the racemic ketamine, or a pharmaceutically acceptable salt thereof, and reduced by 5 to 10% or by 0 to 5% at about 24 hours after intranasal administration of the racemic ketamine, or a pharmaceutically acceptable salt thereof.

In some embodiments, PWC-20 score of the subject is 0 to 5 units about 24 hours after intranasal administration of the racemic ketamine, or a pharmaceutically acceptable salt thereof. In some embodiments, PWC-20 score of the subject is 5 to 10 units about 24 hours after intranasal administration of the racemic ketamine, or a pharmaceutically acceptable salt thereof. In some embodiments, PWC-20 score of the subject is 10 to 20 units about 24 hours after intranasal administration of the racemic ketamine, or a pharmaceutically acceptable salt thereof. In some embodiments, PWC-20 score of the subject is 20 to 30 units about 24 hours after intranasal administration of the racemic ketamine, or a pharmaceutically acceptable salt thereof. In some embodiments, PWC-20 score of the subject is 30 to 40 units about 24 hours after intranasal administration of the racemic ketamine, or a pharmaceutically acceptable salt thereof. In some embodiments, PWC-20 score of the subject is 40 to 50 units about 24 hours after intranasal administration of the racemic ketamine, or a pharmaceutically acceptable salt thereof. In some embodiments, PWC-20 score of the subject is 50 to 60 units about 24 hours after intranasal administration of the racemic ketamine, or a pharmaceutically acceptable salt thereof.

In some embodiments, PWC-20 score of the subject is reduced by up to 5 units after intranasal administration of the racemic ketamine, or a pharmaceutically acceptable salt thereof. In some embodiments, PWC-20 score of the subject is reduced 5 to 10 units after intranasal administration of the racemic ketamine, or a pharmaceutically acceptable salt thereof. In some embodiments, PWC-20 score of the subject is reduced 10 to 20 units after intranasal administration of the racemic ketamine, or a pharmaceutically acceptable salt thereof. In some embodiments, PWC-20 score of the subject is reduced 20 to 30 units after intranasal administration of the racemic ketamine, or a pharmaceutically acceptable salt thereof. In some embodiments, PWC-20 score of the subject is reduced 30 to 40 units after intranasal administration of the racemic ketamine, or a pharmaceutically acceptable salt thereof. In some embodiments, PWC-20 score of the subject is reduced 40 to 50 units after intranasal administration of the racemic ketamine, or a pharmaceutically acceptable salt thereof. In some embodiments, PWC-20 score of the subject is reduced up to 60 units after intranasal administration of the racemic ketamine, or a pharmaceutically acceptable salt thereof.

In some embodiments, PWC-20 score of the subject is reduced as described herein about 5 minutes to about 24 hours after intranasal administration of the racemic ketamine, or a pharmaceutically acceptable salt thereof. For example, about 5 minutes to 1 hour, about 5 minutes to 6 hours, about 5 minutes to 12 hours, about 5 minutes to 18 hours, about 18 hours to 24 hours, about 12 hours to 24 hours, or about 6 hours to 24 hours after intranasal administration of the racemic ketamine, or a pharmaceutically acceptable salt thereof. In some embodiments, PWC-20 score of the subject is reduced as described herein about 1 hour to about 4 hours, about 1.5 hours to about 5 hours, about 2 hours to about 6 hours, or about 5 hours to about 10 hours, after intranasal administration of the racemic ketamine, or a pharmaceutically acceptable salt thereof (e.g., relative to prior to intranasal administration of the racemic ketamine, or a pharmaceutically acceptable salt thereof). In some embodiments, a subject is administered the Physician Withdrawal Checklist 20-item (PWC-20) prior to intranasal administration of racemic ketamine as described herein. For example, the Physician Withdrawal Checklist 20-item (PWC-20) can be administered to the subject about 1 hour to about 6 months prior to intranasal administration of racemic ketamine as described herein.

In some embodiments, the Physician Withdrawal Checklist 20-item (PWC-20) is administered to the subject about 1 hour to about 6 hours, about 1 hour to about 1 day, about 1 hour to about 1 week, about 1 hour to about 1 month, about 1 hour about 3 months, about 3 months to about 6 months, about 1 month to about 6 months, about 1 week to about 5 months, or about 1 day to about 6 months prior to intranasal administration of racemic ketamine as described herein.

The European Quality of Life Group, 5-Dimension, 5-Level (EQ-5D-5L) is a self-report survey that measures quality of life across 5 domains: mobility, self-care, usual activities, pain/discomfort, and anxiety/depression. The EQ-5D-5L essentially consists of 2 parts: the EQ-5D descriptive system and the EQ visual analogue scale (EQ VAS). The subject is asked to indicate his/her health state by checking in the box against the most appropriate statement in each of the 5 dimensions; the statements include 1=no problems, 2=slight problems, 3=moderate problems, 4=severe problems, and 5=extreme problems. This decision results in a 1-digit number expressing the level selected for that dimension. The visual analogue scale (EQ VAS) shows a scale from 0 to 100, where the subject is asked to mark an “X” on the scale to indicate how their health is today. A unique health state is defined by combining 1 level from each of the 5 dimensions. A total of 3125 possible health states is defined in this way. Each state is referred to in terms of a 5 digit code. For example, state 11111 indicates no problems on any of the 5 dimensions, while state 12345 indicates no problems with mobility, slight problems with washing or dressing, moderate problems with doing usual activities, severe pain or discomfort and extreme anxiety or depression. The EQ-5D-5L health states, defined by the EQ-5D-5L descriptive system, may be converted into a single index value. These index values, are specific to each country and depend on the subject's age and sex.

While there are multiple ways to compare data, one way of presenting data as a health profile is by making a table with the frequency or the proportion of reported problems for each level for each dimension. These tables can be broken down to include the proportions per subgroup, such as age, before vs. after treatment, treatment vs. comparator or placebo, and so on. As a rule, the percentage of the general population in various age groups, e.g., 18-29, 30-39, 40-49, 50-59, 60-69, and 70+ report high percentages of 1 (no problems) in all 5 domains: mobility, self-care, usual activities, pain/discomfort, and anxiety/depression, with these percentages decreasing with increasing age. Put another way, a higher percentage of the general population tends to report more problems (higher health state score per domain) across the 5 domains as they age.

In some embodiments, the EQ-5D-5L index value of the subject is reduced by at least 80% relative to baseline (screening) at about 24 hours after intranasal administration of the racemic ketamine, or a pharmaceutically acceptable salt thereof. In some embodiments, the EQ-5D-5L index value of the subject is reduced by at least 70% relative to baseline at about 24 hours after intranasal administration of the racemic ketamine, or a pharmaceutically acceptable salt thereof. In some embodiments, the EQ-5D-5L index value of the subject is reduced by at least 60% relative to baseline at about 24 hours after intranasal administration of the racemic ketamine, or a pharmaceutically acceptable salt thereof. In some embodiments, the EQ-5D-5L index value of the subject is reduced by at least 50% relative to baseline at about 24 hours after intranasal administration of the racemic ketamine, or a pharmaceutically acceptable salt thereof. In some embodiments, the EQ-5D-5L index value of the subject is reduced by at least 40% relative to baseline at about 24 hours after intranasal administration of the racemic ketamine, or a pharmaceutically acceptable salt thereof. In some embodiments, the EQ-5D-5L index value of the subject is reduced by at least 30% relative to baseline at about 24 hours after intranasal administration of the racemic ketamine, or a pharmaceutically acceptable salt thereof. In some embodiments, the EQ-5D-5L index value of the subject is reduced by at least 20% relative to baseline at about 24 hours after intranasal administration of the racemic ketamine, or a pharmaceutically acceptable salt thereof. In some embodiments, the EQ-5D-5L index value of the subject is reduced by at least 10% relative to baseline at about 24 hours after intranasal administration of the racemic ketamine, or a pharmaceutically acceptable salt thereof. In some embodiments, the EQ-5D-5L index value of the subject is reduced by 5 to 10% or 0 to 5% relative to baseline at about 24 hours after intranasal administration of the racemic ketamine, or a pharmaceutically acceptable salt thereof.

In some embodiments, the EQ-5D-5L health state in one or more of 5 domains: mobility, self-care, usual activities, pain/discomfort, and anxiety/depression is reduced by 1 unit about 24 hours after intranasal administration of the racemic ketamine, or a pharmaceutically acceptable salt thereof. In some embodiments, the EQ-5D-5L health state in one or more of 5 domains: mobility, self-care, usual activities, pain/discomfort, and anxiety/depression is reduced by 2 units about 24 hours after intranasal administration of the racemic ketamine, or a pharmaceutically acceptable salt thereof. In some embodiments, the EQ-5D-5L health state in one or more of 5 domains: mobility, self-care, usual activities, pain/discomfort, and anxiety/depression is reduced by 3 units about 24 hours after intranasal administration of the racemic ketamine, or a pharmaceutically acceptable salt thereof. In some embodiments, the EQ-5D-5L health state in one or more of 5 domains: mobility, self-care, usual activities, pain/discomfort, and anxiety/depression is reduced by 4 units about 24 hours after intranasal administration of the racemic ketamine, or a pharmaceutically acceptable salt thereof. In some embodiments, the EQ-5D-5L health state in one or more of 5 domains: mobility, self-care, usual activities, pain/discomfort, and anxiety/depression is reduced by 5 units about 24 hours after intranasal administration of the racemic ketamine, or a pharmaceutically acceptable salt thereof.

In some embodiments, the EQ-5D-5L health state in two or more of 5 domains: mobility, self-care, usual activities, pain/discomfort, and anxiety/depression is reduced by 1 unit about 24 hours after intranasal administration of the racemic ketamine, or a pharmaceutically acceptable salt thereof. In some embodiments, the EQ-5D-5L health state in two or more of 5 domains: mobility, self-care, usual activities, pain/discomfort, and anxiety/depression is reduced by 2 units about 24 hours after intranasal administration of the racemic ketamine, or a pharmaceutically acceptable salt thereof. In some embodiments, the EQ-5D-5L health state in two or more of 5 domains: mobility, self-care, usual activities, pain/discomfort, and anxiety/depression is reduced by 3 units about 24 hours after intranasal administration of the racemic ketamine, or a pharmaceutically acceptable salt thereof. In some embodiments, the EQ-5D-5L health state in two or more of 5 domains: mobility, self-care, usual activities, pain/discomfort, and anxiety/depression is reduced by 4 units about 24 hours after intranasal administration of the racemic ketamine, or a pharmaceutically acceptable salt thereof. In some embodiments, the EQ-5D-5L health state in two or more of 5 domains: mobility, self-care, usual activities, pain/discomfort, and anxiety/depression is reduced by 5 units about 24 hours after intranasal administration of the racemic ketamine, or a pharmaceutically acceptable salt thereof.

In some embodiments, the EQ-5D-5L health state in three or more of 5 domains: mobility, self-care, usual activities, pain/discomfort, and anxiety/depression is reduced by 1 unit about 24 hours after intranasal administration of the racemic ketamine, or a pharmaceutically acceptable salt thereof. In some embodiments, the EQ-5D-5L health state in three or more of 5 domains: mobility, self-care, usual activities, pain/discomfort, and anxiety/depression is reduced by 2 units about 24 hours after intranasal administration of the racemic ketamine, or a pharmaceutically acceptable salt thereof. In some embodiments, the EQ-5D-5L health state in three or more of 5 domains: mobility, self-care, usual activities, pain/discomfort, and anxiety/depression is reduced by 3 units about 24 hours after intranasal administration of the racemic ketamine, or a pharmaceutically acceptable salt thereof. In some embodiments, the EQ-5D-5L health state in three or more of 5 domains: mobility, self-care, usual activities, pain/discomfort, and anxiety/depression is reduced by 4 units about 24 hours after intranasal administration of the racemic ketamine, or a pharmaceutically acceptable salt thereof. In some embodiments, the EQ-5D-5L health state in three or more of 5 domains: mobility, self-care, usual activities, pain/discomfort, and anxiety/depression is reduced by 5 units about 24 hours after intranasal administration of the racemic ketamine, or a pharmaceutically acceptable salt thereof.

In some embodiments, the EQ-5D-5L health state in four to five of 5 domains: mobility, self-care, usual activities, pain/discomfort, and anxiety/depression is reduced by 1 unit about 24 hours after intranasal administration of the racemic ketamine, or a pharmaceutically acceptable salt thereof. In some embodiments, the EQ-5D-5L health state in four to five of 5 domains: mobility, self-care, usual activities, pain/discomfort, and anxiety/depression is reduced by 2 units about 24 hours after intranasal administration of the racemic ketamine, or a pharmaceutically acceptable salt thereof. In some embodiments, the EQ-5D-5L health state in four to five of 5 domains: mobility, self-care, usual activities, pain/discomfort, and anxiety/depression is reduced by 3 units about 24 hours after intranasal administration of the racemic ketamine, or a pharmaceutically acceptable salt thereof. In some embodiments, the EQ-5D-5L health state in four to five of 5 domains: mobility, self-care, usual activities, pain/discomfort, and anxiety/depression is reduced by 4 units about 24 hours after intranasal administration of the racemic ketamine, or a pharmaceutically acceptable salt thereof. In some embodiments, the EQ-5D-5L health state in four to five of 5 domains: mobility, self-care, usual activities, pain/discomfort, and anxiety/depression is reduced by 5 units about 24 hours after intranasal administration of the racemic ketamine, or a pharmaceutically acceptable salt thereof.

In some embodiments, the EQ-5D-5L health state in one or more of 5 domains: mobility, self-care, usual activities, pain/discomfort, and anxiety/depression is reduced by at least 1 unit after intranasal administration of the racemic ketamine, or a pharmaceutically acceptable salt thereof. In some embodiments, the EQ-5D-5L health state in one or more of 5 domains: mobility, self-care, usual activities, pain/discomfort, and anxiety/depression is reduced by at least 2, at least 3, at least 4, or at least 5 after intranasal administration of the racemic ketamine, or a pharmaceutically acceptable salt thereof.

In some embodiments, the EQ-5D-5L health state in one or more of 5 domains: mobility, self-care, usual activities, pain/discomfort, and anxiety/depression is reduced by at least 1 unit after intranasal administration of the racemic ketamine, or a pharmaceutically acceptable salt thereof about 5 minutes to about 24 hours after intranasal administration of the racemic ketamine, or a pharmaceutically acceptable salt thereof. For example, about 5 minutes to 1 hour, about 5 minutes to 6 hours, about 5 minutes to 12 hours, about 5 minutes to 18 hours, about 18 hours to 24 hours, about 12 hours to 24 hours, or about 6 hours to 24 hours after intranasal administration of the racemic ketamine, or a pharmaceutically acceptable salt thereof. In some embodiments, the EQ-5D-5L health state in one or more of 5 domains: mobility, self-care, usual activities, pain/discomfort, and anxiety/depression is reduced by at least 2 units, or at least 3, at least 4, or at least 5 units after intranasal administration of the racemic ketamine, or a pharmaceutically acceptable salt thereof about 5 minutes to about 24 hours after intranasal administration of the racemic ketamine, or a pharmaceutically acceptable salt thereof. For example, about 5 minutes to 1 hour, about 5 minutes to 6 hours, about 5 minutes to 12 hours, about 5 minutes to 18 hours, about 18 hours to 24 hours, about 12 hours to 24 hours, or about 6 hours to 24 hours after intranasal administration of the racemic ketamine, or a pharmaceutically acceptable salt thereof.

In some embodiments, the EQ-5D-5L index value of the subject is reduced as described herein about 1 hour to about 4 hours, about 1.5 hours to about 5 hours, about 2 hours to about 6 hours, or about 5 hours to about 10 hours, after intranasal administration of the racemic ketamine, or a pharmaceutically acceptable salt thereof (e.g., relative to prior to intranasal administration of the racemic ketamine, or a pharmaceutically acceptable salt thereof).

In some embodiments, a subject is administered the EQ-5D-5L prior to intranasal administration of racemic ketamine as described herein. For example, the EQ-5D-5L can be administered to the subject about 1 hour to about 6 months prior to intranasal administration of racemic ketamine as described herein. In some embodiments, the EQ-5D-5L is administered to the subject about 1 hour to about 6 hours, about 1 hour to about 1 day, about 1 hour to about 1 week, about 1 hour to about 1 month, about 1 hour about 3 months, about 3 months to about 6 months, about 1 month to about 6 months, about 1 week to about 5 months, or about 1 day to about 6 months prior to intranasal administration of racemic ketamine as described herein.

The Treatment Satisfaction Questionnaire for Medication (TSQM-9) is a 9-item generic patient-reported outcome instrument to assess a subject's satisfaction with medication. It is derived from the longer TSQM Version 1.4 and covers 3 domains of effectiveness, convenience and global satisfaction. The instrument is scored by domain with scores ranging from 0-100 where a lower score indicates lower satisfaction. The recall period is “the last 2-3 weeks”.

In some embodiments, the TSQM-9 score of the subject is up to 100 units prior to intranasal administration of the racemic ketamine, or a pharmaceutically acceptable salt thereof. In some embodiments, the TSQM-9 score of the subject is up to 75 units prior to intranasal administration of the racemic ketamine, or a pharmaceutically acceptable salt thereof. In some embodiments, the TSQM-9 score of the subject is up to 50 units prior to intranasal administration of the racemic ketamine, or a pharmaceutically acceptable salt thereof.

In some embodiments, the TSQM-9 score of the subject is up to 25 units prior to intranasal administration of the racemic ketamine, or a pharmaceutically acceptable salt thereof. In some embodiments, the TSQM-9 score of the subject is up to 5, 10, 15, or 20 units prior to intranasal administration of the racemic ketamine, or a pharmaceutically acceptable salt thereof.

In some embodiments, the TSQM-9 score of the subject is 10 or less, 10 to 20, 20 to 30, 30 to 40, or 40 to 50 prior to intranasal administration of the racemic ketamine, or a pharmaceutically acceptable salt thereof, and increased by up to 99% at about 24 hours after intranasal administration of the racemic ketamine, or a pharmaceutically acceptable salt thereof. In some embodiments, the TSQM-9 score of the subject is 10 or less, 10 to 20, 20 to 30, 30 to 40, 40 to 50, 50 to 55 prior to intranasal administration of the racemic ketamine, or a pharmaceutically acceptable salt thereof, and increased by up to 80% at about 24 hours after intranasal administration of the racemic ketamine, or a pharmaceutically acceptable salt thereof. In some embodiments, the TSQM-9 score of the subject is 10 or less, 10 to 20, 20 to 30, 30 to 40, 40 to 50, 50 to 60, 60 to 66 prior to intranasal administration of the racemic ketamine, or a pharmaceutically acceptable salt thereof, and is increased by up to 50% at about 24 hours after intranasal administration of the racemic ketamine, or a pharmaceutically acceptable salt thereof. In some embodiments, the TSQM-9 score of the subject is 10 or less, 10 to 20, 20 to 30, 30 to 40, 40 to 50, 50 to 60, 60 to 70, 70 to 80 prior to intranasal administration of the racemic ketamine, or a pharmaceutically acceptable salt thereof, and is increased by up to 25% at about 24 hours after intranasal administration of the racemic ketamine, or a pharmaceutically acceptable salt thereof. In some embodiments, the TSQM-9 score of the subject is 10 or less, 10 to 20, 20 to 30, 30 to 40, 40 to 50, 50 to 60, 60 to 70, 70 to 80, or 80 to 90 prior to intranasal administration of the racemic ketamine, or a pharmaceutically acceptable salt thereof, and is increased by up to 10% at about 24 hours after intranasal administration of the racemic ketamine, or a pharmaceutically acceptable salt thereof. In some embodiments, the TSQM-9 score of the subject is 10 or less, 10 to 20, 20 to 30, 30 to 40, 40 to 50, 50 to 60, 60 to 70, 70 to 80, or 80 to 95 prior to intranasal administration of the racemic ketamine, or a pharmaceutically acceptable salt thereof, and is increased by up to 5% at about 24 hours after intranasal administration of the racemic ketamine, or a pharmaceutically acceptable salt thereof.

In some embodiments, the TSQM-9 score of the subject is up to 100 about 24 hours after intranasal administration of the racemic ketamine, or a pharmaceutically acceptable salt thereof. In some embodiments, the TSQM-9 score of the subject is 10 to 80 units about 24 hours after intranasal administration of the racemic ketamine, or a pharmaceutically acceptable salt thereof. In some embodiments, the TSQM-9 score of the subject is 20 to 70 units about 24 hours after intranasal administration of the racemic ketamine, or a pharmaceutically acceptable salt thereof. In some embodiments, the TSQM-9 score of the subject is 30 to 50 units about 24 hours after intranasal administration of the racemic ketamine, or a pharmaceutically acceptable salt thereof. In some embodiments, the TSQM-9 score of the subject is 0 to 10 units about 24 hours after intranasal administration of the racemic ketamine, or a pharmaceutically acceptable salt thereof. In some embodiments, the TSQM-9 score of the subject is 10 to 20 units about 24 hours after intranasal administration of the racemic ketamine, or a pharmaceutically acceptable salt thereof. In some embodiments, the TSQM-9 score of the subject is 20 to 30 units about 24 hours after intranasal administration of the racemic ketamine, or a pharmaceutically acceptable salt thereof. In some embodiments, the TSQM-9 score of the subject is 30 to 40 units about 24 hours after intranasal administration of the racemic ketamine, or a pharmaceutically acceptable salt thereof. In some embodiments, the TSQM-9 score of the subject is 40 to 50 units about 24 hours after intranasal administration of the racemic ketamine, or a pharmaceutically acceptable salt thereof. In some embodiments, the TSQM-9 score of the subject is 50 to 60 units about 24 hours after intranasal administration of the racemic ketamine, or a pharmaceutically acceptable salt thereof. In some embodiments, the TSQM-9 score of the subject is 60 to 70 units about 24 hours after intranasal administration of the racemic ketamine, or a pharmaceutically acceptable salt thereof. In some embodiments, the TSQM-9 score of the subject is 70 to 80 units about 24 hours after intranasal administration of the racemic ketamine, or a pharmaceutically acceptable salt thereof. In some embodiments, the TSQM-9 score of the subject is up to 80, up to 90, up to 95, or up to 100 units about 24 hours after intranasal administration of the racemic ketamine, or a pharmaceutically acceptable salt thereof.

In some embodiments, the TSQM-9 score of the subject is increased as described herein about 5 minutes to about 24 hours after intranasal administration of the racemic ketamine, or a pharmaceutically acceptable salt thereof. For example, about 5 minutes to 1 hour, about 5 minutes to 6 hours, about 5 minutes to 12 hours, about 5 minutes to 18 hours, about 18 hours to 24 hours, about 12 hours to 24 hours, or about 6 hours to 24 hours after intranasal administration of the racemic ketamine, or a pharmaceutically acceptable salt thereof. In some embodiments, the TSQM-9 score of the subject is increased as described about 1 hour to about 4 hours, about 1.5 hours to about 5 hours, about 2 hours to about 6 hours, or about 5 hours to about 10 hours, after intranasal administration of the racemic ketamine, or a pharmaceutically acceptable salt thereof (e.g., relative to prior to intranasal administration of the racemic ketamine, or a pharmaceutically acceptable salt thereof).

In some embodiments, a subject is administered the TSQM-9 prior to intranasal administration of racemic ketamine as described herein. In some embodiments, the TSQM-9 is administered to the subject about 1 hour to about 6 hours, about 1 hour to about 1 day, about 1 hour to about 1 week, about 1 hour to about 1 month, about 1 hour about 3 months, about 3 months to about 6 months, about 1 month to about 6 months, about 1 week to about 5 months, or about 1 day to about 6 months prior to intranasal administration of racemic ketamine as described herein.

Quality of Life In Depression Scale (QLDS) is a depression-specific patient-reported measure of quality of life in patients in need of anti-depressant therapy, i.e., it captures the impact of depression and its treatment from the patient's perspective. The instrument has a recall period of “at the moment”, and contains 34 items with “true”/“not true” response options. The score range is from 0 (good quality of life) to 34 (very poor quality of life).

In some embodiments, the Quality of Life In Depression Scale (QLDS) score of the subject is up to 34 units prior to intranasal administration of the racemic ketamine, or a pharmaceutically acceptable salt thereof. In some embodiments, the QLDS score of the subject is at least 25 units prior to intranasal administration of the racemic ketamine, or a pharmaceutically acceptable salt thereof. In some embodiments, the QLDS score of the subject is at least 15 units prior to intranasal administration of the racemic ketamine, or a pharmaceutically acceptable salt thereof. In some embodiments, the QLDS score of the subject is 5 to 10 units prior to intranasal administration of the racemic ketamine, or a pharmaceutically acceptable salt thereof. In some embodiments, the QLDS score of the subject is up to 5 units prior to intranasal administration of the racemic ketamine, or a pharmaceutically acceptable salt thereof. In some embodiments, the QLDS score of the subject is 5 to 34 units prior to intranasal administration of the racemic ketamine, or a pharmaceutically acceptable salt thereof. In some embodiments, the QLDS score of the subject is 10 to 15 units prior to intranasal administration of the racemic ketamine, or a pharmaceutically acceptable salt thereof. In some embodiments, the QLDS score of the subject is 0 to 15 units prior to intranasal administration of the racemic ketamine, or a pharmaceutically acceptable salt thereof. In some embodiments, the QLDS score of the subject is 15 to 34 units prior to intranasal administration of the racemic ketamine, or a pharmaceutically acceptable salt thereof.

In some embodiments, the QLDS score of the subject is 0 to 5, 5 to 10, 10 to 20, 20 to 25, or 25 to 34 prior to intranasal administration of the racemic ketamine, or a pharmaceutically acceptable salt thereof, and is reduced by at least 80% at about 24 hours after intranasal administration of the racemic ketamine, or a pharmaceutically acceptable salt thereof. In some embodiments, the QLDS score of the subject is 0 to 5, 5 to 10, 10 to 20, 20 to 25, or 25 to 34 prior to intranasal administration of the racemic ketamine, or a pharmaceutically acceptable salt thereof, and is reduced by at least 70% at about 24 hours after intranasal administration of the racemic ketamine, or a pharmaceutically acceptable salt thereof. In some embodiments, the QLDS score of the subject is 0 to 5, 5 to 10, 10 to 20, 20 to 25, or 25 to 34 prior to intranasal administration of the racemic ketamine, or a pharmaceutically acceptable salt thereof, and is reduced by at least 60% at about 24 hours after intranasal administration of the racemic ketamine, or a pharmaceutically acceptable salt thereof. In some embodiments, the QLDS score of the subject is 0 to 5, 5 to 10, 10 to 20, 20 to 25, or 25 to 34 prior to intranasal administration of the racemic ketamine, or a pharmaceutically acceptable salt thereof, and is reduced by at least 50% at about 24 hours after intranasal administration of the racemic ketamine, or a pharmaceutically acceptable salt thereof. In some embodiments, the QLDS score of the subject is 0 to 5, 5 to 10, 10 to 20, 20 to 25, or 25 to 34 prior to intranasal administration of the racemic ketamine, or a pharmaceutically acceptable salt thereof, and is reduced by at least 40% at about 24 hours after intranasal administration of the racemic ketamine, or a pharmaceutically acceptable salt thereof. In some embodiments, the QLDS score of the subject is 0 to 5, 5 to 10, 10 to 20, 20 to 25, or 25 to 34 prior to intranasal administration of the racemic ketamine, or a pharmaceutically acceptable salt thereof, and is reduced by at least 30% at about 24 hours after intranasal administration of the racemic ketamine, or a pharmaceutically acceptable salt thereof. In some embodiments, the QLDS score of the subject is 0 to 5, 5 to 10, 10 to 20, 20 to 25, or 25 to 34 prior to intranasal administration of the racemic ketamine, or a pharmaceutically acceptable salt thereof, and is reduced by at least 20% at about 24 hours after intranasal administration of the racemic ketamine, or a pharmaceutically acceptable salt thereof. In some embodiments, the QLDS score of the subject is 0 to 5, 5 to 10, 10 to 20, 20 to 25, or 25 to 34 prior to intranasal administration of the racemic ketamine, or a pharmaceutically acceptable salt thereof, and is reduced by at least 10% at about 24 hours after intranasal administration of the racemic ketamine, or a pharmaceutically acceptable salt thereof. In some embodiments, the QLDS score of the subject is 0 to 5, 5 to 10, 10 to 20, 20 to 25, or 25 to 34 prior to intranasal administration of the racemic ketamine, or a pharmaceutically acceptable salt thereof, and is reduced by at least 5% at about 24 hours after intranasal administration of the racemic ketamine, or a pharmaceutically acceptable salt thereof.

In some embodiments, the QLDS score of the subject is a least 1, at least 2, at least 3, at least 4, or at least 5 units about 24 hours after intranasal administration of the racemic ketamine, or a pharmaceutically acceptable salt thereof. In some embodiments, the QLDS score of the subject is at least 10 units about 24 hours after intranasal administration of the racemic ketamine, or a pharmaceutically acceptable salt thereof. In some embodiments, the QLDS score of the subject is at least 15 units about 24 hours after intranasal administration of the racemic ketamine, or a pharmaceutically acceptable salt thereof. In some embodiments, the QLDS score of the subject is at least 20 units about 24 hours after intranasal administration of the racemic ketamine, or a pharmaceutically acceptable salt thereof. In some embodiments, the QLDS score of the subject is at least 25 units about 24 hours after intranasal administration of the racemic ketamine, or a pharmaceutically acceptable salt thereof. In some embodiments, the QLDS score of the subject is 34 or less units about 24 hours after intranasal administration of the racemic ketamine, or a pharmaceutically acceptable salt thereof.

In some embodiments, the QLDS score of the subject is decreased as described herein about 5 minutes to about 24 hours after intranasal administration of the racemic ketamine, or a pharmaceutically acceptable salt thereof. For example, about 5 minutes to 1 hour, about 5 minutes to 6 hours, about 5 minutes to 12 hours, about 5 minutes to 18 hours, about 18 hours to 24 hours, about 12 hours to 24 hours, or about 6 hours to 24 hours after intranasal administration of the racemic ketamine, or a pharmaceutically acceptable salt thereof. In some embodiments, the QLDS score of the subject is decreased as described about 1 hour to about 4 hours, about 1.5 hours to about 5 hours, about 2 hours to about 6 hours, or about 5 hours to about 10 hours, after intranasal administration of the racemic ketamine, or a pharmaceutically acceptable salt thereof (e.g., relative to prior to intranasal administration of the racemic ketamine, or a pharmaceutically acceptable salt thereof).

In some embodiments, a subject is administered the Quality of Life In Depression Scale (QLDS) prior to intranasal administration of racemic ketamine as described herein. In some embodiments, the Quality of Life In Depression Scale (QLDS) is administered to the subject about 1 hour to about 6 hours, about 1 hour to about 1 day, about 1 hour to about 1 week, about 1 hour to about 1 month, about 1 hour about 3 months, about 3 months to about 6 months, about 1 month to about 6 months, about 1 week to about 5 months, or about 1 day to about 6 months prior to intranasal administration of racemic ketamine as described herein.

Healthcare Resource Use Questionnaire (HRUQ) is an instrument designed to capture medical resource utilization data. The HRUQ includes information regarding utilization of healthcare services (including the timing and type of serves), enabling changes in level and quantity of services to be considered as a variable in economic models. The HRUQ is not a fixed questionnaire and may vary depending the circumstances and trial design.

In some embodiments, a subject is administered the HRUQ prior to intranasal administration of racemic ketamine as described herein. In some embodiments, HRUQ is administered to the subject about 1 hour to about 6 hours, about 1 hour to about 1 day, about 1 hour to about 1 week, about 1 hour to about 1 month, about 1 hour about 3 months, about 3 months to about 6 months, about 1 month to about 6 months, about 1 week to about 5 months, or about 1 day to about 6 months prior to intranasal administration of racemic ketamine as described herein.

In some embodiments, the HRUQ indicates a net reduction in utilization of healthcare services after intranasal administration of the racemic ketamine, or a pharmaceutically acceptable salt thereof, relative to the level and extent recorded prior to intranasal administration of the racemic ketamine, or a pharmaceutically acceptable salt thereof.

In some embodiments, the HRUQ indicates a net reduction as described herein about 1 to 24 hours after intranasal administration of the racemic ketamine, or a pharmaceutically acceptable salt thereof. For example, about 1 hour, about 1 to 6 hours, about 1 to 12 hours, about 1 to 18 hours, about 18 hours to 24 hours, about 12 hours to 24 hours, or about 6 hours to 24 hours after intranasal administration of the racemic ketamine, or a pharmaceutically acceptable salt thereof. In some embodiments, the HRUQ indicates a net reduction as described herein about 1 day to about 4 days, about 1 day to about 5 days, about 5 days to about 10 days, about 10 days to about 30 days, about 1 month to 6 months, or 6 months to 12 months after intranasal administration of the racemic ketamine, or a pharmaceutically acceptable salt thereof (relative to prior to intranasal administration of the racemic ketamine, or a pharmaceutically acceptable salt thereof).

The Beck Hopelessness Scale (BHS) is a patient-reported measure to assess the level of a subject's negative expectations or pessimism regarding the future, and as such, is an important future predictor of suicide. The BHS consists of 20 true-false items that examine the respondent's attitude over the past week by either endorsing a pessimistic statement or denying an optimistic statement; 9 are keyed false and 11 are keyed true. These items fall within 3 domains: (1) feelings about the
future; (2) loss of motivation; and (3) future expectations. For every statement, each response is assigned a score of 0 or 1. The total BHS score is a sum of item responses and can range from 0 to 20, with a higher score representing a higher level of hopelessness. Total scores that range from 0 to 3 are considered within the normal range, scores 4 to 8 identify mild hopelessness, scores 9 to 14 identify moderate hopelessness, and scores greater than 14 identify severe hopelessness.

In some embodiments, the BHS score of the subject is up to 20 units prior to intranasal administration of the racemic ketamine, or a pharmaceutically acceptable salt thereof. In some embodiments, the BHS score of the subject is at least 15 units prior to intranasal administration of the racemic ketamine, or a pharmaceutically acceptable salt thereof. In some embodiments, the BHS score of the subject is at least 10 units prior to intranasal administration of the racemic ketamine, or a pharmaceutically acceptable salt thereof. In some embodiments, the BHS score of the subject is at least 4, at least 5, at least 6, at least 7, at least 8, or at least 9 units prior to intranasal administration of the racemic ketamine, or a pharmaceutically acceptable salt thereof. In some embodiments the BHS score of the subject is from 0 to 20 units prior to intranasal administration of the racemic ketamine, or a pharmaceutically acceptable salt thereof. In some embodiments, the BHS score of the subject is from 3 to 18 units prior to intranasal administration of the racemic ketamine, or a pharmaceutically acceptable salt thereof. In some embodiments, the BHS score of the subject is from 5 to 15 units prior to intranasal administration of the racemic ketamine, or a pharmaceutically acceptable salt thereof. In some embodiments, the BHS score of the subject is from 9 to 12 units prior to intranasal administration of the racemic ketamine, or a pharmaceutically acceptable salt thereof. In some embodiments the BHS score of the subject is from 4 to 20 units prior to intranasal administration of the racemic ketamine, or a pharmaceutically acceptable salt thereof. In some embodiments the BHS score of the subject is from 8 to 20 units prior to intranasal administration of the racemic ketamine, or a pharmaceutically acceptable salt thereof.

In some embodiments, the BHS score of the subject is 0 to 5, 5 to 10, 10 to 15, or 15 to 20 prior to intranasal administration of the racemic ketamine, or a pharmaceutically acceptable salt thereof, and is reduced by at least 80% at about 24 hours after intranasal administration of the racemic ketamine, or a pharmaceutically acceptable salt thereof. In some embodiments, the BHS score of the subject is 0 to 5, 5 to 10, 10 to 15, or 15 to 20 prior to intranasal administration of the racemic ketamine, or a pharmaceutically acceptable salt thereof, and is reduced by at least 70% at about 24 hours after intranasal administration of the racemic ketamine, or a pharmaceutically acceptable salt thereof. In some embodiments, the BHS score of the subject is 0 to 5, 5 to 10, 10 to 15, or 15 to 20 prior to intranasal administration of the racemic ketamine, or a pharmaceutically acceptable salt thereof, and is reduced by at least 60% at about 24 hours after intranasal administration of the racemic ketamine, or a pharmaceutically acceptable salt thereof. In some embodiments, the BHS score of the subject is 0 to 5, 5 to 10, 10 to 15, or 15 to 20 prior to intranasal administration of the racemic ketamine, or a pharmaceutically acceptable salt thereof, and is reduced by at least 50% at about 24 hours after intranasal administration of the racemic ketamine, or a pharmaceutically acceptable salt thereof. In some embodiments, the BHS score of the subject is 0 to 5, 5 to 10, 10 to 15, or 15 to 20 prior to intranasal administration of the racemic ketamine, or a pharmaceutically acceptable salt thereof, and is reduced by at least 40% at about 24 hours after intranasal administration of the racemic ketamine, or a pharmaceutically acceptable salt thereof. In some embodiments, the BHS score of the subject is 0 to 5, 5 to 10, 10 to 15, or 15 to 20 prior to intranasal administration of the racemic ketamine, or a pharmaceutically acceptable salt thereof, and is reduced by at least 30% at about 24 hours after intranasal administration of the racemic ketamine, or a pharmaceutically acceptable salt thereof. In some embodiments, the BHS score of the subject is 0 to 5, 5 to 10, 10 to 15, or 15 to 20 prior to intranasal administration of the racemic ketamine, or a pharmaceutically acceptable salt thereof, and is reduced by at least 20% at about 24 hours after intranasal administration of the racemic ketamine, or a pharmaceutically acceptable salt thereof. In some embodiments, the BHS score of the subject is 0 to 5, 5 to 10, 10 to 15, or 15 to 20 prior to intranasal administration of the racemic ketamine, or a pharmaceutically acceptable salt thereof, and is reduced by at least 10% at about 24 hours after intranasal administration of the racemic ketamine, or a pharmaceutically acceptable salt thereof. In some embodiments, the BHS score of the subject is 0 to 5, 5 to 10, 10 to 15, or 15 to 20 prior to intranasal administration of the racemic ketamine, or a pharmaceutically acceptable salt thereof, and is reduced by 5 to 10% at about 24 hours after intranasal administration of the racemic ketamine, or a pharmaceutically acceptable salt thereof. In some embodiments, the BHS score of the subject is 0 to 5, 5 to 10, 10 to 15, or 15 to 20 prior to intranasal administration of the racemic ketamine, or a pharmaceutically acceptable salt thereof, and is reduced by up to 5% at about 24 hours after intranasal administration of the racemic ketamine, or a pharmaceutically acceptable salt thereof.

In some embodiments, the BHS score of the subject is up to 1, at least 2, at least 3, at least 4, or at least 5 units about 24 hours after intranasal administration of the racemic ketamine, or a pharmaceutically acceptable salt thereof. In some embodiments, the BHS score of the subject is at least 6, at least 7, at least 8, at least 9, at least 10, or at least 11 units about 24 hours after intranasal administration of the racemic ketamine, or a pharmaceutically acceptable salt thereof. In some embodiments, the BHS score of the subject is at least 12, at least 13, at least 14, at least 16, or at least 16 units about 24 hours after intranasal administration of the racemic ketamine, or a pharmaceutically acceptable salt thereof. In some embodiments, the BHS score of the subject is at least 17, at least 18, at least 19 units about 24 hours after intranasal administration of the racemic ketamine, or a pharmaceutically acceptable salt thereof. In some embodiments, the BHS score of the subject is 20 or less units about 24 hours after intranasal administration of the racemic ketamine, or a pharmaceutically acceptable salt thereof. In some embodiments, the BHS score of the subject is 14 or less units about 24 hours after intranasal administration of the racemic ketamine, or a pharmaceutically acceptable salt thereof. In some embodiments, the BHS score of the subject is 9 or less units about 24 hours after intranasal administration of the racemic ketamine, or a pharmaceutically acceptable salt thereof. In some embodiments, the BHS score of the subject is 4 or less units about 24 hours after intranasal administration of the racemic ketamine, or a pharmaceutically acceptable salt thereof.

In some embodiments, the BHS score of the subject is decreased as described herein about 5 minutes to about 24 hours after intranasal administration of the racemic ketamine, or a pharmaceutically acceptable salt thereof. For example, about 5 minutes to 1 hour, about 5 minutes to 6 hours, about 5 minutes to 12 hours, about 5 minutes to 18 hours, about 18 hours to 24 hours, about 12 hours to 24 hours, or about 6 hours to 24 hours after intranasal administration of the racemic ketamine, or a pharmaceutically acceptable salt thereof. In some embodiments, the BHS score of the subject is decreased as described about 1 hour to about 4 hours, about 1.5 hours to about 5 hours, about 2 hours to about 6 hours, or about 5 hours to about 10 hours, after intranasal administration of the racemic ketamine, or a pharmaceutically acceptable salt thereof (e.g., relative to prior to intranasal administration of the racemic ketamine, or a pharmaceutically acceptable salt thereof).

In some embodiments, a subject is administered the Beck Hopelessness Scale (BHS) prior to intranasal administration of racemic ketamine as described herein. In some embodiments, the Beck Hopelessness Scale (BHS) is administered to the subject about 1 hour to about 6 hours, about 1 hour to about 1 day, about 1 hour to about 1 week, about 1 hour to about 1 month, about 1 hour about 3 months, about 3 months to about 6 months, about 1 month to about 6 months, about 1 week to about 5 months, or about 1 day to about 6 months prior to intranasal administration of racemic ketamine as described herein.

Bladder Pain/Interstitial Cystitis Symptom Score (BPIC-SS) is a psychometrically validated and reliable questionnaire with 8 questions concerning bladder pain over the previous 7 days. Questions 1 to 5 assess urinary symptoms (how often urinated because of pain, need to urinate just after previous urination, urination to avoid pain, pressure in the bladder, and pain in the bladder) and are rated 0 (never) to 4 (always). Questions 6 and 7 assess the impact of bladder pain (bothered by frequent urination during daytime and nighttime) and are rated 0 (not at all) to 4 (a great deal). Question 8 assesses the worst pain on a 0 (no bladder pain) to 10 (worst possible bladder pain) numerical pain scale. The BPIC-SS total score is the sum of the individual question scores and range from 0 to 38, with higher scores indicating a worse situation. A score of 19 or more represents moderate/severe disease activity. A negative change indicates a reduction/improvement from baseline.

In some embodiments, the BPIC-SS total score of the subject is up to 38 units prior to intranasal administration of the racemic ketamine, or a pharmaceutically acceptable salt thereof. In some embodiments, the BPIC-SS total score of the subject from 38 to 32 units prior to intranasal administration of the racemic ketamine, or a pharmaceutically acceptable salt thereof. In some embodiments, the BPIC-SS total score of the subject from 32 to 27 units prior to intranasal administration of the racemic ketamine, or a pharmaceutically acceptable salt thereof. In some embodiments, the BPIC-SS total score of the subject is from 27 to 22 units prior to intranasal administration of the racemic ketamine, or a pharmaceutically acceptable salt thereof. In some embodiments, the BPIC-SS total score of the subject is from 22 to 17 units, 17 to 13, 13 to 11, or 11 to 9 units prior to intranasal administration of the racemic ketamine, or a pharmaceutically acceptable salt thereof. In some embodiments, the BPIC-SS total score of the subject from 8 to 6 units, 6 to 4, 4 to 2, or 1 to 0 units prior to intranasal administration of the racemic ketamine, or a pharmaceutically acceptable salt thereof.

In some embodiments, the BPIC-SS total score of the subject is 0 to 5, 5 to 15, 15 to 25, 25 to 35, or 35 to 38 prior to intranasal administration of the racemic ketamine, or a pharmaceutically acceptable salt thereof, and is reduced by at least 80% at about 24 hours after intranasal administration of the racemic ketamine, or a pharmaceutically acceptable salt thereof. In some embodiments, the BPIC-SS total score of the subject is 0 to 5, 5 to 15, 15 to 25, 25 to 35, or 35 to 38 prior to intranasal administration of the racemic ketamine, or a pharmaceutically acceptable salt thereof, and is reduced by at least 70% at about 24 hours after intranasal administration of the racemic ketamine, or a pharmaceutically acceptable salt thereof. In some embodiments, the BPIC-SS total score of the subject is 0 to 5, 5 to 15, 15 to 25, 25 to 35, or 35 to 38 prior to intranasal administration of the racemic ketamine, or a pharmaceutically acceptable salt thereof, and is reduced by at least 60% at about 24 hours after intranasal administration of the racemic ketamine, or a pharmaceutically acceptable salt thereof. In some embodiments, the BPIC-SS total score of the subject is 0 to 5, 5 to 15, 15 to 25, 25 to 35, or 35 to 38 prior to intranasal administration of the racemic ketamine, or a pharmaceutically acceptable salt thereof, and is reduced by at least 50% at about 24 hours after intranasal administration of the racemic ketamine, or a pharmaceutically acceptable salt thereof. In some embodiments, the BPIC-SS total score of the subject is 0 to 5, 5 to 15, 15 to 25, 25 to 35, or 35 to 38 prior to intranasal administration of the racemic ketamine, or a pharmaceutically acceptable salt thereof, and is reduced by at least 40% at about 24 hours after intranasal administration of the racemic ketamine, or a pharmaceutically acceptable salt thereof. In some embodiments, the BPIC-SS total score of the subject is 0 to 5, 5 to 15, 15 to 25, 25 to 35, or 35 to 38 prior to intranasal administration of the racemic ketamine, or a pharmaceutically acceptable salt thereof, and is reduced by at least 30% at about 24 hours after intranasal administration of the racemic ketamine, or a pharmaceutically acceptable salt thereof. In some embodiments, the BPIC-SS total score of the subject is 0 to 5, 5 to 15, 15 to 25, 25 to 35, or 35 to 38 prior to intranasal administration of the racemic ketamine, or a pharmaceutically acceptable salt thereof, and is reduced by at least 20% at about 24 hours after intranasal administration of the racemic ketamine, or a pharmaceutically acceptable salt thereof. In some embodiments, the BPIC-SS total score of the subject is 0 to 5, 5 to 15, 15 to 25, 25 to 35, or 35 to 38 prior to intranasal administration of the racemic ketamine, or a pharmaceutically acceptable salt thereof, and is reduced by at least 10% at about 24 hours after intranasal administration of the racemic ketamine, or a pharmaceutically acceptable salt thereof. In some embodiments, the BPIC-SS total score of the subject is 0 to 5, 5 to 15, 15 to 25, 25 to 35, or 35 to 38 prior to intranasal administration of the racemic ketamine, or a pharmaceutically acceptable salt thereof, and is reduced by 10 to 5% or less at about 24 hours after intranasal administration of the racemic ketamine, or a pharmaceutically acceptable salt thereof.

In some embodiments, the BPIC-SS total score of the subject is a most 1 to 2, at most 3 to 4, at most 4 to 5, or at most 5 to 6 units about 24 hours after intranasal administration of the racemic ketamine, or a pharmaceutically acceptable salt thereof. In some embodiments, the BPIC-SS total score of the subject is at most 7 to 8, at most 9 to 10, at most 11 to 12, at most 13 to 14, at most 15 to 16, or at most 17 to 18 units about 24 hours after intranasal administration of the racemic ketamine, or a pharmaceutically acceptable salt thereof. In some embodiments, the BPIC-SS total score of the subject is at most 19, at most 20 to 21, at most 22 to 23, at most 24 to 25, or at most 26 to 27 units about 24 hours after intranasal administration of the racemic ketamine, or a pharmaceutically acceptable salt thereof. In some embodiments, the BPIC-SS total score of the subject is at most 28, at most 29, at most 30 to 31, or at most 32 units about 24 hours after intranasal administration of the racemic ketamine, or a pharmaceutically acceptable salt thereof. In some embodiments, the BPIC-SS total score of the subject is at most 33 to 34, at most 35 to 36, or at most 36 to 38 units about 24 hours after intranasal administration of the racemic ketamine, or a pharmaceutically acceptable salt thereof.

In some embodiments, the BPIC-SS total score of the subject is decreased as described herein about 5 minutes to about 24 hours after intranasal administration of the racemic ketamine, or a pharmaceutically acceptable salt thereof. For example, about 5 minutes to 1 hour, about 5 minutes to 6 hours, about 5 minutes to 12 hours, about 5 minutes to 18 hours, about 18 hours to 24 hours, about 12 hours to 24 hours, or about 6 hours to 24 hours after intranasal administration of the racemic ketamine, or a pharmaceutically acceptable salt thereof. In some embodiments, the BPIC-SS total score of the subject is decreased as described about 1 hour to about 4 hours, about 1.5 hours to about 5 hours, about 2 hours to about 6 hours, or about 5 hours to about 10 hours, after intranasal administration of the racemic ketamine, or a pharmaceutically acceptable salt thereof (e.g., relative to prior to intranasal administration of the racemic ketamine, or a pharmaceutically acceptable salt thereof).

In some embodiments, a subject is administered the BPIC-SS total prior to intranasal administration of racemic ketamine as described herein. In some embodiments, the BPIC-SS total is administered to the subject about 1 hour to about 6 hours, about 1 hour to about 1 day, about 1 hour to about 1 week, about 1 hour to about 1 month, about 1 hour about 3 months, about 3 months to about 6 months, about 1 month to about 6 months, about 1 week to about 5 months, or about 1 day to about 6 months prior to intranasal administration of racemic ketamine as described herein.

Complex regional pain syndrome (CRPS) is a chronic neurologic condition resulting from a traumatic insult. The clinical diagnosis of CRPS is a dichotomous (yes/no) categorization necessary for clinical decision-making. The CRPS Severity Score (CSS) is a tool to quantify clinical features associated with CRPS based on the presence/absence of 16 clinically-assessed signs (8) and symptoms (8), with one point being given to each. It is a continuous outcome measure that corresponds with and complements the dichotomous (yes/no) IASP diagnostic criteria for CRPS (the “Budapest criteria”). The test comprises a checklist of signs and symptoms common with CRPS including self-reported symptoms such as allodynia, bilateral temperature asymmetry, skin color asymmetry, sweating asymmetry, trophic changes, motor changes, decreased range of motion, asymmetric edema, and clinical signs observed by an examiner such as hyperpathia to pinprick, allodynia, temperature asymmetry to palpation, skin color asymmetry, sweating asymmetry, asymmetric edema, trophic changes, motor changes, and decreased active range of motion. Higher scores indicate a greater CRPS severity.

In some embodiments, the CSS score of the subject is 14 or greater (e.g., 14, 15, or 16) prior to intranasal administration of the racemic ketamine, or a pharmaceutically acceptable salt thereof. In some embodiments, the CSS score of the subject is 12 or greater prior to intranasal administration of the racemic ketamine, or a pharmaceutically acceptable salt thereof. In some embodiments, the CSS score of the subject is 10 or greater prior to intranasal administration of the racemic ketamine, or a pharmaceutically acceptable salt thereof. In some embodiments, the CSS score of the subject is 8 or greater prior to intranasal administration of the racemic ketamine, or a pharmaceutically acceptable salt thereof. In some embodiments, the CSS score of the subject is 6 or greater prior to intranasal administration of the racemic ketamine, or a pharmaceutically acceptable salt thereof. In some embodiments the CSS score of the subject is 4 or greater prior to intranasal administration of the racemic ketamine, or a pharmaceutically acceptable salt thereof. In some embodiments, the CSS score of the subject is 1 or greater prior to intranasal administration of the racemic ketamine, or a pharmaceutically acceptable salt thereof.

In some embodiments, the CSS score of the subject is 14 or greater, about 5 minutes to about 24 hours prior to intranasal administration of the racemic ketamine, or a pharmaceutically acceptable salt thereof. For example, about 5 minutes to 1 hour, about 5 minutes to 6 hours, about 5 minutes to 12 hours, about 5 minutes to 18 hours, about 18 hours to 24 hours, about 12 hours to 24 hours, or about 6 hours to 24 hours prior to intranasal administration of the racemic ketamine, or a pharmaceutically acceptable salt thereof. In some embodiments, the CSS score of the subject is 12 or greater, about 5 minutes to about 24 hours prior to intranasal administration of the racemic ketamine, or a pharmaceutically acceptable salt thereof. For example, about 5 minutes to 1 hour, about 5 minutes to 6 hours, about 5 minutes to 12 hours, about 5 minutes to 18 hours, about 18 hours to 24 hours, about 12 hours to 24 hours, or about 6 hours to 24 hours prior to intranasal administration of the racemic ketamine, or a pharmaceutically acceptable salt thereof. In some embodiments, the CSS score of the subject is 10 or greater, about 5 minutes to about 24 hours prior to intranasal administration of the racemic ketamine, or a pharmaceutically acceptable salt thereof. For example, about 5 minutes to 1 hour, about 5 minutes to 6 hours, about 5 minutes to 12 hours, about 5 minutes to 18 hours, about 18 hours to 24 hours, about 12 hours to 24 hours, or about 6 hours to 24 hours prior to intranasal administration of the racemic ketamine, or a pharmaceutically acceptable salt thereof. In some embodiments, the CSS score of the subject is 6 or greater, about 5 minutes to about 24 hours prior to intranasal administration of the racemic ketamine, or a pharmaceutically acceptable salt thereof. For example, about 5 minutes to 1 hour, about 5 minutes to 6 hours, about 5 minutes to 12 hours, about 5 minutes to 18 hours, about 18 hours to 24 hours, about 12 hours to 24 hours, or about 6 hours to 24 hours prior to intranasal administration of the racemic ketamine, or a pharmaceutically acceptable salt thereof. In some embodiments, the CSS score of the subject is 4 or greater, about 5 minutes to about 24 hours prior to intranasal administration of the racemic ketamine, or a pharmaceutically acceptable salt thereof. For example, about 5 minutes to 1 hour, about 5 minutes to 6 hours, about 5 minutes to 12 hours, about 5 minutes to 18 hours, about 18 hours to 24 hours, about 12 hours to 24 hours, or about 6 hours to 24 hours prior to intranasal administration of the racemic ketamine, or a pharmaceutically acceptable salt thereof. In some embodiments, the CSS score of the subject is 1 or greater, about 5 minutes to about 24 hours prior to intranasal administration of the racemic ketamine, or a pharmaceutically acceptable salt thereof. For example, about 5 minutes to 1 hour, about 5 minutes to 6 hours, about 5 minutes to 12 hours, about 5 minutes to 18 hours, about 18 hours to 24 hours, about 12 hours to 24 hours, or about 6 hours to 24 hours prior to intranasal administration of the racemic ketamine, or a pharmaceutically acceptable salt thereof.

In some embodiments, the CSS score of the subject decreases by 1 to 16 (e.g., by 1 to 16 units) after intranasal administration of the racemic ketamine, or a pharmaceutically acceptable salt thereof. In some embodiments, the CSS score of the subject decreases by 4 to 14 (e.g., by 4 to 14 units) after intranasal administration of the racemic ketamine, or a pharmaceutically acceptable salt thereof. In some embodiments, the CSS score of the subject decreases by 6 to 12 (e.g., by 6 to 12 units) after intranasal administration of the racemic ketamine, or a pharmaceutically acceptable salt thereof. In some embodiments, the CSS score of the subject decreases by 4 to 6 (e.g., 4 to 6 units) after intranasal administration of the racemic ketamine, or a pharmaceutically acceptable salt thereof. In some embodiments, the CSS score of the subject decreases by 2 to 4 (e.g., 2 to 4 units) after intranasal administration of the racemic ketamine, or a pharmaceutically acceptable salt thereof. In some embodiments, the CSS score of the subject decreases by 1 to 2 (e.g., 1 to 2 units) after intranasal administration of the racemic ketamine, or a pharmaceutically acceptable salt thereof.

Patient-Reported Outcomes Measurement Information System 29 (PROMIS-29) is a screening tool for monitoring the level of interference of pain with various activities. The PROMIS-29 v2.0 profile assesses pain intensity using a single 1-10 numeric rating scale and seven health domains (physical function, fatigue, pain interference, depressive symptoms, anxiety, ability to participate in social roles and activities, and sleep disturbance) using four activities per domain. Each of the four activities per domain is ranked 1-5 as 1=″not at all (meaning the subject feels no pain interference doing the activity)“, 2=″a little bit”, 3=′somewhat“, 4=″quite a bit”, and 5=″very much (meaning the subject experiences a lot of pain interference doing the activity)”. A higher PROMIS score represents more of the concept being measured. The sum of the PROMIS results in the subject's raw domain score, which ranges from 4 to 20.

In some embodiments, the PROMIS-29 raw score of the subject is 18 or greater prior to intranasal administration of the racemic ketamine, or a pharmaceutically acceptable salt thereof. In some embodiments, the PROMIS-29 raw score of the subject is 16 or greater prior to intranasal administration of the racemic ketamine, or a pharmaceutically acceptable salt thereof. In some embodiments, the PROMIS-29 raw score of the subject is 12 or greater prior to intranasal administration of the racemic ketamine, or a pharmaceutically acceptable salt thereof. In some embodiments, the PROMIS-29 raw score of the subject is 10 or greater prior to intranasal administration of the racemic ketamine, or a pharmaceutically acceptable salt thereof. In some embodiments, the PROMIS-29 raw score of the subject is 8 or greater prior to intranasal administration of the racemic ketamine, or a pharmaceutically acceptable salt thereof. In some embodiments, the PROMIS-29 raw score of the subject is 6 or greater prior to intranasal administration of the racemic ketamine, or a pharmaceutically acceptable salt thereof. In some embodiments, the PROMIS-29 raw score of the subject is 4 or greater prior to intranasal administration of the racemic ketamine, or a pharmaceutically acceptable salt thereof.

In some embodiments, the PROMIS-29 raw score of the subject is 18 or greater, about 5 minutes to about 24 hours prior to intranasal administration of the racemic ketamine, or a pharmaceutically acceptable salt thereof. For example, about 5 minutes to 1 hour, about 5 minutes to 6 hours, about 5 minutes to 12 hours, about 5 minutes to 18 hours, about 18 hours to 24 hours, about 12 hours to 24 hours, or about 6 hours to 24 hours prior to intranasal administration of the racemic ketamine, or a pharmaceutically acceptable salt thereof. In some embodiments, the PROMIS-29 raw score of the subject is 16 or greater, about 5 minutes to about 24 hours prior to intranasal administration of the racemic ketamine, or a pharmaceutically acceptable salt thereof. For example, about 5 minutes to 1 hour, about 5 minutes to 6 hours, about 5 minutes to 12 hours, about 5 minutes to 18 hours, about 18 hours to 24 hours, about 12 hours to 24 hours, or about 6 hours to 24 hours prior to intranasal administration of the racemic ketamine, or a pharmaceutically acceptable salt thereof. In some embodiments, the PROMIS-29 raw score of the subject is 12 or greater, about 5 minutes to about 24 hours prior to intranasal administration of the racemic ketamine, or a pharmaceutically acceptable salt thereof. For example, about 5 minutes to 1 hour, about 5 minutes to 6 hours, about 5 minutes to 12 hours, about 5 minutes to 18 hours, about 18 hours to 24 hours, about 12 hours to 24 hours, or about 6 hours to 24 hours prior to intranasal administration of the racemic ketamine, or a pharmaceutically acceptable salt thereof. In some embodiments, the PROMIS-29 raw score of the subject is 10 or greater, about 5 minutes to about 24 hours prior to intranasal administration of the racemic ketamine, or a pharmaceutically acceptable salt thereof. For example, about 5 minutes to 1 hour, about 5 minutes to 6 hours, about 5 minutes to 12 hours, about 5 minutes to 18 hours, about 18 hours to 24 hours, about 12 hours to 24 hours, or about 6 hours to 24 hours prior to intranasal administration of the racemic ketamine, or a pharmaceutically acceptable salt thereof. In some embodiments, the PROMIS-29 raw score of the subject is 8 or greater, about 5 minutes to about 24 hours prior to intranasal administration of the racemic ketamine, or a pharmaceutically acceptable salt thereof. For example, about 5 minutes to 1 hour, about 5 minutes to 6 hours, about 5 minutes to 12 hours, about 5 minutes to 18 hours, about 18 hours to 24 hours, about 12 hours to 24 hours, or about 6 hours to 24 hours prior to intranasal administration of the racemic ketamine, or a pharmaceutically acceptable salt thereof. In some embodiments, the PROMIS-29 raw score of the subject is 6 or greater, about 5 minutes to about 24 hours prior to intranasal administration of the racemic ketamine, or a pharmaceutically acceptable salt thereof. For example, about 5 minutes to 1 hour, about 5 minutes to 6 hours, about 5 minutes to 12 hours, about 5 minutes to 18 hours, about 18 hours to 24 hours, about 12 hours to 24 hours, or about 6 hours to 24 hours prior to intranasal administration of the racemic ketamine, or a pharmaceutically acceptable salt thereof. In some embodiments, the PROMIS-29 raw score of the subject is 4 or greater, about 5 minutes to about 24 hours prior to intranasal administration of the racemic ketamine, or a pharmaceutically acceptable salt thereof. For example, about 5 minutes to 1 hour, about 5 minutes to 6 hours, about 5 minutes to 12 hours, about 5 minutes to 18 hours, about 18 hours to 24 hours, about 12 hours to 24 hours, or about 6 hours to 24 hours prior to intranasal administration of the racemic ketamine, or a pharmaceutically acceptable salt thereof.

In some embodiments, the PROMIS-29 raw score of the subject decreases by 2 to 16 (e.g., by 1 to 16 units) after intranasal administration of the racemic ketamine, or a pharmaceutically acceptable salt thereof. In some embodiments, the PROMIS-29 raw score of the subject decreases by 4 to 14 (e.g., by 4 to 14 units) after intranasal administration of the racemic ketamine, or a pharmaceutically acceptable salt thereof. In some embodiments, the PROMIS-29 raw score of the subject decreases by 6 to 12 (e.g., by 6 to 12 units) after intranasal administration of the racemic ketamine, or a pharmaceutically acceptable salt thereof. In some embodiments, the PROMIS-29 raw score of the subject decreases by 4 to 6 (e.g., 4 to 6 units) after intranasal administration of the racemic ketamine, or a pharmaceutically acceptable salt thereof. In some embodiments, the PROMIS-29 raw score of the subject decreases by 2 to 4 (e.g., 2 to 4 units) after intranasal administration of the racemic ketamine, or a pharmaceutically acceptable salt thereof. In some embodiments, the PROMIS-29 raw score of the subject decreases by 1 to 2 (e.g., 1 to 2 units) after intranasal administration of the racemic ketamine, or a pharmaceutically acceptable salt thereof. 20 The CGI-Severity (CGIS) asks the clinician one question: “Considering your total clinical experience with this particular population, how mentally ill is the subject/patient at this time?” which is rated on the following seven-point scale: 1=normal, not at all ill; 2=borderline mentally ill; 3=mildly ill; 4=moderately ill; 5=markedly ill; 6=severely ill; 7=among the most extremely ill subject/patients. This rating is based upon observed and reported symptoms, behavior, and function since the previous visit. The 25 CGIS can also be measured on a five-point scale and the values described herein can be adjusted accordingly.

In some embodiments, the CGIS score of the subject is 2 or greater prior to intranasal administration of the racemic ketamine, or a pharmaceutically acceptable salt thereof. In some embodiments, the CGIS score is 3 or greater, about 5 minutes to about 24 hours prior to intranasal administration of the racemic ketamine, or a pharmaceutically acceptable salt thereof. For example, about 5 minutes to 1 hour, about 5 minutes to 6 hours, about 5 minutes to 12 hours, about 5 minutes to 18 hours, about 18 hours to 24 hours, about 12 hours to 24 hours, or about 6 hours to 24 hours prior to intranasal administration of the racemic ketamine, or a pharmaceutically acceptable salt thereof. In some embodiments, the CGIS score of the subject is 3, 4, or 5, about 1 hour to about 4 hours, about 1.5 hours to about 5 hours, about 2 hours to about 6 hours, or about 5 hours to about 10 hours, prior to intranasal administration of the racemic ketamine, or a pharmaceutically acceptable salt thereof. In some embodiments, the CGIS score of the subject is 4 or 5, about 1 hour to about 4 hours, about 1.5 hours to about 5 hours, about 2 hours to about 6 hours, or about 5 hours to about 10 hours, prior to intranasal administration of the racemic ketamine, or a pharmaceutically acceptable salt thereof.

In some embodiments, the CGIS score decreases by 1 to 5 (e.g., by 1 to 5 units) after intranasal administration of the racemic ketamine, or a pharmaceutically acceptable salt thereof. In some embodiments, the CGIS score decreases by 1 to 5 (e.g., by 1 to 5 units) after intranasal administration of the racemic ketamine, or a pharmaceutically acceptable salt thereof. In some embodiments, the CGIS score decreases by 1 to 4 (e.g., by 1 to 4 units) after intranasal administration of the racemic ketamine, or a pharmaceutically acceptable salt thereof. In some embodiments, the CGIS score decreases by 1 to 3 (e.g., 1 to 3 units) after intranasal administration of the racemic ketamine, or a pharmaceutically acceptable salt thereof. In some embodiments, the CGIS score decreases by 1 to 2 (e.g., 1 to 2 units) after intranasal administration of the racemic ketamine, or a pharmaceutically acceptable salt thereof.

In some embodiments, the CGIS score is 2 or greater (e.g., 3, 4, or 5) prior to intranasal administration of the racemic ketamine, or a pharmaceutically acceptable salt thereof, and decreases by 1 to 5 after intranasal administration of the racemic ketamine, or a pharmaceutically acceptable salt thereof. In some embodiments, the CGIS score is 3 or greater (e.g., 3, 4, or 5) prior to intranasal administration of the racemic ketamine, or a pharmaceutically acceptable salt thereof, and decreases by 1 to 5, e.g., by 1, 2, 3, 4, or 5 after intranasal administration of the racemic ketamine, or a pharmaceutically acceptable salt thereof. In some embodiments, the CGIS score is 4 or 5 prior to intranasal administration of the racemic ketamine, or a pharmaceutically acceptable salt thereof, and decreases by 1 to 4, e.g., by 1, 2, 3, or 4 after intranasal administration of the racemic ketamine, or a pharmaceutically acceptable salt thereof. In some embodiments, the CGIS score is 5 prior to intranasal administration of the racemic ketamine, or a pharmaceutically acceptable salt thereof, and decreases by 1 to 3, e.g., by 1, 2, or 3 after intranasal administration of the racemic ketamine, or a pharmaceutically acceptable salt thereof.

In some embodiments, the CGIS score of the subject is 2 or more, e.g., 2, 3, 4, or 5, about 5 minutes to about 24 hours prior to intranasal administration of the racemic ketamine, or a pharmaceutically acceptable salt thereof, and decreases by 1, 2, 3, 4, or 5, about 5 minutes to about 24 hours after intranasal administration of the racemic ketamine, or a pharmaceutically acceptable salt thereof. In some embodiments, the CGIS score of the subject is 2 or more, e.g., 2, 3, 4, or 5, between about 1 hour to about 4 hours, about 1.5 hours to about 5 hours, about 2 hours to about 6 hours, or about 5 hours to about 10 hours, prior to intranasal administration of the racemic ketamine, or a pharmaceutically acceptable salt thereof, and decreases by 1, 2, 3, 4, or 5, about 1 hour to about 4 hours, about 1.5 hours to about 5 hours, about 2 hours to about 6 hours, or about 5 hours to about 10 hours, after intranasal administration of the racemic ketamine, or a pharmaceutically acceptable salt thereof. In some embodiments, the CGIS score of the subject is decreased by 1, 2, 3, 4, or 5, points 24 hours after intranasal administration of racemic ketamine. In some embodiments, the CGIS score of the subject is decreased by 2 points. In some embodiments, the CGIS score of the subject is decreased by 3 points. In some embodiments, the CGIS score of the subject is decreased by 4 points. In some embodiments, the CGIS score of the subject is decreased by 5 points. In some embodiments, the CGIS score of the subject is decreased by 6 points.

Clinical Global Impression of Change in Suicidal Ideation and Behavior (CGIC-SI/B) and Clinical Global Impression of Severity of Suicidal Ideation and Behavior (CGIS-SI/B) are two companion measures of Clinical Global Impression (CGI) rating the following: (a) change from the initiation of treatment and (b) severity of psychopathology (in this case Suicidal Ideation and Behavior) on a scale of 1 to 5. The CGI captures clinical impressions and tracks clinical progress across time. CGI has been shown to correlate well with standard, well-known research drug efficacy scales (e.g., Hamilton Rating Scale for Depression, Hamilton Rating Scale for Anxiety, Positive and Negative Syndrome Scale, Leibowitz Social Anxiety Scale, Brief Psychiatric Rating Scale, Scale for the Assessment of Negative Symptoms, and others) across a wide range of psychiatric indications.

The CGI-Change (CGIC) is a single-item measure of change with treatment. Each time the subject/patient is seen after medication has been initiated, the clinician compares the subject/patient's overall clinical condition to the one week period just prior to the initiation of medication use (the so-called baseline visit). The CGIC is rated on a seven-point scale: “Compared to the subject/patient's condition at baseline [prior to medication initiation], this subject/patient's condition is: 1=very much improved since the initiation of treatment; 2=much improved; 3=minimally improved; 4=no change from baseline (the initiation of treatment); 5=minimally worse; 6=much worse; 7=very much worse since the initiation of treatment.”

In some embodiments, CGI-S score of the subject is 4 prior to intranasal administration of the racemic ketamine, or a pharmaceutically acceptable salt thereof. In some embodiments, the CGIC score of the subject is 1 after intranasal administration of the racemic ketamine, or a pharmaceutically acceptable salt thereof (e.g., relative to the CGI-S score). In some embodiments, the CGIC score of the subject is 2 after intranasal administration of the racemic ketamine, or a pharmaceutically acceptable salt thereof. In some embodiments, the CGIC score of the subject is 3 after intranasal administration of the racemic ketamine, or a pharmaceutically acceptable salt thereof. In some embodiments, the CGIC score of the subject is 4 after intranasal administration of the racemic ketamine, or a pharmaceutically acceptable salt thereof. In some embodiments, the CGIC score of the subject is 5 after intranasal administration of the racemic ketamine, or a pharmaceutically acceptable salt thereof.

In some embodiments, the CGIC score of the subject is reduced by 1 (e.g., 1 unit) after intranasal administration of the racemic ketamine, or a pharmaceutically acceptable salt thereof. In some embodiments, the CGIC score of the subject is reduced by 2 (e.g., 2 unit) after intranasal administration of the racemic ketamine, or a pharmaceutically acceptable salt thereof. In some embodiments, the CGIC score of the subject is reduced by 3 (e.g., 3 unit) after intranasal administration of the racemic ketamine, or a pharmaceutically acceptable salt thereof. In some embodiments, the CGIC score of the subject is reduced by 5 (e.g., 5 unit) after intranasal administration of the racemic ketamine, or a pharmaceutically acceptable salt thereof.

The Patient's Global Impression of Severity in Suicidal Ideation and Behavior (PGIS-SI/B) is a 5-point, subject-rated scale for assessing a subject's view of the general severity of their illness, which is rated on a single-item Likert-type scale ranging from 1 (not at all suicidal) to 5 (extremely suicidal). The PGIS-SI/B can be administered at various time points (e.g., before intranasal administration of racemic ketamine as described herein or after one or more doses of the racemic ketamine, wherein the latter provide the “change” score described below). See, e.g., Mohebbi et al. Eur Psychiatry. 2018; 53:17-22.

Patient's Global Impression of Change in Suicidal Ideation and Behavior (PGIC-SI/B) is a 7-point scale that rates the subject/patient's impression of the effects to treatment on the following scale based on the question “how much have your suicidal impulses, thoughts, and behaviors changed compared with your condition at baseline?”:
1 Very much better
2 Much better
3 A little better
4 No change
5 A little worse
6 Much worse
7 Very much worse

The PGIC-SI/B scale can be administered at various time points after one or more doses of intranasal racemic ketamine, as described herein, relative to the PGIS-SI/B score determined prior to administration of intranasal racemic ketamine, as described herein. See, e.g., Mohebbi et al. Eur Psychiatry. 2018; 53:17-22.

In some embodiments, the PGIS-SI/B score of the subject is 3 or greater prior to intranasal administration of the racemic ketamine, or a pharmaceutically acceptable salt thereof. In some embodiments, the PGIS-SI/B score is 3 or greater, about 5 minutes to about 24 hours prior to intranasal administration of the racemic ketamine, or a pharmaceutically acceptable salt thereof. For example, about 5 minutes to 1 hour, about 5 minutes to 6 hours, about 5 minutes to 12 hours, about 5 minutes to 18 hours, about 18 hours to 24 hours, about 12 hours to 24 hours, or about 6 hours to 24 hours prior to intranasal administration of the racemic ketamine, or a pharmaceutically acceptable salt thereof. In some embodiments, the PGIS-SI/B score of the subject is 3, 4, or 5, about 1 hour to about 4 hours, about 1.5 hours to about 5 hours, about 2 hours to about 6 hours, or about 5 hours to about 10 hours, prior to intranasal administration of the racemic ketamine, or a pharmaceutically acceptable salt thereof.

In some embodiments, the PGIS-SI/B score decreases by 1 to 4 (e.g., by 1 to 4 units) after intranasal administration of the racemic ketamine, or a pharmaceutically acceptable salt thereof (i.e., the PGIC-SI/B score). In some embodiments, the PGIS-SI/B score decreases by 1 to 3 (e.g., 1 to 3 units) after intranasal administration of the racemic ketamine, or a pharmaceutically acceptable salt thereof. In some embodiments, the PGIS-SI/B score decreases by 1 to 2 (e.g., 1 to 2 units) after intranasal administration of the racemic ketamine, or a pharmaceutically acceptable salt thereof.

In some embodiments, the PGIS-SI/B score is 3 or greater (e.g., 3, 4, or 5) prior to intranasal administration of the racemic ketamine, or a pharmaceutically acceptable salt thereof, and decreases by 1 to 6 after intranasal administration of the racemic ketamine, or a pharmaceutically acceptable salt thereof. In some embodiments, the PGIS-SI/B score is 4 or greater (e.g., 4 or 5) prior to intranasal administration of the racemic ketamine, or a pharmaceutically acceptable salt thereof, and decreases by 1 to 4, e.g., by 1, 2, 3, or 4, after intranasal administration of the racemic ketamine, or a pharmaceutically acceptable salt thereof. In some embodiments, the PGIS-SI/B score is 3 to 5 (e.g., 3, 4, or 5) prior to intranasal administration of the racemic ketamine, or a pharmaceutically acceptable salt thereof, and decreases by 1 to 4, e.g., by 1, 2, 3, or 4 after intranasal administration of the racemic ketamine, or a pharmaceutically acceptable salt thereof. In some embodiments, the PGIS-SI/B score of the subject is 2, 3, 4, or 5, about 5 minutes to about 24 hours prior to intranasal administration of the racemic ketamine, or a pharmaceutically acceptable salt thereof, and decreases by 1, 2, 3, or 4, about 5 minutes to about 24 hours after intranasal administration of the racemic ketamine, or a pharmaceutically acceptable salt thereof. In some embodiments, the PGIS-SI/B score of the subject is 2, 3, 4, or 5 at about 1 hour to about 4 hours, about 1.5 hours to about 5 hours, about 2 hours to about 6 hours, or about 5 hours to about 10 hours, prior to intranasal administration of the racemic ketamine, or a pharmaceutically acceptable salt thereof, and decreases by 1, 2, 3, or 4, about 1 hour to about 4 hours, about 1.5 hours to about 5 hours, about 2 hours to about 6 hours, or about 5 hours to about 10 hours, after intranasal administration of the racemic ketamine, or a pharmaceutically acceptable salt thereof. In some embodiments, the PGIS-SI/B score of the subject is decreased by 3 or 4 points 24 hours after intranasal administration of racemic ketamine. In some embodiments, the PGIS-SI/B score of the subject is decreased by 3 points. In some embodiments, the PGIS-SI/B score of the subject is decreased by 4 points.

In some embodiments, the PGIS-SI/B score is 2 prior to intranasal administration of the racemic ketamine, or a pharmaceutically acceptable salt thereof, and decreases by 1 or 2, after intranasal administration of the racemic ketamine, or a pharmaceutically acceptable salt thereof. In some embodiments, the PGIS-SI/B score of the subject is 1 or 2 at about 5 minutes to about 24 hours prior to intranasal administration of the racemic ketamine, or a pharmaceutically acceptable salt thereof, and decreases by 1 or 2, about 5 minutes to about 24 hours after intranasal administration of the racemic ketamine, or a pharmaceutically acceptable salt thereof. In some embodiments, the PGIS-SI/B score of the subject is 1, 2, at about 1 hour to about 4 hours, about 1.5 hours to about 5 hours, about 2 hours to about 6 hours, or about 5 hours to about 10 hours, prior to intranasal administration of the racemic ketamine, or a pharmaceutically acceptable salt thereof, and decreases by 1 or 2 about 1 hour to about 4 hours, about 1.5 hours to about 5 hours, about 2 hours to about 6 hours, or about 5 hours to about 10 hours, after intranasal administration of the racemic ketamine, or a pharmaceutically acceptable salt thereof.

In some embodiments, a first PGIS-SI/B score is determined about 1 hour to about 12 hours prior to intranasal administration of the racemic ketamine, or a pharmaceutically acceptable salt thereof; and a second PGIS-SI/B score (i.e., the PGIC-SI/B score) is determined about 1 hour to about 24 hours after to intranasal administration of the racemic ketamine, or a pharmaceutically acceptable salt thereof. In some embodiments, the first PGIS-SI/B score from the subject is determined about 4 hours to about 8 hours prior to intranasal administration of the racemic ketamine, or a pharmaceutically acceptable salt thereof. In some embodiments, the second PGIS-SI/B score from the subject is determined about 4 hours to about 24 hours after intranasal administration of the racemic ketamine, or a pharmaceutically acceptable salt thereof. In some embodiments, the first PGIS-SI/B score and the second PGIS-SI/B score are determined at equivalent times before, and after, administration of the racemic ketamine, or a pharmaceutically acceptable salt thereof, such as 4 hours. In some embodiments, the first PGIS-SI/B score and the second PGIS-SI/B score are determined at different times before, and after, administration of the racemic ketamine, or a pharmaceutically acceptable salt thereof, such as 4 hours before, and 12 hours after, administration of the racemic ketamine, or a pharmaceutically acceptable salt thereof. The change in score, described below, is the PGIC-SI/B.

In some embodiments, the PGIS-SI/B score of the subject is 1 or greater prior to intranasal administration of the racemic ketamine, or a pharmaceutically acceptable salt thereof. In some embodiments, the PGIC-SI/B score of the subject is 1 or greater, about 5 minutes to about 24 hours prior to intranasal administration of the racemic ketamine, or a pharmaceutically acceptable salt thereof. For example, about 5 minutes to 1 hour, about 5 minutes to 6 hours, about 5 minutes to 12 hours, about 5 minutes to 18 hours, about 18 hours to 24 hours, about 12 hours to 24 hours, or about 6 hours to 24 hours prior to intranasal administration of the racemic ketamine, or a pharmaceutically acceptable salt thereof. In some embodiments, the PGIC-SI/B score of the subject is 2, 3, or 4, about 1 hour to about 4 hours, about 1.5 hours to about 5 hours, about 2 hours to about 6 hours, or about 5 hours to about 10 hours, about 5 minutes to 18 hours, about 18 hours to 24 hours, or about 6 hours to 24 hours, prior to intranasal administration of the racemic ketamine, or a pharmaceutically acceptable salt thereof. In some embodiments, the PGIC-SI/B score of the subject is 5, 6, or 7, about 1 hour to about 4 hours, about 1.5 hours to about 5 hours, about 2 hours to about 6 hours, or about 5 hours to about 10 hours, about 5 minutes to 18 hours, about 18 hours to 24 hours, or about 6 hours to 24 hours, prior to intranasal administration of the racemic ketamine, or a pharmaceutically acceptable salt thereof.

In some embodiments, the PGIC-SI/B score of the subject decreases by 1 to 6 (e.g., by 1 to 6 units) relative to the PGIS-SI/B score after intranasal administration of the racemic ketamine, or a pharmaceutically acceptable salt thereof. In some embodiments, PGIC-SI/B score of the subject decreases by 1 to 5 (e.g., by 1 to 5 units) after intranasal administration of the racemic ketamine, or a pharmaceutically acceptable salt thereof. In some embodiments, the PGIC-SI/B score of the subject decreases by 1 to 4 (e.g., by 1 to 4 units) after intranasal administration of the racemic ketamine, or a pharmaceutically acceptable salt thereof. In some embodiments, the PGIC-SI/B score of the subject decreases by 1 to 3 (e.g., 1 to 3 units) after intranasal administration of the racemic ketamine, or a pharmaceutically acceptable salt thereof. In some embodiments, the PGIC-SI/B score of the subject decreases by 1 to 2 (e.g., 1 to 2 units) after intranasal administration of the racemic ketamine, or a pharmaceutically acceptable salt thereof.

The Patient Global Impression of Change (PGIC) is a single-item measure of global improvement with treatment on which participants use a seven-point scale (+3=“very much improved” to −3=“very much worse”, and 0=“no change”). The questionnaire contains a question phrased as “what is your overall impression of change after receiving [study test article].

In some embodiments, the PGIS score of the subject is 0 prior to intranasal administration of the racemic ketamine, or a pharmaceutically acceptable salt thereof. In some embodiments, the Patient Global Impression of Change (PGIC) score increases by 3 (e.g., by 3 units) after intranasal administration of the racemic ketamine, or a pharmaceutically acceptable salt thereof (relative to the PGIS score). In some embodiments, the PGIC score increases by 2 (e.g., by 2 units) after intranasal administration of the racemic ketamine, or a pharmaceutically acceptable salt thereof. In some embodiments, the PGIC score increases by 1 (e.g., by 1 units) after intranasal administration of the racemic ketamine, or a pharmaceutically acceptable salt thereof. In some embodiments, the PGIC score increases by 0 (i.e., no change) after intranasal administration of the racemic ketamine, or a pharmaceutically acceptable salt thereof.

The Mini-international neuropsychiatric interview (MINI) is a short structured clinical interview which enables clinicians and researchers to assess the 17 most common psychiatric disorders in DSM-III-R, DSM-IV and DSM-5 and ICD-10. The clinician or researcher asks the subject a number of questions relating to each psychiatric disorders, and depending on the number of binary responses (yes/no) to these questions to diagnose a psychiatric disorder, such as the psychiatric disorders described herein. For example, the MINI for Suicidality Disorders for DSM-5 is a semi-structured clinical interview that can be administered (e.g., at screening) to confirm a primary diagnosis of MDD, to evaluate the presence or absence of current suicidal ideation and behavior (SI/B), and to assess for comorbid neuropsychiatric disorders. The MINI can inform and complement a full psychiatric intake examination when administered by a qualified and trained. See, e.g., Sheehan et al. J. Clin Psychiatry, 1998; 59(suppl 20): 22-33; Sheehan and Giddens (2015). Suicidality: A Roadmap for Assessment and Treatment. (1st ed.). Tampa, FL: Harm Research Press. November 2015 (Available from: HarmResearch.org) ISBN: 978-0-9969729-0-1; and Sheehan and Giddens. (2016). Suicidality Assessment and Documentation for Healthcare Providers: A Brief, Practical Guide. (1st ed.). Tampa, FL: Harm Research Press. April 2016. (Available from: HarmResearch.org) ISBN: 978-0-9969729-1-8).

In some embodiments, the subject does not have or has not been diagnosed with Impulse Attack Suicidality Disorder and/or Homicidal Suicidality Disorder, e.g., by the MINI Version 7.02 for Suicidality Disorders. In some embodiments, the subject does not have or has not been determined to have a score of 9 or 10 on the STS-CMCM Clinician Judgement of Patient Risk of Suicide Attempt or Death. In some embodiments, the subject does not score about 4 or higher on MADRS item 10 without about 30 days of initiation of treatment as described herein. In some embodiments, the subject does not have a score of 2, 3, or 4 on the C-SSRS for Actual Lethality/Medical Damage.

In some embodiments, the subject has history of chronic (>3 months) intermittent non-impulsive suicidality. In some embodiments, the subject has current suicidal ideation with intent, e.g., by the MINI Suicidality Module at screening and baseline, specifically a positive response related to present symptoms on Question B3, as well as a positive response related to symptoms within the past 24 hours on Question B10 or B11. In some embodiments, the subject has a score of about 8 or less on the STS-CMCM Clinician Judgement of Patient Risk of Suicide Attempt or Death.

Antidepressant Treatment Response Questionnaire (ATRQ) is a form used to determine treatment resistance in major depressive disorder (MDD). The subject is asked to answer a number of questions relating to their experience with prior treatment with an extensive list of antidepressant medications including tricyclics, monoamine oxidase inhibitors, selective serotonin reuptake inhibitors, serotonin-norepinephrine reuptake inhibitors, and other antidepressants. The ATRQ includes the following questions: (1) Have you received any treatment with medications in the PAST FIVE YEARS? Please circle the correct answer. YES NO (2) If YES, please review the list on page 2 and put a check next to any medication(s) that you have taken for at least 6 weeks during the PAST FIVE YEARS. (3) Of those medication(s) that you have checked from the list on page 2, please put a check in the second column next to those that you have taken at a dosage equal to or greater than the minimum dosage listed for that medication. (4) Of the medications that you have checked on page 2, please rate how much you feel each helped you with your depression. Compare to a time when you felt well (prior to the current episode or period of depression). Use the following scale to rate your improvement in the last column. If a rating of 100% is “completely improved” and 0 is “not improved at all,” how close to 100% did you get on this medication? 1=Less than 25% improved 2=Between 25% and 49% improved 3=Between 50% and 75% improved 4=More than 75% improved.

The Sheehan Disability Scale (SDS) is a 5-item self-report tool that assesses functional impairment in work/school, social life, and family life. The SDS is a disability or functional impairment scale that uses a discan metric. This discan metric anchors the response options by simultaneously using visual-spatial, numeric and verbal descriptive anchors to assess disability or functional impairment across three domains: work/school, social life/leisure activities and family life/home responsibilities. Each domain is scored from 0 (not at all) to 10 (extremely). The three domains can be summarized to evaluate global functional impairment by adding the scores of each of the three domains, resulting in global SDS score ranges from 0 (unimpaired) to 30 (highly impaired).

In some embodiments, the SDS score of the subject is 25 to 30 prior to intranasal administration of the racemic ketamine, or a pharmaceutically acceptable salt thereof. In some embodiments, the SDS score of the subject is 20 or greater prior to intranasal administration of the racemic ketamine, or a pharmaceutically acceptable salt thereof. In some embodiments, the SDS score of the subject is 15 or greater prior to intranasal administration of the racemic ketamine, or a pharmaceutically acceptable salt thereof. In some embodiments, the SDS score of the subject is 10 or greater prior to intranasal administration of the racemic ketamine, or a pharmaceutically acceptable salt thereof. In some embodiments, the SDS score of the subject is 5 or greater prior to intranasal administration of the racemic ketamine, or a pharmaceutically acceptable salt thereof. In some embodiments, the SDS score of the subject is 1 or greater prior to intranasal administration of the racemic ketamine, or a pharmaceutically acceptable salt thereof.

In some embodiments, the SDS score is 25 or greater, about 5 minutes to about 24 hours prior to intranasal administration of the racemic ketamine, or a pharmaceutically acceptable salt thereof. For example, about 5 minutes to 1 hour, about 5 minutes to 6 hours, about 5 minutes to 12 hours, about 5 minutes to 18 hours, about 18 hours to 24 hours, about 12 hours to 24 hours, or about 6 hours to 24 hours prior to intranasal administration of the racemic ketamine, or a pharmaceutically acceptable salt thereof. In some embodiments, t the SDS score is 20 or greater, about 5 minutes to about 24 hours prior to intranasal administration of the racemic ketamine, or a pharmaceutically acceptable salt thereof. For example, about 5 minutes to 1 hour, about 5 minutes to 6 hours, about 5 minutes to 12 hours, about 5 minutes to 18 hours, about 18 hours to 24 hours, about 12 hours to 24 hours, or about 6 hours to 24 hours prior to intranasal administration of the racemic ketamine, or a pharmaceutically acceptable salt thereof. In some embodiments, the SDS score is 15 or greater, about 5 minutes to about 24 hours prior to intranasal administration of the racemic ketamine, or a pharmaceutically acceptable salt thereof. For example, about 5 minutes to 1 hour, about 5 minutes to 6 hours, about 5 minutes to 12 hours, about 5 minutes to 18 hours, about 18 hours to 24 hours, about 12 hours to 24 hours, or about 6 hours to 24 hours prior to intranasal administration of the racemic ketamine, or a pharmaceutically acceptable salt thereof. In some embodiments, the SDS score is 10 or greater, about 5 minutes to about 24 hours prior to intranasal administration of the racemic ketamine, or a pharmaceutically acceptable salt thereof. For example, about 5 minutes to 1 hour, about 5 minutes to 6 hours, about 5 minutes to 12 hours, about 5 minutes to 18 hours, about 18 hours to 24 hours, about 12 hours to 24 hours, or about 6 hours to 24 hours prior to intranasal administration of the racemic ketamine, or a pharmaceutically acceptable salt thereof. In some embodiments, the SDS score is 5 or greater, about 5 minutes to about 24 hours prior to intranasal administration of the racemic ketamine, or a pharmaceutically acceptable salt thereof. For example, about 5 minutes to 1 hour, about 5 minutes to 6 hours, about 5 minutes to 12 hours, about 5 minutes to 18 hours, about 18 hours to 24 hours, about 12 hours to 24 hours, or about 6 hours to 24 hours prior to intranasal administration of the racemic ketamine, or a pharmaceutically acceptable salt thereof. In some embodiments, the SDS score is 1 or greater, about 5 minutes to about 24 hours prior to intranasal administration of the racemic ketamine, or a pharmaceutically acceptable salt thereof. For example, about 5 minutes to 1 hour, about 5 minutes to 6 hours, about 5 minutes to 12 hours, about 5 minutes to 18 hours, about 18 hours to 24 hours, about 12 hours to 24 hours, or about 6 hours to 24 hours prior to intranasal administration of the racemic ketamine, or a pharmaceutically acceptable salt thereof.

In some embodiments, the SDS score decreases by 1 to 30 (e.g., by 1 to 30 units) after intranasal administration of the racemic ketamine, or a pharmaceutically acceptable salt thereof. In some embodiments, the SDS score decreases by 1 to 25 (e.g., by 1 to 25 units) after intranasal administration of the racemic ketamine, or a pharmaceutically acceptable salt thereof. In some embodiments, the SDS score decreases by 1 to 20 (e.g., by 1 to 20 units) after intranasal administration of the racemic ketamine, or a pharmaceutically acceptable salt thereof. In some embodiments, the SDS score decreases by 1 to 15 (e.g., 1 to 15 units) after intranasal administration of the racemic ketamine, or a pharmaceutically acceptable salt thereof. In some embodiments, the SDS score decreases by 1 to 10 (e.g., 1 to 10 units) after intranasal administration of the racemic ketamine, or a pharmaceutically acceptable salt thereof. In some embodiments, the SDS score decreases by 1 to 5 (e.g., 1 to 5 units) after intranasal administration of the racemic ketamine, or a pharmaceutically acceptable salt thereof. In some embodiments, the SDS score decreases by 15 to 25 (e.g., 15 to 25 units) after intranasal administration of the racemic ketamine, or a pharmaceutically acceptable salt thereof. In some embodiments, the SDS score decreases by 10 to 30 (e.g., 10 to 30 units) after intranasal administration of the racemic ketamine, or a pharmaceutically acceptable salt thereof. In some embodiments, the SDS score decreases by 5 to 10 (e.g., 5 to 10 units) after intranasal administration of the racemic ketamine, or a pharmaceutically acceptable salt thereof.

The Pain Self-Efficacy Questionnaire (PSEQ) is a 10-item questionnaire designed to assess the confidence people with ongoing pain have in performing activities while in pain. The PSEQ is applicable to all persisting pain presentations. It covers a range of functions, including household chores, socializing, work, as well as coping with pain without medication. People are asked to rate how confidently they can perform the activities described, at present, despite their pain. Answers are indicated on a 7-point Likert scale under each item, where 0=not at all confident and 6=completely confident. A total score, ranging from 0 to 60, is calculated by adding the scores for each item. Higher scores reflect stronger self-efficacy beliefs.

In some embodiments, the PSEQ score of the subject is 5 or less prior to intranasal administration of the racemic ketamine, or a pharmaceutically acceptable salt thereof. In some embodiments, the PSEQ score of the subject is 10 or less prior to intranasal administration of the racemic ketamine, or a pharmaceutically acceptable salt thereof. In some embodiments, the PSEQ score of the subject is 20 or less prior to intranasal administration of the racemic ketamine, or a pharmaceutically acceptable salt thereof. In some embodiments, the PSEQ score of the subject is 30 or less prior to intranasal administration of the racemic ketamine, or a pharmaceutically acceptable salt thereof. In some embodiments, the PSEQ score of the subject is 40 or less prior to intranasal administration of the racemic ketamine, or a pharmaceutically acceptable salt thereof. In some embodiments, the PSEQ score of the subject is 50 or less prior to intranasal administration of the racemic ketamine, or a pharmaceutically acceptable salt thereof. In some embodiments, the PSEQ score of the subject is 55 or less prior to intranasal administration of the racemic ketamine, or a pharmaceutically acceptable salt thereof.

In some embodiments, the PSEQ score is 5 or less, about 5 minutes to about 24 hours prior to intranasal administration of the racemic ketamine, or a pharmaceutically acceptable salt thereof. For example, about 5 minutes to 1 hour, about 5 minutes to 6 hours, about 5 minutes to 12 hours, about 5 minutes to 18 hours, about 18 hours to 24 hours, about 12 hours to 24 hours, or about 6 hours to 24 hours prior to intranasal administration of the racemic ketamine, or a pharmaceutically acceptable salt thereof. In some embodiments, the PSEQ score is 10 or less, about 5 minutes to about 24 hours prior to intranasal administration of the racemic ketamine, or a pharmaceutically acceptable salt thereof. For example, about 5 minutes to 1 hour, about 5 minutes to 6 hours, about 5 minutes to 12 hours, about 5 minutes to 18 hours, about 18 hours to 24 hours, about 12 hours to 24 hours, or about 6 hours to 24 hours prior to intranasal administration of the racemic ketamine, or a pharmaceutically acceptable salt thereof. In some embodiments, the PSEQ score is 20 or less, about 5 minutes to about 24 hours prior to intranasal administration of the racemic ketamine, or a pharmaceutically acceptable salt thereof. For example, about 5 minutes to 1 hour, about 5 minutes to 6 hours, about 5 minutes to 12 hours, about 5 minutes to 18 hours, about 18 hours to 24 hours, about 12 hours to 24 hours, or about 6 hours to 24 hours prior to intranasal administration of the racemic ketamine, or a pharmaceutically acceptable salt thereof. In some embodiments, the PSEQ score is 30 or less, about 5 minutes to about 24 hours prior to intranasal administration of the racemic ketamine, or a pharmaceutically acceptable salt thereof. For example, about 5 minutes to 1 hour, about 5 minutes to 6 hours, about 5 minutes to 12 hours, about 5 minutes to 18 hours, about 18 hours to 24 hours, about 12 hours to 24 hours, or about 6 hours to 24 hours prior to intranasal administration of the racemic ketamine, or a pharmaceutically acceptable salt thereof. In some embodiments, the PSEQ score is 40 or less, about 5 minutes to about 24 hours prior to intranasal administration of the racemic ketamine, or a pharmaceutically acceptable salt thereof. For example, about 5 minutes to 1 hour, about 5 minutes to 6 hours, about 5 minutes to 12 hours, about 5 minutes to 18 hours, about 18 hours to 24 hours, about 12 hours to 24 hours, or about 6 hours to 24 hours prior to intranasal administration of the racemic ketamine, or a pharmaceutically acceptable salt thereof. In some embodiments, the PSEQ score is 50 or less, about 5 minutes to about 24 hours prior to intranasal administration of the racemic ketamine, or a pharmaceutically acceptable salt thereof. For example, about 5 minutes to 1 hour, about 5 minutes to 6 hours, about 5 minutes to 12 hours, about 5 minutes to 18 hours, about 18 hours to 24 hours, about 12 hours to 24 hours, or about 6 hours to 24 hours prior to intranasal administration of the racemic ketamine, or a pharmaceutically acceptable salt thereof. In some embodiments, the PSEQ score is 55 or less, about 5 minutes to about 24 hours prior to intranasal administration of the racemic ketamine, or a pharmaceutically acceptable salt thereof. For example, about 5 minutes to 1 hour, about 5 minutes to 6 hours, about 5 minutes to 12 hours, about 5 minutes to 18 hours, about 18 hours to 24 hours, about 12 hours to 24 hours, or about 6 hours to 24 hours prior to intranasal administration of the racemic ketamine, or a pharmaceutically acceptable salt thereof.

In some embodiments, the PSEQ score increases by 5 to 55 (e.g., by 5 to 55 units) after intranasal administration of the racemic ketamine, or a pharmaceutically acceptable salt thereof. In some embodiments, the PSEQ score increases by 10 to 50 (e.g., by 10 to 50 units) after intranasal administration of the racemic ketamine, or a pharmaceutically acceptable salt thereof. In some embodiments, the PSEQ score increases by 20 to 40 (e.g., by 20 to 40 units) after intranasal administration of the racemic ketamine, or a pharmaceutically acceptable salt thereof. In some embodiments, the PSEQ score increases by 25 to 35 (e.g., 5 to 10 units) after intranasal administration of the racemic ketamine, or a pharmaceutically acceptable salt thereof. In some embodiments, the PSEQ score increases by 5 to 10 (e.g., 1 to 10 units) after intranasal administration of the racemic ketamine, or a pharmaceutically acceptable salt thereof.

The Richmond Agitation-Sedation Scale (RASS) is a medical scale used to measure the agitation or sedation level of a person. The RASS is often used in hospitalized subject/patients to describe their level of alertness or agitation. It is a 10-point scale, with four levels of anxiety or agitation (+1 to +4 [combative]), one level to denote a calm and alert state (0), and 5 levels of sedation (−1 to −5) culminating in unarousable (−5). See Table A.

TABLE A
Richmond Agtitation-Sedation Scale (RASS)

| | | |
|---|---|---|
| +4 | combative | violent, immediate danger to staff |
| +3 | very agitated | pulls or removes tube(s)/catheters, |
| | | aggressive |
| +2 | agitated | frequent nonpurposeful movement, |
| | | fight ventilator |
| +1 | restless | anxious, apprehensive, movements, |
| | | not aggressive or vigorous |
| 0 | alert and calm | |
| −1 | drowsy | not fully alert, but has sustained |
| | | awakening to voice (eye contact ≥10 sec |
| −2 | light sedation | briefly awakens to voice (eye opening |
| | | and contact <10 sec) |
| −3 | moderate | movement or eye opening to voice |
| | sedation | (but no eye contact) |
| −4 | deep sedation | no response to voice, but movement |
| | | or eye opening to physical stimulation |
| −5 | unarousable | no response to voice or |
| | | physical stimulation |

In some embodiments, the RASS score of the subject is 0 to 4 prior to intranasal administration of the racemic ketamine, or a pharmaceutically acceptable salt thereof. In some embodiments, the RASS score of the subject is 0 to 3 prior to intranasal administration of the racemic ketamine, or a pharmaceutically acceptable salt thereof. In some embodiments, the RASS score of the subject is 0 to 2 prior to intranasal administration of the racemic ketamine, or a pharmaceutically acceptable salt thereof. In some embodiments, the RASS score of the subject is 0 to 1 prior to intranasal administration of the racemic ketamine, or a pharmaceutically acceptable salt thereof. In some embodiments, the RASS score of the subject is 0 to −3 prior to intranasal administration of the racemic ketamine, or a pharmaceutically acceptable salt thereof. In some embodiments, the RASS score of the subject is 0 to −2 prior to intranasal administration of the racemic ketamine, or a pharmaceutically acceptable salt thereof. In some embodiments, the RASS score of the subject is 0 to −1 prior to intranasal administration of the racemic ketamine, or a pharmaceutically acceptable salt thereof.

In some embodiments, the RASS score decreases by 0 to 8 (e.g., by 0 to 8 units) after intranasal administration of the racemic ketamine, or a pharmaceutically acceptable salt thereof. In some embodiments, the RASS score decreases by 0 to 6 (e.g., by 0 to 6 units) after intranasal administration of the racemic ketamine, or a pharmaceutically acceptable salt thereof. In some embodiments, the RASS score decreases by 0 to 4 (e.g., by 0 to 4 units) after intranasal administration of the racemic ketamine, or a pharmaceutically acceptable salt thereof. In some embodiments, the RASS score decreases by 0 to 2 (e.g., 0 to 2 units) after intranasal administration of the racemic ketamine, or a pharmaceutically acceptable salt thereof. In some embodiments the RASS score decreases by 0 to 1 (e.g., 0 to 1 units) after intranasal administration of the racemic ketamine, or a pharmaceutically acceptable salt thereof. In some embodiments, the RASS score decreases by 1-2 (e.g., 1 to 2 units) after intranasal administration of the racemic ketamine, or a pharmaceutically acceptable salt thereof. In some embodiments, the RASS score decreases by 2-4 (e.g., 1 to 2 units) after intranasal administration of the racemic ketamine, or a pharmaceutically acceptable salt thereof.

In some embodiments, the RASS score increases by 0 to 1 (e.g., 0 to 1 units) after intranasal administration of the racemic ketamine, or a pharmaceutically acceptable salt thereof. In some embodiments, the RASS score increases by 1-2 (e.g., 1 to 2 units) after intranasal administration of the racemic ketamine, or a pharmaceutically acceptable salt thereof. In some embodiments, if the RASS score is −3 to −4, the next dose of intranasal racemic ketamine, or a pharmaceutically acceptable salt thereof, may be delayed until sedation lessens.

Numeric Pain Rating Scale (NPRS) is an 11-point measure of pain intensity on the following scale: 0=no pain, 1=very light barely noticeable, 2=minor but discomforting, 3=tolerable, but very noticeable, 4=distressing strong and deep, 5=very distressing strong, deep, and piercing, 6=intense, strong, deep and piercing, 7=very intense and completely dominates your senses, causing you to think unclearly about half the time, 8=so intense you can no longer think clearly, 9-excruciating pain, so intense you cannot tolerate it and demand pain killers or surgery, no matter what the side effects or risk, 10=unimaginable, so intense you will go unconscious shortly. The NPRS questionnaire is divided into three bins, each with the above described pain scale: (1) average pain in the last 24 h, (2) least pain the last 24 h, and (3) worst pain in the last 24 h.

In some embodiments, the subject has a NPRS score of about 1 to about 6 within about 1 minute to about 10 days prior to the initiation of administration of racemic ketamine, or a pharmaceutically acceptable salt thereof. In some embodiments, the subject has a NPRS score of about 6 to about 10 within about 1 minute to about 10 days prior to the initiation of administration of racemic ketamine, or a pharmaceutically acceptable salt thereof. In some embodiments, the subject has an average daily pain intensity score of ≥6 on the NPRS over the 7 days prior to randomization within the study.

In some embodiments, the NPRS score of the subject decreases by 1 to 10 (e.g., by 1 to 10 units) after intranasal administration of the racemic ketamine, or a pharmaceutically acceptable salt thereof. In some embodiments, the NPRS score of the subject decreases by 1 to 9 (e.g., by 1 to 9 units) after intranasal administration of the racemic ketamine, or a pharmaceutically acceptable salt thereof. In some embodiments, the NPRS score of the subject decreases by 1 to 7 (e.g., by 1 to 7 units) after intranasal administration of the racemic ketamine, or a pharmaceutically acceptable salt thereof. In some embodiments, the NPRS score of the subject decreases by 1 to 5 (e.g., by 1 to 5 units) after intranasal administration of the racemic ketamine, or a pharmaceutically acceptable salt thereof. In some embodiments, the NPRS score of the subject decreases by 1 to 3 (e.g., by 1 to 3 units) after intranasal administration of the racemic ketamine, or a pharmaceutically acceptable salt thereof. In some embodiments, the NPRS score of the subject decreases by 2 to 10 (e.g., by 2 to 10 units) after intranasal administration of the racemic ketamine, or a pharmaceutically acceptable salt thereof. In some embodiments, the NPRS score of the subject decreases by 2 to 5 (e.g., by 2 to 5 units) after intranasal administration of the racemic ketamine, or a pharmaceutically acceptable salt thereof. In some embodiments, the NPRS score of the subject decreases by 3 to 9 (e.g., by 3 to 9 units) after intranasal administration of the racemic ketamine, or a pharmaceutically acceptable salt thereof. In some embodiments, the NPRS score of the subject decreases by 4 to 6 (e.g., by 4 to 6 units) after intranasal administration of the racemic ketamine, or a pharmaceutically acceptable salt thereof.

The short-form McGill Pain Questionnaire (SF-MPQ) is a shorter version of the original MPQ, and was created to assess both the intensity and quality of pain. The pain rating index is comprised of 15 descriptors of pain including 11 from sensory categories and 4 from affective categories. Each of these descriptors are rated on an intensity scale as 0=none, 1=mild, 2=moderate and 3=severe. The total pain scores are is derived from the sum of the intensity rank values, which give the scale a range from 0-45.

In some embodiments, the SF-MPQ total score of the subject is 40 to 45 prior to intranasal administration of the racemic ketamine, or a pharmaceutically acceptable salt thereof. In some embodiments, the SF-MPQ total score of the subject is 35 or greater prior to intranasal administration of the racemic ketamine, or a pharmaceutically acceptable salt thereof. In some embodiments, the SF-MPQ total score of the subject is 25 or greater prior to intranasal administration of the racemic ketamine, or a pharmaceutically acceptable salt thereof. In some embodiments, the SF-MPQ total score of the subject is 15 or greater prior to intranasal administration of the racemic ketamine, or a pharmaceutically acceptable salt thereof. In some embodiments, the SF-MPQ total score of the subject is 10 or greater prior to intranasal administration of the racemic ketamine, or a pharmaceutically acceptable salt thereof. In some embodiments, the SF-MPQ total score of the subject is 5 or greater prior to intranasal administration of the racemic ketamine, or a pharmaceutically acceptable salt thereof.

In some embodiments, the SF-MPQ total score is 40 or greater, about 5 minutes to about 24 hours prior to intranasal administration of the racemic ketamine, or a pharmaceutically acceptable salt thereof. For example, about 5 minutes to 1 hour, about 5 minutes to 6 hours, about 5 minutes to 12 hours, about 5 minutes to 18 hours, about 18 hours to 24 hours, about 12 hours to 24 hours, or about 6 hours to 24 hours prior to intranasal administration of the racemic ketamine, or a pharmaceutically acceptable salt thereof. In some embodiments, the SF-MPQ total score is 30 or greater, about 5 minutes to about 24 hours prior to intranasal administration of the racemic ketamine, or a pharmaceutically acceptable salt thereof. For example, about 5 minutes to 1 hour, about 5 minutes to 6 hours, about 5 minutes to 12 hours, about 5 minutes to 18 hours, about 18 hours to 24 hours, about 12 hours to 24 hours, or about 6 hours to 24 hours prior to intranasal administration of the racemic ketamine, or a pharmaceutically acceptable salt thereof. In some embodiments, the SF-MPQ total score is 20 or greater, about 5 minutes to about 24 hours prior to intranasal administration of the racemic ketamine, or a pharmaceutically acceptable salt thereof. For example, about 5 minutes to 1 hour, about 5 minutes to 6 hours, about 5 minutes to 12 hours, about 5 minutes to 18 hours, about 18 hours to 24 hours, about 12 hours to 24 hours, or about 6 hours to 24 hours prior to intranasal administration of the racemic ketamine, or a pharmaceutically acceptable salt thereof. In some embodiments, the SF-MPQ total score is 10 or greater, about 5 minutes to about 24 hours prior to intranasal administration of the racemic ketamine, or a pharmaceutically acceptable salt thereof. For example, about 5 minutes to 1 hour, about 5 minutes to 6 hours, about 5 minutes to 12 hours, about 5 minutes to 18 hours, about 18 hours to 24 hours, about 12 hours to 24 hours, or about 6 hours to 24 hours prior to intranasal administration of the racemic ketamine, or a pharmaceutically acceptable salt thereof. In some embodiments, the SF-MPQ total score is 5 or greater, about 5 minutes to about 24 hours prior to intranasal administration of the racemic ketamine, or a pharmaceutically acceptable salt thereof. For example, about 5 minutes to 1 hour, about 5 minutes to 6 hours, about 5 minutes to 12 hours, about 5 minutes to 18 hours, about 18 hours to 24 hours, about 12 hours to 24 hours, or about 6 hours to 24 hours prior to intranasal administration of the racemic ketamine, or a pharmaceutically acceptable salt thereof.

In some embodiments, the SF-MPQ total score decreases by 5 to 45 (e.g., by 5 to 45 units) after intranasal administration of the racemic ketamine, or a pharmaceutically acceptable salt thereof. In some embodiments, the SF-MPQ total score decreases by 10 to 35 (e.g., by 10 to 35 units) after intranasal administration of the racemic ketamine, or a pharmaceutically acceptable salt thereof. In some embodiments, the SF-MPQ total score decreases by 15 to 25 (e.g., by 15 to 25 units) after intranasal administration of the racemic ketamine, or a pharmaceutically acceptable salt thereof. In some embodiments, the SF-MPQ total score decreases by 5 to 10 (e.g., 5 to 10 units) after intranasal administration of the racemic ketamine, or a pharmaceutically acceptable salt thereof. In some embodiments, the SF-MPQ total score decreases by 1 to 5 (e.g., 1 to 5 units) after intranasal administration of the racemic ketamine, or a pharmaceutically acceptable salt thereof.

Pain Catastrophizing Scale (PCS) is a means to quantify an individual's pain experience, asking about how they feel and what they think about when they are in pain. Compared to other ways of measuring pain-related thoughts, this questionnaire is unique in that the individual does not need to be in pain while completing it. Pain catastrophizing is characterized by the tendency to magnify the threat value of a pain stimulus and to feel helpless in the presence of pain, as well as by a relative inability to prevent or inhibit pain-related thoughts in anticipation of, during, or following a painful event. People are asked to indicate the degree to which they have 13 distinct thoughts and feelings when they are experiencing pain using the 0 (not at all) to 4 (all the time) scale. A total score is yielded (ranging from 0-52), along with three subscale scores assessing rumination, magnification and helplessness.

In some embodiments, the Pain Catastrophizing Scale (PCS) score of the subject is 45 to 52 prior to intranasal administration of the racemic ketamine, or a pharmaceutically acceptable salt thereof. In some embodiments, the PCS score of the subject is 35 or greater prior to intranasal administration of the racemic ketamine, or a pharmaceutically acceptable salt thereof. In some embodiments, the PCS score of the subject is 25 or greater prior to intranasal administration of the racemic ketamine, or a pharmaceutically acceptable salt thereof. In some embodiments, the PCS score of the subject is 15 or greater prior to intranasal administration of the racemic ketamine, or a pharmaceutically acceptable salt thereof. In some embodiments, the PCS score of the subject is 10 or greater prior to intranasal administration of the racemic ketamine, or a pharmaceutically acceptable salt thereof. In some embodiments, the PCS score of the subject is 5 or greater prior to intranasal administration of the racemic ketamine, or a pharmaceutically acceptable salt thereof.

In some embodiments, the PCS score is 40 or greater, about 5 minutes to about 24 hours prior to intranasal administration of the racemic ketamine, or a pharmaceutically acceptable salt thereof. For example, about 5 minutes to 1 hour, about 5 minutes to 6 hours, about 5 minutes to 12 hours, about 5 minutes to 18 hours, about 18 hours to 24 hours, about 12 hours to 24 hours, or about 6 hours to 24 hours prior to intranasal administration of the racemic ketamine, or a pharmaceutically acceptable salt thereof. In some embodiments, the PCS score is 30 or greater, about 5 minutes to about 24 hours prior to intranasal administration of the racemic ketamine, or a pharmaceutically acceptable salt thereof. For example, about 5 minutes to 1 hour, about 5 minutes to 6 hours, about 5 minutes to 12 hours, about 5 minutes to 18 hours, about 18 hours to 24 hours, about 12 hours to 24 hours, or about 6 hours to 24 hours prior to intranasal administration of the racemic ketamine, or a pharmaceutically acceptable salt thereof. In some embodiments, the PCS score is 20 or greater, about 5 minutes to about 24 hours prior to intranasal administration of the racemic ketamine, or a pharmaceutically acceptable salt thereof. For example, about 5 minutes to 1 hour, about 5 minutes to 6 hours, about 5 minutes to 12 hours, about 5 minutes to 18 hours, about 18 hours to 24 hours, about 12 hours to 24 hours, or about 6 hours to 24 hours prior to intranasal administration of the racemic ketamine, or a pharmaceutically acceptable salt thereof. In some embodiments, the PCS score is 10 or greater, about 5 minutes to about 24 hours prior to intranasal administration of the racemic ketamine, or a pharmaceutically acceptable salt thereof. For example, about 5 minutes to 1 hour, about 5 minutes to 6 hours, about 5 minutes to 12 hours, about 5 minutes to 18 hours, about 18 hours to 24 hours, about 12 hours to 24 hours, or about 6 hours to 24 hours prior to intranasal administration of the racemic ketamine, or a pharmaceutically acceptable salt thereof. In some embodiments, the PCS score is 5 or greater, about 5 minutes to about 24 hours prior to intranasal administration of the racemic ketamine, or a pharmaceutically acceptable salt thereof. For example, about 5 minutes to 1 hour, about 5 minutes to 6 hours, about 5 minutes to 12 hours, about 5 minutes to 18 hours, about 18 hours to 24 hours, about 12 hours to 24 hours, or about 6 hours to 24 hours prior to intranasal administration of the racemic ketamine, or a pharmaceutically acceptable salt thereof. 20 In some embodiments, the PCS score decreases by 5 to 52 (e.g., by 5 to 52 units) after intranasal administration of the racemic ketamine, or a pharmaceutically acceptable salt thereof. In some embodiments, the PCS score decreases by 10 to 35 (e.g., by 10 to 35 units) after intranasal administration of the racemic ketamine, or a pharmaceutically acceptable salt thereof. In some embodiments, the PCS score decreases by 15 to 25 (e.g., by 15 to 25 units) after intranasal administration of the racemic ketamine, or a pharmaceutically acceptable salt thereof. In some embodiments, the PCS score decreases by 5 to 10 (e.g., 5 to 10 units) after intranasal administration of the racemic ketamine, or a pharmaceutically acceptable salt thereof. In some embodiments, the PCS score decreases by 1 to 5 (e.g., 1 to 5 units) after intranasal administration of the racemic ketamine, or a pharmaceutically acceptable salt thereof.

The Pelvic Pain and Urgency/Frequency (PUF) patient symptom scale is a diagnostic tool used to screen subject/patients with chronic pelvic pain. The PUF self-report questionnaire uses a symptom score (which measures how often a subject/patient experiences problems) as well as a bother score (which notes the degree to which the symptoms bother the subject/patient); the bother and symptom score combine for a total PUF score. The questions, of which there are 8, include things like: “How many times do you go to the bathroom during the day (or night)?”, “Does your pain bother you?”, and “Does your urgency bother you?” Scores range from 0 and 35, and studies have indicated that a score greater than 12 is indicative of significant symptoms.

In some embodiments, the PUF score of the subject is 25 to 35 prior to intranasal administration of the racemic ketamine, or a pharmaceutically acceptable salt thereof. In some embodiments, the PUF score of the subject is 20 or more prior to intranasal administration of the racemic ketamine, or a pharmaceutically acceptable salt thereof. In some embodiments, the PUF score of the subject is 15 or more prior to intranasal administration of the racemic ketamine, or a pharmaceutically acceptable salt thereof. In some embodiments, the PUF score of the subject is 10 or more prior to intranasal administration of the racemic ketamine, or a pharmaceutically acceptable salt thereof. In some embodiments, the PUF score of the subject is 5 or more prior to intranasal administration of the racemic ketamine, or a pharmaceutically acceptable salt thereof. In some embodiments, the PUF score of the subject is 5 or less prior to intranasal administration of the racemic ketamine, or a pharmaceutically acceptable salt thereof.

In some embodiments, the PUF score decreases by 5 to 35 (e.g., by 5 to 35 units) after intranasal administration of the racemic ketamine, or a pharmaceutically acceptable salt thereof. In some embodiments, the PUF score decreases by 10 to 25 (e.g., by 10 to 25 units) after intranasal administration of the racemic ketamine, or a pharmaceutically acceptable salt thereof. In some embodiments, the PUF score decreases by 15 to 20 (e.g., by 15 to 20 units) after intranasal administration of the racemic ketamine, or a pharmaceutically acceptable salt thereof. In some embodiments, the PUF score decreases by 5 to 10 (e.g., 5 to 10 units) after intranasal administration of the racemic ketamine, or a pharmaceutically acceptable salt thereof. In some embodiments, the PUF score decreases by 5 to 0 (e.g., 1 to 0 units) after intranasal administration of the racemic ketamine, or a pharmaceutically acceptable salt thereof.

In some embodiments, the subject has a NPSR score of about 6 to about 11 within about 1 minute to about 10 days prior to the initiation of administration of racemic ketamine, or a pharmaceutically acceptable salt thereof.

### Reduced Side Effects

Some embodiments provide a method for reducing one or more side effects of ketamine in a subject in need thereof, the method comprising intranasally administering a therapeutically effective amount of racemic ketamine, or a pharmaceutically acceptable salt thereof, to the subject.

Some embodiments provide a reduced side-effect profile after administration of ketamine, specifically, after administration of the intranasal racemic ketamine, or a pharmaceutically acceptable salt thereof described herein, relative to administration of (S)-ketamine or intravenous racemic ketamine, or a pharmaceutically acceptable salt of either of the foregoing.

In some embodiments, no clinically meaningful sedation is observed in the subject within about 24 hours after intranasal administration of the racemic ketamine, or a pharmaceutically acceptable salt thereof. In some embodiments, no clinically meaningful sedation is observed in the subject at about 4 hours after intranasal administration of the racemic ketamine, or a pharmaceutically acceptable salt thereof. In some embodiments, no clinically meaningful sedation is observed in the subject at about 1 hour after intranasal administration of the racemic ketamine, or a pharmaceutically acceptable salt thereof.

In some embodiments, no clinically meaningful dissociation is observed in the subject within about 24 hours after intranasal administration of the racemic ketamine, or a pharmaceutically acceptable salt thereof. In some embodiments, no clinically meaningful dissociation is observed in the subject at about 4 hours after intranasal administration of the racemic ketamine, or a pharmaceutically acceptable salt thereof. In some embodiments, no clinically meaningful dissociation is observed in the subject at about 1 hour after intranasal administration of the racemic ketamine, or a pharmaceutically acceptable salt thereof.

In some embodiments, the subject has been previously diagnosed with and/or is currently suffering from, post-traumatic stress disorder. In some embodiments, the subject exhibits one or more of the following characteristics: unwanted upsetting memories, nightmares, flashbacks, emotional distress after exposure to traumatic reminders, or physical reactivity after exposure to traumatic reminders; and one or more of trauma-related thoughts or feelings and trauma-related external reminders. In some embodiments, the subject exhibits two or more of the following characteristics: inability to recall key features of a traumatic event, overly negative thoughts and assumptions about oneself or the world, exaggerated blame of self or others for causing a traumatic event, negative affect, decreased interest in activities, feeling isolated, and difficulty experiencing positive affect. In some embodiments, the subject exhibits one or more of the following characteristics: irritability or aggression, risky or destructive behavior, hypervigilance, heightened startle reaction, difficulty concentrating, and difficulty sleeping. In some embodiments, the characteristics are present for more than about 1 month, create distress and/or functional impairment in social or occupational situations, and are not due to medication or substance abuse. In some embodiments, the characteristics are present for at least about 1 month up to about 12 months.

In some embodiments, the subject has been previously diagnosed with and/or is currently suffering from, major depressive disorder. In some embodiments, the subject has not been diagnosed with, or is not currently suffering from suicidality. In some embodiments, the subject has not been diagnosed with, or is not currently suffering from suicidal ideation.

In some embodiments, the subject has been previously diagnosed with and/or is currently suffering from, treatment-resistant depression. In some embodiments, the treatment-resistant depression is Stage I to Stage IV. In some embodiments, the treatment resistant depression is Stage V. In some embodiments, the subject has not been diagnosed with, or is not currently suffering from suicidality. In some embodiments, the subject has not been diagnosed with, or is not currently suffering from suicidal ideation.

In some embodiments, the subject has been previously diagnosed with and/or is currently suffering from, bipolar depression. In some embodiments, the subject has been previously diagnosed with and/or is currently suffering from, post-partum depression. In some embodiments, the subject has been previously diagnosed with and/or is currently suffering from, post-partum depression and is not currently breastfeeding. In some embodiments, the subject has been previously diagnosed with and/or is currently suffering from, chronic pain. In some embodiments, the subject has been previously diagnosed with and/or is currently suffering from, neuropathic pain. In some embodiments, the subject has been previously diagnosed with and/or is currently suffering from, Rett syndrome. In some embodiments, the subject has been previously diagnosed with and/or is currently suffering from, epilepsy. In some embodiments, the subject has been previously diagnosed with and/or is currently suffering from, agitation associated with dementia. In some embodiments, the subject has been previously diagnosed with and/or is currently suffering from, agitation associated with schizophrenia. In some embodiments, the subject has been previously diagnosed with and/or is currently suffering from, agitation associated with bipolar disorder.

In some embodiments, one or more side effects of the intranasal racemic ketamine, or a pharmaceutically acceptable salt thereof, and/or the one or more additional therapies, required to provide a therapeutic effect is reduced relative to the side effects of each individual agent when administered alone. In some embodiments, one or more side effects of the ketamine, or a pharmaceutically acceptable salt thereof, is reduced. In some embodiments, one or more side effects of the one or more additional therapies is reduced. In some embodiments, one or more side effects of the one or more additional therapies is reduced relative to the one or more side effects observed after administration with an equivalent dose of intravenous racemic ketamine, or a pharmaceutically acceptable salt thereof. In some embodiments, one or more side effects of the one or more additional therapies is reduced relative to the one or more side effects observed after administration with an equivalent dose of (S)-ketamine, or a pharmaceutically acceptable salt thereof. In some embodiments, one or more side effects of both the ketamine, or a pharmaceutically acceptable salt thereof, and the one or more additional therapies are reduced. In some embodiments, the total number of side effects is reduced. In some embodiments, the magnitude of one or more side effects is reduced. In some embodiments, both the total number of side effects is reduced and the magnitude of one or more remaining side effects is also reduced.

In some embodiments, the one or more side effects of ketamine comprise cognitive impairment, motor impairment, vertigo, nausea, vomiting, sweating, increased blood pressure, ulcerative cystitis, or interstitial cystitis. In some embodiments, the one or more side effects of ketamine consist of cognitive impairment, motor impairment, vertigo, nausea, vomiting, sweating, increased blood pressure, ulcerative cystitis, or interstitial cystitis.

In some embodiments, the cognitive impairment comprises one or more of psychotomimetic effects, dizziness, dysgeusia, sedation, dissociation, euphoria, changes in hearing, changes in vision, and hallucinations. In some embodiments, the cognitive impairment consists of one or more of psychotomimetic effects, dizziness, dysgeusia, sedation, dissociation, euphoria, changes in hearing, changes in vision, and hallucinations. In some embodiments, the cognitive impairment comprises sedation. In some embodiments, the cognitive impairment is sedation. In some embodiments, the motor impairment comprises tremors, issues with balance, or dystonic movements. In some embodiments, the motor impairment consists of one or more of tremors, issues with balance, and dystonic movements.

Many methods can be used to assess side effects associated with ketamine. Non-limiting examples of such methods include the Modified Observer's Assessment of Alertness/Sedation Scale (MOAA/S), Bowdle Visual Analog Scale (VAS), the Clinician Administered Dissociative States Scale (CADSS), the Profile of Mood States (POMS), Choice Reaction Time (CRT) Test, Sternberg Short-Term Memory (SSTM) Task, and the Subject-Rated Assessment of Intranasal Irritation (SRAII©) (for intranasal administration of ketamine).

The Modified Observer's Assessment of Alertness/Sedation (MOAA/S) Scale is a 6-point scale that is based on responsiveness to voice and touch, speech, facial expression, and eye ptosis. The MOAA/S scale ranges from 0 to 6, where 0 indicates the patient has no response after a painful trapezius squeeze; 1 indicates the subject responds only after a painful trapezius squeeze; 2 indicates the patient responds only after mild prodding or shaking; 3 indicates the subject responds only after name is called loudly and/or repeatedly; 4 indicates the subject has a lethargic response to name spoken in normal tone; 5 indicates the subject has a slightly lethargic response to name spoken in normal tone; and 6 indicates the subject readily responds to name spoken in normal tone.

In some embodiments, the MOAA/S can be used to measure sedation in a subject. See, e.g., Kim et al., Br J Anaesth, Vol. 115, No. 4, pp. 569-577 (2015), which is incorporated by reference herein in its entirety. In some embodiments, the MOAA/S score of the subject is 5 units prior to intranasal administration of the racemic ketamine, or a pharmaceutically acceptable salt thereof. In some embodiments, the MOAA/S score of the subject is 4 or 5 units from about 15 minutes to about 6 hours after intranasal administration of the racemic ketamine, or a pharmaceutically acceptable salt thereof.

In some embodiments, the MOAA/S of the subject is 5 or 6 prior to intranasal administration of racemic ketamine, or a pharmaceutically acceptable salt thereof, as described herein. In some embodiments, the MOAA/S score of the subject is measured at about 5 minutes to about 24 hours prior to intranasal administration of the racemic ketamine, or a pharmaceutically acceptable salt thereof. For example, about 5 minutes to 1 hour, about 5 minutes to 6 hours, about 5 minutes to 12 hours, about 5 minutes to 18 hours, about 18 hours to 24 hours, about 12 hours to 24 hours, or about 6 hours to 24 hours prior to intranasal administration of the racemic ketamine, or a pharmaceutically acceptable salt thereof. In some embodiments, the MOAA/S score of the subject is as described herein about 1 hour to about 4 hours, about 1.5 hours to about 5 hours, about 2 hours to about 6 hours, about 5 hours to about 10 hours, or about 45 minutes to about 24 hours prior to intranasal administration of the racemic ketamine, or a pharmaceutically acceptable salt thereof.

In some embodiments, the MOAA/S of the subject is 5 or 6 after intranasal administration of racemic ketamine, or a pharmaceutically acceptable salt thereof, as described herein. In some embodiments, the MOAA/S score of the subject is measured at about 5 minutes to about 24 hours after intranasal administration of the racemic ketamine, or a pharmaceutically acceptable salt thereof. For example, about 5 minutes to 1 hour, about 5 minutes to 6 hours, about 5 minutes to 12 hours, about 5 minutes to 18 hours, about 18 hours to 24 hours, about 12 hours to 24 hours, or about 6 hours to 24 hours after intranasal administration of the racemic ketamine, or a pharmaceutically acceptable salt thereof. In some embodiments, the MOAA/S score of the subject is as described herein about 1 hour to about 4 hours, about 1.5 hours to about 5 hours, about 2 hours to about 6 hours, about 5 hours to about 10 hours, or about 45 minutes to about 24 hours after intranasal administration of the racemic ketamine, or a pharmaceutically acceptable salt thereof.

In some embodiments, the MOAA/S of the subject is 5 or 6 prior to intranasal administration of racemic ketamine, or a pharmaceutically acceptable salt thereof, and the MOAA/S of the subject is 5 or 6 after intranasal administration of racemic ketamine, or a pharmaceutically acceptable salt thereof. In some embodiments, the MOAA/S score of the subject is measured at about 5 minutes to about 24 hours prior to intranasal administration of the racemic ketamine, or a pharmaceutically acceptable salt thereof, and about 5 minutes to about 24 hours after to intranasal administration of the racemic ketamine, or a pharmaceutically acceptable salt thereof. For example, about 5 minutes to 1 hour, about 5 minutes to 6 hours, about 5 minutes to 12 hours, about 5 minutes to 18 hours, about 18 hours to 24 hours, about 12 hours to 24 hours, or about 6 hours to 24 hours prior to intranasal administration of the racemic ketamine, or a pharmaceutically acceptable salt thereof, and about 5 minutes to 1 hour, about 5 minutes to 6 hours, about 5 minutes to 12 hours, about 5 minutes to 18 hours, about 18 hours to 24 hours, about 12 hours to 24 hours, or about 6 hours to 24 hours after intranasal administration of the racemic ketamine, or a pharmaceutically acceptable salt thereof. In some embodiments, the MOAA/S score of the subject is 5 or 6 at about 1 hour to about 4 hours, about 1.5 hours to about 5 hours, about 2 hours to about 6 hours, about 5 hours to about 10 hours, or about 45 minutes to about 24 hours prior to intranasal administration of the racemic ketamine, or a pharmaceutically acceptable salt thereof, and the MOAA/S score of the subject is 5 or 6 at about 1 hour to about 4 hours, about 1.5 hours to about 5 hours, about 2 hours to about 6 hours, about 5 hours to about 10 hours, or about 45 minutes to about 24 hours after intranasal administration of the racemic ketamine, or a pharmaceutically acceptable salt thereof.

In some embodiments, the MOAA/S score of the subject is substantially the same prior to and after intranasal administration of the racemic ketamine, or a pharmaceutically acceptable salt thereof. For example, the MOAA/S score of the subject does not change (i.e., does not increase or decrease) after intranasal administration of the racemic ketamine, or a pharmaceutically acceptable salt thereof, relative to prior to intranasal administration of the racemic ketamine, or a pharmaceutically acceptable salt thereof. In some embodiments, the MOAA/S score of the subject is substantially the same about 5 minutes to about 24 hours prior to intranasal administration of the racemic ketamine, or a pharmaceutically acceptable salt thereof, as about 5 minutes to about 24 hours after intranasal administration of the racemic ketamine, or a pharmaceutically acceptable salt thereof. For example, about 5 minutes to 1 hour, about 5 minutes to 6 hours, about 5 minutes to 12 hours, about 5 minutes to 18 hours, about 18 hours to 24 hours, about 12 hours to 24 hours, about 6 hours to 24 hours, 1 hour to about 4 hours, about 1.5 hours to about 5 hours, about 2 hours to about 6 hours, or about 5 hours to about 10 hours prior to intranasal administration of the racemic ketamine, or a pharmaceutically acceptable salt thereof, and about 5 minutes to 1 hour, about 5 minutes to 6 hours, about 5 minutes to 12 hours, about 5 minutes to 18 hours, about 18 hours to 24 hours, about 12 hours to 24 hours, about 6 hours to 24 hours, 1 hour to about 4 hours, about 1.5 hours to about 5 hours, about 2 hours to about 6 hours, or about 5 hours to about 10 hours after intranasal administration of the racemic ketamine, or a pharmaceutically acceptable salt thereof.

In some embodiments, the MOAA/S score of the subject is reduced by about 1 to about 5 after intranasal administration of the racemic ketamine, or a pharmaceutically acceptable salt thereof. For example, the MOAA/S score of the subject can be reduced by 1, 2, 3, 4, or 5 after intranasal administration of the racemic ketamine, or a pharmaceutically acceptable salt thereof, relative to administration of an equivalent dose of (S)-ketamine, or a pharmaceutically acceptable salt thereof, and/or intravenous administration of an equivalent dose of (S)-ketamine, or a pharmaceutically acceptable salt thereof. In some embodiments, the MOAA/S score of the subject is reduced as described herein about 5 minutes to about 24 hours after intranasal administration of the racemic ketamine, or a pharmaceutically acceptable salt thereof. For example, about 5 minutes to 1 hour, about 5 minutes to 6 hours, about 5 minutes to 12 hours, about 5 minutes to 18 hours, about 18 hours to 24 hours, about 12 hours to 24 hours, or about 6 hours to 24 hours after intranasal administration of the racemic ketamine, or a pharmaceutically acceptable salt thereof. In some embodiments, the MOAA/S score of the subject is as described herein about 1 hour to about 4 hours, about 1.5 hours to about 5 hours, about 2 hours to about 6 hours, or about 5 hours to about 10 hours, after intranasal administration of the racemic ketamine, or a pharmaceutically acceptable salt thereof, (e.g., relative to administration of an equivalent dose of (S)-ketamine, or a pharmaceutically acceptable salt thereof, and/or intravenous administration of an equivalent dose of (S)-ketamine, or a pharmaceutically acceptable salt thereof).

In some embodiments, the Bowdle Visual Analog Scale (VAS) can be used to measure psychedelic effects in a subject. See, e.g., Bowdle, et al. Anesthesiology, Vol. 88, No. 1, pp. 82-88 (1998), which is incorporated herein by reference in its entirety. The Bowdle VAS a 13-item structured clinical interview to assess psychedelic side effects. The VAS consists of 13 items for which the subject is asked to rate their current feelings. Each item will be scored from 0 to 100, with 0 reflecting “Not at all” and 100 reflecting “Extremely.” Lower individual and overall scores indicate fewer psychedelic effects. The individual items of the questionnaire are listed below:
1. My body or body parts seemed to change their shape or position (BODY)
2. My surroundings seemed to change in size, depth, or shape (SURROUNDINGS)
3. The passing of time was altered (TIME)
4. I had feelings of unreality (REALITY)
5. It was difficult to control my thoughts (THOUGHTS)
6. The intensity of colors changed (COLORS)
7. The intensity of sound changed (SOUND)
8. I heard voices or sounds that were not real (VOICES)
9. I had the idea that events, objects, or other people had particular meaning that was specific for me (MEANING)
10. I had suspicious ideas or the belief that others were against me (SUSPICIOUS)
11. I felt anxious (ANXIOUS)
12. I felt high (HIGH)
13. I felt drowsy (DROWSY)

Items 1, 2, 3, 5, 6, and 7 are combined to assess the derived variable “subjective external perception.” Items 4, 8, 9, 10, and 11 are combined to assess the derived variable “subjective internal perception.” Items 12 and 13 will be assessed as individual VAS items. If one of the items is missing, the related score will not be calculated.

In some embodiments, items 1, 2, 3, 5, 6, and 7 are combined to assess the derived variable “subjective external perception.” In some embodiments, items 4, 8, 9, 10, and 11 are combined to assess the derived variable “subjective internal perception.” In some embodiments, items 12 and 13 are assessed as individual VAS items.

In some embodiments, the Bowdle VAS of the subject 0 to 50 prior to intranasal administration of racemic ketamine, or a pharmaceutically acceptable salt thereof, as described herein. In some embodiments, the Bowdle VAS of the subject is 25 to 75 prior to intranasal administration of racemic ketamine, or a pharmaceutically acceptable salt thereof, as described herein. In some embodiments, the Bowdle VAS of the subject is 50 to 100 prior to intranasal administration of racemic ketamine, or a pharmaceutically acceptable salt thereof, as described herein. In some embodiments, the Bowdle VAS of the subject is 0 to 10, 0 to 20, 0 to 30, 0 to 40, 0 to 50, 0 to 60, 0 to 70, 0 to 80, 0 to 90, 90 to 100, 80 to 100, 70 to 100, 60 to 100, 50 to 100, 40 to 100, 30 to 100, 20 to 100, or 10 to 100 prior to intranasal administration of racemic ketamine as described herein. In some embodiments, the Bowdle VAS of the subject is 5 to 20, 15 to 40, 10 to 50, or 20 to 60 prior to intranasal administration of racemic ketamine as described herein. In some embodiments, the Bowdle VAS score of the subject is measured at about 5 minutes to about 24 hours prior to intranasal administration of the racemic ketamine, or a pharmaceutically acceptable salt thereof. For example, about 5 minutes to 1 hour, about 5 minutes to 6 hours, about 5 minutes to 12 hours, about 5 minutes to 18 hours, about 18 hours to 24 hours, about 12 hours to 24 hours, or about 6 hours to 24 hours prior to intranasal administration of the racemic ketamine, or a pharmaceutically acceptable salt thereof. In some embodiments, the Bowdle VAS score of the subject is measured at about 1 hour to about 4 hours, about 1.5 hours to about 5 hours, about 2 hours to about 6 hours, about 5 hours to about 10 hours, or about 45 minutes to about 24 hours prior to intranasal administration of the racemic ketamine, or a pharmaceutically acceptable salt thereof.

In some embodiments, the Bowdle VAS of the subject is 0 to 50 after intranasal administration of racemic ketamine, or a pharmaceutically acceptable salt thereof, as described herein. In some embodiments, the Bowdle VAS of the subject is 25 to 75 after intranasal administration of racemic ketamine, or a pharmaceutically acceptable salt thereof, as described herein. In some embodiments, the Bowdle VAS of the subject is 50 to 100 after intranasal administration of racemic ketamine, or a pharmaceutically acceptable salt thereof, as described herein. In some embodiments, the Bowdle VAS of the subject is 0 to 10, 0 to 20, 0 to 30, 0 to 40, 0 to 50, 0 to 60, 0 to 70, 0 to 80, 0 to 90, 90 to 100, 80 to 100, 70 to 100, 60 to 100, 50 to 100, 40 to 100, 30 to 100, 20 to 100, or 10 to 100 after intranasal administration of racemic ketamine as described herein. In some embodiments, the Bowdle VAS of the subject is 5 to 20, 15 to 40, 10 to 50, or 20 to 60 after intranasal administration of racemic ketamine as described herein. In some embodiments, the Bowdle VAS score of the subject is measured at about 5 minutes to about 24 hours after intranasal administration of the racemic ketamine, or a pharmaceutically acceptable salt thereof. For example, about 5 minutes to 1 hour, about 5 minutes to 6 hours, about 5 minutes to 12 hours, about 5 minutes to 18 hours, about 18 hours to 24 hours, about 12 hours to 24 hours, or about 6 hours to 24 hours after intranasal administration of the racemic ketamine, or a pharmaceutically acceptable salt thereof. In some embodiments, the Bowdle VAS score of the subject is measured at about 1 hour to about 4 hours, about 1.5 hours to about 5 hours, about 2 hours to about 6 hours, about 5 hours to about 10 hours, or about 45 minutes to about 24 hours after intranasal administration of the racemic ketamine, or a pharmaceutically acceptable salt thereof.

In some embodiments, the Bowdle VAS of the subject is 0 to 50 prior to intranasal administration of racemic ketamine, or a pharmaceutically acceptable salt thereof, as described herein, and the Bowdle VAS of the subject is 0 to 50 after intranasal administration of racemic ketamine, or a pharmaceutically acceptable salt thereof. In some embodiments, the Bowdle VAS of the subject is 25 to 75 prior to intranasal administration of racemic ketamine, or a pharmaceutically acceptable salt thereof, as described herein, and the Bowdle VAS of the subject is 25 to 75 after intranasal administration of racemic ketamine, or a pharmaceutically acceptable salt thereof, as described herein. In some embodiments, the Bowdle VAS of the subject is 50 to 100 prior to intranasal administration of racemic ketamine, or a pharmaceutically acceptable salt thereof, as described herein and the Bowdle VAS of the subject is 50 to 100 after intranasal administration of racemic ketamine, or a pharmaceutically acceptable salt thereof, as described herein. In some embodiments, the Bowdle VAS of the subject is 0 to 10, 0 to 20, 0 to 30, 0 to 40, 0 to 50, 0 to 60, 0 to 70, 0 to 80, 0 to 90, 90 to 100, 80 to 100, 70 to 100, 60 to 100, 50 to 100, 40 to 100, 30 to 100, 20 to 100, or 10 to 100 prior to intranasal administration of racemic ketamine as described herein and 0 to 10, 0 to 20, 0 to 30, 0 to 40, 0 to 50, 0 to 60, 0 to 70, 0 to 80, 0 to 90, 90 to 100, 80 to 100, 70 to 100, 60 to 100, 50 to 100, 40 to 100, 30 to 100, 20 to 100, or 10 to 100 after intranasal administration of racemic ketamine as described herein. In some embodiments, the Bowdle VAS of the subject is 5 to 20, 15 to 40, 10 to 50, or 20 to 60 prior to intranasal administration of racemic ketamine as described herein and the Bowdle VAS of the subject is 5 to 20, 15 to 40, 10 to 50, or 20 to 60 after intranasal administration of racemic ketamine as described herein. In some embodiments, the Bowdle VAS score of the subject is measured at about 5 minutes to about 24 hours prior to intranasal administration of the racemic ketamine, or a pharmaceutically acceptable salt thereof, and the Bowdle VAS score of the subject is measured at about 5 minutes to about 24 hours after intranasal administration of the racemic ketamine, or a pharmaceutically acceptable salt thereof. For example, about 5 minutes to 1 hour, about 5 minutes to 6 hours, about 5 minutes to 12 hours, about 5 minutes to 18 hours, about 18 hours to 24 hours, about 12 hours to 24 hours, or about 6 hours to 24 hours prior to intranasal administration of the racemic ketamine, or a pharmaceutically acceptable salt thereof, and about 5 minutes to 1 hour, about 5 minutes to 6 hours, about 5 minutes to 12 hours, about 5 minutes to 18 hours, about 18 hours to 24 hours, about 12 hours to 24 hours, or about 6 hours to 24 hours after intranasal administration of the racemic ketamine, or a pharmaceutically acceptable salt thereof. In some embodiments, the Bowdle VAS score of the subject is measured at about 1 hour to about 4 hours, about 1.5 hours to about 5 hours, about 2 hours to about 6 hours, about 5 hours to about 10 hours, or about 45 minutes to about 24 hours prior to intranasal administration of the racemic ketamine, or a pharmaceutically acceptable salt thereof, and the Bowdle VAS score of the subject is measured at about 1 hour to about 4 hours, about 1.5 hours to about 5 hours, about 2 hours to about 6 hours, about 5 hours to about 10 hours, or about 45 minutes to about 24 hours after intranasal administration of the racemic ketamine, or a pharmaceutically acceptable salt thereof.

In some embodiments, the Bowdle VAS score of the subject is substantially the same prior to and after intranasal administration of the racemic ketamine, or a pharmaceutically acceptable salt thereof. For example, the Bowdle VAS score of the subject changes (i.e., increases or decreases) by 0 to 10 after intranasal administration of the racemic ketamine, or a pharmaceutically acceptable salt thereof, relative to the Bowdle VAS score of the subject prior to intranasal administration of the racemic ketamine, or a pharmaceutically acceptable salt thereof. In some embodiments, the Bowdle VAS score of the subject is substantially the same about 5 minutes to about 24 hours prior to intranasal administration of the racemic ketamine, or a pharmaceutically acceptable salt thereof, as about 5 minutes to about 24 hours after intranasal administration of the racemic ketamine, or a pharmaceutically acceptable salt thereof. For example, about 5 minutes to 1 hour, about 5 minutes to 6 hours, about 5 minutes to 12 hours, about 5 minutes to 18 hours, about 18 hours to 24 hours, about 12 hours to 24 hours, about 6 hours to 24 hours, 1 hour to about 4 hours, about 1.5 hours to about 5 hours, about 2 hours to about 6 hours, or about 5 hours to about 10 hours prior to intranasal administration of the racemic ketamine, or a pharmaceutically acceptable salt thereof, and about 5 minutes to 1 hour, about 5 minutes to 6 hours, about 5 minutes to 12 hours, about 5 minutes to 18 hours, about 18 hours to 24 hours, about 12 hours to 24 hours, about 6 hours to 24 hours, 1 hour to about 4 hours, about 1.5 hours to about 5 hours, about 2 hours to about 6 hours, or about 5 hours to about 10 hours after intranasal administration of the racemic ketamine, or a pharmaceutically acceptable salt thereof.

In some embodiments, the Bowdle VAS score of the subject is reduced by 10 to 1300 after intranasal administration of the racemic ketamine, or a pharmaceutically acceptable salt thereof. For example, the Bowdle VAS score of the subject can be reduced by 10 to 1300 after intranasal administration of the racemic ketamine, or a pharmaceutically acceptable salt thereof, relative to the score observed after administration of an equivalent dose of (S)-ketamine, or a pharmaceutically acceptable salt thereof, and/or intravenous administration of an equivalent dose of racemic ketamine, or a pharmaceutically acceptable salt thereof. In some embodiments, the Bowdle VAS score of the subject is reduced by 10 to 100, 10 to 200, 10 to 300, 10 to 400, 10 to 500, 10 to 600, 10 to 700, 10 to 800, 10 to 900, 10 to 1000, 10 to 1100, 10 to 1200, 1200 to 1300, 1100 to 1300, 1000 to 1300, 900 to 1300, 800 to 1300, 700 to 1300, 600 to 1300, 500 to 1300, 400 to 1300, 300 to 1300, 200 to 1300, or 100 to 1300 after intranasal administration of the racemic ketamine, or a pharmaceutically acceptable salt thereof. In some embodiments, the Bowdle VAS score of the subject is reduced as described herein 5 minutes to 24 hours after intranasal administration of the racemic ketamine, or a pharmaceutically acceptable salt thereof. For example, about 5 minutes to 1 hour, about 5 minutes to 6 hours, about 5 minutes to 12 hours, about 5 minutes to 18 hours, about 18 hours to 24 hours, about 12 hours to 24 hours, or about 6 hours to 24 hours after intranasal administration of the racemic ketamine, or a pharmaceutically acceptable salt thereof. In some embodiments, the Bowdle VAS score of the subject is measured at about 1 hour to about 4 hours, about 1.5 hours to about 5 hours, about 2 hours to about 6 hours, or about 5 hours to about 10 hours, after intranasal administration of the racemic ketamine, or a pharmaceutically acceptable salt thereof, (or after administration of an equivalent dose of (S)-ketamine, or a pharmaceutically acceptable salt thereof, or intravenous administration of an equivalent dose of racemic ketamine, or a pharmaceutically acceptable salt thereof).

CADSS is a 23-item questionnaire assessing the dissociative state of the subject. See, e.g., Luckenbaugh, et al. J. Affect. Disord., Vol. 159, pp. 56-61 (2014), which is incorporated herein by reference in its entirety. Each question records a response from the subject from a scale of 0 to 4, 0=not at all, 1=mild, 2=moderate, 3=severe, and 4-extreme; the questions ask about things like “do things seem like they are moving in slow motion”, “do you feel as if you are watching the situation as an observer or a spectator?”, “do things seem very real, as if there is a special sense of clarity?”, and the like. The total score is 0-92, with the higher score being a subject in a high dissociative state.

The CADSS assessment includes, but are not limited to, statements such as things in slow motion, things seem unreal, feel separated from what is happening, out of body experience, feel as a spectator or observer, feel disconnected from the body, sense of body changed, people seem motionless/dead/mechanical, objects look different, colors are diminished in intensity, seeing things as if in a tunnel/wide-angle lens, things taking longer, things happening quickly, things happen that can't account for, losing track of what is going on, sounds change in intensity, special sense of clarity, as if looking through a fog, and colors seem brighter. Typically, CADSS will include 23 statements that the subject rates from 0-4, for a score ranging from 0 (no dissociation) to 92 (extreme dissociation). A score of 0 reflects that the subject did not feel at all as described in the item whereas a score of 4 reflects that the subject agreed with the question posed to the maximum level. e.g., 0 reflect not at all, 1 reflects mild agreement, 2 reflects moderate agreement, 3 reflects severe agreement, and 4 reflects the maximum level of agreement with the indicated question. Thus, lower individual and overall scores indicate less dissociation. In some embodiments, a portion of the scale is completed by the subject. In some embodiments, a portion of the scale is completed by a trained observer of the subject.

In some embodiments, the CADSS of the subject is 0 to 10 prior to intranasal administration of racemic ketamine, or a pharmaceutically acceptable salt thereof, as described herein. For example, 0 to 2, 0 to 3, 0 to 4, 0 to 5, 0 to 6, 0 to 7, 0 to 8, 0 to 9, 9 to 10, 8 to 10, 7 to 10, 6 to 10, 5 to 10, 4 to 10, 3 to 10, 2 to 10, or 1 to 10 prior to intranasal administration of racemic ketamine as described herein. In some embodiments, the CADSS of the subject is 2 to 6, 3 to 7, or 4 to 8 prior to intranasal administration of racemic ketamine as described herein. In some embodiments, the CADSS score of the subject is measured at about 5 minutes to about 24 hours prior to intranasal administration of the racemic ketamine, or a pharmaceutically acceptable salt thereof. For example, about 5 minutes to 1 hour, about 5 minutes to 6 hours, about 5 minutes to 12 hours, about 5 minutes to 18 hours, about 18 hours to 24 hours, about 12 hours to 24 hours, or about 6 hours to 24 hours prior to intranasal administration of the racemic ketamine, or a pharmaceutically acceptable salt thereof. In some embodiments, the CADSS Scale score of the subject is measured at about 1 hour to about 4 hours, about 1.5 hours to about 5 hours, about 2 hours to about 6 hours, about 5 hours to about 10 hours, or about 45 minutes to about 24 hours prior to intranasal administration of the racemic ketamine, or a pharmaceutically acceptable salt thereof.

In some embodiments, the CADSS of the subject is 0 to 10 after intranasal administration of racemic ketamine, or a pharmaceutically acceptable salt thereof, as described herein. For example, 0 to 2, 0 to 3, 0 to 4, 0 to 5, 0 to 6, 0 to 7, 0 to 8, 0 to 9, 9 to 10, 8 to 10, 7 to 10, 6 to 10, 5 to 10, 4 to 10, 3 to 10, 2 to 10, or 1 to 10 after intranasal administration of racemic ketamine as described herein. In some embodiments, the CADSS of the subject is 2 to 6, 3 to 7, or 4 to 8 after intranasal administration of racemic ketamine as described herein. In some embodiments, the CADSS score of the subject is measured at about 5 minutes to about 24 hours after intranasal administration of the racemic ketamine, or a pharmaceutically acceptable salt thereof. For example, about 5 minutes to 1 hour, about 5 minutes to 6 hours, about 5 minutes to 12 hours, about 5 minutes to 18 hours, about 18 hours to 24 hours, about 12 hours to 24 hours, or about 6 hours to 24 hours after intranasal administration of the racemic ketamine, or a pharmaceutically acceptable salt thereof. In some embodiments, the CADSS Scale score of the subject is measured at about 1 hour to about 4 hours, about 1.5 hours to about 5 hours, about 2 hours to about 6 hours, about 5 hours to about 10 hours, or about 45 minutes to about 24 hours after intranasal administration of the racemic ketamine, or a pharmaceutically acceptable salt thereof.

In some embodiments, the CADSS of the subject is 0 to 10 prior to intranasal administration of racemic ketamine, or a pharmaceutically acceptable salt thereof, and the CADSS of the subject is 0 to 10 after intranasal administration of racemic ketamine, or a pharmaceutically acceptable salt thereof. For example, 0 to 2, 0 to 3, 0 to 4, 0 to 5, 0 to 6, 0 to 7, 0 to 8, 0 to 9, 9 to 10, 8 to 10, 7 to 10, 6 to 10, 5 to 10, 4 to 10, 3 to 10, 2 to 10, or 1 to 10 prior to intranasal administration of racemic ketamine as described herein and 0 to 2, 0 to 3, 0 to 4, 0 to 5, 0 to 6, 0 to 7, 0 to 8, 0 to 9, 9 to 10, 8 to 10, 7 to 10, 6 to 10, 5 to 10, 4 to 10, 3 to 10, 2 to 10, or 1 to 10 after intranasal administration of racemic ketamine. In some embodiments, the CADSS of the subject is 2 to 6, 3 to 7, or 4 to 8 prior to intranasal administration of racemic ketamine as described herein and the CADSS of the subject is 2 to 6, 3 to 7, or 4 to 8 after intranasal administration of racemic ketamine as described herein. In some embodiments, the CADSS score of the subject is measured at about 5 minutes to about 24 hours prior to intranasal administration of the racemic ketamine, or a pharmaceutically acceptable salt thereof, and the CADSS score of the subject is measured at about 5 minutes to about 24 hours after intranasal administration of the racemic ketamine, or a pharmaceutically acceptable salt thereof. For example, about 5 minutes to 1 hour, about 5 minutes to 6 hours, about 5 minutes to 12 hours, about 5 minutes to 18 hours, about 18 hours to 24 hours, about 12 hours to 24 hours, or about 6 hours to 24 hours prior to intranasal administration of the racemic ketamine, or a pharmaceutically acceptable salt thereof, and about 5 minutes to 1 hour, about 5 minutes to 6 hours, about 5 minutes to 12 hours, about 5 minutes to 18 hours, about 18 hours to 24 hours, about 12 hours to 24 hours, or about 6 hours to 24 hours after intranasal administration of the racemic ketamine, or a pharmaceutically acceptable salt thereof. In some embodiments, the CADSS Scale score of the subject is measured at about 1 hour to about 4 hours, about 1.5 hours to about 5 hours, about 2 hours to about 6 hours, about 5 hours to about 10 hours, or about 45 minutes to about 24 hours prior to intranasal administration of the racemic ketamine, or a pharmaceutically acceptable salt thereof, and the CADSS Scale score of the subject is measured at about 1 hour to about 4 hours, about 1.5 hours to about 5 hours, about 2 hours to about 6 hours, about 5 hours to about 10 hours, or about 45 minutes to about 24 hours after intranasal administration of the racemic ketamine, or a pharmaceutically acceptable salt thereof.

In some embodiments, the CADSS score of the subject is substantially the same prior to and after intranasal administration of the racemic ketamine, or a pharmaceutically acceptable salt thereof. For example, the CADSS score of the subject changes (i.e., increases or decreases) by 0 to 5 after intranasal administration of the racemic ketamine, or a pharmaceutically acceptable salt thereof, relative to prior to intranasal administration of the racemic ketamine, or a pharmaceutically acceptable salt thereof. In some embodiments, the CADSS score of the subject is substantially the same about 5 minutes to about 24 hours prior to intranasal administration of the racemic ketamine, or a pharmaceutically acceptable salt thereof, as about 5 minutes to about 24 hours after intranasal administration of the racemic ketamine, or a pharmaceutically acceptable salt thereof. For example, about 5 minutes to 1 hour, about 5 minutes to 6 hours, about 5 minutes to 12 hours, about 5 minutes to 18 hours, about 18 hours to 24 hours, about 12 hours 20 to 24 hours, about 6 hours to 24 hours, 1 hour to about 4 hours, about 1.5 hours to about 5 hours, about 2 hours to about 6 hours, or about 5 hours to about 10 hours prior to intranasal administration of the racemic ketamine, or a pharmaceutically acceptable salt thereof, and about 5 minutes to 1 hour, about 5 minutes to 6 hours, about 5 minutes to 12 hours, about 5 minutes to 18 hours, about 18 hours to 24 hours, about 12 hours to 24 hours, about 6 hours to 24 hours, 1 hour to about 4 hours, about 1.5 hours to about 5 hours, about 2 hours to about 6 hours, or about 5 hours to about 10 hours after intranasal administration of the racemic ketamine, or a pharmaceutically acceptable salt thereof.

In some embodiments, the CADSS score of the subject is reduced by about 1 to about 91 after intranasal administration of the racemic ketamine, or a pharmaceutically acceptable salt thereof, relative to the score observed after administration of an equivalent dose of (S)-ketamine, or a pharmaceutically acceptable salt thereof, and/or intravenous administration of an equivalent dose of racemic ketamine, or a pharmaceutically acceptable salt thereof. For example, the CADDS score of the subject can be reduced by about 10 to about 91 after intranasal administration of the racemic ketamine, or a pharmaceutically acceptable salt thereof, relative to the score observed after administration of an equivalent dose of (S)-ketamine, or a pharmaceutically acceptable salt thereof, and/or intravenous administration of an equivalent dose of racemic ketamine, or a pharmaceutically acceptable salt thereof. In some embodiments, the CADSS score of the subject is reduced by about 1 to about 90, about 1 to about 80, about 1 to about 70, about 1 to about 60, about 1 to about 50, about 1 to about 40, about 1 to about 30, about 1 to about 20, about 1 to about 10, about 80 to about 91, about 70 to about 91, about 60 to about 91, about 50 to about 91, about 40 to about 91, about 30 to about 91, about 20 to about 91, or about 10 to about 91 after intranasal administration of the racemic ketamine, or a pharmaceutically acceptable salt thereof relative to the score observed after administration of an equivalent dose of (S)-ketamine, or a pharmaceutically acceptable salt thereof, and/or intravenous administration of an equivalent dose of racemic ketamine, or a pharmaceutically acceptable salt thereof. In some embodiments, the CADSS score of the subject is reduced about 5 minutes to about 24 hours after intranasal administration of the racemic ketamine, or a pharmaceutically acceptable salt thereof, relative to the score observed after administration of an equivalent dose of (S)-ketamine, or a pharmaceutically acceptable salt thereof, and/or intravenous administration of an equivalent dose of racemic ketamine, or a pharmaceutically acceptable salt thereof. For example, about 5 minutes to 1 hour, about 5 minutes to 6 hours, about 5 minutes to 12 hours, about 5 minutes to 18 hours, about 18 hours to 24 hours, about 12 hours to 24 hours, or about 6 hours to 24 hours after intranasal administration of the racemic ketamine, or a pharmaceutically acceptable salt thereof, relative to the score observed after administration of an equivalent dose of (S)-ketamine, or a pharmaceutically acceptable salt thereof, and/or intravenous administration of an equivalent dose of racemic ketamine, or a pharmaceutically acceptable salt thereof. In some embodiments, the CADDS score of the subject is measured at about 1 hour to about 4 hours, about 1.5 hours to about 5 hours, about 2 hours to about 6 hours, or about 5 hours to about 10 hours, after intranasal administration of the racemic ketamine, or a pharmaceutically acceptable salt thereof (or after administration of an equivalent dose of (S)-ketamine, or a pharmaceutically acceptable salt thereof, or intravenous administration of an equivalent dose of racemic ketamine, or a pharmaceutically acceptable salt thereof).

In some embodiments, the Clinician Administered Dissociative States Scale (CADSS) score of the subject is zero units prior to intranasal administration of the racemic ketamine, or a pharmaceutically acceptable salt thereof. In some embodiments, the CADSS score of the subject is zero units from about 1 hour to about 6 hours after intranasal administration of the racemic ketamine, or a pharmaceutically acceptable salt thereof.

The Profile of Mood States (POMS) is a psychological rating scale used to assess transient, distinct mood states. The POMS measures six different dimensions of mood swings over a period of time, including: Tension or Anxiety, Anger or Hostility, Vigor or Activity, Fatigue or Inertia, Depression or Dejection, Confusion or Bewilderment. Scores for each item are recorded as 0 for ‘Not at all’ up to 4 for ‘extremely’, except for the two Esteem-related Affect subscales which are reverse-scored prior to being combined with the other items.

In some embodiments, the Profile of Mood States (POMS) (e.g., POMS 2nd edition) can be used to measure transient feelings and mood in a subject. See, e.g., Lin, et al. JPA Vol. 32, No. 3, pp. 273-277 (2014), which is incorporated herein by reference in its entirety. The Profile of Mood States can includes items to monitor mood change in the subject.

In some embodiments, the Profile of Mood States score of the subject is substantially the same prior to and after intranasal administration of the racemic ketamine, or a pharmaceutically acceptable salt thereof. In some embodiments, the Profile of Mood States score of the subject is substantially the same about 5 minutes to about 24 hours prior to intranasal administration of the racemic ketamine, or a pharmaceutically acceptable salt thereof, as about 5 minutes to about 24 hours after intranasal administration of the racemic ketamine, or a pharmaceutically acceptable salt thereof. For example, about 5 minutes to 1 hour, about 5 minutes to 6 hours, about 5 minutes to 12 hours, about 5 minutes to 18 hours, about 18 hours to 24 hours, about 12 hours to 24 hours, about 6 hours to 24 hours, 1 hour to about 4 hours, about 1.5 hours to about 5 hours, about 2 hours to about 6 hours, or about 5 hours to about 10 hours prior to intranasal administration of the racemic ketamine, or a pharmaceutically acceptable salt thereof, and about 5 minutes to 1 hour, about 5 minutes to 6 hours, about 5 minutes to 12 hours, about 5 minutes to 18 hours, about 18 hours to 24 hours, about 12 hours to 24 hours, about 6 hours to 24 hours, 1 hour to about 4 hours, about 1.5 hours to about 5 hours, about 2 hours to about 6 hours, or about 5 hours to about 10 hours after intranasal administration of the racemic ketamine, or a pharmaceutically acceptable salt thereof.

The Choice reaction time (CRT) test assesses reaction times to a stimulus after the participant has been given some information about what kind of stimulus will appear next. This is also known as “go/no go” reaction test since it addresses whether the participant pressed a button or not.

In some embodiments, the Choice Reaction Time (CRT) test can be used to measure psychomotor performance in a subject. See, e.g., Hindmarch, et al. Br. J. Clin. Pharmcol., Vol. 49, No. 2, pp. 118-125 (2000), which is incorporated herein by reference in its entirety. The choice reaction time test is administered using a computer, where the subject is presented with an onscreen equivalent of a numeric keypad. When a key is illuminated on the screen, the subject presses the corresponding button on a separate keypad. For a given trial, four to eight numbered squares will be illuminated on the computer screen that correspond spatially to the keys on the keypad. The sequence of key illumination can be random. In some embodiments, the sequence of key illumination follows a pattern that alternates between the center button and any button that is part of the stimuli set of buttons. In some embodiments, the stimulus set size progresses from 4 to 6 to 8 during the test. The number of alternative choices can increase over blocks of responses in each cycle. The CRT test can include three outcome variables: recognition reaction time (RRT) is the time it takes for a subject to notice the light (e.g., the time between stimulus onset and the subject lifting his or her finger from the start button); motor reaction time (MRT) is the time between the subject lifting his or her finger from the start button and touching the response button; and total reaction time (TRT) is the sum of RRT and MRT. The accuracy of responses can also be recorded.

In some embodiments, the CRT test score of the subject is substantially the same prior to and after intranasal administration of the racemic ketamine, or a pharmaceutically acceptable salt thereof. In some embodiments, the CRT test score of the subject is substantially the same about 5 minutes to about 24 hours prior to intranasal administration of the racemic ketamine, or a pharmaceutically acceptable salt thereof, as about 5 minutes to about 24 hours after intranasal administration of the racemic ketamine, or a pharmaceutically acceptable salt thereof. For example, about 5 minutes to 1 hour, about 5 minutes to 6 hours, about 5 minutes to 12 hours, about 5 minutes to 18 hours, about 18 hours to 24 hours, about 12 hours to 24 hours, about 6 hours to 24 hours, 1 hour to about 4 hours, about 1.5 hours to about 5 hours, about 2 hours to about 6 hours, or about 5 hours to about 10 hours prior to intranasal administration of the racemic ketamine, or a pharmaceutically acceptable salt thereof, and about 5 minutes to 1 hour, about 5 minutes to 6 hours, about 5 minutes to 12 hours, about 5 minutes to 18 hours, about 18 hours to 24 hours, about 12 hours to 24 hours, about 6 hours to 24 hours, 1 hour to about 4 hours, about 1.5 hours to about 5 hours, about 2 hours to about 6 hours, or about 5 hours to about 10 hours after intranasal administration of the racemic ketamine, or a pharmaceutically acceptable salt thereof.

The Sternberg short-term memory (SSTM) task is designed to assess how a subject's working memory, i.e., how well they store and retrieve random information from short-term memory. Studying how individuals store and retrieve information from short-term memory provides an important window into more general cognitive processing and human functioning.

In some embodiments, the Sternberg short-term memory (SSTM) task can be used to measure immediate memory in a subject. See, e.g., Sternberg, Science, Vol. 153, Issue. 3736, pp. 652-654 (1966), which is incorporated herein by reference in its entirety. The SSTM can include asking a subject to remember a series of digits that are rapidly presented on a computer screen. For example, the SSTM can include rapid presentation of target lists of 2, 4, and 6 stimulus digits (e.g., at 1.2 seconds/digit), and two seconds after presentation of each list of digits, a series of 24 probe digits is presented. The subject is to identify as quickly as possible whether or not each probe appeared in the target list by pressing buttons on a response box corresponding to “yes” or “no.” Probes that appeared on the target list can be called “positive,” while probes that did not appear on the target list can be called “negative.” The SSTM can include three trials with digit sequence size lengths of 2, 4, and 6. Performance can be assessed by measures of response latency and accuracy.

In some embodiments, the SSTM score of the subject is substantially the same prior to and after intranasal administration of the racemic ketamine, or a pharmaceutically acceptable salt thereof. In some embodiments, the SSTM score of the subject is substantially the same about 5 minutes to about 24 hours prior to intranasal administration of the racemic ketamine, or a pharmaceutically acceptable salt thereof, as about 5 minutes to about 24 hours after intranasal administration of the racemic ketamine, or a pharmaceutically acceptable salt thereof. For example, about 5 minutes to 1 hour, about 5 minutes to 6 hours, about 5 minutes to 12 hours, about 5 minutes to 18 hours, about 18 hours to 24 hours, about 12 hours to 24 hours, about 6 hours to 24 hours, 1 hour to about 4 hours, about 1.5 hours to about 5 hours, about 2 hours to about 6 hours, or about 5 hours to about 10 hours prior to intranasal administration of the racemic ketamine, or a pharmaceutically acceptable salt thereof, and about 5 minutes to 1 hour, about 5 minutes to 6 hours, about 5 minutes to 12 hours, about 5 minutes to 18 hours, about 18 hours to 24 hours, about 12 hours to 24 hours, about 6 hours to 24 hours, 1 hour to about 4 hours, about 1.5 hours to about 5 hours, about 2 hours to about 6 hours, or about 5 hours to about 10 hours after intranasal administration of the racemic ketamine, or a pharmaceutically acceptable salt thereof.

The Subject-Rated Assessment of Intranasal Irritation (SRAII) can be used to determine intranasal irritation in a subject administered a drug (e.g., racemic ketamine or (S)-ketamine), or a pharmaceutically acceptable salt thereof. For example, the SRAII can be used to assess subjective effects of intranasal administration a drug such as ketamine, or a pharmaceutically acceptable salt thereof, as described herein. The SRAII includes five categories for which a subject is asked to provide a rating. For example, the categories can include: 1) burning sensation; 2) need to blow nose/sneeze; 3) runny nose and/or nasal discharge; 4) facial pressure or pain; and 5) nasal congestion. In some embodiments, each item is scored on a 6-point scale. For example, 0 refers to no difficulty at all; 1 refers to very mild difficulty; 2 refers to mild/slight difficulty; 3 refers to moderate difficulty; 4 refers to severe difficulty; and 5 refers to very severe difficulty, e.g., the worst possible.

In some embodiments, the SRAII of the subject is 0 to 5 after intranasal administration of racemic ketamine, or a pharmaceutically acceptable salt thereof, as described herein. For example, 0 to 1, 0 to 2, 0 to 3, 0 to 4, 4 to 5, 3 to 5, or 2 to 5 after intranasal administration of racemic ketamine as described herein. In some embodiments, the SRAII of the subject is 1, 2, 3, 4, or 5 after administration of ketamine, or a pharmaceutically acceptable salt thereof, as described herein. In some embodiments, the SRAII score of the subject is measured at about 5 minutes to about 24 hours after intranasal administration of the racemic ketamine, or a pharmaceutically acceptable salt thereof. For example, about 5 minutes to 1 hour, about 5 minutes to 6 hours, about 5 minutes to 12 hours, about 5 minutes to 18 hours, about 18 hours to 24 hours, about 12 hours to 24 hours, or about 6 hours to 24 hours after intranasal administration of the racemic ketamine, or a pharmaceutically acceptable salt thereof. In some embodiments, the SRAII score of the subject is measured at about 1 hour to about 4 hours, about 1.5 hours to about 5 hours, about 2 hours to about 6 hours, about 5 hours to about 10 hours, or about 45 minutes to about 24 hours after intranasal administration of the racemic ketamine, or a pharmaceutically acceptable salt thereof.

In some embodiments, the SRAII score of the subject is substantially the same prior to and after intranasal administration of the racemic ketamine, or a pharmaceutically acceptable salt thereof. For example, SRAII score of the subject changes (i.e., increases or decreases) by 0 to 5 after intranasal administration of the racemic ketamine, or a pharmaceutically acceptable salt thereof, relative to prior to intranasal administration of the racemic ketamine, or a pharmaceutically acceptable salt thereof. In some embodiments, the SRAII score of the subject is substantially the same about 5 minutes to about 24 hours prior to intranasal administration of the racemic ketamine, or a pharmaceutically acceptable salt thereof, as about 5 minutes to about 24 hours after intranasal administration of the racemic ketamine, or a pharmaceutically acceptable salt thereof. For example, about 5 minutes to 1 hour, about 5 minutes to 6 hours, about 5 minutes to 12 hours, about 5 minutes to 18 hours, about 18 hours to 24 hours, about 12 hours to 24 hours, about 6 hours to 24 hours, 1 hour to about 4 hours, about 1.5 hours to about 5 hours, about 2 hours to about 6 hours, or about 5 hours to about 10 hours prior to intranasal administration of the racemic ketamine, or a pharmaceutically acceptable salt thereof, and about 5 minutes to 1 hour, about 5 minutes to 6 hours, about 5 minutes to 12 hours, about 5 minutes to 18 hours, about 18 hours to 24 hours, about 12 hours to 24 hours, about 6 hours to 24 hours, 1 hour to about 4 hours, about 1.5 hours to about 5 hours, about 2 hours to about 6 hours, or about 5 hours to about 10 hours after intranasal administration of the racemic ketamine, or a pharmaceutically acceptable salt thereof.

In some embodiments, the SRAII score of the subject is reduced by about 5 to about 25 after intranasal administration of the racemic ketamine, or a pharmaceutically acceptable salt thereof, relative to intranasal administration of an equivalent dose of (S)-ketamine, or a pharmaceutically acceptable salt thereof. For example, the SRAII score of the subject can be reduced by about 5 to about 25 after intranasal administration of the racemic ketamine, or a pharmaceutically acceptable salt thereof, relative to the score observed after administration of an equivalent dose of (S)-ketamine, or a pharmaceutically acceptable salt thereof. In some embodiments, the SRAII score of the subject is reduced by about 5 to about 20, about 5 to about 15, about 5 to about 10, about 20 to about 25, about 15 to about 25, or about 10 to about 25 after intranasal administration of the racemic ketamine, or a pharmaceutically acceptable salt thereof. In some embodiments, the SRAII score of the subject is reduced as described herein about 5 minutes to about 24 hours after intranasal administration of the racemic ketamine, or a pharmaceutically acceptable salt thereof. For example, about 5 minutes to 1 hour, about 5 minutes to 6 hours, about 5 minutes to 12 hours, about 5 minutes to 18 hours, about 18 hours to 24 hours, about 12 hours to 24 hours, or about 6 hours to 24 hours after intranasal administration of the racemic ketamine, or a pharmaceutically acceptable salt thereof. In some embodiments, the SRAII score of the subject is measured at about 1 hour to about 4 hours, about 1.5 hours to about 5 hours, about 2 hours to about 6 hours, or about 5 hours to about 10 hours, after intranasal administration of the racemic ketamine, or a pharmaceutically acceptable salt thereof (or after administration of an equivalent dose of (S)-ketamine, or a pharmaceutically acceptable salt thereof).

### Dosage

In some embodiments, about 30 mg to about 90 mg of racemic ketamine, or a pharmaceutically acceptable salt thereof, is intranasally administered to the subject. For example, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, or any value in between. In some embodiments, about 30 mg to about 60 mg of racemic ketamine, or a pharmaceutically acceptable salt thereof, is intranasally administered to the subject. In some embodiments, about 45 mg to about 75 mg of racemic ketamine, or a pharmaceutically acceptable salt thereof, is intranasally administered to the subject. In some embodiments, about 60 mg to about 90 mg of racemic ketamine, or a pharmaceutically acceptable salt thereof, is intranasally administered to the subject. In some embodiments, about 30 mg, about 60 mg, about 75 mg, or about 90 mg of racemic ketamine, or a pharmaceutically acceptable salt thereof, is intranasally administered to the subject. In some embodiments, the formulation provides about 30 mg of racemic ketamine, or a pharmaceutically acceptable salt thereof, per dose. In some embodiments, the formulation provides about 60 mg of racemic ketamine, or a pharmaceutically acceptable salt thereof, per dose. In some embodiments, the formulation provides about 75 mg of racemic ketamine, or a pharmaceutically acceptable salt thereof, per dose. In some embodiments, the formulation provides about 90 mg of racemic ketamine, or a pharmaceutically acceptable salt thereof, per dose.

### Pharmacokinetics

Some embodiments provide a method of treating MDD in a subject in need thereof, the method comprising intranasally administering to the subject a therapeutically effective amount of racemic ketamine, or a pharmaceutically acceptable salt thereof, wherein intranasal administration of the racemic ketamine exhibits one or more of:
an AUC₀₋ₜ of norketamine that is at least 1.5 times higher than the AUC₀₋ₜ of norketamine exhibited by intravenous administration of an equivalent dose of racemic ketamine;
an AUC_{0-inf} of norketamine that is at least 1.5 times higher than the AUC_{0-inf} of norketamine exhibited by intravenous administration of an equivalent dose of racemic ketamine; and
a Cₘₐₓ of norketamine that is at least 2 times higher than the Cₘₐₓ of norketamine exhibited by intravenous administration of an equivalent dose of racemic ketamine.

Some embodiments provide a method for treating TRD in a subject in need thereof, the method comprising intranasally administering to the subject a therapeutically effective amount of racemic ketamine, or a pharmaceutically acceptable salt thereof; wherein intranasal administration of the racemic ketamine exhibits one or more of:
an AUC₀₋ₜ of norketamine that is at least 1.5 times higher than the AUC₀₋ₜ of norketamine exhibited by intravenous administration of an equivalent dose of racemic ketamine;
an AUC_{0-inf} of norketamine that is at least 1.5 times higher than the AUC_{0-inf} of norketamine exhibited by intravenous administration of an equivalent dose of racemic ketamine; and
a Cₘₐₓ of norketamine that is at least 2 times higher than the Cₘₐₓ of norketamine exhibited by intravenous administration of an equivalent dose of racemic ketamine.

Some embodiments provide a method for treating PTSD in a subject in need thereof, the method comprising intranasally administering to the subject a therapeutically effective amount of racemic ketamine, or a pharmaceutically acceptable salt thereof; wherein intranasal administration of the racemic ketamine exhibits one or more of:
an AUC₀₋ₜ of norketamine that is at least 1.5 times higher than the AUC₀₋ₜ of norketamine exhibited by intravenous administration of an equivalent dose of racemic ketamine;
an AUC_{0-inf} of norketamine that is at least 1.5 times higher than the AUC_{0-inf} of norketamine exhibited by intravenous administration of an equivalent dose of racemic ketamine; and
a Cₘₐₓ of norketamine that is at least 2 times higher than the Cₘₐₓ of norketamine exhibited by intravenous administration of an equivalent dose of racemic ketamine.

In some embodiments, intranasal administration of the racemic ketamine exhibits an AUC₀₋₁ of norketamine that is about 1.7 to about 2.5 times higher than the AUC₀₋ₜ of norketamine exhibited by intravenous administration of an equivalent dose of racemic ketamine. In some embodiments, intranasal administration of the racemic ketamine exhibits an AUC₀₋ₜ of norketamine that is about 1.9 to about 2.3 times higher than the AUC₀₋₁ of norketamine exhibited by intravenous administration of an equivalent dose of racemic ketamine. In some embodiments, intranasal administration of the racemic ketamine exhibits an AUC₀₋ₜ of norketamine that is about 2.0 times higher than the AUC₀₋ₜ of norketamine exhibited by intravenous administration of an equivalent dose of racemic ketamine.

In some embodiments, intranasal administration of the racemic ketamine exhibits an AUC_{0-inf} of norketamine that is about 1.5 to about 2.5 times higher than the AUC_{0-inf} of norketamine exhibited by intravenous administration of an equivalent dose of racemic ketamine. In some embodiments, intranasal administration of the racemic ketamine exhibits an AUC_{0-inf} of norketamine that is about 1.8 to about 2.2 times higher than the AUC_{0-inf} of norketamine exhibited by intravenous administration of an equivalent dose of racemic ketamine. In some embodiments, intranasal administration of the racemic ketamine exhibits an AUC_{0-inf} of norketamine that is about 2.0 times higher than the AUC_{0-inf} of norketamine exhibited by intravenous administration of an equivalent dose of racemic ketamine.

In some embodiments, intranasal administration of the racemic ketamine exhibits a Cₘₐₓ of norketamine that is about 2.2 to about 3.5 times higher than the Cₘₐₓ of norketamine exhibited by intravenous administration of an equivalent dose of racemic ketamine. In some embodiments, intranasal administration of the racemic ketamine exhibits a Cₘₐₓ of norketamine that is about 2.4 to about 3.2 times higher than the Cₘₐₓ of norketamine exhibited by intravenous administration of an equivalent dose of racemic ketamine. In some embodiments, intranasal administration of the racemic ketamine exhibits a Cₘₐₓ of norketamine that is about 2.9 times higher than the Cₘₐₓ of norketamine exhibited by intravenous administration of an equivalent dose of racemic ketamine.

In some embodiments, intranasal administration of the racemic ketamine exhibits a Tₘₐₓ of norketamine that is about 80% to about 125% of the Tₘₐₓ of norketamine exhibited by an intravenous administration of an equivalent dose of racemic ketamine. In some embodiments, intranasal administration of the racemic ketamine exhibits a Tₘₐₓ of norketamine that is about 90% to about 110% of the Tₘₐₓ of norketamine exhibited by an intravenous administration of an equivalent dose of racemic ketamine.

In some embodiments, one or more of the AUC₀₋ₜ, AUC_{0-inf}, Cₘₐₓ, and Tₘₐₓ of norketamine is determined following one dose of racemic ketamine. In some embodiments, one or more of the AUC₀₋ₜ, AUC_{0-inf}, Cₘₐₓ, and Tₘₐₓ of norketamine is determined following two doses of racemic ketamine. In some embodiments, one or more of the AUC₀₋ₜ, AUC_{0-inf}, Cₘₐₓ, and Tₘₐₓ of norketamine is determined following three doses of racemic ketamine.

Some embodiments provide a method of treating MDD in a subject in need thereof, the method comprising intranasally administering to the subject a therapeutically effective amount of racemic ketamine, or a pharmaceutically acceptable salt thereof, wherein intranasal administration of the racemic ketamine exhibits one or more of:
an AUC₀₋ₜ of hydroxynorketamine that is at least 1.5 times higher than the AUC₀₋ₜ of hydroxynorketamine exhibited by intravenous administration of an equivalent dose of racemic ketamine;
an AUC_{0-inf} of hydroxynorketamine that is at least 1.2 times higher than the AUC_{0-inf} of hydroxynorketamine exhibited by intravenous administration of an equivalent dose of racemic ketamine; and
a Cₘₐₓ of hydroxynorketamine that is at least 2 times higher than the Cₘₐₓ of norketamine exhibited by intravenous administration of an equivalent dose of racemic ketamine.

Some embodiments provide a method for treating TRD in a subject in need thereof, the method comprising intranasally administering to the subject a therapeutically effective amount of racemic ketamine, or a pharmaceutically acceptable salt thereof, wherein intranasal administration of the racemic ketamine exhibits one or more of:
an AUC₀₋ₜ of hydroxynorketamine that is at least 1.5 times higher than the AUC₀₋ₜ of hydroxynorketamine exhibited by intravenous administration of an equivalent dose of racemic ketamine;
an AUC_{0-inf} of hydroxynorketamine that is at least 1.2 times higher than the AUC_{0-inf} of hydroxynorketamine exhibited by intravenous administration of an equivalent dose of racemic ketamine; and
a Cₘₐₓ of hydroxynorketamine that is at least 2 times higher than the Cₘₐₓ of norketamine exhibited by intravenous administration of an equivalent dose of racemic ketamine.

Some embodiments provide a method for treating PTSD in a subject in need thereof, the method comprising intranasally administering to the subject a therapeutically effective amount of racemic ketamine, or a pharmaceutically acceptable salt thereof, wherein intranasal administration of the racemic ketamine exhibits one or more of:
an AUC₀₋ₜ of hydroxynorketamine that is at least 1.5 times higher than the AUC₀₋ₜ of hydroxynorketamine exhibited by intravenous administration of an equivalent dose of racemic ketamine;
an AUC_{0-inf} of hydroxynorketamine that is at least 1.2 times higher than the AUC_{0-inf} of hydroxynorketamine exhibited by intravenous administration of an equivalent dose of racemic ketamine; and
a Cₘₐₓ of hydroxynorketamine that is at least 2 times higher than the Cₘₐₓ of norketamine exhibited by intravenous administration of an equivalent dose of racemic ketamine.

Some embodiments provide a method for treating PTSD at imminent risk of suicide in a subject in need thereof, the method comprising intranasally administering to the subject a therapeutically effective amount of racemic ketamine, or a pharmaceutically acceptable salt thereof, wherein intranasal administration of the racemic ketamine exhibits one or more of:
an AUC₀₋ₜ of hydroxynorketamine that is at least 1.5 times higher than the AUC₀₋ₜ of hydroxynorketamine exhibited by intravenous administration of an equivalent dose of racemic ketamine;
an AUC_{0-inf} of hydroxynorketamine that is at least 1.2 times higher than the AUC_{0-inf} of hydroxynorketamine exhibited by intravenous administration of an equivalent dose of racemic ketamine; and
a Cₘₐₓ of hydroxynorketamine that is at least 2 times higher than the Cₘₐₓ of norketamine exhibited by intravenous administration of an equivalent dose of racemic ketamine.

Some embodiments provide a method for treating Moderate or Severe Major Depressive Episode (Bipolar Depression) in a subject in need thereof, the method comprising intranasally administering to the subject a therapeutically effective amount of racemic ketamine, or a pharmaceutically acceptable salt thereof, wherein intranasal administration of the racemic ketamine exhibits one or more of:

an AUC₀₋ₜ of hydroxynorketamine that is at least 1.5 times higher than the AUC₀₋ₜ of hydroxynorketamine exhibited by intravenous administration of an equivalent dose of racemic ketamine;
an AUC_{0-inf} of hydroxynorketamine that is at least 1.2 times higher than the AUC_{0-inf} of hydroxynorketamine exhibited by intravenous administration of an equivalent dose of racemic ketamine; and
a Cₘₐₓ of hydroxynorketamine that is at least 2 times higher than the Cₘₐₓ of norketamine exhibited by intravenous administration of an equivalent dose of racemic ketamine.

Some embodiments provide a method for treating Complex Regional Pain Syndrome in a subject in need thereof, the method comprising intranasally administering to the subject a therapeutically effective amount of racemic ketamine, or a pharmaceutically acceptable salt thereof, wherein intranasal administration of the racemic ketamine exhibits one or more of:
an AUC₀₋ₜ of hydroxynorketamine that is at least 1.5 times higher than the AUC₀₋ₜ of hydroxynorketamine exhibited by intravenous administration of an equivalent dose of racemic ketamine;
an AUC_{0-inf} of hydroxynorketamine that is at least 1.2 times higher than the AUC_{0-inf} of hydroxynorketamine exhibited by intravenous administration of an equivalent dose of racemic ketamine; and
a Cₘₐₓ of hydroxynorketamine that is at least 2 times higher than the Cₘₐₓ of norketamine exhibited by intravenous administration of an equivalent dose of racemic ketamine.

In some embodiments, intranasal administration of the racemic ketamine exhibits an AUC₀₋ₜ of hydroxynorketamine that is about 1.7 to about 2.5 times higher than the AUC₀₋ₜ of hydroxynorketamine exhibited by intravenous administration of an equivalent dose of racemic ketamine. In some embodiments, intranasal administration of the racemic ketamine exhibits an AUC₀₋₁ of hydroxynorketamine that is about 1.9 to about 2.3 times higher than the AUC₀₋ₜ of hydroxynorketamine exhibited by intravenous administration of an equivalent dose of racemic ketamine. In some embodiments, intranasal administration of the racemic ketamine exhibits an AUC₀₋ₜ of hydroxynorketamine that is about 2.1 times higher than the AUC₀₋ₜ of hydroxynorketamine exhibited by intravenous administration of an equivalent dose of racemic ketamine.

In some embodiments, intranasal administration of the racemic ketamine exhibits an AUC_{0-inf} of hydroxynorketamine that is about 1.5 to about 2.5 times higher than the AUC_{0-inf} of hydroxynorketamine exhibited by intravenous administration of an equivalent dose of racemic ketamine. In some embodiments, intranasal administration of the racemic ketamine exhibits an AUC₀₋ₜ of hydroxynorketamine that is about 1.7 to about 2.1 times higher than the AUC_{0-inf} of hydroxynorketamine exhibited by intravenous administration of an equivalent dose of racemic ketamine. In some embodiments, intranasal administration of the racemic ketamine exhibits an AUC₀₋ₜ of hydroxynorketamine that is about 1.9 times higher than the AUC_{0-inf} of hydroxynorketamine exhibited by intravenous administration of an equivalent dose of racemic ketamine.

In some embodiments, intranasal administration of the racemic ketamine exhibits a Cₘₐₓ of hydroxynorketamine that is about 2.2 to about 3.2 times higher than the Cₘₐₓ of hydroxynorketamine exhibited by intravenous administration of an equivalent dose of racemic ketamine. In some embodiments, intranasal administration of the racemic ketamine exhibits a Cₘₐₓ of hydroxynorketamine that is about 2.4 to about 2.8 times higher than the Cₘₐₓ of hydroxynorketamine exhibited by intravenous administration of an equivalent dose of racemic ketamine. In some embodiments, intranasal administration of the racemic ketamine exhibits a Cₘₐₓ of hydroxynorketamine that is about 2.6 times higher than the Cₘₐₓ of hydroxynorketamine exhibited by intravenous administration of an equivalent dose of racemic ketamine.

In some embodiments, intranasal administration of the racemic ketamine exhibits a Tₘₐₓ of hydroxynorketamine that is about 80% to about 125% of the Tₘₐₓ of hydroxynorketamine exhibited by an intravenous administration of an equivalent dose of racemic ketamine. In some embodiments intranasal administration of the racemic ketamine exhibits a Tₘₐₓ of hydroxynorketamine that is about 90% to about 110% of the Tₘₐₓ of hydroxynorketamine exhibited by an intravenous administration of an equivalent dose of racemic ketamine.

In some embodiments, the relative ratios and percentages described herein are measured from about 15 minutes to about 8 hours after intranasal administration of the racemic ketamine, or a pharmaceutically acceptable salt thereof. For example, in some embodiments, the ratios described herein are measured at about 15 minutes, about 20 minutes, about 25 minutes, about 30 minutes, about 35 minutes, about 40 minutes, about 45 minutes, about 50 minutes, about 55 minutes, about 60 minutes, about 75 minutes, about 90 minutes, about 105 minutes, about 2 hours, about 2.5 hours, about 3 hours, about 3.5 hours, about 4 hours, about 4.5 hours, about 5 hours, about 5.5 hours, about 6 hours, about 6.5 hours, about 7 hours, about 7.5 hours, about 8 hours, or any value in between. In some embodiments, the relative ratios and percentages described herein are measured for about 24 hours to about 1 month. In some embodiments, the relative ratios and percentages described herein are measured for about 24 hours to about 2 weeks. For example, about 24 hours, about 2 days, about 3 days, about 4 days, about 5 days, about 6 days, about 7 days, about 8 days, about 9 days, about 10 days, about 11 days, about 12 days, about 13 days, about 2 weeks, or any value in between. In some embodiments, the racemic ketamine, or a pharmaceutically acceptable salt thereof, is intranasally administered twice daily, once daily, once every other day, once every three days, once every four days, once every five days, once every six days, or once a week, or a combination thereof, for the measurement period (for example, about 24 hours to about 1 month).

In some embodiments, the Tₘₐₓ of the ketamine is from about 20 minutes to about 120 minutes, following the intranasal administration of racemic ketamine, for example about 20 minutes, about 30 minutes, about 40 minutes, about 50 minutes, about 60 minutes, about 70 minutes, about 80 minutes, about 90 minutes, about 100 minutes, about 110 minutes, about 120 minutes, or any value in between. In some embodiments, the Tₘₐₓ of the ketamine is from about 20 minutes to about 90 minutes, following the intranasal administration of racemic ketamine, for example about 20 minutes, about 30 minutes, about 40 minutes, about 50 minutes, about 60 minutes, about 70 minutes, about 80 minutes, about 90 minutes, or any value in between. In some embodiments, the Tₘₐₓ of the ketamine is from about 30 minutes to about 90 minutes following the intranasal administration of racemic ketamine.

In some embodiments, the Tₘₐₓ of norketamine is from about 45 minutes to about 360 minutes, following the intranasal administration of racemic ketamine, for example, about 45 minutes, about 60 minutes, about 75 minutes, about 90 minutes, about 105 minutes, about 120 minutes, about 135 minutes, about 150 minutes, about 165 minutes, about 180 minutes, about 195 minutes, about 210 minutes, about 225 minutes, about 240 minutes, about 255 minutes, about 270 minutes, about 285 minutes, about 300 minutes, about 315 minutes, about 315 minutes, about 330 minutes, about 345 minutes, about 360 minutes, or any value in between. In some embodiments, the Tₘₐₓ of norketamine is from about 100 minutes to about 250 minutes following the intranasal administration of racemic ketamine

In some embodiments, the Tₘₐₓ of 6-hydroxynorketamine is from about 45 minutes to about 8 hours, following the intranasal administration of racemic ketamine, for example, about 45 minutes, about 1 hour, about 2 hours, about 3 hours, about 4 hours, about 5 hours, about 6 hours, about 7 hours, about 8 hours, about 9 hours, about 10 hours, or any value in between. In some embodiments, the Tₘₐₓ of 6-hydroxynorketamine is from about 2 hours to about 4 hours, following the intranasal administration of racemic ketamine, for example, about 2 hours, about 2.5 hours, about 3 hours, about 3.5 hours, about 4 hours, or any value in between. In some embodiments, the Tₘₐₓ of 6-hydroxynorketamine is from about 3 hours to about 4 hours following the intranasal administration of racemic ketamine.

In some embodiments, the Cₘₐₓ of ketamine is from about 15 ng/ml to about 225 ng/ml, or any value in between, following the intranasal administration of racemic ketamine. In some embodiments, the Cₘₐₓ of ketamine following the intranasal administration of racemic ketamine is from about 25 ng/ml to about 225 ng/ml, for example, about 25 ng/mL, about 35 ng/ml, about 45 ng/ml, about 55 ng/mL, about 65 ng/ml, about 75 ng/ml, about 85 ng/mL, about 95 ng/ml, about 105 ng/ml, about 115 ng/ml, about 125 ng/ml, about 135 ng/ml, about 145 ng/ml, about 155 ng/ml, about 165 ng/ml, about 175 ng/ml, about 185 ng/mL, about 195 ng/ml, about 205 ng/mL, about 215 ng/ml, about 225 ng/ml, or any value in between. In some embodiments, the Cₘₐₓ of ketamine is from about 70 ng/ml to about 205 ng/ml, following the intranasal administration of racemic ketamine, for example, about 85 ng/ml, about 95 ng/mL, about 105 ng/ml, about 115 ng/ml, about 125 ng/ml, about 135 ng/ml, about 145 ng/ml, about 155 ng/ml, about 165 ng/ml, about 175 ng/ml, about 185 ng/mL, about 195 ng/ml, about 205 ng/ml, or any value in between. In some embodiments, the Cₘₐₓ of ketamine is from about 75 ng/ml to about 175 ng/mL, following the intranasal administration of racemic ketamine, for example, about 75 ng/ml, about 100 ng/ml, about 125 ng/mL, about 150 ng/ml, about 175 ng/ml, or any value in between. In some embodiments, the Cₘₐₓ of ketamine is from about 95 ng/mL to about 145 ng/mL, following the intranasal administration of racemic ketamine, for example, about 95 ng/ml, about 105 ng/ml, about 115 ng/mL, about 125 ng/mL, about 135 ng/mL, about 145 ng/mL, or any value in between.

In some embodiments, the Cₘₐₓ of norketamine is from about 40 ng/mL to about 375 ng/mL, or any value in between, following the intranasal administration of racemic ketamine. In some embodiments, the Cₘₐₓ of norketamine is from about 50 ng/mL to about 275 ng/ml, following the intranasal administration of racemic ketamine, for example, about 50 ng/ml, about 75 ng/ml, about 100 ng/mL, about 125 ng/ml, about 150 ng/ml, about 175 ng/ml, about 200 ng/ml, about 225 ng/ml, about 250 ng/mL, about 275 ng/mL, or any value in between. In some embodiments, the Cₘₐₓ of norketamine is from about 90 ng/ml to about 180 ng/mL, following the intranasal administration of racemic ketamine, for example, about 90 ng/ml, about 100 ng/mL, about 110 ng/ml, about 120 ng/ml, about 130 ng/ml, about 140 ng/ml, about 150 ng/ml, about 160 ng/mL, about 170 ng/mL, about 180 ng/mL, or any value in between. In some embodiments, the Cₘₐₓ of norketamine is from about 70 ng/ml to about 85 ng/ml following the intranasal administration of racemic ketamine. In some embodiments, the Cₘₐₓ of norketamine is from about 90 ng/ml to about 130 ng/ml following the intranasal administration of racemic ketamine. In some embodiments, the Cₘₐₓ of norketamine is from about 120 ng/ml to about 150 ng/ml following the intranasal administration of racemic ketamine. In some embodiments, the Cₘₐₓ of norketamine is from about 160 ng/mL to about 195 ng/mL following the intranasal administration of racemic ketamine. In some embodiments, the Cₘₐₓ of norketamine is from about 140 ng/ml to about 180 ng/ml following the intranasal administration of racemic ketamine. In some embodiments, the Cₘₐₓ of norketamine is from about 215 ng/ml to about 225 ng/ml following the intranasal administration of racemic ketamine.

In some embodiments, the Cₘₐₓ of 6-hydroxynorketamine is from about 15 ng/mL to about 275 ng/ml, or any value in between, following the intranasal administration of racemic ketamine. In some embodiments, the Cₘₐₓ of 6-hydroxynorketamine is from about 40 ng/ml to about 275 ng/ml, or any value in between, following the intranasal administration of racemic ketamine. In some embodiments, the Cₘₐₓ of 6-hydroxynorketamine is from about 55 ng/ml to about 245 ng/ml, following the intranasal administration of racemic ketamine, for example, about 55 ng/mL, about 65 ng/ml, about 75 ng/ml, about 85 ng/ml, about 95 ng/ml, about 105 ng/ml, about 115 ng/ml, about 125 ng/ml, about 135 ng/ml, about 145 ng/mL, about 155 ng/ml, about 165 ng/ml, about 175 ng/mL, about 185 ng/ml, about 195 ng/ml, about 205 ng/mL, about 215 ng/ml, about 225 ng/ml, about 235 ng/mL, about 245 ng/ml, or any value in between. In some embodiments, the Cₘₐₓ of 6-hydroxynorketamine is from about 55 ng/ml to about 100 ng/ml following the intranasal administration of racemic ketamine. In some embodiments, the Cₘₐₓ of 6-hydroxynorketamine is from about 95 ng/ml to about 135 ng/mL following the intranasal administration of racemic ketamine. In some embodiments, the Cₘₐₓ of 6-hydroxynorketamine is from about 130 ng/ml to about 155 ng/ml following the intranasal administration of racemic ketamine. In some embodiments, the Cₘₐₓ of 6-hydroxynorketamine is from about 150 ng/ml to about 185 ng/ml following the intranasal administration of racemic ketamine. In some embodiments, the Cₘₐₓ of 6-hydroxynorketamine is from about 175 ng/mL to about 215 ng/mL following the intranasal administration of racemic ketamine. In some embodiments, the Cₘₐₓ of 6-hydroxynorketamine is from about 210 ng/ml to about 245 ng/mL following the intranasal administration of racemic ketamine.

In some embodiments, the t½ for ketamine is about 2 hours to about 9 hours, following the intranasal administration of racemic ketamine, for example, about 2 hours, about 2.5 hours, about 3 hours, about 3.5 hours, about 4 hours, about 4.5 hours, about 5 hours, about 5.5 hours, about 6 hours, about 6.5 hours, about 7 hours, about 7.5 hours, about 8 hours, about 8.5 hours, about 9 hours, or any value in between. In some embodiments, the t½ for ketamine is about 4 hours to about 7 hours, following the intranasal administration of racemic ketamine, for example, about 4 hours, about 4.5 hours, about 5 hours, about 5.5 hours, about 6 hours, about 6.5 hours, about 7 hours, or any value in between.

In some embodiments, the t½ for norketamine is about 4.5 hours to about 12.5 hours, following the intranasal administration of racemic ketamine, for example, about 4.5 hours, about 5 hours, about 5.5 hours, about 6 hours, about 6.5 hours, about 7 hours, about 7.5 hours, about 8 hours, about 8.5 hours, about 9 hours, about 9.5 hours, about 10 hours, about 10.5 hours, about 11 hours, about 11.5 hours, about 12 hours, about 12.5 hours, or any value in between. In some embodiments, the t½ for norketamine is about 5 hours to about 10 hours, following the intranasal administration of racemic ketamine, for example, about 5 hours, about 5.5 hours, about 6 hours, about 6.5 hours, about 7 hours, about 7.5 hours, about 8 hours, about 8.5 hours, about 9 hours, about 0.5 hours, about 10 hours, or any value in between. In some embodiments, the t½ for norketamine is about 7 hours to about 8 hours, following the intranasal administration of racemic ketamine

In some embodiments, the t½ for 6-hydroxynorketamine is about 5.5 hours to about 22.5 hours, following the intranasal administration of racemic ketamine, for example, about 5.5 hours, about 6 hours, about 6.5 hours, about 7 hours, about 7.5 hours, about 8 hours, about 8.5 hours, about 9 hours, about 9.5 hours, about 10 hours, about 10.5 hours, about 11 hours, about 11.5 hours, about 12 hours, about 12.5 hours, about 13 hours, about 13.5 hours, about 14 hours, about 14.5 hours, about 15 hours, about 15.5 hours, about 16 hours, about 16.5 hours, about 17 hours, about 18.5 hours, about 19 hours, about 19.5 hours, about 20 hours, about 20.5 hours, about 21 hours, about 21.5 hours, about 22 hours, about 22.5 hours, or any value in between. In some embodiments, the t½ for 6-hydroxynorketamine is about 8 hours and about 15 hours, following the intranasal administration of racemic ketamine, for example, about 8 hours, about 8.5 hours, about 9 hours, about 9.5 hours, about 10 hours, about 10.5 hours, about 11 hours, about 11.5 hours, about 12 hours, about 12.5 hours, about 13 hours, about 13.5 hours, about 14 hours, about 14.5 hours, about 15 hours, or any value in between. In some embodiments, the t½ for 6-hydroxynorketamine is about 10 hours and about 12 hours, following the intranasal administration of racemic ketamine

In some embodiments, the apparent clearance for ketamine is from about 150 L/h to about 350 L/h, following the intranasal administration of racemic ketamine, for example, about 150 L/h, about 175 L/h, about 200 L/hr, about 225 L/h, about 250 L/h, about 275 L/h, about 300 L/hr, about 325 L/h, about 350 L/h, or any value in between. In some embodiments, the apparent clearance for ketamine is from about 200 L/h to about 300 L/h, following the intranasal administration of racemic ketamine, for example, about 200 L/hr, about 225 L/h, about 250 L/h, about 275 L/h, about 300 L/hr, or any value in between. In some embodiments, the apparent clearance for ketamine is from about 195 L/h to about 245 L/h, following the intranasal administration of racemic ketamine, for example, about 195 L/hr, about 200 L/hr, about 225 L/h, about 230 L/h, about 235 L/h, about 240 L/h, about 245 L/h, or any value in between.

In some embodiments, the apparent V_{d}/F for ketamine is from about 2.5 L/kg to about 6 L/kg, or any value in between following the intranasal administration of racemic ketamine. In some embodiments, the apparent V_{d}/F for ketamine is from about 1,000 L to about 2,500 L, following the intranasal administration of racemic ketamine, for example, about 1,000 L, about 1,250 L, about 1,500 L, about 1,750 L, about 2,000 L, about 2,250 L, about 2,500 L, or any value in between. In some embodiments, the apparent V_{d}/F for ketamine is from about 1,250 L to about 1,750 L, following the intranasal administration of racemic ketamine, for example, about 1,250 L, about 1,300 L, about 1,350 L, about 1,400 L, about 1,450 L, about 1,500 L, about 1,550 L, about 1,600 L, about 1,650 L, about 1,700 L, about 1,750 L, or any value in between.

In some embodiments, the elimination rate constant (Kₑₗ (1/h or h⁻¹)) for ketamine is from about 0.1 h⁻¹ to about 0.25 h⁻¹, following the intranasal administration of racemic ketamine, for example, about 0.1 h⁻¹, about 0.15 h⁻¹, about 0.2 h⁻¹, about 0.25 h⁻¹, or any value in between.

In some embodiments, the elimination rate constant (Kₑₗ (1/h or h⁻¹)) for norketamine is from about 0.05 h⁻¹ to about 0.15 h⁻¹, following the intranasal administration of racemic ketamine, for example, about 0.05 h⁻¹, about 0.1 h⁻¹, about 0.15 h⁻¹, or any value in between. In some embodiments, the elimination rate constant (Kₑₗ (1/h or h⁻¹)) for norketamine is from about 0.09 h⁻¹ to about 0.1 h⁻¹, following the intranasal administration of racemic ketamine

In some embodiments, the elimination rate constant (Kₑₗ (1/h or h⁻¹)) for 6-hydroxynorketamine is from about 0.05 h⁻¹ to about 0.15 h⁻¹, following the intranasal administration of racemic ketamine, for example, about 0.05 h⁻¹, about 0.1 h⁻¹, about 0.15 h⁻¹, or any value in between. In some embodiments, the elimination rate constant (Kₑₗ (1/h or h⁻¹)) for 6-hydroxynorketamine is from about 0.09 h⁻¹ to about 0.1 h⁻¹, following the intranasal administration of racemic ketamine

In some embodiments, the AUC₀₋ₜ for ketamine is from about 70 ng*h/mL to about 675 ng*h/mL, following the intranasal administration of racemic ketamine, for example, about 70 ng*h/mL, about 100 ng*h/mL, about 125 ng*h/mL, about 150 ng*h/mL, about 175 ng*h/mL, about 200 ng*h/mL, about 225 ng*h/mL, about 250 ng*h/mL, about 275 ng*h/mL, about 300 ng*h/mL, about 325 ng*h/mL, about 350 ng*h/mL, about 375 ng*h/mL, about 400 ng*h/mL, about 425 ng*h/mL, about 450 ng*h/mL, about 475 ng*h/mL, about 500 ng*h/mL, about 525 ng*h/mL, about 550 ng*h/mL, about 575 ng*h/mL, about 600, ng*h/mL, about 625 ng*h/mL, about 650 ng*h/mL, about 675 ng*h/mL, or any value in between. In some embodiments, the AUC₀₋ₜ for ketamine is from about 70 ng*h/mL to about 250 ng*h/mL following the intranasal administration of racemic ketamine. In some embodiments, the AUC₀₋ₜ for ketamine is from about 200 ng*h/mL to about 450 ng*h/mL following the intranasal administration of racemic ketamine. In some embodiments, the AUC₀₋ₜ for ketamine is from about 400 ng*h/mL to about 675 ng*h/mL following the intranasal administration of racemic ketamine. In some embodiments, the AUC₀₋ₜ for ketamine is from about 600 ng*h/mL to about 675 ng*h/mL following the intranasal administration of racemic ketamine.

In some embodiments, about 30 mg of racemic ketamine is intranasally administered and the AUC₀₋ₜ for ketamine from about 70 ng*h/mL to about 350 ng*h/mL following the intranasal administration of racemic ketamine, for example, about 70 ng*h/mL, about 100 ng*h/mL, about 125 ng*h/mL about 150 ng*h/mL, about, 175 ng*h/mL, about 200 ng*h/mL, about 225 ng*h/mL, about 250 ng*h/mL, about 275 ng*h/mL, about 300 ng*h/mL, about 325 ng*h/mL, about 350 ng*h/mL, or any value in between. In some embodiments, about 30 mg of racemic ketamine is intranasally administered and the AUC₀₋ₜ for ketamine from about 70 ng*h/mL to about 150 ng*h/mL. In some embodiments, about 30 mg of racemic ketamine is intranasally administered and the AUC₀₋ₜ for ketamine from about 100 ng*h/mL to about 250 ng*h/mL. In some embodiments, about 30 mg of racemic ketamine is intranasally administered and the AUC₀₋ₜ for ketamine from about 200 ng*h/mL to about 350 ng*h/mL.

In some embodiments, about 75 mg of racemic ketamine is intranasally administered and the AUC₀₋ₜ for ketamine from about 225 ng*h/mL to about 525 ng*h/mL following the intranasal administration of racemic ketamine, for example, about 225 ng*h/mL, about 250 ng*h/mL, about 275 ng*h/mL about 300 ng*h/mL, about, 325 ng*h/mL, about 350 ng*h/mL, about 375 ng*h/mL, about 400 ng*h/mL, about 425 ng*h/mL, about 450 ng*h/mL, about 475 ng*h/mL, about 500 ng*h/mL, about 525 ng*h/mL, or any value in between. In some embodiments, about 75 mg of racemic ketamine is intranasally administered and the AUC₀₋ₜ for ketamine from about 225 ng*h/mL to about 325 ng*h/mL. In some embodiments, about 75 mg of racemic ketamine is intranasally administered and the AUC₀₋ₜ for ketamine from about 300. ng*h/mL to about 425 ng*h/mL. In some embodiments, about 75 mg of racemic ketamine is intranasally administered and the AUC₀₋ₜ for ketamine from about 400 ng*h/mL to about 525 ng*h/mL.

In some embodiments, about 90 mg of racemic ketamine is intranasally administered and the AUC₀₋ₜ for ketamine from about 220 ng*h/mL to about 675 ng*h/mL following the intranasal administration of racemic ketamine, for example, about 220 ng*h/mL, about 250 ng*h/mL, about 275 ng*h/mL about 300 ng*h/mL, about, 325 ng*h/mL, about 350 ng*h/mL, about 375 ng*h/mL, about 400 ng*h/mL, about 425 ng*h/mL, about 450 ng*h/mL, about 475 ng*h/mL, about 500 ng*h/mL, about 525 ng*h/mL, about 550 ng*h/mL, about 575 ng*h/mL, about 600 ng*h/mL, about 625 ng*h/mL, about 650 ng*h/mL, about 675 ng*h/mL, or any value in between. In some embodiments, about 90 mg of racemic ketamine is intranasally administered and the AUC₀₋ₜ for ketamine from about 220 ng*h/mL to about 375 ng*h/mL. In some embodiments, about 90 mg of racemic ketamine is intranasally administered and the AUC₀₋ₜ for ketamine from about 350 ng*h/mL to about 525 ng*h/mL. In some embodiments, about 90 mg of racemic ketamine is intranasally administered and the AUC₀₋ₜ for ketamine from about 500 ng*h/mL to about 675 ng*h/mL.

In some embodiments, the AUC₀₋ₜ for norketamine is from about 250 ng*h/mL to about 2,200 ng*h/mL, following the intranasal administration of racemic ketamine, for example, about 250 ng*h/mL, about 350 ng*h/mL, about 450 ng*h/mL, about 550 ng*h/mL, about 650 ng*h/mL, about 750 ng*h/mL, about 850 ng*h/mL, about 950 ng*h/mL, about 1,050 ng*h/mL, about 1,150 ng*h/mL, about 1,250 ng*h/mL, about 1,350 ng*h/mL, about 1,450 ng*h/mL, about 1,550 ng*h/mL, about 1,650 ng*h/mL, about 1750 ng*h/mL, about 1,850 ng*h/mL, about 1,950 ng*h/mL, about 2,050 ng*h/mL, about 2,200 ng*h/mL, or any value in between. In some embodiments, the AUC₀₋ₜ for norketamine is from about 250 ng*h/mL to about 950 ng*h/mL following the intranasal administration of racemic ketamine. In some embodiments, the AUC₀₋ₜ for norketamine is from about 900 ng*h/mL to about 1,550 ng*h/mL following the intranasal administration of racemic ketamine. In some embodiments, the AUC₀₋ₜ for norketamine is from about 1,500 ng*h/mL to about 2,200 ng*h/mL following the intranasal administration of racemic ketamine.

In some embodiments, about 30 mg of racemic ketamine is intranasally administered and the AUC₀₋ₜ for norketamine is from about 250 ng*h/mL to about 725 ng*h/mL, following the intranasal administration of racemic ketamine, for example, about 250 ng*h/mL, about 300 ng*h/mL, about 350 ng*h/mL, about 400 ng*h/mL, about 450 ng*h/mL, about 500 ng*h/mL, about 550 ng*h/mL, about 600 ng*h/mL, about 650 ng*h/mL, about 700 ng*h/mL, about 725 ng*h/mL, or any value in between. In some embodiments, about 30 mg of racemic ketamine is intranasally administered and the AUC₀₋ₜ for norketamine is from about 250 ng*h/mL to about 400 ng*h/mL. In some embodiments, about 30 mg of racemic ketamine is intranasally administered and the AUC₀₋₁ for norketamine from about 375 ng*h/mL to about 550 ng*h/mL. In some embodiments, about 30 mg of racemic ketamine is intranasally administered and the AUC₀₋ₜ for norketamine from about 500 ng*h/mL to about 725 ng*h/mL.

In some embodiments, about 75 mg of racemic ketamine is intranasally administered and the AUC₀₋ₜ for norketamine is from about 675 ng*h/mL to about 1,800 ng*h/mL, following the intranasal administration of racemic ketamine, for example, about 675 ng*h/mL, about 800 ng*h/mL, about 900 ng*h/mL, about 1,000 ng*h/mL, about 1,100 ng*h/mL, about 1,200 ng*h/mL, about 1,300 ng*h/mL, about 1,400 ng*h/mL, about 1,500 ng*h/mL, about 1,600 ng*h/mL, about 1,700 ng*h/mL, about 1,800 ng*h/mL, or any value in between. In some embodiments, about 75 mg of racemic ketamine is intranasally administered and the AUC₀₋ₜ for norketamine is from about 675 ng*h/mL to about 1,050 ng*h/mL. In some embodiments, about 75 mg of racemic ketamine is intranasally administered and the AUC₀₋ₜ for norketamine from about 1,000 ng*h/mL to about 1,450 ng*h/mL. In some embodiments, about 75 mg of racemic ketamine is intranasally administered and the AUC₀₋ₜ for norketamine from about 1,400 ng*h/mL to about 1,800 ng*h/mL.

In some embodiments, about 90 mg of racemic ketamine is intranasally administered and the AUC₀₋ₜ for norketamine is from about 850 ng*h/mL to about 2,200 ng*h/mL, following the intranasal administration of racemic ketamine, for example, about 850 ng*h/mL, about 950 ng*h/mL, about 1,050 ng*h/mL, about 1,150 ng*h/mL, about 1,250 ng*h/mL, about 1,350 ng*h/mL, about 1,450 ng*h/mL, about 1,550 ng*h/mL, about 1,650 ng*h/mL, about 1,750 ng*h/mL, about 1850 ng*h/mL, about 1,950 ng*h/mL, about 2,050 ng*h/mL, about 2,200 ng*h/mL, or any value in between. In some embodiments, about 90 mg of racemic ketamine is intranasally administered and the AUC₀₋ₜ for norketamine is from about 850 ng*h/mL to about 1,350 ng*h/mL. In some embodiments, about 90 mg of racemic ketamine is intranasally administered and the AUC₀₋ₜ for norketamine from about 1,300 ng*h/mL to about 1,850 ng*h/mL. In some embodiments, about 90 mg of racemic ketamine is intranasally administered and the AUC₀₋ₜ for norketamine from about 1,800 ng*h/mL to about 2,200 ng*h/mL.

In some embodiments, the AUC₀₋ₜ for 6-hydroxynorketamine is from about 300 ng*h/mL to about 3,100 ng*h/mL, following the intranasal administration of racemic ketamine, for example, about 300 ng*h/mL, about 450 ng*h/mL, about 600 ng*h/mL, about 750 ng*h/mL, about 900 ng*h/mL, about 1,050 ng*h/mL, about 1,200 ng*h/mL, about 1,350 ng*h/mL, about 1,500 ng*h/mL, about 1,750 ng*h/mL, about 1,900 ng*h/mL, about 2,050 ng*h/mL, about 2,200 ng*h/mL, about 2,450 ng*h/mL, about 2,600 ng*h/mL, about 2,750 ng*h/mL, about 2,900 ng*h/mL, about 3,100 ng*h/mL, or any value in between. In some embodiments, the AUC₀₋ₜ for 6-hydroxynorketamine is from about 300 ng*h/mL to about 700 ng*h/mL following the intranasal administration of racemic ketamine. In some embodiments, the AUC₀₋ₜ for 6-hydroxynorketamine is from about 700 ng*h/mL to about 900 ng*h/mL following the intranasal administration of racemic ketamine. In some embodiments, the AUC₀₋₁ for 6-hydroxynorketamine is from about 850 ng*h/mL to about 950 ng*h/mL following the intranasal administration of racemic ketamine. In some embodiments, the AUC₀₋ₜ for 6-hydroxynorketamine is from about 900 ng*h/mL to about 1,100 ng*h/mL following the intranasal administration of racemic ketamine. In some embodiments, the AUC₀₋ₜ for 6-hydroxynorketamine is from about 1,100 ng*h/mL to about 1,300 ng*h/mL following the intranasal administration of racemic ketamine. In some embodiments, the AUC₀₋₁ for 6-hydroxynorketamine is from about 1,300 ng*h/mL to about 1,700 ng*h/mL following the intranasal administration of racemic ketamine. In some embodiments, the AUC₀₋ₜ for 6-hydroxynorketamine is from about 1,700 ng*h/mL to about 2,400 ng*h/mL following the intranasal administration of racemic ketamine. In some embodiments, the AUC₀₋ₜ for 6-hydroxynorketamine is from about 2,400 ng*h/mL to about 3,100 ng*h/mL following the intranasal administration of racemic ketamine.

In some embodiments, about 30 mg of racemic ketamine is intranasally administered and the AUC₀₋ₜ for 6-hydroxynorketamine is from about 300 ng*h/mL to about 825 ng*h/mL, following the intranasal administration of racemic ketamine, for example, about 300 ng*h/mL, about 400 ng*h/mL, about 500 ng*h/mL, about 600 ng*h/mL, about 700 ng*h/mL, about 800 ng*h/mL, about 825 ng*h/mL, or any value in between. In some embodiments, about 30 mg of racemic ketamine is intranasally administered and the AUC₀₋ₜ for 6-hydroxynorketamine is from about 300 ng*h/mL to about 450 ng*h/mL following the intranasal administration of racemic ketamine. In some embodiments, about 30 mg of racemic ketamine is intranasally administered and the AUC₀₋₁ for 6-hydroxynorketamine is from about 400 ng*h/mL to about 550 ng*h/mL following the intranasal administration of racemic ketamine. In some embodiments, about 30 mg of racemic ketamine is intranasally administered and the AUC₀₋ₜ for 6-hydroxynorketamine is from about 500 ng*h/mL to about 825 ng*h/mL following the intranasal administration of racemic ketamine.

In some embodiments, about 75 mg of racemic ketamine is intranasally administered and the AUC₀₋ₜ for 6-hydroxynorketamine is from about 650 ng*h/mL to about 1,900 ng*h/mL, following the intranasal administration of racemic ketamine, for example, about 650 ng*h/mL, about 750 ng*h/mL, about 850 ng*h/mL, about 950 ng*h/mL, about 1,050 ng*h/mL, about 1,150 ng*h/mL, about 1,250 ng*h/mL, about 1,350 ng*h/mL, about 1,450 ng*h/mL, about 1,550 ng*h/mL, about 1,650 ng*h/mL, about 1,750 ng*h/mL, about 1,900 ng*h/mL, or any value in between. In some embodiments, about 75 mg of racemic ketamine is intranasally administered and the AUC₀₋₁ for 6-hydroxynorketamine is from about 450 ng*h/mL to about 950 ng*h/mL following the intranasal administration of racemic ketamine. In some embodiments, about 75 mg of racemic ketamine is intranasally administered and the AUC₀₋ₜ for 6-hydroxynorketamine is from about 900 ng*h/mL to about 1,400 ng*h/mL following the intranasal administration of racemic ketamine. In some embodiments, about 75 mg of racemic ketamine is intranasally administered and the AUC₀₋ₜ for 6-hydroxynorketamine is from about 1,350 ng*h/mL to about 1,900 ng*h/mL following the intranasal administration of racemic ketamine.

In some embodiments, about 90 mg of racemic ketamine is intranasally administered and the AUC₀₋ₜ for 6-hydroxynorketamine is from about 1,050 ng*h/mL to about 3,100 ng*h/mL, following the intranasal administration of racemic ketamine, for example, about 1,050 ng*h/mL, about 1,200 ng*h/mL, about 1,350 ng*h/mL, about 1,500 ng*h/mL, about 1,750 ng*h/mL, about 1,900 ng*h/mL, about 2,050 ng*h/mL, about 2,200 ng*h/mL, about 2,450 ng*h/mL, about 2,600 ng*h/mL, about 2,750 ng*h/mL, about 2,900 ng*h/mL, about 3100 ng*h/mL, or any value in between. In some embodiments, about 90 mg of racemic ketamine is intranasally administered and the AUC₀₋ₜ for 6-hydroxynorketamine is from about 1,050 ng*h/mL to about 1,850 ng*h/mL following the intranasal administration of racemic ketamine. In some embodiments, about 90 mg of racemic ketamine is intranasally administered and the AUC₀₋ₜ for 6-hydroxynorketamine is from about 1,800 ng*h/mL to about 2,600 ng*h/mL following the intranasal administration of racemic ketamine. In some embodiments, about 90 mg of racemic ketamine is intranasally administered and the AUC₀₋ₜ for 6-hydroxynorketamine is from about 2,550 ng*h/mL to about 3,100 ng*h/mL following the intranasal administration of racemic ketamine.

In some embodiments, the AUC₀₋ₜ for ketamine is from about 70 ng*h/mL to about 675 ng*h/mL, following the intranasal administration of racemic ketamine, for example, about 70 ng*h/mL, about 100 ng*h/mL, about 125 ng*h/mL, about 150 ng*h/mL, about 175 ng*h/mL, about 200 ng*h/mL, about 225 ng*h/mL, about 250 ng*h/mL, about 275 ng*h/mL, about 300 ng*h/mL, about 325 ng*h/mL, about 350 ng*h/mL, about 375 ng*h/mL, about 400 ng*h/mL, about 425 ng*h/mL, about 450 ng*h/mL, about 475 ng*h/mL, about 500 ng*h/mL, about 525 ng*h/mL, about 550 ng*h/mL, about 575 ng*h/mL, about 600, ng*h/mL, about 625 ng*h/mL, about 650 ng*h/mL, about 675 ng*h/mL, and the Cₘₐₓ of ketamine is from about 15 ng/ml to about 225 ng/ml, or any value in between following the intranasal administration of racemic ketamine. In some embodiments, the AUC₀₋ₜ for ketamine is from about 70 ng*h/mL to about 675 ng*h/mL, following the intranasal administration of racemic ketamine, for example, about 70 ng*h/mL, about 100 ng*h/mL, about 125 ng*h/mL, about 150 ng*h/mL, about 175 ng*h/mL, about 200 ng*h/mL, about 225 ng*h/mL, about 250 ng*h/mL, about 275 ng*h/mL, about 300 ng*h/mL, about 325 ng*h/mL, about 350 ng*h/mL, about 375 ng*h/mL, about 400 ng*h/mL, about 425 ng*h/mL, about 450 ng*h/mL, about 475 ng*h/mL, about 500 ng*h/mL, about 525 ng*h/mL, about 550 ng*h/mL, about 575 ng*h/mL, about 600, ng*h/mL, about 625 ng*h/mL, about 650 ng*h/mL, about 675 ng*h/mL, and the Cₘₐₓ of ketamine is from about 70 ng/ml to about 205 ng/mL, following the intranasal administration of racemic ketamine, for example, about 70 ng/ml, about 85 ng/ml, about 95 ng/ml, about 105 ng/ml, about 115 ng/ml, about 125 ng/ml, about 135 ng/ml, about 145 ng/ml, about 155 ng/ml, about 165 ng/mL, about 185 ng/ml, about 205 ng/mL, or any value in between. In some embodiments, the AUC₀₋ₜ for ketamine is from about 70 ng*h/mL to about 250 ng*h/mL, about 200 ng*h/mL to about 450 ng*h/mL, about 400 ng*h/mL to about 675 ng*h/mL, about 600 ng*h/mL to about 675 ng*h/mL following the intranasal administration of racemic ketamine, and the Cₘₐₓ of ketamine is from about 75 ng/ml to about 1755 ng/ml, for example, about 75 ng/ml, about 100 ng/ml, about 125 ng/ml, about 150 ng/ml, about 175 ng/ml, or any value in between following the intranasal administration of racemic ketamine.

In some embodiments, the AUC₀₋ₜ for norketamine is from about 250 ng*h/mL to about 2,200 ng*h/mL, following the intranasal administration of racemic ketamine, for example, about 250 ng*h/mL, about 350 ng*h/mL, about 450 ng*h/mL, about 550 ng*h/mL, about 650 ng*h/mL, about 750 ng*h/mL, about 850 ng*h/mL, about 950 ng*h/mL, about 1,050 ng*h/mL, about 1,150 ng*h/mL, about 1,250 ng*h/mL, about 1,350 ng*h/mL, about 1,450 ng*h/mL, about 1,550 ng*h/mL, about 1,650 ng*h/mL, about 1750 ng*h/mL, about 1,850 ng*h/mL, about 1,950 ng*h/mL, about 2,050 ng*h/mL, about 2,200 ng*h/mL, or any value in between, and the Cₘₐₓ of norketamine is from about 40 ng/ml to about 375 ng/ml, or any value in between following the intranasal administration of racemic ketamine. In some embodiments, the AUC₀₋ₜ for norketamine is from about 250 ng*h/mL to about 2,200 ng*h/mL, following the intranasal administration of racemic ketamine, for example, about 250 ng*h/mL, about 350 ng*h/mL, about 450 ng*h/mL, about 550 ng*h/mL, about 650 ng*h/mL, about 750 ng*h/mL, about 850 ng*h/mL, about 950 ng*h/mL, about 1,050 ng*h/mL, about 1,150 ng*h/mL, about 1,250 ng*h/mL, about 1,350 ng*h/mL, about 1,450 ng*h/mL, about 1,550 ng*h/mL, about 1,650 ng*h/mL, about 1,750 ng*h/mL, about 1,850 ng*h/mL, about 1,950 ng*h/mL, about 2,050 ng*h/mL, about 2,200 ng*h/mL, or any value in between, and the Cₘₐₓ of norketamine is from about 50 ng/ml to about 275 ng/ml, following the intranasal administration of racemic ketamine, for example, about 50 ng/ml, about 75 ng/ml, about 100 ng/ml, about 125 ng/ml, about 150 ng/mL, about 175 ng/ml, about 200 ng/ml, about 225 ng/ml, about 250 ng/ml, about 275 ng/ml, or any value in between. In some embodiments, the Cₘₐₓ of norketamine is from about 50 ng/ml to about 135 ng/ml, about 130 ng/ml to about 15 ng/ml, about 210 ng/ml to about 245 ng/ml, about 240 ng/ml to about 275 ng/mL, following the intranasal administration of racemic ketamine, and the AUC₀₋ₜ for norketamine is from about 250 ng*h/mL to about 950 ng*h/mL, about 900 ng*h/mL to about 1,550 ng*h/mL, or about 1,500 ng*h/mL to about 2,200 ng*h/mL following the intranasal administration of racemic ketamine. In some embodiments, the AUC₀₋ₜ for 6-hydroxynorketamine is from about 300 ng*h/mL to about 3,100 ng*h/mL, following the intranasal administration of racemic ketamine, for example, about 300 ng*h/mL, about 450 ng*h/mL, about 600 ng*h/mL, about 750 ng*h/mL, about 900 ng*h/mL, about 1,050 ng*h/mL, about 1,200 ng*h/mL, about 1,350 ng*h/mL, about 1,500 ng*h/mL, about 1,750 ng*h/mL, about 1,900 ng*h/mL, about 2,050 ng*h/mL, about 2,200 ng*h/mL, about 2,450 ng*h/mL, about 2,600 ng*h/mL, about 2,750 ng*h/mL, about 2,900 ng*h/mL, about 3100 ng*h/mL, or any value in between, and the Cₘₐₓ of 6-hydroxynorketamine is from about 15 ng/ml to about 275 ng/mL, or any value in between following the intranasal administration of racemic ketamine. In some embodiments, the AUC₀₋ₜ for 6-hydroxynorketamine is from about 300 ng*h/mL to about 3,100 ng*h/mL, following the intranasal administration of racemic ketamine, for example, about 300 ng*h/mL, about 450 ng*h/mL, about 600 ng*h/mL, about 750 ng*h/mL, about 900 ng*h/mL, about 1,050 ng*h/mL, about 1,200 ng*h/mL, about 1,350 ng*h/mL, about 1,500 ng*h/mL, about 1,750 ng*h/mL, about 1,900 ng*h/mL, about 2,050 ng*h/mL, about 2,200 ng*h/mL, about 2,450 ng*h/mL, about 2,600 ng*h/mL, about 2,750 ng*h/mL, about 2,900 ng*h/mL, about 3,100 ng*h/mL, or any value in between, following the intranasal administration of racemic ketamine, and the Cₘₐₓ of 6-hydroxynorketamine is from about 55 ng/ml to about 245 ng/ml, for example, about 55 ng/ml, about 65 ng/ml, about 75 ng/ml, about 85 ng/ml, about 95 ng/ml, about 105 ng/ml, about 115 ng/ml, about 125 ng/ml, about 135 ng/ml, about 145 ng/ml, about 155 ng/ml, about 165 ng/ml, about 175 ng/ml, about 185 ng/ml, about 195 ng/ml, about 205 ng/ml, about 215 ng/ml, about 225 ng/ml, about 235 ng/mL, about 245 ng/ml, or any value in between following the intranasal administration of racemic ketamine. In some embodiments, the AUC₀₋ₜ for 6-hydroxynorketamine is from about 700 ng*h/mL to about 1,550 ng*h/mL, about 1,500 ng*h/mL to about 2,050 ng*h/mL, about 2,000 ng*h/mL to about 2,550 ng*h/mL, about 2,500 ng*h/mL to about 3,100 ng*h/mL following the intranasal administration of racemic ketamine, and the Cₘₐₓ of 6-hydroxynorketamine is from about 55 ng/mL to about 125 ng/ml, about 120 ng/mL to about 180 ng/mL, or about 175 ng/mL to about 245 ng/ml following the intranasal administration of racemic ketamine.

In some embodiments, the AUC_{0-inf} for ketamine is from about 80 ng*h/mL to about 675 ng*h/mL, following the intranasal administration of racemic ketamine, for example, about 80 ng*h/mL, about 125 ng*h/mL, about 175 ng*h/mL, about 225 ng*h/mL, about 275 ng*h/mL, about 325 ng*h/mL, about 475 ng*h/mL, about 525 ng*h/mL, about 575 ng*h/mL, about 625 ng*h/mL, about 675 ng*h/mL, or any value in between. In some embodiments, the AUC_{0-inf} for ketamine is from about 80 ng*h/mL to about 175 ng*h/mL following the intranasal administration of racemic ketamine. In some embodiments, the AUC_{0-inf} for ketamine is from about 150 ng*h/mL to about 275 ng*h/mL following the intranasal administration of racemic ketamine. In some embodiments, the AUC_{0-inf} for ketamine is from about 250 ng*h/mL to about 375 ng*h/mL following the intranasal administration of racemic ketamine. In some embodiments, the AUC_{0-inf} for ketamine is from about 350 ng*h/mL to about 475 ng*h/mL following the intranasal administration of racemic ketamine. In some embodiments, the AUC_{0-inf} for ketamine is from about 450 ng*h/mL to about 575 ng*h/mL following the intranasal administration of racemic ketamine. In some embodiments, the AUC_{0-inf} for ketamine is from about 550 ng*h/mL to about 675 ng*h/mL following the intranasal administration of racemic ketamine.

In some embodiments, the AUC_{0-inf} for norketamine is from about 250 ng*h/mL to about 875 ng*h/mL, following the intranasal administration of racemic ketamine, for example, about 250 ng*h/mL, about 275 ng*h/mL, about 300 ng*h/mL, about 325 ng*h/mL, about 350 ng*h/mL, about 375 ng*h/mL, about 400 ng*h/mL, about 425 ng*h/mL, about 450 ng*h/mL, about 475 ng*h/mL, about 500 ng*h/mL, about 525 ng*h/mL, about 550 ng*h/mL, about 575 ng*h/mL, about 600 ng*h/mL, about 625 ng*h/mL, about 650 ng*h/mL, about 675 ng*h/mL, about 700 ng*h/mL, about 725 ng*h/mL, about 750 ng*h/mL, about 775 ng*h/mL, about 800 ng*h/mL, about 825 ng*h/mL, about 850 ng*h/mL, about 875 ng*h/mL or any value in between. In some embodiments, the AUC_{0-inf} for norketamine is from about 250 ng*h/mL to about 475 ng*h/mL following the intranasal administration of racemic ketamine. In some embodiments, the AUC_{0-inf} for norketamine is from about 450 ng*h/mL to about 675 ng*h/mL following the intranasal administration of racemic ketamine. In some embodiments, the AUC_{0-inf} for norketamine is from about 650 ng*h/mL to about 875 ng*h/mL following the intranasal administration of racemic ketamine.

In some embodiments, the AUC_{0-inf} for 6-hydroxynorketamine is from about 375 ng*h/mL to about 3,700 ng*h/mL, following the intranasal administration of racemic ketamine, for example, about 375 ng*h/mL, about 500 ng*h/mL, about 650 ng*h/mL, about 900 ng*h/mL, about 1,050 ng*h/mL, about 1,200 ng*h/mL, about 1,350 ng*h/mL, about 1,500 ng*h/mL, about 1,650 ng*h/mL, about 1,800 ng*h/mL, about 1,950 ng*h/mL, about 2,100 ng*h/mL, about 2,250 ng*h/mL, about 2,400 ng*h/mL, about 2,550 ng*h/mL, about 2,700 ng*h/mL, about 2,850 ng*h/mL, about 3,000 ng*h/mL, about 3,150 ng*h/mL, about 3,300 ng*h/mL, about 3,450 ng*h/mL, about 3,600 ng*h/mL, about 3,700 ng*h/mL, or any value in between. In some embodiments, the AUC_{0-inf} for 6-hydroxynorketamine is from about 375 ng*h/mL to about 1,250 ng*h/mL following the intranasal administration of racemic ketamine. In some embodiments, the AUC_{0-inf} for 6-hydroxynorketamine is from about 1,200 ng*h/mL to about 1,400 ng*h/mL following the intranasal administration of racemic ketamine. In some embodiments, the AUC_{0-inf} for 6-hydroxynorketamine is from about 1,350 ng*h/mL to about 2,700 ng*h/mL following the intranasal administration of racemic ketamine. In some embodiments, the AUC_{0-inf} for 6-hydroxynorketamine is from about 2,600 ng*h/mL to about 3,700 ng*h/mL following the intranasal administration of racemic ketamine.

In some embodiments, the residual area for ketamine is about 2.5% to about 8%, for example, about 2.5%, about 3%, about 3.5%, about 4%, about 4.5%, about 5%, about 5.5%, about 6%, about 6.5%, about 7%, about 7.5%, about 8%, or any value in between following the intranasal administration of racemic ketamine. In some embodiments, the residual area for ketamine is about 2.5% to about 5%, following the intranasal administration of racemic ketamine. In some embodiments, the residual area for ketamine is about 4% to about 6% following the intranasal administration of racemic ketamine. In some embodiments, the residual area for ketamine is about 5% to about 8% following the intranasal administration of racemic ketamine.

In some embodiments, the residual area for norketamine is about 6% to about 15%, following the intranasal administration of racemic ketamine, for example, about 6%, about 6.5%, about 7%, about 7.5%, about 8%, about 8.5%, about 9%, about 9.5%, about 10%, about 10.5%, about 11%, about 11.5%, about 12%, about 12.5%, about 13%, about 13.5%, about 14%, about 14.5%, about 15%, or any value in between. In some embodiments, the residual area for norketamine is about 2.5% to about 5% following the intranasal administration of racemic ketamine. In some embodiments, the residual area for norketamine is about 4% to about 6% following the intranasal administration of racemic ketamine. In some embodiments, the residual area for norketamine is about 5% to about 8% following the intranasal administration of racemic ketamine.

In some embodiments, the residual area for 6-hydroxynorketamine is about 16% to about 34%, following the intranasal administration of racemic ketamine, for example, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, or any value in between. In some embodiments, the residual area for 6-hydroxynorketamine is about 16% to about 24% following the intranasal administration of racemic ketamine. In some embodiments, the residual area for 6-hydroxynorketamine is about 20% to about 30% following the intranasal administration of racemic ketamine. In some embodiments, the residual area for 6-hydroxynorketamine is about 26% to about 34% following the intranasal administration of racemic ketamine.

In some embodiments, the AUC₀₋ₜ for the intranasal racemic ketamine is about 80% to about 125% of the AUC₀₋ₜ for an equivalent dose of intravenous racemic ketamine. For example, about 80%, about 85%, about 90%, about 95%, about 100%, about 105%, about 110%, about 115%, about 120%, about 125%, or any value in between. In some embodiments, the AUC₀₋ₜ for the intranasal racemic ketamine is about 80% to about 95% of the AUC₀₋ₜ for an equivalent dose of intravenous racemic ketamine. In some embodiments, the AUC_{0-inf} for the intranasal racemic ketamine is about 80% to about 125% of the AUC_{0-inf} for an equivalent dose of intravenous racemic ketamine. For example, about 80%, about 85%, about 90%, about 95%, about 100%, about 105%, about 110%, about 115%, about 120%, about 125%, or any value in between. In some embodiments, the AUC_{0-inf} for the intranasal racemic ketamine is about 80% to about 95% of the AUC_{0-inf} for an equivalent dose of intravenous racemic ketamine.

In some embodiments, the AUC₀₋ₜ of norketamine after administration of the intranasal racemic ketamine is about 1.1 to about 4.0 times greater than the AUC₀₋ₜ of norketamine after administration of an equivalent dose of intravenous racemic ketamine. In some embodiments, the AUC_{0-inf} of norketamine after administration of the intranasal racemic ketamine is about 1.1 to about 4.0 times greater than the AUC_{0-inf} of norketamine after administration of an equivalent dose of intravenous racemic ketamine. For example, about 1.1, about 1.2, about 1.3, about 1.4, about 1.5, about 1.6, about 1.7, about 1.8, about 1.9, about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, or about 4.0, times greater, or any value in between.

In some embodiments, the AUC₀₋ₜ of 6-hydroxynorketamine after administration of the intranasal racemic ketamine is about 1.3 to about 3.6 times greater than the AUC₀₋ₜ of norketamine after administration of an equivalent dose of intravenous racemic ketamine. For example, about 1.3, about 1.4, about 1.5, about 1.6, about 1.7, about 1.8, about 1.9, about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, or about 3.6, times greater, or any value in between. In some embodiments, the AUC_{0-inf} of 6-hydroxynorketamine after administration of the intranasal racemic ketamine is about 1.1 to about 3.1 times greater than the AUC_{0-inf} of norketamine after administration of an equivalent dose of intravenous racemic ketamine. For example, about 1.1, about 1.2, about 1.3, about 1.4, about 1.5, about 1.6, about 1.7, about 1.8, about 1.9, about 2.0, about 2.1, about 2.2, about 2.3, about 2.4, about 2.5, about 2.6, about 2.7, about 2.8, about 2.9, about 3.0, or about 3.1, times greater, or any value in between.

In some embodiments, the Cₘₐₓ for the intranasal racemic ketamine is about 25% to about 125% of the Cₘₐₓ for an equivalent dose of intravenous racemic ketamine. For example, about 25%, about 30%, about 35%, about 40%, about 45%, about 50%, about 55%, about 60%, about 65%, about 70%, about 75%, about 80%, about 85%, about 90%, about 95%, about 100%, about 105%, about 110%, about 115%, about 120%, about 125%, or any value in between. In some embodiments, the Cₘₐₓ for the intranasal racemic ketamine is about 25% to about 100% of the Cₘₐₓ for an equivalent dose of intravenous racemic ketamine. In some embodiments, the Cₘₐₓ for the intranasal racemic ketamine is about 30% to about 75% of the Cₘₐₓ for an equivalent dose of intravenous racemic ketamine. In some embodiments, the Cₘₐₓ for the intranasal racemic ketamine is about 50% to about 70% of the Cₘₐₓ for an equivalent dose of intravenous racemic ketamine.

In some embodiments, the Cₘₐₓ of norketamine after administration of the intranasal racemic ketamine is about 1.5 to about 6.0 times greater than the Cₘₐₓ of norketamine after administration of an equivalent dose of intravenous racemic ketamine. For example, about 1.5, about 1.6, about 1.7, about 1.8, about 1.9, about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0, about 4.1, about 4.2, about 4.3, about 4.4, about 4.5, about 4.6, about 4.7, about 4.8, about 4.9, about 5.0, about 5.1, about 5.2, about 5.3, about 5.4, about 5.5, about 5.6, about 5.7, about 5.8, about 5.9, or about 6.0 times greater, or any value in between.

In some embodiments, the Cₘₐₓ of 6-hydroxynorketamine after administration of the intranasal racemic ketamine is about 1.4 to about 5.0 times greater than the Cₘₐₓ of 6-hydroxynorketamine after administration of an equivalent dose of intravenous racemic ketamine. For example, about 1.4, about 1.5, about 1.6, about 1.7, about 1.8, about 1.9, about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0, about 4.1, about 4.2, about 4.3, about 4.4, about 4.5, about 4.6, about 4.7, about 4.8, about 4.9, or about 5.0 times greater, or any value in between.

In some embodiments, the Tₘₐₓ for the intranasal racemic ketamine is about 1.6 to about 6.0 times greater than the Tₘₐₓ for an equivalent dose of intravenous racemic ketamine. For example, about 1.6, about 1.7, about 1.8, about 1.9, about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0, about 4.1, about 4.2, about 4.3, about 4.4, about 4.5, about 4.6, about 4.7, about 4.8, about 4.9, about 5.0, about 5.1, about 5.2, about 5.3, about 5.4, about 5.5, about 5.6, about 5.7, about 5.8, about 5.9, or about 6.0 times greater, or any value in between.

In some embodiments, the Tₘₐₓ of norketamine after administration of the intranasal racemic ketamine is about 30% to about 550% of the Tₘₐₓ of norketamine after administration of an equivalent dose of intravenous racemic ketamine. For example, about 30%, about 45%, about 60%, about 75%, about 90%, about 105%, about 120%, about 140%, about 155%, about 170%, about 185%, about 200%, about 215%, about 230%, about 245%, about 260%, about 275%, about 290%, about 305%, about 320%, about 340%, about 355%, about 370%, about 385%, about 400%, about 415%, about 430%, about 445%, about 460%, about 475%, about 490%, about 505%, about 520%, about 540%, about 550%, or any value in between.

In some embodiments, the Tₘₐₓ of norketamine after administration of the intranasal racemic ketamine is about 80% to about 240% of the Tₘₐₓ of norketamine after administration of an equivalent dose of intravenous racemic ketamine. For example, about 80%, about 100%, about 120%, about 140%, about 160%, about 180%, about 200%, about 220%, about 240%, or any value in between. In some embodiments, the Tₘₐₓ of norketamine after administration of the intranasal racemic ketamine is about 90% to about 180% of the Tₘₐₓ of norketamine after administration of an equivalent dose of intravenous racemic ketamine. For example, about 90%, about 100%, about 110%, about 120%, about 130%, about 140%, about 150%, about 160%, about 170%, about 180%, or any value in between.

In some embodiments, the Tₘₐₓ of 6-hydroxynorketamine after administration of the intranasal racemic ketamine is about 20% to about 200% of the Tₘₐₓ of 6-hydroxynorketamine after administration of an equivalent dose of intravenous racemic ketamine. For example, about 20%, about 30%, about 40%, about 50%, about 60%, about 70%, about 80%, about 90%, about 100%, about 110%, about 120%, about 130%, about 140%, about 150%, about 160%, about 170%, about 180%, about 190%, about 200%, or any value in between. In some embodiments, the Tₘₐₓ of 6-hydroxynorketamine after administration of the intranasal racemic ketamine is about 50% to about 100% of the Tₘₐₓ of 6-hydroxynorketamine after administration of an equivalent dose of intravenous racemic ketamine. For example, about 50%, about 60%, about 70%, about 80%, about 90%, about 100%, or any value in between. In some embodiments, the Tₘₐₓ of 6-hydroxynorketamine after administration of the intranasal racemic ketamine is about 65% to about 85% of the Tₘₐₓ of 6-hydroxynorketamine after administration of an equivalent dose of intravenous racemic ketamine. For example, about 65%, about 70%, about 75%, about 80%, about 85%, or any value in between.

In some embodiments, administration of intranasal racemic ketamine provides higher exposure and/or higher plasma concentrations of one or more active metabolites of ketamine relative to an equivalent dose of intravenous racemic ketamine. In some embodiments, the one or more active metabolites are selected from norketamine, 6-hydroxynorketamine, and a combination thereof.

In some embodiments, administration of intranasal racemic ketamine provides higher exposure and/or higher plasma concentrations of norketamine, relative to an equivalent dose of intravenous racemic ketamine. In some embodiments, administration of intranasal racemic ketamine provides higher exposure and/or higher plasma concentrations of 6-hydroxynorketamine, relative to an equivalent dose of intravenous racemic ketamine. In some embodiments, administration of intranasal racemic ketamine provides higher exposure and/or higher plasma concentrations of norketamine and 6-hydroxynorketamine, relative to an equivalent dose of intravenous racemic ketamine.

In some embodiments, “equivalent” doses of intranasal racemic ketamine and intravenous racemic ketamine and/or intravenous (S)-ketamine, or a pharmaceutically acceptable salt of any of the foregoing, is determined by the equivalence of the AUC_{0-inf} values.

In some embodiments, intranasal administration of the racemic ketamine exhibits one or more of:
an AUC₀₋ₜ of norketamine that is at least 1.5 times higher than the AUC₀₋ₜ of norketamine exhibited by intravenous administration of an equivalent dose of racemic ketamine;
an AUC_{0-inf} of norketamine that is at least 1.5 times higher than the AUC_{0-inf} of norketamine exhibited by intravenous administration of an equivalent dose of racemic ketamine; and
a Cₘₐₓ of norketamine that is at least 2 times higher than the Cₘₐₓ of norketamine exhibited by intravenous administration of an equivalent dose of racemic ketamine.

In some embodiments, intranasal administration of the racemic ketamine exhibits an AUC₀₋ₜ of norketamine that is about 1.7 to about 2.5 times higher than the AUC₀₋ₜ of norketamine exhibited by intravenous administration of an equivalent dose of racemic ketamine. In some embodiments, intranasal administration of the racemic ketamine exhibits an AUC₀₋ₜ of norketamine that is about 1.9 to about 2.3 times higher than the AUC₀₋₁ of norketamine exhibited by intravenous administration of an equivalent dose of racemic ketamine. In some embodiments, intranasal administration of the racemic ketamine exhibits an AUC₀₋ₜ of norketamine that is at least 1.8 times higher (e.g., at least 1.9 times higher or at least 2 times higher) than the AUC₀₋ₜ of norketamine exhibited by intravenous administration of an equivalent dose of racemic ketamine.

In some embodiments, intranasal administration of the racemic ketamine exhibits an AUC_{0-inf} of norketamine that is about 1.5 to about 2.5 times higher than the AUC_{0-inf} of norketamine exhibited by intravenous administration of an equivalent dose of racemic ketamine. In some embodiments, intranasal administration of the racemic ketamine exhibits an AUC₀₋ₜ of norketamine that is about 1.8 to about 2.2 times higher than the AUC_{0-inf} of norketamine exhibited by intravenous administration of an equivalent dose of racemic ketamine. In some embodiments, intranasal administration of the racemic ketamine exhibits an AUC₀₋ₜ of norketamine that is at least 1.7 time higher (e.g., at least 1.8 times higher, at least 1.9 times higher, or at least 2 times higher) than the AUC_{0-inf} of norketamine exhibited by intravenous administration of an equivalent dose of racemic ketamine.

In some embodiments, intranasal administration of the racemic ketamine exhibits a Cₘₐₓ of norketamine that is about 2.2 to about 3.5 times higher than the Cₘₐₓ of norketamine exhibited by intravenous administration of an equivalent dose of racemic ketamine. In some embodiments, intranasal administration of the racemic ketamine exhibits a Cₘₐₓ of norketamine that is about 2.4 to about 3.2 times higher than the Cₘₐₓ of norketamine exhibited by intravenous administration of an equivalent dose of racemic ketamine. In some embodiments, intranasal administration of the racemic ketamine exhibits a Cₘₐₓ of norketamine that is at least 2.5 times higher (e.g., at least 2.8 times higher or at least 3 times higher) than the Cₘₐₓ of norketamine exhibited by intravenous administration of an equivalent dose of racemic ketamine. In some embodiments, intranasal administration of the racemic ketamine exhibits a Tₘₐₓ of norketamine that is about 80% to about 125% of the Tₘₐₓ of norketamine exhibited by an intravenous administration of an equivalent dose of racemic ketamine. In some embodiments, intranasal administration of the racemic ketamine exhibits a Tₘₐₓ of norketamine that is about 90% to about 110% of the Tₘₐₓ of norketamine exhibited by an intravenous administration of an equivalent dose of racemic ketamine. In some embodiments, intranasal administration of the racemic ketamine exhibits a Tₘₐₓ of norketamine that is about 95% to about 105% of the Tₘₐₓ of norketamine exhibited by an intravenous administration of an equivalent dose of racemic ketamine.

In some embodiments, one or more of the AUC₀₋ₜ, AUC_{0-inf}, Cₘₐₓ, and Tₘₐₓ of norketamine is determined following one dose of racemic ketamine. In some embodiments, one or more of the AUC₀₋ₜ, AUC_{0-inf}, Cₘₐₓ, and Tₘₐₓ of norketamine is determined following two doses of racemic ketamine. In some embodiments, one or more of the AUC₀₋ₜ, AUC_{0-inf}, Cₘₐₓ, and Tₘₐₓ of norketamine is determined following three doses of racemic ketamine.

In some embodiments, intranasal administration of the racemic ketamine exhibits one or more of:
an AUC₀₋ₜ of hydroxynorketamine that is at least 1.5 times higher than the AUC₀₋ₜ of hydroxynorketamine exhibited by intravenous administration of an equivalent dose of racemic ketamine;
an AUC_{0-inf} of hydroxynorketamine that is at least 1.2 times higher than the AUC_{0-inf} of hydroxynorketamine exhibited by intravenous administration of an equivalent dose of racemic ketamine; and
a Cₘₐₓ of hydroxynorketamine that is at least 2 times higher than the Cₘₐₓ of norketamine exhibited by intravenous administration of an equivalent dose of racemic ketamine.

In some embodiments, intranasal administration of the racemic ketamine exhibits an AUC₀₋ₜ of hydroxynorketamine that is about 1.7 to about 2.5 times higher than the AUC₀₋ₜ of hydroxynorketamine exhibited by intravenous administration of an equivalent dose of racemic ketamine. In some embodiments, intranasal administration of the racemic ketamine exhibits an AUC₀₋ₜ of hydroxynorketamine that is about 1.9 to about 2.3 times higher than the AUC₀₋ₜ of hydroxynorketamine exhibited by intravenous administration of an equivalent dose of racemic ketamine. In some embodiments, intranasal administration of the racemic ketamine exhibits an AUC₀₋ₜ of hydroxynorketamine that is at least 1.9 times higher (e.g., at least 2 times higher or at least 2.1 times higher) than the AUC₀₋ₜ of hydroxynorketamine exhibited by intravenous administration of an equivalent dose of racemic ketamine.

In some embodiments, intranasal administration of the racemic ketamine exhibits an AUC_{0-inf} of hydroxynorketamine that is about 1.5 to about 2.5 times higher than the AUC_{0-inf} of hydroxynorketamine exhibited by intravenous administration of an equivalent dose of racemic ketamine. In some embodiments, intranasal administration of the racemic ketamine exhibits an AUC₀₋ₜ of hydroxynorketamine that is about 1.7 to about 2.1 times higher than the AUC_{0-inf} of hydroxynorketamine exhibited by intravenous administration of an equivalent dose of racemic ketamine. In some embodiments, intranasal administration of the racemic ketamine exhibits an AUC₀₋ₜ of hydroxynorketamine that is at least 1.7 times higher (e.g., at least 1.8 times higher or at least 1.9 times higher) than the AUC_{0-inf} of hydroxynorketamine exhibited by intravenous administration of an equivalent dose of racemic ketamine.

In some embodiments, intranasal administration of the racemic ketamine exhibits a Cₘₐₓ of hydroxynorketamine that is about 2.2 to about 3.2 times higher than the Cₘₐₓ of hydroxynorketamine exhibited by intravenous administration of an equivalent dose of racemic ketamine. In some embodiments, intranasal administration of the racemic ketamine exhibits a Cₘₐₓ of hydroxynorketamine that is about 2.4 to about 2.8 times higher than the Cₘₐₓ of hydroxynorketamine exhibited by intravenous administration of an equivalent dose of racemic ketamine. In some embodiments, intranasal administration of the racemic ketamine exhibits a Cₘₐₓ of hydroxynorketamine that is at least 2.4 times higher (e.g., at least 2.5 times higher or at least 2.6 times higher) than the Cₘₐₓ of hydroxynorketamine exhibited by intravenous administration of an equivalent dose of racemic ketamine.

In some embodiments, intranasal administration of the racemic ketamine exhibits a Tₘₐₓ of hydroxynorketamine that is about 80% to about 125% of the Tₘₐₓ of hydroxynorketamine exhibited by an intravenous administration of an equivalent dose of racemic ketamine. In some embodiments, intranasal administration of the racemic ketamine exhibits a Tₘₐₓ of hydroxynorketamine that is about 90% to about 110% of the Tₘₐₓ of hydroxynorketamine exhibited by an intravenous administration of an equivalent dose of racemic ketamine. In some embodiments, intranasal administration of the racemic ketamine exhibits a Tₘₐₓ of hydroxynorketamine that is about 95% to about 105% of the Tₘₐₓ of hydroxynorketamine exhibited by an intravenous administration of an equivalent dose of racemic ketamine.

In some embodiments, one or more of the AUC₀₋ₜ, AUC_{0-inf}, Cₘₐₓ, and Tₘₐₓ of hydroxynorketamine is determined following one dose of racemic ketamine. In some embodiments, one or more of the AUC₀₋ₜ, AUC_{0-inf}, Cₘₐₓ, and Tₘₐₓ of hydroxynorketamine is determined following two doses of racemic ketamine. In some embodiments, one or more of the AUC₀₋ₜ, AUC_{0-inf}, Cₘₐₓ, and Tₘₐₓ of hydroxynorketamine is determined following three doses of racemic ketamine.

### Further Methods and Embodiments

Some embodiments provide a method of treating MDD in a subject in need thereof, comprising:
(a) determining if the subject has one or more of:
(i) a MADRS Total Score of at least 20 units;
(ii) a MADRS Item 10 Score of 1-3 units;
(iii) a CGIS-SI/B score of 2 or 3 units;
(iv) a STS-CMCM score of at least 10 units;
(v) a STS-CMCM Risk of Suicide at Within the Next 7 Days score of at least 3 units; and
(vi) a C-SSRS score of 0-2; and
(b) intranasally administering to the subject a therapeutically effective amount of racemic ketamine, or a pharmaceutically acceptable salt thereof;
   wherein intranasal administration of the racemic ketamine exhibits one or more of:
   an AUC₀₋ₜ of norketamine that is at least 1.5 times higher than the AUC₀₋ₜ of norketamine exhibited by intravenous administration of an equivalent dose of racemic ketamine;
   an AUC_{0-inf} of norketamine that is at least 1.5 times higher than the AUC_{0-inf} of norketamine exhibited by intravenous administration of an equivalent dose of racemic ketamine; and
   a Cₘₐₓ of norketamine that is at least 2 times higher than the Cₘₐₓ of norketamine exhibited by intravenous administration of an equivalent dose of racemic ketamine.

Some embodiments provide a method of treating TRD in a subject in need thereof, comprising:
(a) determining if the subject has one or more of:
(i) a MADRS Total Score of at least 20 units;
(ii) a MADRS Item 10 Score of 1-3 units;
(iii) a CGIS-SI/B score of 2 or 3 units;
(iv) a STS-CMCM score of at least 10 units;
(v) a STS-CMCM Risk of Suicide at Within the Next 7 Days score of at least 3 units; and
(vi) a C-SSRS score of 0-2; and
   (b) intranasally administering to the subject a therapeutically effective amount of racemic ketamine, or a pharmaceutically acceptable salt thereof;
   wherein intranasal administration of the racemic ketamine exhibits one or more of:
   an AUC₀₋ₜ of norketamine that is at least 1.5 times higher than the AUC₀₋ₜ of norketamine exhibited by intravenous administration of an equivalent dose of racemic ketamine;
   an AUC_{0-inf} of norketamine that is at least 1.5 times higher than the AUC_{0-inf} of norketamine exhibited by intravenous administration of an equivalent dose of racemic ketamine; and
   a Cₘₐₓ of norketamine that is at least 2 times higher than the Cₘₐₓ of norketamine exhibited by intravenous administration of an equivalent dose of racemic ketamine.

Some embodiments provide a method of treating PTSD in a subject in need thereof, comprising intranasally administering to the subject a therapeutically effective amount of racemic ketamine, or a pharmaceutically acceptable salt thereof;
wherein intranasal administration of the racemic ketamine exhibits one or more of:
an AUC₀₋ₜ of norketamine that is at least 1.5 times higher than the AUC₀₋ₜ of norketamine exhibited by intravenous administration of an equivalent dose of racemic ketamine;
an AUC_{0-inf} of norketamine that is at least 1.5 times higher than the AUC_{0-inf} of norketamine exhibited by intravenous administration of an equivalent dose of racemic ketamine; and
a Cₘₐₓ of norketamine that is at least 2 times higher than the Cₘₐₓ of norketamine exhibited by intravenous administration of an equivalent dose of racemic ketamine.

Some embodiments provide a method of treating MDD in a subject in need thereof, comprising:
(a) determining if the subject has one or more of:
(i) a MADRS Total Score of at least 20 units;
(ii) a MADRS Item 10 Score of 1-3 units;
(iii) a CGIS-SI/B score of 2 or 3 units;
(iv) a STS-CMCM score of at least 10 units;
(v) a STS-CMCM Risk of Suicide at Within the Next 7 Days score of at least 3 units; and
(vi) a C-SSRS score of 0-2; and
   (b) intranasally administering to the subject a therapeutically effective amount of racemic ketamine, or a pharmaceutically acceptable salt thereof;
   wherein intranasal administration of the racemic ketamine exhibits one or more of:
   an AUC₀₋ₜ of hydroxynorketamine that is at least 1.5 times higher than the AUC₀₋ₜ of hydroxynorketamine exhibited by intravenous administration of an equivalent dose of racemic ketamine;
   an AUC_{0-inf} of hydroxynorketamine that is at least 1.2 times higher than the AUC_{0-inf} of hydroxynorketamine exhibited by intravenous administration of an equivalent dose of racemic ketamine; and
   a Cₘₐₓ of hydroxynorketamine that is at least 2 times higher than the Cₘₐₓ of norketamine exhibited by intravenous administration of an equivalent dose of racemic ketamine.

Some embodiments provide a method of treating TRD in a subject in need thereof, comprising:
(a) determining if the subject has one or more of:
(i) a MADRS Total Score of at least 20 units;
(ii) a MADRS Item 10 Score of 1-3 units;
(iii) a CGIS-SI/B score of 2 or 3 units;
(iv) a STS-CMCM score of at least 10 units;
(v) a STS-CMCM Risk of Suicide at Within the Next 7 Days score of at least 3 units; and
(vi) a C-SSRS score of 0-2; and
   (b) intranasally administering to the subject a therapeutically effective amount of racemic ketamine, or a pharmaceutically acceptable salt thereof;
   wherein intranasal administration of the racemic ketamine exhibits one or more of:
   an AUC₀₋ₜ of hydroxynorketamine that is at least 1.5 times higher than the AUC₀₋ₜ of hydroxynorketamine exhibited by intravenous administration of an equivalent dose of racemic ketamine;
   an AUC_{0-inf} of hydroxynorketamine that is at least 1.2 times higher than the AUC_{0-inf} of hydroxynorketamine exhibited by intravenous administration of an equivalent dose of racemic ketamine; and
   a Cₘₐₓ of hydroxynorketamine that is at least 2 times higher than the Cₘₐₓ of norketamine exhibited by intravenous administration of an equivalent dose of racemic ketamine.

Some embodiments provide a method of treating PTSD in a subject in need thereof, comprising intranasally administering to the subject a therapeutically effective amount of racemic ketamine, or a pharmaceutically acceptable salt thereof;
wherein intranasal administration of the racemic ketamine exhibits one or more of:
an AUC₀₋ₜ of hydroxynorketamine that is at least 1.5 times higher than the AUC₀₋ₜ of hydroxynorketamine exhibited by intravenous administration of an equivalent dose of racemic ketamine;
an AUC_{0-inf} of hydroxynorketamine that is at least 1.2 times higher than the AUC_{0-inf} of hydroxynorketamine exhibited by intravenous administration of an equivalent dose of racemic ketamine; and
a Cₘₐₓ of hydroxynorketamine that is at least 2 times higher than the Cₘₐₓ of norketamine exhibited by intravenous administration of an equivalent dose of racemic ketamine.

Some embodiments provide a method of treating MDD in a subject in need thereof, comprising:
(a) determining if the subject has one or more of:
(i) a MADRS Total Score of at least 20 units;
(ii) a MADRS Item 10 Score of 4, 5, or 6 units;
(iii) a CGIS-SI/B score of 4 or 5 units;
(iv) a STS-CMCM score of at least 15 units;
(v) a STS-CMCM Risk of Suicide at Within the Next 7 Days score of at least 5 units; and
(vi) a C-SSRS score of at least 2; and
(b) intranasally administering to the subject a therapeutically effective amount of racemic ketamine, or a pharmaceutically acceptable salt thereof;
   wherein intranasal administration of the racemic ketamine exhibits one or more of:
   an AUC₀₋ₜ of norketamine that is at least 1.5 times higher than the AUC₀₋ₜ of norketamine exhibited by intravenous administration of an equivalent dose of racemic ketamine;
   an AUC_{0-inf} of norketamine that is at least 1.5 times higher than the AUC_{0-inf} of norketamine exhibited by intravenous administration of an equivalent dose of racemic ketamine; and
   a Cₘₐₓ of norketamine that is at least 2 times higher than the Cₘₐₓ of norketamine exhibited by intravenous administration of an equivalent dose of racemic ketamine.

Some embodiments provide a method of treating MDD in a subject in need thereof, comprising:
(a) determining if the subject has one or more of:
(i) a MADRS Total Score of at least 20 units;
(ii) a MADRS Item 10 Score of 1-3 units;
(iii) a CGIS-SI/B score of 2 or 3 units;
(iv) a STS-CMCM score of at least 10 units;
(v) a STS-CMCM Risk of Suicide at Within the Next 7 Days score of at least 3 units; and
(vi) a C-SSRS score of 0-2; and
(b) intranasally administering to the subject a therapeutically effective amount of racemic ketamine, or a pharmaceutically acceptable salt thereof;
   wherein intranasal administration of the racemic ketamine exhibits one or more of:
   an AUC₀₋ₜ of norketamine that is at least 1.5 times higher than the AUC₀₋ₜ of norketamine exhibited by intravenous administration of an equivalent dose of racemic ketamine;
   an AUC_{0-inf} of norketamine that is at least 1.5 times higher than the AUC_{0-inf} of norketamine exhibited by intravenous administration of an equivalent dose of racemic ketamine; and
   a Cₘₐₓ of norketamine that is at least 2 times higher than the Cₘₐₓ of norketamine exhibited by intravenous administration of an equivalent dose of racemic ketamine.

Some embodiments provide a method of treating TRD in a subject in need thereof, comprising:
(a) determining if the subject has one or more of:
(i) a MADRS Total Score of at least 20 units;
(ii) a MADRS Item 10 Score of 1-3 units;
(iii) a CGIS-SI/B score of 2 or 3 units;
(iv) a STS-CMCM score of at least 10 units;
(v) a STS-CMCM Risk of Suicide at Within the Next 7 Days score of at least 3 units; and
(vi) a C-SSRS score of 0-2; and
   (b) intranasally administering to the subject a therapeutically effective amount of racemic ketamine, or a pharmaceutically acceptable salt thereof;
   wherein intranasal administration of the racemic ketamine exhibits one or more of:
   an AUC₀₋ₜ of norketamine that is at least 1.5 times higher than the AUC₀₋ₜ of norketamine exhibited by intravenous administration of an equivalent dose of racemic ketamine;
   an AUC_{0-inf} of norketamine that is at least 1.5 times higher than the AUC_{0-inf} of norketamine exhibited by intravenous administration of an equivalent dose of racemic ketamine; and
   a Cₘₐₓ of norketamine that is at least 2 times higher than the Cₘₐₓ of norketamine exhibited by intravenous administration of an equivalent dose of racemic ketamine.

Some embodiments provide a method of treating PTSD in a subject in need thereof, comprising intranasally administering to the subject a therapeutically effective amount of racemic ketamine, or a pharmaceutically acceptable salt thereof;
wherein intranasal administration of the racemic ketamine exhibits one or more of:
an AUC₀₋ₜ of norketamine that is at least 1.5 times higher than the AUC₀₋ₜ of norketamine exhibited by intravenous administration of an equivalent dose of racemic ketamine;
an AUC_{0-inf} of norketamine that is at least 1.5 times higher than the AUC_{0-inf} of norketamine exhibited by intravenous administration of an equivalent dose of racemic ketamine; and
a Cₘₐₓ of norketamine that is at least 2 times higher than the Cₘₐₓ of norketamine exhibited by intravenous administration of an equivalent dose of racemic ketamine.

Some embodiments provide a method of treating MDD in a subject in need thereof, comprising:
(a) determining if the subject has one or more of:
(i) a MADRS Total Score of at least 20 units;
(ii) a MADRS Item 10 Score of 1-3 units;
(iii) a CGIS-SI/B score of 2 or 3 units;
(iv) a STS-CMCM score of at least 10 units;
(v) a STS-CMCM Risk of Suicide at Within the Next 7 Days score of at least 3 units; and
(vi) a C-SSRS score of 0-2; and
   (b) intranasally administering to the subject a therapeutically effective amount of racemic ketamine, or a pharmaceutically acceptable salt thereof;
      wherein no clinically meaningful sedation is observed in the subject within about 24 hours after intranasal administration of the racemic ketamine, or a pharmaceutically acceptable salt thereof. In some embodiments, no clinically meaningful sedation is observed in the subject at about 4 hours after intranasal administration of the racemic ketamine, or a pharmaceutically acceptable salt thereof. In some embodiments, no clinically meaningful sedation is observed in the subject at about 1 hour after intranasal administration of the racemic ketamine, or a pharmaceutically acceptable salt thereof.

Some embodiments provide a method of treating MDD in a subject in need thereof, comprising:
(a) determining if the subject has a MADRS total score of at least 28 prior to the first administration of intranasal racemic ketamine; and
(b) intranasally administering to the subject a therapeutically effective amount of racemic ketamine, or a pharmaceutically acceptable salt thereof;
wherein no clinically meaningful sedation is observed in the subject within about 24 hours after intranasal administration of the racemic ketamine, or a pharmaceutically acceptable salt thereof. In some embodiments, no clinically meaningful sedation is observed in the subject at about 4 hours after intranasal administration of the racemic ketamine, or a pharmaceutically acceptable salt thereof. In some embodiments, no clinically meaningful sedation is observed in the subject at about 1 hour after intranasal administration of the racemic ketamine, or a pharmaceutically acceptable salt thereof; and
(c) optionally determining the MADRS total score of the subject about 24 hours after the first administration of intranasal racemic ketamine; and
(d) optionally determining one or more of the following of the subject:
(1) MADRS total score at Day 16;
(2) Clinical Global Impression Severity (CGIS) at 24 hours;
(3) Patient Global Impression Severity (PGIS) at 24 hours;
(4) CGIS at Day 16;
(5) PGIS at Day 16;
(6) MADRS Total Score at week 6;
(7) CGIS at week 6;
(8) PGIS at week 6;
(9) Clinical Global Impression Change (CGIC) at Day 16 and week 6;
(10) Patient Global Impression Change (PGIC) at Day 16 and week 6;
(11) Sheehan Disability Scale at Day 16 and week 6; and
(12) Posttreatment Phase—time to relapse for responders in the treatment phase, who subsequently relapse during the 4 week safety follow-up (Relapse is defined as MADRS total score ≥22 for two consecutive weeks);
   wherein three of more of (1)-(12) are improved relative to baseline (prior to administration of intranasal racemic ketamine, or a pharmaceutically acceptable salt thereof).

In some embodiments, four or more of (1)-(12) are improved relative to baseline (prior to administration of intranasal racemic ketamine, or a pharmaceutically acceptable salt thereof). In some embodiments, five or more of (1)-(12) are improved relative to baseline (prior to administration of intranasal racemic ketamine, or a pharmaceutically acceptable salt thereof). In some embodiments, six or more of (1)-(12) are improved relative to baseline (prior to administration of intranasal racemic ketamine, or a pharmaceutically acceptable salt thereof). In some embodiments, seven or more of (1)-(12) are improved relative to baseline (prior to administration of intranasal racemic ketamine, or a pharmaceutically acceptable salt thereof). In some embodiments, eight or more of (1)-(12) are improved relative to baseline (prior to administration of intranasal racemic ketamine, or a pharmaceutically acceptable salt thereof). In some embodiments, nine or more of (1)-(12) are improved relative to baseline (prior to administration of intranasal racemic ketamine, or a pharmaceutically acceptable salt thereof). In some embodiments, ten or more of (1)-(12) are improved relative to baseline (prior to administration of intranasal racemic ketamine, or a pharmaceutically acceptable salt thereof). In some embodiments, eleven of (1)-(12) are improved relative to baseline (prior to administration of intranasal racemic ketamine, or a pharmaceutically acceptable salt thereof). In some embodiments, each of (1)-(12) are improved relative to baseline (prior to administration of intranasal racemic ketamine, or a pharmaceutically acceptable salt thereof).

Some embodiments provide a method of treating TRD in a subject in need thereof, comprising:
(a) determining if the subject has one or more of:
(i) a MADRS Total Score of at least 20 units;
(ii) a MADRS Item 10 Score of 1-3 units;
(iii) a CGIS-SI/B score of 2 or 3 units;
(iv) a STS-CMCM score of at least 10 units;
(v) a STS-CMCM Risk of Suicide at Within the Next 7 Days score of at least 3 units; and
(vi) a C-SSRS score of 0-2; and
(b) intranasally administering to the subject a therapeutically effective amount of racemic ketamine, or a pharmaceutically acceptable salt thereof;
wherein no clinically meaningful dissociation is observed in the subject within about 24 hours after intranasal administration of the racemic ketamine, or a pharmaceutically acceptable salt thereof. In some embodiments, no clinically meaningful dissociation is observed in the subject at about 4 hours after intranasal administration of the racemic ketamine, or a pharmaceutically acceptable salt thereof. In some embodiments, no clinically meaningful dissociation is observed in the subject at about 1 hour after intranasal administration of the racemic ketamine, or a pharmaceutically acceptable salt thereof.

Some embodiments provide a method of treating PTSD in a subject in need thereof, comprising:
(a) intranasally administering to the subject a therapeutically effective amount of racemic ketamine, or a pharmaceutically acceptable salt thereof;
wherein no clinically meaningful dissociation is observed in the subject within about 24 hours after intranasal administration of the racemic ketamine, or a pharmaceutically acceptable salt thereof. In some embodiments, no clinically meaningful dissociation is observed in the subject at about 4 hours after intranasal administration of the racemic ketamine, or a pharmaceutically acceptable salt thereof. In some embodiments, no clinically meaningful dissociation is observed in the subject at about 1 hour after intranasal administration of the racemic ketamine, or a pharmaceutically acceptable salt thereof.
(b) Determining if the subject has one or more of the following after administration of the intranasal racemic ketamine:
(i) STS-CMCM Clinician Judgement of Patient Risk of Suicide—At This Time
(ii) STS-CMCM Total Score;
(iii) STS-CMCM Most Time Spent Per Day with Any Suicidal Impulses, Thoughts or Actions—past 24 hours;
(iv) STS-CMCM Clinician Global Severity Rating;
(v) STS-CMCM Patient Clinically Meaningful Change Measure;
(vi) CAPS-5 score of at least; and
(vii) MADRS score;
   wherein two or more of (i)-(vii) are improved relative to baseline (prior to administration of intranasal racemic ketamine, or a pharmaceutically acceptable salt thereof).

In some embodiments, three of more of (i)-(vii) are improved relative to baseline (prior to administration of intranasal racemic ketamine, or a pharmaceutically acceptable salt thereof). In some embodiments, four or more of (i)-(vii) are improved relative to baseline (prior to administration of intranasal racemic ketamine, or a pharmaceutically acceptable salt thereof). In some embodiments, five or more of (i)-(vii) are improved relative to baseline (prior to administration of intranasal racemic ketamine, or a pharmaceutically acceptable salt thereof). In some embodiments, six of (i)-(vii) are improved relative to baseline (prior to administration of intranasal racemic ketamine, or a pharmaceutically acceptable salt thereof). In some embodiments, each of (i)-(vii) are improved relative to baseline (prior to administration of intranasal racemic ketamine, or a pharmaceutically acceptable salt thereof).

In some embodiments, the STS-CMCM Clinician Judgement of Patient Risk of Suicide-Current, is measured at 24 hours following the Day 0 dose of the intranasal racemic ketamine, or a pharmaceutically acceptable salt thereof.

Some embodiments provide a method of treating PTSD in a subject in need thereof, comprising:
(a) intranasally administering to the subject a therapeutically effective amount of racemic ketamine, or a pharmaceutically acceptable salt thereof;
wherein no clinically meaningful dissociation is observed in the subject within about 24 hours after intranasal administration of the racemic ketamine, or a pharmaceutically acceptable salt thereof. In some embodiments, no clinically meaningful dissociation is observed in the subject at about 4 hours after intranasal administration of the racemic ketamine, or a pharmaceutically acceptable salt thereof. In some embodiments, no clinically meaningful dissociation is observed in the subject at about 1 hour after intranasal administration of the racemic ketamine, or a pharmaceutically acceptable salt thereof, and
(a) Determining if the subject has an improved Clinician Administered PTSD Scale DSM-5 (CAPS-5) score at 12 weeks.
(b) Determine if the subject has one or more of the following scores:
(1) Number and % of CAPS-5 responders (total score reduction of 12 points from baseline) at each efficacy timepoint;
(2) Number and % of persistent CAPS-5 responders from onset at each timepoint and maintained through Week 12;
(3) Number and % of persistent CAPS-5 responders from onset at each timepoint and maintained through Week 16;
(4) CGIS at week 12;
(5) PGIS at week 12;
(6) Clinical Global Impression Change (CGIC) at week 16;
(7) Patient Global Impression Change (PGIC) at week 16;
(8) Sheehan Disability Scale (SDS) at week 16; and
(9) Posttreatment Phase—time to relapse for responders in the treatment phase, who subsequently relapse during the 4-week safety follow-up. (Relapse is defined as a CAPS-5 increase of ≥12 points for two consecutive weeks).

In some embodiments, three of more of (1)-(9) are improved relative to baseline (prior to administration of intranasal racemic ketamine, or a pharmaceutically acceptable salt thereof). In some embodiments, four or more of (1)-(9) are improved relative to baseline (prior to administration of intranasal racemic ketamine, or a pharmaceutically acceptable salt thereof). In some embodiments, five or more of (1)-(9) are improved relative to baseline (prior to administration of intranasal racemic ketamine, or a pharmaceutically acceptable salt thereof). In some embodiments, six or more (1)-(9) are improved relative to baseline (prior to administration of intranasal racemic ketamine, or a pharmaceutically acceptable salt thereof). In some embodiments, seven or more (1)-(9) are improved relative to baseline (prior to administration of intranasal racemic ketamine, or a pharmaceutically acceptable salt thereof). In some embodiments, eight of (1)-(9) are improved relative to baseline (prior to administration of intranasal racemic ketamine, or a pharmaceutically acceptable salt thereof). In some embodiments, each of (1)-(9) are improved relative to baseline (prior to administration of intranasal racemic ketamine, or a pharmaceutically acceptable salt thereof).

Some embodiments provide a method of treating PTSD in a subject in need thereof, comprising intranasally administering to the subject a therapeutically effective amount of racemic ketamine, or a pharmaceutically acceptable salt thereof;
wherein no clinically meaningful sedation and no clinically meaningful dissociation is observed in the subject within about 24 hours after intranasal administration of the racemic ketamine, or a pharmaceutically acceptable salt thereof. In some embodiments, no clinically meaningful sedation and no clinically meaningful dissociation is observed in the subject at about 4 hours after intranasal administration of the racemic ketamine, or a pharmaceutically acceptable salt thereof. In some embodiments, no clinically meaningful sedation and no clinically meaningful dissociation is observed in the subject at about 1 hour after intranasal administration of the racemic ketamine, or a pharmaceutically acceptable salt thereof.

Some embodiments provide a method of treating Adults with Bipolar I or II Disorder with a Moderate or Severe Major Depressive Episode (Bipolar Depression) in a subject in need thereof, comprising:
(a) Determining if the subject has a MADRS total score of >28 predose on Day 1
(b) intranasally administering to the subject a therapeutically effective amount of racemic ketamine, or a pharmaceutically acceptable salt thereof;
wherein no clinically meaningful sedation is observed in the subject within about 24 hours after intranasal administration of the racemic ketamine, or a pharmaceutically acceptable salt thereof. In some embodiments, no clinically meaningful sedation is observed in the subject at about 4 hours after intranasal administration of the racemic ketamine, or a pharmaceutically acceptable salt thereof. In some embodiments, no clinically meaningful sedation is observed in the subject at about 1 hour after intranasal administration of the racemic ketamine, or a pharmaceutically acceptable salt thereof.
(c) Determining if the MADRS total score at 24 h after initial dose is at least (X)
(d) Determining if the subject shows a change from baseline in the following:
(1) MADRS total score at Day 16;
(2) Clinical Global Impression Severity (CGIS) at 24 hours;
(3) Patient Global Impression Severity (PGIS) at 24 hours;
(4) CGIS at Day 16; or
(5) PGIS at Day 16.

In some embodiments, two of more of (1)-(5) are improved relative to baseline (prior to administration of intranasal racemic ketamine, or a pharmaceutically acceptable salt thereof). In some embodiments, three of more of (1)-(5) are improved relative to baseline (prior to administration of intranasal racemic ketamine, or a pharmaceutically acceptable salt thereof). In some embodiments, four of (1)-(5) are improved relative to baseline (prior to administration of intranasal racemic ketamine, or a pharmaceutically acceptable salt thereof). In some embodiments, each of (1)-(5) are improved relative to baseline (prior to administration of intranasal racemic ketamine, or a pharmaceutically acceptable salt thereof).

With additional Secondary Endpoints:

The additional secondary endpoints are the change from baseline at 24 hours, as well as at Day 16, and end of week 6 in the following:
(1) MADRS response (total score ≥50% reduction from baseline),
(2) Onset of clinical response (MADRS total score >50% reduction from baseline by 24 hrs and maintained to Day 16),
(3) MADRS remission (total score ≤12),
(4) Improved MADRS Total Score at end of week 6;
(5) Improved Clinical Global Impression Change (CGIC) at end of week 6;
(6) Improved Patient Global Impression Change (PGIC) at end of week 6;
(7) Improved Sheehan Disability Scale (not assessed at 24 hr);
(8) Improved Young Mania Rating Scale;
(9) Posttreatment Phase—time to relapse for patients who were in remission in the treatment phase, who subsequently relapse during the 4 week safety follow-up, and
(10) Posttreatment Phase—time to relapse for patients who were responders in the treatment phase, who subsequently relapse during the 4 week safety follow-up (Relapse is defined as MADRS total score ≥22 for two consecutive weeks).

In some embodiments, one or more of the MADRS Total, MADRS Item 10, CGIS-SI/B, STS-CMCM Risk of Suicide at Within the Next 7 Days, and C-SSRS scores is reduced by at least 50% about 24 hours after intranasal administration of racemic ketamine, or a pharmaceutically acceptable salt thereof.

In some embodiments, one or more of the MADRS Total, MADRS Item 10, CGIS-SI/B, STS-CMCM Risk of Suicide at Within the Next 7 Days, and C-SSRS scores is reduced by at least 50% about 48 hours after intranasal administration of racemic ketamine, or a pharmaceutically acceptable salt thereof.

In some embodiments, one or more of the MADRS Total, MADRS Item 10, CGIS-SI/B, STS-CMCM Risk of Suicide at Within the Next 7 Days, and C-SSRS scores is reduced by at least 50% about 96 hours after intranasal administration of racemic ketamine, or a pharmaceutically acceptable salt thereof.

In some embodiments, one or more of the MADRS Total, MADRS Item 10, CGIS-SI/B, STS-CMCM Risk of Suicide at Within the Next 7 Days, and C-SSRS scores is below the standard for remission about 24 hours after intranasal administration of racemic ketamine, or a pharmaceutically acceptable salt thereof.

In some embodiments, one or more of the MADRS Total, MADRS Item 10, CGIS-SI/B, STS-CMCM Risk of Suicide at Within the Next 7 Days, and C-SSRS scores is below the standard for remission about 48 hours after intranasal administration of racemic ketamine, or a pharmaceutically acceptable salt thereof.

In some embodiments, one or more of the MADRS Total, MADRS Item 10, CGIS-SI/B, STS-CMCM Risk of Suicide at Within the Next 7 Days, and C-SSRS scores is below the standard for remission about 96 hours after intranasal administration of racemic ketamine, or a pharmaceutically acceptable salt thereof.

In some embodiments, the subject has a MADRS Total Score of at least 20 units. In some embodiments, the subject has a MADRS Item 10 Score of 4, 5, or 6 units. In some embodiments, the subject has a CGIS-SI/B score of 4 or 5 units. In some embodiments, the subject has a STS-CMCM score of at least 15 units. In some embodiments, the subject has a STS-CMCM Risk of Suicide at Within the Next 7 Days score of at least 5 units. In some embodiments, the subject has a C-SSRS score of at least 2. In some embodiments, the subject has a MADRS Total Score of at least 20 units and a CGIS-SI/B score of 4 or 5 units. In some embodiments, the subject has a MADRS Total Score of at least 20 units and a STS-CMCM score of at least 15 units. In some embodiments, the subject has a MADRS Total Score of at least 20 units; a CGIS-SI/B score of 4 or 5 units; and a STS-CMCM score of at least 15 units.

In some embodiments, the subject has a MADRS Total Score of at least 20 units. In some embodiments, the subject has a MADRS Item 10 Score of 1-3 units. In some embodiments, the subject has a CGIS-SI/B score of 2 or 3 units. In some embodiments, the subject has a STS-CMCM score of at least 10 units. In some embodiments, the subject has a STS-CMCM Risk of Suicide at Within the Next 7 Days score of at least 3 units. In some embodiments, the subject has a C-SSRS score of 0-2. In some embodiments, the subject has a MADRS Total Score of at least 20 units and a CGIS-SI/B score of 2 or 3 units. In some embodiments, the subject has a MADRS Total Score of at least 20 units and a STS-CMCM score of at least 10 units. In some embodiments, the subject has a MADRS Total Score of at least 20 units; a CGIS-SI/B score of 2 or 3 units; and a STS-CMCM score of at least 10 units.

In some embodiments, the subject has a MADRS Total Score of at least 20 units. In some embodiments, the subject has a MADRS Item 10 Score of 3 or 4 units. In some embodiments, the subject has a CGIS-SI/B score of 2 or 3 units. In some embodiments, the subject has a STS-CMCM score of at least 10 units. In some embodiments, the subject has a STS-CMCM Risk of Suicide at Within the Next 7 Days score of at least 3 units. In some embodiments, the subject has a C-SSRS score of 0-2. In some embodiments, the subject has a MADRS Total Score of at least 20 units and a CGIS-SI/B score of 2 or 3 units. In some embodiments, the subject has a MADRS Total Score of at least 20 units and a STS-CMCM score of at least 10 units. In some embodiments, the subject has a MADRS Total Score of at least 20 units; a CGIS-SI/B score of 2 or 3 units; and a STS-CMCM score of at least 10 units.

In some embodiments, the racemic ketamine, or a pharmaceutically acceptable salt thereof, is intranasally administered from about once per day to about once per month, for example, once per day, once every other day, twice per week, or once per week. In some embodiments, the racemic ketamine, or a pharmaceutically acceptable salt thereof, is intranasally administered from about once per day to about once every two weeks. In some embodiments, the racemic ketamine, or a pharmaceutically acceptable salt thereof, is intranasally administered from about once per day to about once per week. In some embodiments, the racemic ketamine, or a pharmaceutically acceptable salt thereof, is intranasally administered from about once per week to about twice per week. In some embodiments, the racemic ketamine, or a pharmaceutically acceptable salt thereof, is intranasally administered twice per week. In some embodiments, the racemic ketamine, or a pharmaceutically acceptable salt thereof, is intranasally administered once per day, once every other day, three times per week, twice per week, or once per week. In some embodiments, the racemic ketamine, or a pharmaceutically acceptable salt thereof, is intranasally administered every four days (e.g., on day 1, day 4, day 8, day 12, day 16, etc.).

Some embodiments described herein provide a comparison between intranasal administration of racemic ketamine, or a pharmaceutically acceptable salt thereof, intravenous administration of racemic ketamine, or a pharmaceutically acceptable salt thereof, and/or administration (e.g., intravenous or intranasal administration) of (S)-ketamine, or a pharmaceutically acceptable salt thereof.

In some embodiments, the intranasal (S)-ketamine is Spravato®. In some embodiments, the intranasal (S)-ketamine is a solution consisting essentially of 32.3 mg of (S)-ketamine hydrochloride (equivalent to 28 mg of (S)-ketamine), citric acid monohydrate, edetate disodium, sodium hydroxide, and water. In some embodiments, the intranasal (S)-ketamine is a clear, colorless aqueous solution with a pH of 4.5. See the Spravato® ((S)-ketamine) Package Insert dated Feb. 11, 2020; www.accessdata.fda.gov/drugsatfda_docs/label/2020/211243s003lbl.pdf, which is hereby incorporated by reference in its entirety.

Some embodiments reference a time “prior to” administration of the intranasal racemic ketamine, or a pharmaceutically acceptable salt thereof. The time prior to administration may be a particular time or range of time as indicated (e.g., about 30 minutes, about 1 hour, from about 1 day to about 1 week, 6 months, etc.), or it may be any time prior to administration if no particular time or range is specified.

### Combination Therapy

The methods of the present disclosure also contemplate treatments comprising administering ketamine, or a pharmaceutically acceptable salt thereof, as described in any of the embodiments of the disclosure, in combination with one or more additional therapies (such as an antidepressant). Accordingly, ketamine, or a pharmaceutically acceptable salt thereof, as described anywhere herein can be administered alone or in combination with one or more additional therapies. When administered in combination with one or more additional therapies, separate dosage forms can be administered to the subject. If administered as a separate dosage form, the one or more additional therapies may be administered simultaneously with the intranasal ketamine dosage form of the present disclosure or sequentially with the ketamine dosage form of the present disclosure, in either order. In some embodiments, the intranasal ketamine dosage form and the one or more additional therapies are administered sequentially on the same or different days. For example, the racemic ketamine, or a pharmaceutically acceptable salt thereof, is intranasally administered twice per week and the one or more additional therapies are administered once per day.

In some embodiments, the methods described herein further comprise administering one or more additional therapies consist of typical antipsychotics, atypical antipsychotics, antidepressants, electroconvulsive therapy, transcranial magnetic stimulation, benzodiazepines, mood stabilizers, and pramipexole.

In some embodiments, the methods described herein further comprise providing cognitive behavior therapy to the subject.

In some embodiments, the one or more additional therapies is a standard of care treatment for MDD. In some embodiments, the one or more additional therapies is a standard of care treatment for TRD. In some embodiments, the one or more additional therapies is a standard of care treatment for PTSD. In some embodiments, the one or more additional therapies is a standard of care treatment for another psychiatric disorder as described herein.

In some embodiments, the subject has been administered a stable dose of the one or more additional therapies for four weeks or longer prior to initiation of treatment with ketamine, or a pharmaceutically acceptable salt thereof.

In some embodiments, the one or more additional therapies is pramipexole.

In some embodiments, the one or more additional therapies is a typical antipsychotic. Representative typical antipsychotics include, but are not limited to chlorpromazine, chlorprothixene, levomepromazine, mesoridazine, periciazine, promazine, loxapine, molindone, perphenazine, thiothixene, droperidol, flupentixol, fluphenazine, haloperidol, pimozide, prochlorperazine, thioproperazine, trifluoperazine, and zuclopenthixol.

In some embodiments, the one or more additional therapies is an atypical antipsychotic. Representative atypical antipsychotics include, but are not limited to aripiprazole, risperidone, olanzapine, quetiapine, asenapine, paliperidone, ziprasidone, or lurasidone.

In some embodiments, the one or more additional therapies is a short acting non-benzodiazepine hypnotics (e.g., zolpidem, zaleplon). In some embodiments, the short acting non-benzodiazepine hypnotics is not administered within about 10 hours prior to, or 10 hours after, administration of the intranasal racemic ketamine.

In some embodiments, the one or more additional therapies is an antidepressant. In some embodiments, the antidepressant is an atypical antidepressant, a selective serotonin reuptake inhibitor, a selective serotonin and norepinephrine reuptake inhibitor, a monoamine oxidase inhibitor, or a selective norepinephrine reuptake inhibitor.

In some embodiments, the antidepressant is an atypical antidepressant. Representative atypical antidepressants include, but are not limited to mirtazapine, mianserin, bupropion, trazodone, nefazodone, tianeptine, opipramol, agomelatine, vilazodone, and vortioxetine.

In some embodiments, the antidepressant is a selective serotonin reuptake inhibitor. Representative selective serotonin reuptake inhibitors include, but are not limited to citalopram, escitalopram, fluoxetine, fluvoxamine, paroxetine, and sertraline.

In some embodiments, the antidepressant is a selective serotonin and norepinephrine reuptake inhibitor. Representative selective serotonin and norepinephrine reuptake inhibitors include, but are not limited to atomoxetine, desvenlafaxine, duloxetine, levomilnacipran, milnacipran, sibutramine, tramadol, and venlafaxine.

In some embodiments, the antidepressant is a monoamine oxidase inhibitor. Representative monoamine oxidase inhibitors include, but are not limited to moclobemide, rasagiline, selegiline, or safinamide.

In some embodiments, the antidepressant is a selective norepinephrine reuptake inhibitor. Representative selective norepinephrine reuptake inhibitors include, but are not limited to reboxetine.

In some embodiments, the one or more additional therapies is a benzodiazepine. Representative benzodiazepines include, but are not limited to alprazolam, bromazepam, chlordiazepoxide, clonazepam, clorazepate, diazepam, flurazepam, lorazepam, oxazepam, temazepam, or triazolam.

In some embodiments, the one or more additional therapies is selective serotonin reuptake inhibitors (SSRIs) or serotonin and norepinephrine reuptake inhibitors (SNRIs), provided the patient has been taking the medication for at least 8 weeks at a therapeutic dose, as defined by the medication label, and the dose has not been changed within 4 weeks prior to screening.

In some embodiments, the one or more additional therapies is a mood stabilizer. Representative mood stabilizers include, but are not limited to lithium, valproic acid, lamotrigine, or carbamazepine. In some embodiments, the one or more additional therapies is electroconvulsive therapy or transcranial magnetic stimulation.

In some embodiments, the one or more additional therapies is sertraline. In some embodiments, the one or more additional therapies is venlafaxine.

In some embodiments, the one or more additional therapies is one additional therapy. In some embodiments, the one or more additional therapies is two, three, or four additional therapies.

In some embodiments, the subject has previously been administered one or more additional therapies consist of typical antipsychotics, atypical antipsychotics, antidepressants, electroconvulsive therapy, transcranial magnetic stimulation, benzodiazepines, mood stabilizers, and pramipexole; wherein the subject was not responsive to the previous one or more therapies.

In some embodiments, the subject has previously been administered a standard of care treatment for major depressive disorder and the subject was not responsive to the previous therapy. In some embodiments, the subject has previously been administered a standard of care treatment for MDD and the subject was not responsive to the previous therapy. In some embodiments, the subject has previously been administered a standard of care treatment for TRD and the subject was not responsive to the previous therapy. In some embodiments, the subject has previously been administered a standard of care treatment for PTSD and the subject was not responsive to the previous therapy. In some embodiments, the subject has previously been administered a standard of care treatment for another psychiatric disorder as described herein, and the subject was not responsive to the previous therapy.

In some embodiments, the subject has previously been administered one or more additional therapies consist of typical antipsychotics, atypical antipsychotics, antidepressants, electroconvulsive therapy, transcranial magnetic stimulation, benzodiazepines, mood stabilizers, and pramipexole, and was not responsive to the previous therapy.

In some embodiments, the subject has previously been administered pramipexole and was not responsive to the previous therapy.

In some embodiments, the subject has previously been administered one or more typical antipsychotics such as chlorpromazine, chlorprothixene, levomepromazine, mesoridazine, periciazine, promazine, loxapine, molindone, perphenazine, thiothixene, droperidol, flupentixol, fluphenazine, haloperidol, pimozide, prochlorperazine, thioproperazine, trifluoperazine, and zuclopenthixol, and was not responsive to the previous therapy.

In some embodiments, the subject has previously been administered one or more atypical antipsychotics, such as aripiprazole, risperidone, olanzapine, quetiapine, asenapine, paliperidone, ziprasidone, or lurasidone, and was not responsive to the previous therapy.

In some embodiments, the subject has previously been administered one or more antidepressants and was not responsive to the previous therapy. In some embodiments, the antidepressant is an atypical antidepressant, a selective serotonin reuptake inhibitor, a selective serotonin and norepinephrine reuptake inhibitor, a monoamine oxidase inhibitor, or a selective norepinephrine reuptake inhibitor, and was not responsive to the previous therapy.

In some embodiments, the subject has previously been administered one or more atypical antidepressants, such as mirtazapine, mianserin, bupropion, trazodone, nefazodone, tianeptine, opipramol, agomelatine, vilazodone, and vortioxetine, and was not responsive to the previous therapy.

In some embodiments, the subject has previously been administered one or more selective serotonin reuptake inhibitors, such as citalopram, escitalopram, fluoxetine, fluvoxamine, paroxetine, and sertraline, and was not responsive to the previous therapy.

In some embodiments, the subject has previously been administered one or more selective serotonin and norepinephrine reuptake inhibitors, such as atomoxetine, desvenlafaxine, duloxetine, levomilnacipran, milnacipran, sibutramine, tramadol, and venlafaxine, and was not responsive to the previous therapy.

In some embodiments, the subject has previously been administered one or more monoamine oxidase inhibitors, such as moclobemide, rasagiline, selegiline, or safinamide, and was not responsive to the previous therapy.

In some embodiments, the subject has previously been administered one or more selective norepinephrine reuptake inhibitors, such as reboxetine, and was not responsive to the previous therapy.

In some embodiments, the subject has previously been administered one or more benzodiazepines, such as alprazolam, bromazepam, chlordiazepoxide, clonazepam, clorazepate, diazepam, flurazepam, lorazepam, oxazepam, temazepam, or triazolam, and was not responsive to the previous therapy.

In some embodiments, the subject has previously been administered one or more mood stabilizers, such as lithium, valproic acid, lamotrigine, or carbamazepine, and was not responsive to the previous therapy.

In some embodiments, the one or more additional therapies is electroconvulsive therapy or transcranial magnetic stimulation, and was not responsive to the previous therapy.

In some embodiments, the subject has previously been administered sertraline, and was not responsive to the previous therapy. In some embodiments, the subject has previously been administered venlafaxine, and was not responsive to the previous therapy.

In some embodiments, the one or more additional therapies previously administered to the subject is one additional therapy. In some embodiments, the one or more additional therapies previously administered to the subject is two additional therapies. In some embodiments, the one or more additional therapies previously administered to the subject is three additional therapies. In some embodiments, the one or more additional therapies previously administered to the subject is four additional therapies. In some embodiments, the one or more additional therapies previously administered to the subject is five, six, seven, eight, nine, or ten additional therapies.

In some embodiments described herein, when the subject is being administered one or more additional therapies, the subject does not experience a clinically significant weight gain relative to the administration of the one or more additional therapies in the absence of intranasal administration of racemic ketamine, or a pharmaceutically acceptable salt thereof. Clinically significant weight gain refers to an increase in body mass of at least about 5% over the course of treatment, for example, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 30%, about 35%, about 40%, about 45%, about 50%, about 55%, about 60%, about 65%, about 70%, about 75%, about 80%, about 85%, about 90%, about 95%, about 100%, or any value in between.

In some embodiments, the subject has not been administered an anxiolytic prior to the first administration of intranasal racemic ketamine, or a pharmaceutically acceptable salt thereof.

In some embodiments, the subject has not been administered an anxiolytic within about 24 hours prior to the first administration of intranasal racemic ketamine, or a pharmaceutically acceptable salt thereof. For example, within about 24 hours, about 18 hours, about 12 hours, about 6 hours, about 4 hours, about 2 hours, or about 1 hour prior to the first administration of intranasal racemic ketamine, or a pharmaceutically acceptable salt thereof.

In some embodiments, the subject has not been administered a benzodiazepine prior to the first administration of intranasal racemic ketamine, or a pharmaceutically acceptable salt thereof.

In some embodiments, the subject has not been administered a benzodiazepine within about 24 hours prior to the first administration of intranasal racemic ketamine, or a pharmaceutically acceptable salt thereof. For example, within about 24 hours, about 18 hours, about 12 hours, about 6 hours, about 4 hours, about 2 hours, or about 1 hour prior to the first administration of intranasal racemic ketamine, or a pharmaceutically acceptable salt thereof.

In some embodiments, the subject does not take an anxiolytic within about 14 days after the final administration of intranasal racemic ketamine, or a pharmaceutically acceptable salt thereof. In some embodiments, the subject does not take a benzodiazepine within about 14 days after the final administration of intranasal racemic ketamine, or a pharmaceutically acceptable salt thereof. For example, about 14 days, about 13 days, about 12 days, about 11 days, about 10 days, about 9 days, about 8 days, about 7 days, about 6 days, about 5 days, about 4 days, about 3 days, about 2 days, or about 1 day after the final administration of intranasal racemic ketamine, or a pharmaceutically acceptable salt thereof.

In some embodiments, the benzodiazepine selected from the group consisting of alprazolam, bromazepam, chlordiazepoxide, clobazam, clonazepam, clorazepate, diazepam, estazolam, flurazepam, halazepam, lorazepam, midazolam, nitrazepam, oxazepam, quazepam, temazepam, and triazolam.

In some embodiments, the benzodiazepine is alprazolam, diazepam, or lorazepam.

In some embodiments, the subject is currently taking an anxiolytic. In some embodiments the subject is currently taking a benzodiazepine. In some embodiments, the subject is currently taking a benzodiazepine at less than or equal to a 2 mg dose of lorazepam per day. In some embodiments, the subject is currently taking less than or equal to 2 mg of lorazepam per day.

In some embodiments, the subject has not been administered a monoamine oxidase inhibitor prior to the first administration of intranasal racemic ketamine, or a pharmaceutically acceptable salt thereof.

In some embodiments, the subject has not been administered a monoamine oxidase inhibitor within about 24 hours prior to the first administration of intranasal racemic ketamine, or a pharmaceutically acceptable salt thereof. For example, within about 24 hours, about 18 hours, about 12 hours, about 6 hours, about 4 hours, about 2 hours, or about 1 hour prior to the first administration of intranasal racemic ketamine, or a pharmaceutically acceptable salt thereof.

In some embodiments, the subject does not take monoamine oxidase inhibitor within about 14 days after the final administration of intranasal racemic ketamine, or a pharmaceutically acceptable salt thereof. In some embodiments, the subject does not take a benzodiazepine within about 14 days after the final administration of intranasal racemic ketamine, or a pharmaceutically acceptable salt thereof. For example, about 14 days, about 13 days, about 12 days, about 11 days, about 10 days, about 9 days, about 8 days, about 7 days, about 6 days, about 5 days, about 4 days, about 3 days, about 2 days, or about 1 day after the final administration of intranasal racemic ketamine, or a pharmaceutically acceptable salt thereof.

Representative monoamine oxidase inhibitors include, but are not limited to moclobemide, rasagiline, selegiline, or safinamide.

In some embodiments, the subject has not been administered opioids or drugs with activity at the opioid receptor prior to the first administration of intranasal racemic ketamine, or a pharmaceutically acceptable salt thereof.

In some embodiments, the subject has not been administered opioids or drugs with activity at the opioid receptor within about 24 hours prior to the first administration of intranasal racemic ketamine, or a pharmaceutically acceptable salt thereof. For example, within about 24 hours, about 18 hours, about 12 hours, about 6 hours, about 4 hours, about 2 hours, or about 1 hour prior to the first administration of intranasal racemic ketamine, or a pharmaceutically acceptable salt thereof.

In some embodiments, the subject has not been administered lamotrigine prior to the first administration of intranasal racemic ketamine, or a pharmaceutically acceptable salt thereof.

In some embodiments, the subject has not been administered lamotrigine within about 24 hours prior to the first administration of intranasal racemic ketamine, or a pharmaceutically acceptable salt thereof. For example, within about 24 hours, about 18 hours, about 12 hours, about 6 hours, about 4 hours, about 2 hours, or about 1 hour prior to the first administration of intranasal racemic ketamine, or a pharmaceutically acceptable salt thereof.

In some embodiments, the subject has not been administered N-methyl-D-aspartate (NMDA) receptor modulators prior to the first administration of intranasal racemic ketamine, or a pharmaceutically acceptable salt thereof.

In some embodiments, the subject has not been administered N-methyl-D-aspartate (NMDA) receptor modulators within about 24 hours prior to the first administration of intranasal racemic ketamine, or a pharmaceutically acceptable salt thereof. For example, within about 24 hours, about 18 hours, about 12 hours, about 6 hours, about 4 hours, about 2 hours, or about 1 hour prior to the first administration of intranasal racemic ketamine, or a pharmaceutically acceptable salt thereof.

In some embodiments, the subject has not been administered nefazodone or fluvoxamine prior to the first administration of intranasal racemic ketamine, or a pharmaceutically acceptable salt thereof. In some embodiments, the subject has not been administered nefazodone or fluvoxamine within about 7-10 days prior to the first administration of intranasal racemic ketamine, or a pharmaceutically acceptable salt thereof. For example, within about 10 days, about 9 days, about 8 days, or about 7 days prior to the first administration of intranasal racemic ketamine, or a pharmaceutically acceptable salt thereof.

In some embodiments, the subject has not been administered St. John's wort prior to the first administration of intranasal racemic ketamine, or a pharmaceutically acceptable salt thereof.

In some embodiments, the subject has not been administered St. John's wort within about 30-45 days prior to the first administration of intranasal racemic ketamine, or a pharmaceutically acceptable salt thereof. For example, within about 30 days, about 35 days, about 40 days, or about 45 days prior to the first administration of intranasal racemic ketamine, or a pharmaceutically acceptable salt thereof.

In some embodiments, the subject has not been administered a lithium or calcium channel blocker prior to the first administration of intranasal racemic ketamine, or a pharmaceutically acceptable salt thereof.

In some embodiments, the subject has not been administered a lithium or calcium channel blocker within about 90-120 days prior to the first administration of intranasal racemic ketamine, or a pharmaceutically acceptable salt thereof. For example, within about 90 days, about 100 days, about 110 days, or about 120 days prior to the first administration of intranasal racemic ketamine, or a pharmaceutically acceptable salt thereof.

In some embodiments, the racemic ketamine, or a pharmaceutically acceptable salt thereof, is intranasally administered acutely. For example, in a subject with PTSD, the racemic ketamine, or a pharmaceutically acceptable salt thereof, can be administered from one to four weeks, or until the PTSD resolves. In some embodiments, the racemic ketamine, or a pharmaceutically acceptable salt thereof, is intranasally administered chronically. For example, in a subject suffering from MDD, the racemic ketamine, or a pharmaceutically acceptable salt thereof, can be administered for several months to several years, or until the depression resolves.

In some embodiments described herein, the (S)-ketamine is administered intravenously. In some embodiments described herein, the (S)-ketamine is administered intranasally.

Some embodiments provide an intranasally administering composition, comprising a therapeutically effective amount of racemic ketamine, or a pharmaceutically acceptable salt thereof, and a pharmaceutically acceptable carrier.

Some embodiments provide the use of racemic ketamine, or a pharmaceutically acceptable salt thereof, in the preparation of an intranasally administering medicament for treating MDD in a subject in need thereof.

Some embodiments provide the use of racemic ketamine, or a pharmaceutically acceptable salt thereof, in the preparation of an intranasally administering medicament for treating TRD in a subject in need thereof.

Some embodiments provide the use of racemic ketamine, or a pharmaceutically acceptable salt thereof, in the preparation of an intranasally administering medicament for treating PTSD in a subject in need thereof.

Some embodiments provide the use of racemic ketamine, or a pharmaceutically acceptable salt thereof, in the preparation of an intranasally administering medicament for reducing one or more side effects of ketamine in a subject in need thereof.

In some embodiments, the methods described herein provide one or more synergistic effects when the racemic ketamine, or a pharmaceutically acceptable salt thereof, is administered in combination with one or more additional therapies.

In some embodiments, the efficacy of the intranasal racemic ketamine, or a pharmaceutically acceptable salt thereof, and the one or more additional therapies, is greater than the sum of the efficacy of each individual agent when administered alone. For example, in some embodiments, the change in the C-SSRS score after administration of the intranasal racemic ketamine, or a pharmaceutically acceptable salt thereof, compared to prior to administration of the intranasal racemic ketamine, or a pharmaceutically acceptable salt thereof, is greater than the change in the C-SSRS score after administration of the intranasal racemic ketamine, or a pharmaceutically acceptable salt thereof, and the one or more additional therapies compared to prior to administration of the intranasal racemic ketamine, or a pharmaceutically acceptable salt thereof, and the one or more additional therapies. In some embodiments, the efficacy of the ketamine, or a pharmaceutically acceptable salt thereof, is increased. In some embodiments, the efficacy of the one or more additional therapies is increased. In some embodiments, the efficacy of both the intranasal racemic ketamine, or a pharmaceutically acceptable salt thereof, and the one or more additional therapies are increased. In some embodiments, the efficacy of the one or more additional therapies administered with the intranasal racemic ketamine, or a pharmaceutically acceptable salt thereof, is increased relative to the efficacy observed following administration with an equivalent dose of intravenous racemic ketamine, or a pharmaceutically acceptable salt thereof. In some embodiments, the efficacy of the one or more additional therapies administered with the intranasal racemic ketamine, or a pharmaceutically acceptable salt thereof, is increased relative to the efficacy observed following administration with an equivalent dose of (S)-ketamine, or a pharmaceutically acceptable salt thereof.

In some embodiments, the dose of the intranasal racemic ketamine, or a pharmaceutically acceptable salt thereof, and/or the one or more additional therapies, required to provide a therapeutic effect is reduced relative to the dose of each individual agent when administered alone. In some embodiments, the dose of the one or more additional therapies required to provide a therapeutic effect is reduced relative to the dose administering with an equivalent dose of intravenous racemic ketamine, or a pharmaceutically acceptable salt thereof. In some embodiments, the dose of the one or more additional therapies required to provide a therapeutic effect is reduced relative to the dose administering with an equivalent dose of (S)-ketamine, or a pharmaceutically acceptable salt thereof. In some embodiments, the dose of the ketamine, or a pharmaceutically acceptable salt thereof, is reduced. In some embodiments, the dose of the one or more additional therapies is reduced. In some embodiments, the dose of both the intranasal racemic ketamine, or a pharmaceutically acceptable salt thereof, and the one or more additional therapies are reduced. For example, the dose of intranasal racemic ketamine, or a pharmaceutically acceptable salt thereof, can be reduced by about 5% to about 95%, or any value in between, such as about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%. Similarly, the dose of the one or more additional therapies can be reduced by about 5% to about 95%, or any value in between, such as about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%.

In some embodiments, the onset of resistance to treatment with the intranasal racemic ketamine, or a pharmaceutically acceptable salt thereof, and the one or more additional therapies, is delayed relative to the onset of resistance to treatment with each individual agent when administered alone. In some embodiments, the onset of resistance to treatment with the one or more additional therapies is delayed relative to the onset of resistance to treatment when administered with intravenous racemic ketamine, or a pharmaceutically acceptable salt thereof. In some embodiments, the onset of resistance to treatment with the one or more additional therapies is delayed relative to the onset of resistance to treatment when administered with (S)-ketamine, or a pharmaceutically acceptable salt thereof.

In some embodiments, the subject is not currently being administered a benzodiazepine. In some embodiments, the subject is being administered a benzodiazepine in an amount of 2 mg or less of lorazepam or dose equivalent per day.

In some embodiments, the subject has not previously been administered electroconvulsive therapy. In some embodiments, the subject has not previously been administered transcranial magnetic stimulation. In some embodiments, the subject is not currently being administered electroconvulsive therapy (i.e., during the same time when the subject is taking intranasal racemic ketamine, or a pharmaceutically acceptable salt thereof). In some embodiments, the subject is not currently being administered transcranial magnetic stimulation(i.e., during the same time when the subject is taking intranasal racemic ketamine, or a pharmaceutically acceptable salt thereof).

### Intranasal Delivery

In some embodiments, the racemic ketamine, or a pharmaceutically acceptable salt thereof, is intranasally administered. The administration can be accomplished via a suitable intranasal delivery device.

In some embodiments, a device (e.g., an intranasal device) can administer one or more doses of racemic ketamine to a nasal cavity of a subject. In some embodiments, the device is designed for a nostril of a subject. In some embodiments, the device is designed to measure a particular amount or a particular dose of racemic ketamine. In some embodiments, the device is designed to be actuated for operation by the breath of the subject. In some embodiments, the device is designed to deliver more than one dose to the nasal cavity of the subject. In some embodiments, the device can spray racemic ketamine into the nostril cavity of the subject.

In some embodiments, the device comprises a nozzle for providing an aerosol through a nosepiece. The nozzle comprises a head which is located, in some embodiments coaxially within the nosepiece, and a delivery tube, which is fluidly connected to the head. In some embodiments, the nozzle can be configured to provide a jet of a substance through the nosepiece. In some embodiments, the device further comprises a substance supply unit for delivering a metered dose of racemic ketamine to the nozzle. In some embodiments, the substance supply unit comprises a mechanical delivery pump, which is fluidly connected to the nozzle and which is configured, on actuation thereof, to deliver a metered dose of racemic ketamine to the nozzle, which nozzle generates an aerosol. The delivery pump is movable relative to the nozzle from a first, non-actuated position to a second, actuated position to deliver a metered dose of racemic ketamine to the nozzle, and hence generate an aerosol.

In some embodiments, the mechanical delivery pump comprises a liquid delivery pump for delivering a metered volume of a liquid comprising racemic ketamine, for example, as a suspension or a solution, to the nozzle on actuation thereof.

In some embodiments, the substance supply unit further comprises a biasing element, in this embodiment a resilient element, particularly a compression spring, for biasing the delivery pump in an actuating direction when in the non-actuated position, and a loading mechanism, and in some embodiments, comprising first and second levers, for loading the biasing element such as to bias the delivery pump, when in the non-actuated position, with an actuation force. In some embodiments, the loading mechanism is movable between a first, rest position in which the biasing element is not loaded thereby, and a second, operative position in which the biasing element, when restrained by the delivery pump, loads the delivery pump with the actuation force.

In some embodiments, the device further comprises a trigger mechanism, which is configured to be actuatable to cause the actuation of the substance supply unit. In some embodiments, the trigger mechanism is configured to be actuatable to cause the actuation of the substance supply unit on the generation of a predetermined pressure in the chamber in the housing. In some embodiments, the trigger mechanism could be configured to be actuatable to cause the actuation of the substance supply unit on the generation of a predetermined flow rate through a mouthpiece.

In some embodiments, the trigger mechanism comprises first and second stop members, and first and second biasing elements, which comprise resilient elements, such as compression springs, which act to bias respective ones of the first and second stop members inwardly to a stop position in which the first and second stop members act to prevent movement of the delivery pump from the non-actuated position to the actuated position.

In some embodiments, the trigger mechanism further comprises first and second arms which are pivotable about respective pivots and coupled at one end thereof to respective ones of the first and second stop members such that pivoting of the arms to a release position causes the respective ones of the stop members, to which the arms are coupled, to be moved outwardly against the bias of the first and second biasing elements to a release position in which the stop members are disposed outwardly of the head of the delivery pump, such that the delivery pump, when biased by the biasing element, is driven to the actuated position. In being driven to the actuated position, a metered dose of racemic ketamine is delivered from the delivery pump to the nozzle, with the nozzle acting to generate an aerosol.

In some embodiments, the trigger mechanism further comprises a diaphragm as the resilient member, which defines a part of the wall of the chamber in the housing. The diaphragm is configured such as, on generation of a pre-determined actuation pressure within the chamber in the housing, to be deflected such as to engage the other, distal ends of the arms, and cause the same to be pivoted to the release position. This actuation pressure cannot be achieved until the nosepiece is sufficiently inserted in a nostril of a subject for effective operation of the device, in which position the escape of exhaled air from the exhalation breath of the subject directly to the atmosphere is prevented. While the nosepiece is not sufficiently inserted into a nostril of a subject as to provide for effective operation of the device, exhaled air from the exhalation breath of the subject escapes to the atmosphere, thereby preventing the development of the actuation pressure within the chamber in the housing.

With this configuration, the device, in being pre-primed and actuatable by the oral exhalation breath of a subject, does not require the application of an actuation force by the subject at the instance of actuation, and provides for the closure of the oropharyngeal velum of the subject.

In some embodiments, the device comprises mechanical liquid delivery pump operated by the manual compression of a chamber containing a volume of liquid to expel a flow of a metered volume of liquid.

In some embodiments, the device further comprises one or more of a filter, a flow meter, a flow regulator, and a nebulizer.

In some embodiments, the nozzle can be configured to deliver an aerosol spray with an asymmetric spray profile, with the aerosol spray having a significantly greater spray angle in the vertical, sagittal plane than in the horizontal plane. Such an aerosol spray has been found to be particularly advantageous in the delivery of substance to posterior regions of the nasal cavities, in particular the olfactory region.

In some embodiments, the spray angle in the vertical, sagittal plane is greater than about 35°, greater than about 40°, greater than about 45°, or greater than about 50°. In some embodiments, the spray angle in the horizontal plane is not more than about 35°, not more than about 30°, not more than about 25°, not more than about 20°, or not more than about 15°.

In some embodiments, the aerosol spray can present an elliptical spray zone. In some embodiments, the aerosol spray can present a substantially rectangular spray zone. In some embodiments, the device further comprises a substance supply unit for delivering metered doses of a composition comprising racemic ketamine, which is fluidly connected to the nozzle to deliver the composition from the nosepiece as an aerosol spray. In some embodiments, the substance supply unit is a multi-dose unit for delivering a plurality of metered doses of the composition. In some embodiments, the substance supply unit is a single-dose unit for delivering a single metered dose of the composition. In some embodiments, the substance supply unit is pre-primeable, by loading a resilient element, which, when released, the resilient element actuates the substance supply unit to deliver a metered dose of the composition through the nozzle. In some embodiments, the device comprises a piston to deliver a metered dose of the composition through the nozzle.

In some embodiments, the device comprises one or more indicators, for example, to indicate a first dose and a second dose. In some embodiments, the indicator can be a color change or a number change. For example, after a dose has been dispensed, the indicator comes to be positioned behind a viewing window such that it is viewable by the subject. In some embodiments, the device comprises one or two viewing windows. In some embodiments, after a first dose has been dispensed, a first viewing window can become red, whereas a second viewing window can remain blank. After a second dose has been dispensed, both viewing windows can be red. Thus, the subject will have no difficulty in quickly determining whether or not the first and/or the second dose has/have been dispensed, and thus does not risk over-dosing and/or under-dosing.

In some embodiments, the device is primeless and can be actuated with one hand. In some embodiments, the device is disposable. In some embodiments, each device provides one or two doses of racemic ketamine. In some embodiments, the device comprises a reservoir containing one or two doses of racemic ketamine, and a dispenser member (such as a piston), mounted to slide into the reservoir. Movement of the dispenser member results in dispensing of a dose of the racemic ketamine. In a dual-dose device, the piston is moved in two successive actuation strokes, thereby dispensing separate first and second doses. In some embodiments, the device further comprises an indicator such that the user can visually determine if (i) no dose has been dispensed; (ii) if only the first dose has been delivered; and (ii) if both the first dose and the second dose have been dispensed. For example, a colored indication zone within a viewing window in the device may change color after the first dose has been dispensed, and change color again (or another colored indication zone, if present, may change color) after the second dose has been dispensed. Similarly, actuation of the dispenser member may cause a first colored indication zone to be obscured after the first dose is dispensed, and may cause a second colored indication zone to be obscured after the second dose is dispensed.

In some embodiments, the device is the Aptar Biodose (BDS) system.

Other suitable intranasal delivery devices are described in U.S. Pat. No. 7,299,949 (see, e.g., FIGS. 1-3); U.S. Pat. No. 9,555,950 (see. e.g., FIGS. 1-3); U.S. Pat. No. 10,099,019 (see, e.g., FIGS. 1-41); U.S. Pat. No. 10,179,216 (see, e.g., FIGS. 1-5); U.S. Pat. No. 10,525,218 (see, e.g., FIGS. 1-26); U.S. Pat. No. 10,549,052 (see, e.g., FIGS. 1-19); U.S. Pat. No. 7,784,460 (see, e.g., FIGS. 1-8); U.S. Pat. No. 8,146,589 (see, e.g., FIGS. 1-5); U.S. Pat. No. 8,875,711 (see, e.g., FIGS. 1); and U.S. Pat. No. 8,985,116 (see, e.g., FIG. 1); and U.S. Publ. Nos. 20040039352; 20090054923; 20120000459; 20120017902; 20130245560; 20140018295; 20150190268; 20170020383; 20170151397; 20170216540; 20180256836; 20180256867; 20180272085; 20180361085; 20190054016; 20190070372; 20190083722; 20190117916; 20190117918; 20190143054; 20190269867; 20190290863; 20190290864; 20190314588; 20190358078; 20190358417; 20200023146; 20200054843; 20200060972; 20200206012; 20200206441; and 20200206547, each of which is incorporated by reference in its entirety, including any drawings.

It is understood that the examples and embodiments described herein are for illustrative purposes only and that various modifications or changes in light thereof will be suggested to persons skilled in the art and are to be included within the spirit and purview of this application and scope of the appended claims. All publications, patents, patent applications, and sequence accession numbers cited herein are hereby incorporated by reference in their entirety for all purposes.

### Examples

### Example 1. A Two-Part Randomized, Double-Blind, Placebo-Controlled, Parallel-Design and Partial Crossover Study Regarding the Pharmacodynamics, Pharmacokinetics, and Safety of Multiple Doses of Intranasal and Intravenous Ketamine in Normal Healthy Volunteers

This example describes a two-part randomized, double-blind, placebo-controlled, parallel-design and partial crossover study to determine the pharmacodynamics, pharmacokinetics, and safety of multiple doses of intranasal and intravenous ketamine in normal healthy volunteers.

### Study Overview

This protocol encompasses two parts. Part A is a randomized, double-blind, placebo-controlled, multiple-dose, parallel-design cohorts to evaluate the psychotomimetic effects, pharmacokinetics, and safety of various doses of intranasal racemic ketamine administered 3 times within 8 days. This part consists of a screening visit, a treatment phase, and a follow-up visit. Within 30 days of screening, eligible subjects are enrolled and randomized into the treatment phase. Subjects are admitted to the clinic 1 day prior to dosing (Day −1) and remain as an inpatient in the research clinic for 10 days (9 nights).

Part A consists of repeated single doses of racemic ketamine or placebo administered intranasally on Days 1, 4, and 8. There are 4 dose levels:
Treatment W: Placebo intranasal
Treatment X: racemic ketamine 30 mg intranasal
Treatment Y: racemic ketamine 75 mg intranasal
Treatment Z: racemic ketamine 90 mg intranasal

The trials included six sequential cohorts, consisting of 8 subjects each. Subjects in each cohort were randomized such that there was an even distribution of subjects per dose level (i.e., 2 subjects received placebo, 2 subjects received 30 mg, 2 subjects received 75 mg, and 2 subjects received 90 mg). After treatment of each cohort, the Safety Review Team assessed the feasibility of continuing with any dose level based on the available safety information.

The pharmacodynamic, pharmacokinetic, and safety assessments were performed up to 24 hours postdose. At the discretion of the investigator, subjects were discharged approximately 24 hours after the last dosing. Subjects return for a safety follow-up visit 12 days (+1 day) following the first drug administration. In case of early withdrawal, study exit procedures were completed at the time of withdrawal (or soon after) and subjects may be asked to return for a safety follow-up visit 12 days (=1 day) following the first drug administration, as per investigator's judgment.

Part B was a randomized, double-blind, double-dummy, placebo-controlled, 2-period partial crossover to evaluate the psychotomimetic effects, PK, and safety of a single dose of intranasal vs IV ketamine administered 3 times within 8 days. Within 30 days of screening, eligible subjects were enrolled and randomized into the treatment phase. Subjects participate in two 10-day (9 nights) inpatient stays in the research clinic separated by a washout of at least 4 days.

Part B consists of a single dose level of 60 mg, randomly administered as racemic ketamine intranasal on Days 1, 4, and 8 in one treatment period and as 0.3 mg/kg IV on Days 1, 4, and 8 in the other period. The IV dose of ketamine 0.3 mg/kg was considered to be equivalent to racemic ketamine 60 mg dose on the basis of anticipated plasma exposure. Subjects were randomized to receive the treatments listed below, such that 2 subjects received placebo and 12 subjects receive the active treatment. To maintain the blind, each study treatment was administered as intranasal and IV.
Treatment A: Placebo (intranasal+IV)
Treatment B: racemic ketamine 60 mg intranasal+placebo IV
Treatment C: Ketamine IV 0.3 mg/kg (dose equivalent to 60 mg intranasal)+placebo intranasal

The PD, PK, and safety assessments were performed up to approximately 24 hours postdose. At the discretion of the investigator, subjects were discharged approximately 24 hours after the last dosing in each treatment period. Subjects returned for a safety follow-up visit 12 days (+1 day) following the first drug administration in treatment period 2. In case of early withdrawal, study exit procedures were completed at the time of withdrawal (or soon after) and subjects were asked to return for a safety follow-up visit 12 days (+1 day) following the last drug administration, as per investigator's judgment.

### Objectives

The primary objective of this study was:
(1) To determine the pharmacodynamic (PD) effects of racemic ketamine (intranasal ketamine HCl) and IV ketamine following multiple doses (repeated single doses), as assessed by rating scales and psychomotor tests for psychotomimetic and dissociative effects.

The secondary objectives were:
(2) To assess the pharmacokinetic (PK) parameters of ketamine and its metabolites (norketamine and 6-hydroxynorketamine) following single and multiple intranasal and IV doses of ketamine;
(3) To compare the bioavailability of racemic ketamine 60 mg and 0.3 mg/kg ketamine IV after single and multiple doses;
(4) To assess the correlation between ketamine plasma concentrations and electrocardiogram parameters; and
(5) To assess the safety of single and multiple intranasal and IV doses of ketamine.

The exploratory objectives were:
(6) To explore the dose proportionality of ketamine and norketamine after intranasal racemic ketamine administration; and
(7) To examine the correlation between PK parameters and various PD effects for ketamine and its metabolites.

### Number of Subjects

A sufficient number of healthy subjects were screened and randomized to the treatment phase in order to ensure evaluable data from at least 9 subjects per dose level in Part A (a total of 36 subjects) and at least 11 subjects in Part B.

Subjects who participated in Part A may be eligible to participate in Part B.

### Criteria for Inclusion

The following were the inclusion criteria. Subjects must meet each of the following inclusion criteria to be eligible:
(1) healthy male or female subjects 20 to 55 years of age, inclusive;
(2) body mass index (BMI) within the range of 18.0 to 35.0 kg/m², inclusive, and a minimum weight of at least 50.0 kg;
(3) nonsmoker for at least 3 months and tests negative on a urine cotinine test;
(4) female subjects of childbearing potential with male sexual partners must be using and willing to continue using medically acceptable contraception for at least 1 month prior to Screening (at least 3 months for oral and transdermal contraceptives) and for at least 1 month after the last study drug administration;
(5) female subjects of non-childbearing potential must be surgically sterile (hysterectomy and/or bilateral oophorectomy/salpingo-oophorectomy, as determined by subject medical history) or congenitally sterile, or must be postmenopausal, where postmenopausal is defined as being amenorrheic for at least 1 year without another cause and an FSH level≥26 IU/L.6;
(6) resting heart rate between 50 and 100 beats per minute inclusive;
(7) able to speak, read, and understand English or French sufficiently to understand the nature of the study, to provide written informed consent, and to allow completion of all study assessments; and
(8) must provide written informed consent prior to the initiation of any protocol-specific procedures.

### Exclusion Criteria

The following were the exclusion criteria. Subjects were not considered eligible to participate in this study if any one of the following exclusion criteria was satisfied at screening:
(1) Self-reported substance or alcohol dependence or abuse (excluding nicotine and caffeine) in their lifetime, and/or has ever participated or plans to participate in a substance or alcohol rehabilitation program to treat their substance or alcohol dependence;
(2) Self-reported abuse of ketamine or phencyclidine (PCP) in their lifetime;
(3) Clinically significant abnormalities as assessed by physical examination, medical history, 12-lead electrocardiogram, vital signs, or laboratory values, as judged by the investigator or designee;
(4) History or presence of any clinically significant illness (e.g., cardiac, pulmonary, hepatic, renal, hematologic, gastrointestinal, endocrine, immunologic, dermatologic, oncologic, or musculoskeletal) or any condition, which in the opinion of the investigator or designee, would jeopardize the safety of the subject or the validity of the study results;
(5) Personal or first-degree family history of psychosis, personal history of any one of: mood disorder, anxiety disorder, obsessive-compulsive disorder, somatoform disorder, and behavior disorder;
(6) Personal history of neurologic disorder in relation to the central nervous system (CNS): congenital malformation of the brain, brain tumor, multiple sclerosis, degenerative disease of the CNS, or inflammatory disease of the CNS within the past year or having resulted in sequelae;
(7) History or present diagnosis of glaucoma;
(8) Known hypertension or blood pressure above 140/90 mmHg (blood pressure may be repeated as per the site's SOPs);
(9) Presence or history of cardiac disorder, including congenital heart disease, ischemic heart disease, cardiac insufficiency, supraventricular and ventricular heart rhythm disorder, prolonged QT syndrome (i.e., QTcF >450 msec) and associated risk factors (i.e., hypokalemia, family history of long QT syndrome);
(10) Any history of suicidal ideation or suicidal behavior (lifetime), as assessed by the Columbia-Suicide Severity Rating Scale (C-SSRS; baseline version);
(11) Current (i.e., within the last 3 months) treatment with any psychotropic medications;
(12) Color blindness (for Bowdle VAS perception of color intensity) based on subject self-report;
(13) Presence of piercing or any medical condition that could interfere with the absorption or pharmacokinetics of intranasal racemic ketamine (e.g., nasal polyps, clinically significant deviated septum [corrected or persistent], or other physical abnormalities of the nose);
(14) Any history of epilepsy or seizures, excluding childhood febrile seizures;
(15) History of severe allergic reaction (including anaphylaxis) to ketamine or related drugs (other NMDA receptor antagonists) or to any food, medication, or bee sting, or previous status asthmaticus;
(16) Use of a prohibited medication;
(17) Use of soft drugs (such as marijuana) within 3 months prior to the screening visit or hard drugs (such as cocaine, crack, opioid derivatives including heroin, and amphetamine derivatives) within 1 year prior to screening;
(18) Positive urine drug screen (UDS);
(19) Regular use of alcohol within 6 months prior to the screening visit (more than 14 units of alcohol per week where 1 unit=150 mL of wine, 360 mL of beer, or 45 mL of 40% alcohol);
(20) Positive breath alcohol test, but subjects may be rescheduled at the discretion of the investigator or designee;
(21) Difficulty with venous access or unsuitable or unwilling to undergo catheter insertion;
(22) Female subjects who have a positive pregnancy test, were currently pregnant or lactating, or who were planning to become pregnant within 30 days of last study drug administration;
(23) Donation of plasma within 7 days prior to dosing or donation or loss of blood (excluding volume drawn at screening) of 50 mL to 499 mL of blood within 30 days, or more than 499 mL within 56 days prior to the first dosing;
(24) Positive for hepatitis B, hepatitis C, or human immunodeficiency virus (HIV);
(25) Treatment with an investigational drug within 5 times the elimination half-life, if known (e.g., a marketed product) or within 30 days (if the elimination half-life was unknown), or within 90 days for a biologic, prior to first drug administration or was concurrently enrolled in any research judged not to be scientifically or medically compatible with this study;
(26) An employee of the sponsor, clinical research organization, or research site personnel directly affiliated with this study or their immediate family member defined as a spouse, parent, child or sibling, whether biological or legally adopted;
(27) A subject who, in the opinion of the investigator or designee, was considered unsuitable or unlikely to comply with the study protocol for any reason; or
(28) A subject who has pending legal charges or was on probation.

### Dietary and Other Restrictions

Aside from the inclusion and exclusion criteria, the subject must agree to abide by each of the following restrictions for the specified time:
(1) Subjects were required to abstain from alcohol for 24 hours prior to each study visit and abstinence were confirmed with a breath alcohol test;
(2) Subjects were required to abstain from recreational drug use throughout the study, from screening until after the follow-up visit;
(3) Subjects were required to fast (abstain from food) for at least 8 hours prior to dosing and for at least 4 hours postdose, water was permitted ad libitum except for at least 1 hour prior to dosing and for at least 1 hour postdose;
(4) Subjects were asked to abstain from the following foods from 1 week prior to the treatment phase until after the follow-up visit: grapefruit or grapefruit-containing products, pomegranate, pomelo, star fruit juice/products, foods containing poppy seeds, Seville oranges, and orange juice
(5) Subjects were asked not to consume more than 450 mg of caffeine per day (e.g., approximately 5 cups of tea or 3 cups of regular coffee or 8 cans of cola or 2 energy drinks) from 1 week prior to the treatment phase until after the follow-up visit, and subjects were not be permitted to consume caffeine-containing beverages while housed at the research site;
(6) Subjects were asked to refrain from driving, operating machinery, or engaging in hazardous activities until they and the investigator were sure the study drug was not impairing their judgment and/or ability to perform skilled tasks, and subjects were informed that driving under the influence was a criminal offense and that if they were caught driving under the influence, they may be prosecuted to the fullest extent of the law;
(7) Subjects were asked to abstain from strenuous physical activity for 48 hours prior to each study visit, subjects were not be permitted to engage in strenuous exercise during the in-subject stay at the research site, and for safety reasons, subjects were required to remain seated or semi-reclined in bed for the first 4 hours after drug administration;
(8) Subjects were required to abstain from blood donation during the study and for 30 days following the follow-up visit; and
(9) Subjects were required to follow the informed consent form (ICF) and the clinic code of conduct.

### Test Product(s), Dose, and Mode of Administration:

In Part A, subjects were randomized to receive either placebo or racemic ketamine (30 mg, 75 mg, or 90 mg), with an even distribution of subjects per dose level. The study drug (placebo and active) was administered intranasally following an overnight fast of at least 8 hours on Days 1, 4, and 8.

In Part B, the following study treatments were given in a double-dummy fashion, such that each subject will receive the study drug (placebo and/or active) as intranasal and IV:
Treatment A: Placebo intranasal+Placebo IV
Treatment B: racemic ketamine 60 mg intranasal+placebo IV
Treatment C: Ketamine 0.3 mg/kg IV+placebo intranasal

The study treatment was administered on Days 1, 4, and 8 of both treatment periods following an overnight fast of at least 8 hours.

Time of study drug administration was set as the first spray administration in Part A and was set at the start of the infusion in Part B.

### Intranasal Administration

There was a total of 6 sprays per subject per day on dosing days. The subjects were instructed to gently blow their nose prior to drug administration. The start of dosing was considered time zero. The drug was administered by trained study personnel as 2 sprays (0.1 mL per spray), one in each nostril, from each bi-dose device. The study-specific procedure details the required standard position of the subject's head during drug administration and the instructions given to subjects regarding sniffing following administration of the sprays. Nasal cavity check was be done after each dosing in order to confirm proper inhalations.

The administration of drug from each bi-dose device was separated by approximately 5 minutes to ensure optimal absorption of the drug in the nasal cavity due to the 2-fold increase in the volume of spray relative to prior studies. Subjects may not blow their nose for 1 hour following dosing. A smell/taste evaluation was performed on Day 9, approximately 24 hours after administration of the last intranasal drug. Time of study drug administration was set as the first spray administration.

Effort was made to administer the 6 sprays in Part B during the IV infusion period, therefore the first spray was administered after the start of infusion and the last spray would be administered before the end of infusion.

### Intravenous Administration

In Part B, the dose of ketamine IV was based on the subject's weight at admission time. The IV study drugs were administered as an IV infusion administered over approximately 10 minutes. The subject's actual times of the start and end of the infusion were recorded in the source documents. Time of study drug administration was set as the start of study drug infusion.

### Criteria for Evaluation: Pharmacodynamic

The primary pharmacodynamic endpoints were the maximum (peak) effect (Eₘₐₓ), maximum change from baseline (CFBₘₐₓ), and time-averaged were a under the effect curve (TA_AUE), as applicable, for:
(1) Bowdle visual analog scale (VAS); and
   Eₘₐₓ for internal and external perceptions and TA_AUE
(2) Clinician Administered Dissociative State Scale (CADSS)
   Eₘₐₓ and CFBₘₐₓ for total score.

The secondary endpoints include: Eₘₐₓ, minimum effect (Eₘᵢₙ), CFBₘₐₓ, minimum change from baseline (CFBₘᵢₙ), and TA_AUE, as applicable, for:
(3) Choice Reaction Time (CRT);
   CFBₘₐₓ and baseline-adjusted TA_AUE of motor reaction time (MRT), recognition reaction time (RRT), and total reaction time (TRT));
   CFBₘᵢₙ and baseline-adjusted TA_AUE of percentage correct;
(4) Sternberg Short-Term Memory (SSTM) task; and
   Eₘᵢₙ and CFBₘᵢₙ for d′ pooled;
   Eₘₐₓ and CFBₘₐₓ for mean reaction time pooled for all valid responses;
(5) POMS 2 (for Treatment B and Treatment C in Part B only);
   CFBₘₐₓ Total Mood Disturbance;
(6) Subject-Rated Assessment of Intranasal Irritation (SRAII);
   Eₘₐₓ and CFBₘₐₓ.

### Criteria for Evaluation: Pharmacokinetic

The PK parameters that were evaluated in this study for ketamine, norketamine and 6-hydroxynorketamine, (as applicable) include:
(1) Time to maximum observed plasma concentration (Tₘₐₓ);
(2) Maximum observed plasma concentration (Cₘₐₓ);
(3) Area under the plasma concentration-time curve from time zero to the last measurable concentration (AUC₀₋ₜ);
(4) Area under the plasma concentration-time curve extrapolated to infinity (AUC_{0-inf});
(5) First-order rate constant associated with the terminal (log-linear) portion of the curve (2);
(6) Terminal elimination half-life (t½);
(7) Apparent clearance (ketamine only) (CL/F); and
(8) Apparent volume of distribution (ketamine only) (V_{d}/F);

### Criteria for Evaluation: Safety

### Safety Endpoints Include:

(1) Holter monitor to determine whether there was a correlation between electrocardiogram parameters and the PK concentration of intranasal racemic ketamine in plasma;
(2) Adverse events including type, incidence, and severity;
(3) Vital signs (blood pressure, respiratory rate, heart rate, oxygen saturation, and oral temperature);
(4) 12-lead electrocardiogram (ventricular heart rate and the PR, QRS, QT, and QTc intervals);
(5) Clinical laboratory tests;
(6) Physical examination;
(7) Nasal cavity examination; and
(8) Columbia-Suicide Severity Rating Scale (C-SSRS).

### Subject Discontinuation

Any subject who voluntarily withdraws consent or was discontinued (e.g., because of an adverse event) from the study prior to completion was considered as withdrawn from the study. Subjects may be discontinued from the study under any of the following circumstances:
(1) Occurrence of intolerable AE, as assessed by the investigator or designee;
(2) Clinically significant abnormality on vital signs, electrocardiogram, clinical laboratory, or physical examination assessments, as assessed by the investigator or designee;
(3) Withdrawal of consent;
(4) Lost to follow-up;
(5) Administrative reasons;
(6) Sponsor decision;
(7) Major violation of the protocol;
(8) if, in the opinion of the qualified investigator, it was in the best interest of the subject;
(9) Pregnancy;
(10) Noncompliance with study requirements and restrictions (e.g., positive urine cotinine test at any study visit, use of a concomitant medication); and
(11) Termination of the study,

When an event such as a family emergency, a transient intercurrent illness (such as a cold) unrelated to study drug, or a remediable act of noncompliance prevents a subject from participating in a scheduled visit, but the subject wishes to continue in the study, with the agreement of the investigator, the research site staff may attempt to reschedule the visit if feasible for the study and retain the subject in the study.

If a subject was prematurely discontinued from participation in the study for any reason after drug administration, the investigator or designee must make every effort to perform the assessments scheduled for the Follow-up visit. Replacement subjects may be added at the sponsor's discretion, in agreement with the principal investigator.

### Statistical Methods

### Treatment Population Definitions:

The following analysis populations were used for Part A:
Part A Randomized Population: All subjects who were randomized into the study.
Part A Safety Population: All randomized subjects who receive any study treatment.
Part A Pharmacodynamic (PD) Population: All subjects in the Part A Safety Population who receive any study treatment and who have no protocol deviations or other circumstances that would exclude them from PD analysis.
Part A Pharmacokinetic (PK) Population: All subjects in the Part A Safety Population who receive at least 1 dose of the study drug, have evaluable PK data who have no protocol deviations or other circumstances that would exclude them from PK analysis.

The following analysis populations were used for Part B:
Part B Randomized Population: All subjects who were randomized into the study.
Part B Safety Population: All randomized subjects who receive any study treatment.
Part B Completers Population: All subjects in the Part B Safety Population who receive both study treatments (e.g., ketamine 60 mg intranasal and IV), and complete both treatment periods regardless of whether they have protocol deviations.
Part B Pharmacokinetic (PK) Population: All subjects in the Part B Safety Population who receive at least 1 dose of the study drug, have evaluable PK data and who have no protocol deviations or other circumstances that would exclude them from PK analysis.
Part B Bioavailability Population: All subjects in the Part B PK Population who receive both study treatments (e.g., ketamine 60 mg intranasal and IV), and complete both treatment periods.

### Pharmacodynamics and Pharmacokinetics:

PK and PD statistical analyses were performed using SAS® (release 9.4 or higher). PK parameter derivation were performed using Phoenix WinNonlin (version 8.0 or higher on the Windows 7 platform or higher).

For Part A, descriptive statistics were provided for the Part A PD Population. PD data at each time point were summarized by summary statistics including n, mean, standard error (SE), minimum, first quartile (Q₁), median, third quartile (Q₃) and maximum. PD derived endpoints were summarized by treatment using descriptive statistics. PD data were presented graphically (where appropriate), and listed for the Part A Randomized Population.

For Part B, descriptive statistics and inferential analysis were done on the Part B Completers Population. Listings for each PD parameter and endpoint were provided for the Part B Randomized Population.

PD data at each time point was summarized by descriptive statistics including n, mean, standard error (SE), minimum, first quartile (Q₁), median, third quartile (Q₃) and maximum. Derived endpoints were summarized by treatment and paired difference using descriptive statistics. PD data were presented graphically (where appropriate), and listed for the Part B Randomized Population.

For Part A and Part B, PK descriptive statistics were done using the Part A and Part B PK Populations. Descriptive statistics, including n, arithmetic mean, standard deviation (SD), CV %, median, minimum, and maximum, were calculated and presented for each time point by treatment for plasma concentrations of the following analytes: ketamine, norketamine, and 6-hydroxynorketamine. Mean (SD) and individual time course plots of the concentrations (original and log transformed) vs time were generated.

PK parameters for all analytes were calculated using noncompartmental analysis (Phoenix WinNonlin®, version 8.0) and were summarized by treatment using descriptive statistics, including n, arithmetic mean, SD, CV %, median, minimum, maximum, geometric mean, and geometric CV % except Tₘₐₓ, t½, and λ. Tₘₐₓ data were summarized with minimum, Q₁, median, Q₃, and maximum. The t½ and A data were summarized with n, mean, SD, CV, minimum, median, and maximum. The PK blood sampling collected at follow-up visit for both Part A and Part B will not be used for PK parameters calculation.

### Results

The results of Part A of the clinical study described in Example 1 are shown in FIGS. 1-9. FIGS. 1-3 describe the pharmacokinetic parameters for ketamine on Days 1, 4, and 8 (FIG. 1, FIG. 2, and FIG. 3, respectively). FIGS. 4-6 describe the pharmacokinetic parameters for norketamine on Days 1, 4, and 8 (FIG. 4, FIG. 5, and FIG. 6, respectively). FIGS. 7-9 describe the pharmacokinetic parameters for hydroxynorketamine on Days 1, 4, and 8 (FIG. 7, FIG. 8, and FIG. 9, respectively).

The results of Part B of the clinical study described in Example 1 are shown in FIGS. 10A-10C, FIGS. 11A-11C, and FIGS. 12A-12C.

### Example 2. An Open-Label Study Regarding Drug-Drug Interactions of Multiple Doses of Oral Sertraline or Venlafaxine Coadministered with Intranasal Ketamine in Healthy Adult Volunteers

This example describes an open-label study to determine the drug-drug interaction of multiple doses of oral sertraline or venlafaxine coadministered with intranasal ketamine in healthy adult volunteers.

### Study Overview

This is a single-center, open-label study in healthy subjects. Each subject participates in a medical screening visit, a treatment period, and a follow-up visit. The total duration of this study is approximately 7 weeks. Within 30 days of screening, eligible subjects are randomized into 1 of 2 arms:
Arm 1: racemic ketamine (60 mg)+venlafaxine extended release (Effexor® XR)
Arm 2: racemic ketamine (60 mg)+sertraline (Zoloft®)

Subjects are admitted on Day −1 and are confined in the clinical research unit for 13 days (12 nights). On Day 1, subjects are given a single dose of racemic ketamine 60 mg, followed by PK sampling and PD and safety assessments. There are approximately 44 hours between the racemic ketamine and venlafaxine/sertraline doses. On Day 3, subjects are given single oral doses of either venlafaxine or sertraline for 9 consecutive days (Day 3 to Day 11), starting with a lower dose (75 mg venlafaxine, 50 mg sertraline) then increasing to a higher dose (150 mg venlafaxine, 100 mg sertraline) to achieve steady-state concentrations for venlafaxine and sertraline.

The second dose of racemic ketamine 60 mg is intranasally administered 4 hours after venlafaxine or sertraline on Day 11 to correspond with the time of maximum concentration of venlafaxine/sertraline, which would allow for the detection of any drug interaction. Racemic ketamine, or a pharmaceutically acceptable salt thereof, is intranasally administered at least 3 hours after a meal due to the food effect for sertraline and to mimic the fasted conditions implemented in previous studies.

Ketamine PK samples are taken on Day 1 and Day 2 following the first dose, as well as on Day 11 and Day 12 following the second dose of ketamine. Serial PK sampling for venlafaxine or sertraline is performed at the expected steady state, beginning on Day 10 without ketamine and on Day 11 in the presence of ketamine, accompanied by PD and safety assessments. Predose PK samples for venlafaxine or sertraline from Day 3 to Day 10 is used to verify if steady state is achieved for these drugs.

Subjects are discharged on Day 12, with a follow-up visit on Day 15.

### Objectives:

The primary objective of this study is to determine the effect of coadministration of sertraline or venlafaxine on the PK and metabolism of a single dose of racemic ketamine (IN ketamine HCl).

The secondary objectives are:
(1) To assess the effect of a single dose of racemic ketamine and its metabolites on the PK of sertraline or venlafaxine;
(2) To assess the PD interaction (BP) between racemic ketamine and sertraline or venlafaxine; and
(3) To assess the safety and tolerability of racemic ketamine coadministered with either sertraline or venlafaxine.

### Number of Subjects

Approximately 48 healthy subjects (24 subjects per arm) are randomized into one of the 2 study treatment arms with the intent to ensure complete data from at least 14 subjects per treatment arm (total of 28 subjects).

### Criteria for Inclusion:

Subjects are considered eligible to participate in this study if each one of the following inclusion criteria is satisfied at screening:
(1) Healthy male or female subjects 20 to 55 years of age, inclusive;
(2) Body mass index (BMI) within the range of 18.0 to 35.0 kg/m², inclusive, and a minimum weight of at least 50.0 kg;
(3) Nonsmoker for at least 3 months and tests negative on a urine cotinine test;
(4) Female subjects of childbearing potential with male sexual partners must be using and willing to continue using medically acceptable contraception for at least 1 month prior to Screening (at least 3 months for oral and transdermal contraceptives) and for at least 1 month after the last study drug administration;
(5) Female subjects of non-childbearing potential must be surgically sterile (hysterectomy and/or bilateral oophorectomy/salpingo-oophorectomy, as determined by subject medical history) or congenitally sterile, or must be postmenopausal, where postmenopausal is defined as being amenorrheic for at least 1 year without another cause and an FSH level ≥26 IU/L.6;
(6) Male subjects with female partners of childbearing potential must be using and willing to continue using medically acceptable contraception from Screening and for at least 90 days after the last study drug administration;
(7) Resting heart rate between 50 beats per minute and 100 beats per minute, inclusive, and heart rate measurements may be repeated as per the site's standard operating procedure (SOP);
(8) Able to speak, read, and understand English or French sufficiently to understand the nature of the study, to provide written informed consent, and to allow completion of all study assessments;
(9) Must provide written informed consent prior to the initiation of any protocol-specific procedures; and
(10) Must be willing and able to abide by all study requirements and restrictions.

### Exclusion Criteria

Subjects are not considered eligible to participate in this study if any one of the following exclusion criteria is satisfied at screening:
(1) Self-reported substance or alcohol dependence or abuse (excluding nicotine and caffeine) in their lifetime, and/or has ever participated or plans to participate in a substance or alcohol rehabilitation program to treat their substance or alcohol dependence;
(2) Self-reported abuse of ketamine or phencyclidine (PCP) in their lifetime;
(3) Clinically significant abnormalities as assessed by physical examination, medical history, electrocardiogram, vital signs, or laboratory values as judged by the investigator or designee;
(4) History or presence of any clinically significant illness (e.g., cardiac, pulmonary, hepatic, renal, hematologic, gastrointestinal, endocrine, immunologic, dermatologic, oncologic, or musculoskeletal) or any condition, which in the opinion of the investigator or designee would jeopardize the safety of the subject or the validity of the study results;
(5) Personal or first-degree family history of psychosis, personal history of mood disorder, anxiety disorder, obsessive-compulsive disorder, somatoform disorder, behavior disorder;
(6) Personal history of neurologic disorder in relation to the CNS: congenital malformation of the brain, brain tumor, multiple sclerosis, degenerative disease of the CNS, or inflammatory disease of the CNS within the past year or having resulted in sequelae;
(7) History or present diagnosis of glaucoma;
(8) Known hypertension or blood pressure above 140/90 mmHg (blood pressure may be repeated as per the site's SOPs);
(9) Presence or history of cardiac disorder, including congenital heart disease, ischemic heart disease, cardiac insufficiency, supraventricular and ventricular heart rhythm disorder, prolonged QT syndrome (i.e., QTc>450 msec) and associated risk factors (i.e., hypokalemia, family history of long QT syndrome);
(10) Any history of suicidal ideation or suicidal behavior (lifetime), as assessed by the Columbia-Suicide Severity Rating Scale (C-SSRS; baseline version);
(11) Current (i.e., within the last 3 months) treatment with any psychotropic medications;
(12) Presence of piercing or any medical condition that could interfere with the absorption or PK of IN ketamine (e.g., nasal polyps, clinically significant deviated septum [corrected or persistent], or other physical abnormalities of the nose);
(13) Any history of epilepsy or seizures (except childhood febrile seizures);
(14) History of severe allergic reaction (including anaphylaxis) to ketamine or related drugs (other NMDA receptor antagonists), venlafaxine or related drugs (other SNRIs), or sertraline or related drugs (other SSRIs) or to any food, medication, or bee sting, or previous status asthmaticus;
(15) Use of a prohibited medication;
(16) Use of soft drugs (such as marijuana) within 3 months prior to the screening visit or hard drugs (such as cocaine, crack, opioid derivatives including heroin, and amphetamine derivatives) within 1 year prior to screening;
(17) Positive urine drug screen (UDS);
(18) Regular use of alcohol within 6 months prior to the screening visit (more than 14 units of alcohol per week [1 unit=150 mL of wine, 360 mL of beer, or 45 mL of 40% alcohol]);
(19) Positive breath alcohol test, however, subjects may be rescheduled at the discretion of the investigator or designee;
(20) Difficulty with venous access or unsuitable or unwilling to undergo catheter insertion;
(21) Female subjects who are currently pregnant (have a positive pregnancy test), lactating, breastfeeding, or who are planning to become pregnant within 30 days of last study drug administration;
(22) Positive for hepatitis B, hepatitis C, or human immunodeficiency virus (HIV);
(23) Donation of plasma within 7 days prior to dosing or donation or loss of blood (excluding volume drawn at screening) of 50 mL to 499 mL of blood within 30 days, or more than 499 mL within 56 days prior to the first dosing;
(24) Treatment with an investigational drug within 5 times the elimination half-life, if known (e.g., a marketed product), or within 30 days (if the elimination half-life is unknown), or within 90 days for a biologic prior to first drug administration or is concurrently enrolled in any research judged not to be scientifically or medically compatible with this study;
(25) An employee of the sponsor, clinical research organization, or research site personnel directly affiliated with this study or their immediate family member defined as a spouse, parent, child, or sibling, whether biological or legally adopted;
(26) A subject who, in the opinion of the investigator or designee, is considered unsuitable or unlikely to comply with the study protocol for any reason; and
(27) A subject who has pending legal charges or is on probation.

### Dietary and Other Restrictions

Aside from the inclusion and exclusion criteria, the subject must agree to abide by each of the following restrictions for the specified time:
(1) Subjects are required to abstain from alcohol for 24 hours prior to each study visit and abstinence are confirmed with a breath alcohol test;
(2) Subjects are required to abstain from recreational drug use throughout the study, from screening until after the follow-up visit;
(3) Subjects are required to fast (abstain from food) for at least 3 hours prior to ketamine dosing and at least 1 hour after ketamine dosing;
(4) Except for water given with venlafaxine or sertraline and fluids provided with the meal, no fluids are allowed from 1 hour before until 1 hour after venlafaxine or sertraline dosing, however, water is provided ad libitum at all other times;
(5) Subjects are asked to abstain from the following foods from 1 week prior to the treatment phase until after the follow-up visit: grapefruit or grapefruit-containing products, pomegranate, pomelo, star fruit juice/products, foods containing poppy seeds, Seville oranges, and orange juice
(6) Subjects are asked not to consume more than 450 mg of caffeine per day (e.g., approximately 5 cups of tea or 3 cups of regular coffee or 8 cans of cola or 2 energy drinks) from 1 week prior to the treatment phase until after the follow-up visit, and subjects are not be permitted to consume caffeine-containing beverages while housed at the research site;
(7) Subjects are asked to refrain from driving, operating machinery, or engaging in hazardous activities until they and the investigator are sure the study drug is not impairing their judgment and/or ability to perform skilled tasks, and subjects are informed that driving under the influence is a criminal offense and that if they are caught driving under the influence, they may be prosecuted to the fullest extent of the law;
(8) Subjects are asked to abstain from strenuous physical activity for 48 hours prior to each study visit, subjects are not be permitted to engage in strenuous exercise during the in-subject stay at the research site, and for safety reasons, subjects are required to remain seated or semi-reclined in bed for the first 4 hours after intranasal racemic ketamine administration on Day 1 and Day 11, as well as after the first venlafaxine or sertraline administration on Day 3 and after dose increment of venlafaxine or sertraline on Day 7 or Day 5, respectively;
(9) Subjects are required to abstain from blood donation during the study and for 30 days following the follow-up visit; and
(10) Subjects are required to follow the informed consent form (ICF) and the clinic code of conduct.

### Test Product(s), Dose, and Mode of Administration:

Subjects are randomized into 1 of 2 arms:
Arm 1: racemic ketamine 60 mg with venlafaxine
Arm 2: racemic ketamine 60 mg with sertraline

On Day 1, racemic ketamine 60 mg is intranasally administered as 4 sprays (in total) using 2 disposable bi-dose devices. Two sprays (1 spray per nostril) are administered from the first device. After approximately 5 minutes, two additional sprays (1 spray per nostril) are administered from the second disposable device. Subjects may not blow their nose for 1 hour following dosing with racemic ketamine.

Subjects are instructed to gently blow their nose prior to intranasal racemic ketamine administration. Details, including the required standard position of the subject's head during drug administration and the instructions given to subjects regarding sniffing following administration of the sprays, are outlined in a study-specific procedure.

### Arm 1

From Day 3 to Day 6, subjects are given single oral doses of 75 mg venlafaxine (1×75 mg venlafaxine capsule) and the venlafaxine dose is escalated to 150 mg (2×75 mg venlafaxine capsules) from Day 7 to Day 10.

### Arm 2

On Day 3 and Day 4, subjects are administered a single oral dose of 50 mg sertraline (1×50 mg sertraline capsule) and the sertraline dose is escalated to 100 mg (2×50 mg sertraline capsules) from Day 5 to Day 10.

On each day from Day 3 to Day 11, venlafaxine or sertraline is administered approximately 45 minutes after starting a meal and subjects will have approximately 30 minutes to complete the whole meal. Any residual amount of food is recorded. Meals served on the morning of Day 10 and Day 11 are standardized and similar in composition. The study drug is administered with about 240 mL of water and must be swallowed whole within 5 minutes. Time of dosing is set as the first capsule administration, when applicable.

On Day 11, 150 mg venlafaxine or 100 mg sertraline is administered first and the second dose of racemic ketamine 60 mg is given at approximately the same time as on Day 1 (approximately 4 hours following venlafaxine or sertraline dosing), following the same procedure for administration of the nasal sprays. Time of racemic ketamine dosing is set as the first spray administration. Nasal cavity check is done after each racemic ketamine dosing in order to confirm proper inhalations.

### Criteria for Evaluation: Pharmacokinetics

The PK parameters to be evaluated in this study for ketamine, norketamine, and hydroxyketamine, with and without sertraline or venlafaxine, as applicable, include the following:
(1) Cₘₐₓ: maximum observed plasma concentration;
(2) AUC_{0-inf}: area under the plasma concentration-time curve extrapolated to infinity;
(3) AUC₀₋ₜ: area under the plasma concentration-time curve from 0 to last measurable concentration (for doses 1 and 3);
(4) Tₘₐₓ: time to maximum observed plasma concentration;
(5) kₑₗ: first-order rate constant associated with the terminal (log-linear) portion of the curve (for dose 3 with and without sertraline or venlafaxine);
(6) t½: apparent first-order terminal elimination half-life (calculated as 0.693/kₑₗ);
(7) CL/F: apparent clearance (total ketamine only); and
(8) V_{d}/F: apparent volume of distribution (total ketamine only).

Other PK parameters for venlafaxine and sertraline and their corresponding metabolites will include the following:
(9) Cₘₐₓ,
(10) AUC_{τ}: area under the plasma concentration-time curve for the dosing interval;
(11) CLₛₛ: steady-state clearance; and
(12) Tₘₐₓ.

### Criteria for Evaluation: Pharmacodynamics

The PD parameters to be evaluated in this study include:
(13) blood pressure
   maximum change from baseline (CFBₘₐₓ) in blood pressure
   time to CFBₘₐₓ in blood pressure

Other PD endpoints may be evaluated as appropriate.

### Criteria for Evaluation: Safety

Safety endpoints include:
(14) AEs (type, incidence, and severity);
(15) Vital signs (blood pressure, respiratory rate, heart rate, oxygen saturation, and oral temperature);
(16) 12-lead electrocardiogram (electrocardiogram; heart rate and the PR, QRS, QT, and QTc intervals);
(17) Clinical laboratory tests;
(18) Columbia-Suicide Severity Rating Scale (C-SSRS);
(19) Clinician-Administered Dissociative State Scale (CADSS);
(20) physical examination; and
(21) Nasal cavity examination.

### Stopping Criteria

Any subject who voluntarily withdraws consent or is discontinued (e.g., because of an adverse event) from the study prior to completion is considered as withdrawn from the study. Subjects may be discontinued from the study under any of the following circumstances:
(1) Occurrence of intolerable AE, as assessed by the investigator or designee;
(2) Clinically significant abnormality on vital signs, electrocardiogram, clinical laboratory, or physical examination assessments, as assessed by the investigator or designee;
(3) Withdrawal of consent;
(4) Lost to follow-up;
(5) Administrative reasons;
(6) Sponsor decision;
(7) Major violation of the protocol;
(8) If, in the opinion of the qualified investigator, it is in the best interest of the subject;
(9) Pregnancy;
(10) Noncompliance with study requirements and restrictions (e.g., positive urine cotinine test at any study visit, use of a concomitant medication); and
(11) Termination of the study,

Subjects experiencing emesis after dosing with venlafaxine or sertraline may be withdrawn. An evaluation is done on a case-by-case basis.

When an event such as a family emergency, a transient intercurrent illness (such as a cold) unrelated to study drug, or a remediable act of noncompliance prevents a subject from participating in a scheduled visit, but the subject wishes to continue in the study, with the agreement of the investigator, the research site staff may attempt to reschedule the visit if feasible for the study and retain the subject in the study.

If a subject is prematurely discontinued from participation in the study for any reason after drug administration, the investigator or designee must make every effort to perform the assessments scheduled for the Follow-up visit. Replacement subjects may be added at the sponsor's discretion, in agreement with the principal investigator.

### Statistical Methods

### Treatment Population Definitions:

Randomized Population: All subjects who are randomized into one of the treatment arms
Safety Population: All randomized subjects who receive any study drug
Pharmacokinetic (PK) Population: All subjects in the Safety Population who receive at least 1 dose of the study drug, have evaluable PK data and have not experienced any protocol deviations or other circumstances to exclude the subject from the PK population.
Bioavailability Population: All subjects in the Safety Population who complete all treatments for at least one PK parameter. Subjects with more than 3 consecutive samples missing or more than 5 samples missing in total will not be included in the Bioavailability Population
Pharmacodynamic (PD) Population: All subjects in the Safety Population who have any post-baseline BP measurements.

### Analysis of Safety Assessments

Safety analysis is done using the Safety Population. The incidence of adverse events, adverse events resulting in discontinuation, and serious adverse events are summarized by treatment arm, showing the number and percentage of subjects who experienced at least 1 adverse event. These summaries are presented by system organ class and preferred term using the Medical Dictionary for Regulatory Activities (MedDRA, version 22.0 or higher), and by maximum severity and relationship to the study treatment.

Laboratory data are summarized by the laboratory panel, laboratory test, treatment arm and visit. Laboratory abnormalities are summarized by treatment arm and subject in a listing. For each laboratory panel, laboratory data are listed by treatment arm and subject. Vital signs (BP, respiratory rate, heart rate, oxygen saturation) are summarized at each time point by treatment for absolute values and change from baseline. Special consideration is given to blood pressure as this is a known factor in ketamine safety.

12-Lead electrocardiogram data (absolute values and change from baseline in ventricular heart rate and the PR, QRS, QT, and QTc intervals) are summarized by treatment and time point using descriptive statistics. Frequencies (numbers and percentages) are calculated for the overall evaluation by treatment and time point.

The Clinician-Administered Dissociative State Scale (CADSS) is summarized by treatment and time point for the following summary scores:
Subject-rated subscale (sum of Item 1 to 19)
Observer-rated subscale (sum of Item 20 to 23)
Total score (sum of Item 1 to 23)

In addition, responses to the individual CADSS items and summary scores are listed.

### The Columbia-Suicide Severity Rating Scale (C-SSRS).

Any finding or absence of finding relative to each subject's baseline physical examination and nasal cavity examination are documented. Any abnormal finding noted after dosing is documented as an adverse event if judged as a clinically significant change from baseline.

### Analysis of Pharmacokinetics

Pharmacokinetic descriptive statistics are done using the PK Population. The Bioavailability Population are used for inferential analysis. Treatment is defined as Day 1 Ketamine 60 mg, Day 10 Venlafaxine 150 mg or Sertraline 100 mg, Day 11 Ketamine 60 mg, and Day 11 Venlafaxine 150 mg or Sertraline 100 mg.

Plasma concentration data for ketamine, norketamine, and hydroxyketamine are summarized using descriptive statistics for each treatment and time point. Pharmacokinetic parameters are derived using noncompartmental methods for all analytes. Similar analysis is performed for venlafaxine and sertraline and their metabolites (N-desmethylvenlafaxine and O-desmethylvenlafaxine, N-desmethylsertraline).

Descriptive statistics, including n, mean, standard deviation (SD), coefficient of variation (CV), minimum, median, and maximum are summarized by treatment and time point. PK parameters are summarized by treatment. Graphs of the concentration (original and log-transformed) versus time are generated. Concentrations below the limit of quantification (BLQ) are set to zero for the generation of summary statistics and mean concentration time plots

For the calculation of the PK parameters, concentration time data is treated as follows: BLQ concentrations before the first quantifiable concentration is set to zero; BLQ concentrations after the first quantifiable concentration are treated as missing; and predose sampling times relative to dosing are set to zero. The PK blood sampling collected at follow-up visit will not be used for PK parameters calculation. Descriptive statistics, including n, mean, SD, geometric mean, geometric CV, minimum, median, and maximum are calculated by dose level for all PK parameters except Tₘₐₓ, t½, and λ. Tₘₐₓ data are summarized with n, minimum, median, maximum, and quartiles 1 and 3. The t½ and λ data are summarized with n, mean, SD, CV, minimum, median, and maximum.

Pharmacokinetic profiles are analyzed for all subjects, even if some PK sampling time points are missed. The PK parameters are subject to quality control criteria. If quality control criteria are met, the PK parameters are used in analyses and models. Quality control criteria for PK are sufficient to eliminate unreliable data.

Comparison of Cₘₐₓ, AUC_{0-inf}, and CL/F is performed for ketamine alone and in combination with venlafaxine or sertraline. Comparison of Cₘₐₓ, AUC_{τ}, and CLₛₛ is performed for venlafaxine and sertraline with and without ketamine. The comparison is performed using a mixed-effect analysis of variance (ANOVA) model with the logarithm of the parameter as the outcome. Treatment is included as a fixed effect and subject as a random effect.

Metabolic ratios are calculated for ketamine conversion to norketamine and hydroxyketamine using molecular-weight-adjusted metabolite-to-parent ratios. Conversion of venlafaxine and sertraline to their corresponding metabolites, individually and in combination with ketamine, will also be evaluated to provide more insight into the metabolic interaction through different pathways.

### Analysis of Pharmacodynamic Measures

The analysis of PD endpoints are performed using the PD Population. The PD endpoints will include CFBₘₐₓ and time CFBₘₐₓ.

Blood pressure changes are analyzed as CFB on dosing Days 1, 10, and 11. Statistical models are used to compare effect of ketamine or venlafaxine/sertraline alone with the effect of drug combination for each time point and relevant parameters.

The possible PD interaction between ketamine and sertraline and between ketamine and venlafaxine is explored. Pharmacodynamic data at each time point is summarized by descriptive statistics and presented graphically (where appropriate). Derived endpoints are summarized using descriptive statistics.

### Example 3. A Double Blind, Placebo-Controlled Phase 3 Study to Assess the Rapid Response Efficacy, Safety, and Tolerability of Intranasal Racemic Ketamine Administered to Adults with Moderate or Severe Major Depressive Disorder

This example describes a Phase 3, multicenter double blind placebo controlled study to assess the rapid response efficacy, safety, and tolerability of intranasal racemic ketamine in adult subjects with moderate to severe MDD.

### Study Overview

This Phase 3, randomized, double-blind, placebo-controlled multicenter, study in adult subjects diagnosed with MDD will evaluate the rapid response efficacy, safety, and tolerability of intra-nasally administered racemic ketamine. Approximately 240 subjects will be randomized 1:1 to receive either intranasal racemic ketamine (90 mg) or matching placebo.

The study is designed to assess acute rapid response therapy in MDD when co-administered with standard antidepressant treatment. Standard antidepressant treatment includes co-initiation of SSRI or SNRI, or optimization of current antidepressant regimen.

Each subject will participate in a 2- to 7-day screening phase, a 16-day treatment phase including antidepressant standard of care during which study drug will be administered 2 times per week, the standard antidepressant regimen is continued throughout, and a 4-week follow-up phase for a total of approximately 6 weeks of study participation. Subjects will be treated in the clinic as outpatients, and will return to the clinic to receive study drug and to undergo study assessments 2 times per week until Day 16. Subjects will be evaluated for rapid response efficacy using multiple psychometric scales and for safety using clinical laboratory assessments, electrocardiograms (ECGs), vital signs, and physical examinations. After the last dose of study drug, subjects will continue to be monitored for 4 weeks, including 4 in-person safety follow-up visits on Days 23, 30, 37, and 44 (all±1).

### Objectives:

The primary objective of this study is to evaluate the rapid response efficacy of intranasal racemic ketamine on symptoms of depression in adults with moderate or severe major depressive disorder (MDD).

The secondary objectives are:
(1) To evaluate the timing of onset of rapid response and, for responders, the persistence of benefit of intranasal racemic ketamine in adults with MDD; and
(2) To evaluate the safety and tolerability of intranasal racemic ketamine in adults with MDD.

### Number of Subjects

Approximately 240 randomized subjects are planned including 120 subjects per arm.

### Criteria for Inclusion:

Subjects must fulfill all of the following requirements to enter the study:
(1) The subject is able to speak, read, and understand English and/or the language of the investigative staff to sufficiently understand the nature of the study, to provide written informed consent, and to allow the completion of all study assessments.
(2) Subject is 18 to 65 years of age at the time of informed consent.
(3) Male subjects who are sexually active must agree to abstain from sexual activity or be willing to use medically acceptable contraception if they become sexually active from time of consent and for 3 months after the last dose of study drug.
(4) Female subjects of childbearing potential must have a negative serum pregnancy test at screening, and must not be breastfeeding or lactating. Females must be willing to abstain from sexual activity or use medically accepted contraception if they are sexually active from time of consent and for 1 month after the last dose of study drug.
(5) The subject meets Diagnostic and Statistical Manual of Mental Disorders, Fifth Edition (DSM-5) criteria for a diagnosis of current MDD (unipolar without psychotic features), with symptoms present for at least 4 weeks, based on psychiatric intake and confirmed by the Mini International Psychiatric Interview Version 7.02 (MINI).
(6) The subject has a Montgomery Åsberg Depression Rating Scale (MADRS) total score of ≥28 predose on Day 1.
(7) The subject is willing and able to take prescribed non-investigational antidepressant therapy for at least the duration of the study.
(8) The subject is willing to avoid the use of alcohol, CBD oil, and recreational drugs, as well as specific therapies prohibited by the protocol, from screening through the last study visit (Day 44).
(9) The subject is able to complete intranasal administration of study drug.

### Exclusion Criteria

The presence of any of the following criteria excludes a subject from participating in the study:
(1) Subjects who have ongoing sequelae from prior COVID illness, or subjects who have either documented COVID infection or symptoms suggestive of recent COVID infection within 1 month of screening.
(2) The subject has a lifetime diagnosis of bipolar disorder, any mood disorder with psychotic features, schizophrenia or other psychotic disorder, obsessive-compulsive disorder, or antisocial personality disorder, as confirmed by the MINI. (Note subjects who have post-traumatic stress disorder and generalized anxiety disorder (GAD)/panic disorder are not necessarily excluded as long as MDD is the most prominent diagnosis.)
(3) In the investigator's opinion, the subject has chronic, refractory treatment-resistant depression from >4 adequate therapeutic trials of antidepressants (with or without adjuvants and/or electroconvulsive therapy [ECT]) as confirmed by ATRQ.
(4) In the investigator's opinion, the subject has a current diagnosis of borderline personality disorder or, if the subject has not met full diagnostic criteria for borderline personality disorder within the last 5 years, the subject has a history of recurrent non-suicidal self-injury, or self-mutilating behavior.
(5) In the investigator's opinion, the subject is judged to be an acute suicide risk, scoring ≥4 on MADRS item 10 in the past month.
(6) Suicide attempt within the last 12 months, on the C-SSRS, and a score of 2, 3, or 4 for Actual Lethality/Medical Damage.
(7) The subject is diagnosed with intellectual disability, a neurocognitive disorder including dementia, or has a history of moderate or severe traumatic brain injury. Mild traumatic brain injuries are not necessarily exclusionary provided the investigator believes current symptoms would not interfere with conduct or interpretation of safety and/or efficacy assessments.
(8) The subject has a history or current finding of any clinically significant, hematological, hepatic, respiratory, renal, neurological, known positive human immunodeficiency virus (HIV) infection, gastrointestinal disorder, or other disease that might confound the results of safety assessments conducted in the study.
(9) The subject has a history of seizures (other than childhood febrile seizures).
(10) The subject has a body mass index (BMI)>40 or <18 at screening.
(11) The subject has known, uncontrolled hypertension or blood pressure (BP) that, in the investigator's judgment, should exclude the subject at screening or baseline (BP may be repeated as per the site's standard operating procedures [SOPs]).
(12) The subject has a known history or current finding of cardiovascular disease, advanced arteriosclerosis, structural cardiac abnormality, cardiomyopathy, serious heart rhythm abnormalities, coronary artery disease, congenital heart disease, ischemic heart disease, cardiac insufficiency, supraventricular and ventricular heart rhythm disorder, prolonged QT syndrome (i.e., QTcF >450 msec) and associated risk factors (i.e., hypokalemia, family history of long QT syndrome), syncope, cardiac conduction problems (e.g., clinically significant heart block), exercise-related cardiac events including syncope and pre-syncope, clinically significant bradycardia, or other serious cardiac problems.
(13) The subject has a known family history of sudden cardiac death or ventricular arrhythmia.
(14) The subject has any clinically significant abnormality on 12-lead ECG performed at screening or baseline such as serious arrhythmia, cardiac conduction problems, or other abnormalities deemed to be a potential safety issue.
(15) The subject has a concurrent chronic or acute illness, disability, or other condition (e.g., narcolepsy) that might confound the results of safety assessments conducted in the study.
(16) The subject has any medical condition that could interfere with the absorption of IN ketamine (e.g., nasal polyps, clinically significant deviated septum [corrected or persistent], or other physical abnormalities of the nose).
(17) The subject has symptomatic or uncontrolled hyper- or hypothyroidism or has had changes to their treatment for hyper- or hypothyroidism within 90 days prior to Day 1.
(18) The subject meets the DSM-5 criteria for moderate or severe substance use disorder in the 6 months prior to screening, OR in the investigator's opinion, is at risk of withdrawal from substance use (e.g., opiate or alcohol dependent), OR has a lifetime history of ketamine, phencyclidine, lysergic acid diethylamide, or 4-methylenedioxy-methamphetamine hallucinogen-related use disorders. Nicotine use disorder is permitted.
(19) The subject requires daily benzodiazepine use of >2 mg of lorazepam or dose equivalent.
(20) The subject has a positive urine test for phencyclidine (PCP), cocaine, or amphetamines (inclusive of amphetamine, methamphetamine, and 3,4-methylenedioxymethamphetamine [MDMA]) at screening.
(21) The subject has positive hepatitis B, hepatitis C, or HIV results at screening.
(22) The subject has any history of using ketamine or esketamine for any psychiatric treatment.
(23) The subject has known or suspected intolerance or hypersensitivity to the investigational product(s), closely related compounds, or any ingredients.
(24) In the investigator's opinion, the subject has any clinically significant laboratory abnormality including an abnormality that indicates clinically significant hematologic, hepatobiliary, or renal disease. Note, any screening abnormality must be discussed with the Medical Monitor for approval prior to randomization.
(25) The subject has received an investigational product, including investigational vaccines, within 6 months prior to the first dose of study drug.
(26) The subject was previously enrolled in the current study. Subjects previously screened for, but not randomized in, the current study may be rescreened with approval from the study medical monitor.
(27) The subject does not meet or is not willing to comply with the requirements related to prohibited and restricted medications and therapies, as well as required washout periods prior to participation. Prohibited medications and therapies include, but are not limited to, monoamine oxidase inhibitors (MAOIs), opioids or drugs with activity at the opioid receptor, psychostimulants, lamotrigine, N-methyl-D-aspartate (NMDA) receptor modulators, magnesium, electroconvulsive therapy (ECT), transcranial magnetic stimulation (TMS) or any medication/therapy that might confound the results of safety assessments conducted in the study. Subjects who have received any of these prohibited medications within 2 weeks of screening are excluded from the study. Potent CYP 3A4 inhibitors (including nefazodone and fluvoxamine) are not permitted within 1 week of first dose and until at least 1 day after the last dose. Potent CYP 3A4 inducers (including St. John's wort) are not permitted within 30 days of first dose and until at least 1 day after the last dose. Subjects who have recently discontinued lithium or calcium channel blockers within 3 months prior to screening are also excluded.
(28) The subject is an employee of the sponsor, clinical research organization, or research site personnel directly affiliated with this study or their immediate family member defined as a spouse, parent, child, or sibling, whether biological or legally adopted.
(29) The subject has pending legal charges or is on probation.
(30) The subject, in the investigator's opinion, is considered unsuitable or unlikely to comply with the study protocol for any reason.
(31) The subject is legally incapacitated, has been involuntarily hospitalized in the past year, or has another significant mental health issue, physical issue, or life circumstance that could interfere with the conduct or interpretation of study evaluations.

### Concomitant Treatment

Evidence-/empirically based optimization of current antidepressant treatment or initiation of a new SSRI or SNRI antidepressant is required for all subjects during the study, at study start. Standard of care (SOC) should remain stable during the 16-day treatment phase. In addition, subjects are permitted to be participating in SOC psychotherapy, including behavioral therapy. Benzodiazepine (dose equivalent to ≤2 mg/day of lorazepam) medications may be taken on a limited basis as needed, as defined in the protocol restricted medications section, but should not be taken within 24 hours prior to study medication dosing and assessments. Short acting non-benzodiazepine hypnotics (e.g. zolpidem, zaleplon) are permitted, but may never be taken within 10 hours prior to study medication dosing.

### Criteria for Evaluation:

### Primary Efficacy Endpoint:

The primary efficacy endpoint will be the change from baseline in the MADRS total score at 24 hours following the initial dose.

### Secondary Efficacy Endpoints:

The key secondary endpoints (in rank order) will be the change from baseline in:
(1) MADRS total score at Day 16
(2) Clinical Global Impression Severity (CGIS) at 24 hours
(3) Patient Global Impression Severity (PGIS) at 24 hours
(4) CGIS at Day 16
(5) PGIS at Day 16

### Additional Secondary Endpoints:

The additional secondary endpoints will be the change from baseline in the following:
(1) Number and % of MADRS responders (total score ≥50% reduction from baseline) at each efficacy timepoint
(2) Number and % of persistent MADRS responders from onset at each timepoint and maintained through Day 16
(3) Number and % of persistent MADRS responders from onset at each timepoint and maintained through Week 6
(4) MADRS remission (total score ≤12) at each efficacy timepoint
(5) MADRS Total Score at week 6
(6) CGIS at week 6
(7) PGIS at week 6
(8) Clinical Global Impression Change (CGIC) at Day 16 and week 6
(9) Patient Global Impression Change (PGIC) at Day 16 and week 6
(10) Sheehan Disability Scale at Day 16 and week 6
(11) Post-treatment Phase-time to relapse for responders in the treatment phase, who subsequently relapse during the 4 week safety follow-up. (Relapse is defined as MADRS total score ≥22 for two consecutive weeks)

### Safety Endpoints:

The safety and tolerability of intranasal racemic ketamine will be assessed by:
(1) The frequency and severity of treatment-emergent adverse events (TEAEs)
(2) Frequencies of new or worsening clinically significant laboratory, vital signs, ECGs, or physical examination abnormalities
(3) Clinician-Administered Dissociative States Scale (CADSS)
(4) Modified Observer's Assessment of Alertness/Sedation (MOAA/S) Scale
(5) Columbia Suicide Severity Rating Scale (C-SSRS)
(6) Physician Withdrawal Checklist 20-item (PWC-20)

### Statistical Methods

For the analysis of the primary efficacy endpoint, the change in the MADRS total score from baseline at 24 hours following the initial dose will be analyzed using an ANCOVA model including baseline MADRS total score as a covariate, treatment as fixed effect and a random subject effect. A hierarchical testing procedure will be used to maintain the experiment-wise type 1 error rate at 5%. Endpoints starting with the primary endpoint, will be tested in the pre-specified order of importance. Once a p-value greater than 0.05 is obtained, all subsequent hypothesis testing will be stopped. Further details of the statistical analyses, including secondary efficacy, safety, and sensitivity analyses, will be described in the protocol and/or the Statistical Analysis Plan (SAP).

### Sample Size Determination

The sample size planned is calculated assuming an effect size of 0.45, a 2-sided significance level of 0.05, and assuming approximately a 15% dropout rate. Based on these assumptions, 120 subjects will need to be randomized to each treatment group to achieve 90% power. The effect size used in this calculation was based on results of previous ketamine studies and on clinical judgment.

### Study and Treatment Duration

The sequence and maximum duration of the study periods will be as follows:
a 2- to 7-day screening phase
a 16-day treatment phase of investigational study drug which is co-administered with a standard of care (SOC) antidepressant regimen
a 4-week safety follow up during which SOC is continued and optimized as clinically warranted

Therefore, the maximum study duration for each subject is approximately 7 weeks.

All subjects will be referred for follow up post study; this includes subjects who do not qualify for study, subjects who discontinue participation, and those subjects who complete the study.

### Example 4. A 2-Part Phase 2 Study to Assess the Efficacy, Safety, and Tolerability of Intranasal Racemic Ketamine Administered to Adults with Major Depressive Disorder at Imminent Risk of Suicide

This example describes a Phase 2, multicenter, 2-part study to determine the efficacy, safety, and tolerability of intranasally (IN) administered racemic ketamine plus SOC in adult subjects diagnosed with MDD at imminent risk of suicide.

### Study Overview

This Phase 2, multicenter, 2-part study in adult subjects diagnosed with MDD at imminent risk of suicide will evaluate the efficacy, safety, and tolerability of intranasally (IN) administered racemic ketamine plus SOC. In Part 1, 16 subjects will receive open-label racemic ketamine (90 mg). Part 2 of the study is double-blind and 120 subjects will be randomized 1:1 to receive either racemic ketamine (90 mg) or matching placebo.

The study schedule is identical for Parts 1 and 2. After admission to the emergency room or hospital, each subject will participate in a 1 to 2 day screening phase, a 16-day treatment phase including SOC during which study drug will be administered 2 times per week, and a 2-week safety follow-up phase for a total of up to 5 weeks of study participation. Subjects will be treated as inpatients for approximately 7 days (including screening), and assuming the subject meets readiness for discharge criteria, will be discharged on Day 6 to continue the trial as outpatients, provided it is clinically appropriate to do so. Subjects will return to the clinic to receive study drug and to undergo study assessments 2 times per week until Day 16. Subjects will be evaluated for efficacy using multiple psychometric scales and for safety using clinical laboratory assessments, electrocardiograms (ECGs), vital signs, and physical examinations. After the last dose of study drug, subjects will continue to be monitored for safety for 2 weeks, including 4 in-person safety follow-up visits on Days 19, 22, 25/26, and 29/30.

### Objectives:

The primary objective of this study is to evaluate the efficacy of racemic ketamine plus standard of care (SOC) on symptoms of depression in adults with major depressive disorder (MDD) who are at imminent risk of suicide

The secondary objectives are:
(1) To evaluate the efficacy of racemic ketamine plus SOC on symptoms of suicidality in adults with MDD who are at imminent risk of suicide; and
(2) To evaluate the safety and tolerability of racemic ketamine plus SOC in adults with MDD who are at imminent risk of suicide.

The exploratory objective is to evaluate the psychometric properties of the Sheehan-Suicidality Tracking Scale (STS)-Clinically Meaningful Change Measure (CMCM).

### Number of Subjects

Part 1: 16 subjects are planned. Part 2: Approximately 220 randomized subjects are planned including 110 subjects per arm.

### Criteria for Inclusion:

Subjects must fulfill all of the following requirements to enter the study:
(1) The subject is able to speak, read, and understand English and/or the language of the investigative staff to sufficiently understand the nature of the study, to provide written informed consent, and to allow the completion of all study assessments.
(2) Subject is 18 to 65 years of age at the time of informed consent.
(3) Male subjects who are sexually active must agree to abstain from sexual activity or be willing to use medically acceptable contraception if they become sexually active from time of consent and for 3 months after the last dose of study drug.
(4) Female subjects of childbearing potential must have a negative serum pregnancy test at screening, and must not be breastfeeding or lactating. Females must be willing to abstain from sexual activity or use medically accepted contraception if they are sexually active from time of consent and for 1 month after the last dose of study drug.
(5) The subject meets Diagnostic and Statistical Manual of Mental Disorders, Fifth Edition (DSM-5) criteria for a diagnosis of current MDD (unipolar without psychotic features), with symptoms present for at least 4 weeks, based on psychiatric intake and confirmed by the Mini International Psychiatric Interview Version 7.02 for Suicidality Disorders (MINI).
(6) The subject has a Montgomery Åsberg Depression Ration Scale (MADRS) Total of ≥28 units predose on Day 1.
(7) The subject has a score of 5 units or 6 units on item 10 of the MADRS.
(8) The subject has a Clinical Global Impression (CGI) of Severity for Suicidal Ideation and Behavior (SI/B) (CGIS-SI/B) score of >4 predose on Day 1.
(9) In the investigator's opinion, the subject requires psychiatric hospitalization due to significant risk of suicide, has >15 units on the STS-CMCM total score, and a score of 6-9 units (inclusive) on the STS-CMCM Clinician judgment of subject's risk of a suicide attempt or death by suicide at this time predose on Day 1.
(10) In the investigator's opinion, the subject has current suicidal ideation with intent, which is confirmed by the MINI Suicidality Module at screening and baseline, specifically a positive response related to present symptoms on Question B3, as well as a positive response related to symptoms within the past 24 hours on Question B10 or B11. NOTE: Although the full MINI assessment is not conducted at baseline, the investigator must confirm that the subject continues to meet this criterion prior to enrollment (i.e., Questions B3, B10, and B11 should be revisited with the subject predose at baseline).
(11) In the investigator's opinion, if the imminent suicidality is based upon a precipitating event (i.e., a clearly identifiable situational stressor that contributes to initiation or exacerbation of the subject's current suicidality), the active suicidality must be present for >72 hours.
(12) The subject has a history of previous suicide attempt(s), as confirmed on the C-SSRS with a history of at least one actual attempt, or if the attempt was interrupted or aborted, is judged to have been serious in intent in the investigator's opinion.
(13) As part of SOC treatment, the subject agrees to be hospitalized voluntarily for a recommended period of approximately 7 days (screening to Day 6), and fully understands that the duration of hospitalization may be longer if clinically indicated (i.e., he/she is not safe to be discharged on Day 6).
(14) The subject is willing and able to take prescribed non-investigational antidepressant therapy(ies) at investigator's discretion for at least the duration of the study.
(15) The subject is willing and able to maintain other existing treatments and to avoid the use of alcohol and recreational drugs, as well as specific therapies prohibited by the protocol, from screening through the last study visit (Day 29/30). Subjects with acute alcohol intoxication should not be screened (but can be screened once sober); subjects suspected of intoxication can be confirmed by BAC or breathalyzer.
(16) The subject is able to complete IN administration of study drug.

### Exclusion Criteria

The presence of any of the following criteria excludes a subject from participating in the study: The presence of any of the following criteria excludes a subject from participating in the study:
(1) Subjects who have ongoing sequelae from prior Coronavirus disease of 2019 (COVID) illness, or subjects who have either documented COVID infection or symptoms suggestive of recent COVID infection within 1 month of screening.
(2) The subject has a lifetime diagnosis of bipolar disorder, any mood disorder with psychotic features, schizophrenia or other psychotic disorder, obsessive compulsive disorder, or antisocial personality disorder, as confirmed by the MINI. (NOTE: subjects who have post-traumatic stress disorder and generalized anxiety disorder (GAD)/panic disorder are not necessarily excluded as long as MDD is the most prominent diagnosis.) Subject has impulse attack suicidality disorder, homicidal suicidality disorder, or any suicidality disorder associated with the above diagnoses.
(3) In the investigator's opinion, the subject has chronic, refractory treatment-resistant depression from >4 adequate therapeutic trials of antidepressants (with or without adjuvants and/or electroconvulsive therapy [ECT]) as confirmed by ATRQ.
(4) In the investigator's opinion, the subject has a current diagnosis of borderline personality disorder or, if the subject has not met full diagnostic criteria for borderline personality disorder within the last 5 years, the subject has a history of recurrent non-suicidal self-injury, or self-mutilating behavior.
(5) The subject has a score of 10 units on the STS-CMCM Clinician judgment of subject's risk of a suicide attempt or death by suicide at this time (top of STS-CMCM p12).
(6) The subject is diagnosed with intellectual disability, a neurocognitive disorder including dementia, or has a history of moderate or severe traumatic brain injury. Mild traumatic brain injuries are not necessarily exclusionary provided the investigator believes current symptoms would not interfere with conduct or interpretation of safety and/or efficacy assessments.
(7) The subject has a history or current finding of any clinically significant, hematological, hepatic, respiratory, renal, neurological, known positive human immunodeficiency virus (HIV) infection, gastrointestinal disorder, or other disease that might confound the results of safety assessments conducted in the study.
(8) The subject has a history of seizures (other than childhood febrile seizures).
(9) The subject has a body mass index (BMI)>40 or <18 at screening.
(10) The subject has known, uncontrolled hypertension or blood pressure (BP) that, in the investigator's judgment, should exclude the subject at screening or baseline (BP may be repeated as per the site's standard operating procedures [SOPs]).
(11) The subject has a known history or current finding of cardiovascular disease, advanced arteriosclerosis, structural cardiac abnormality, cardiomyopathy, serious heart rhythm abnormalities, coronary artery disease, congenital heart disease, ischemic heart disease, cardiac insufficiency, supraventricular and ventricular heart rhythm disorder, prolonged QT syndrome (i.e., QTcF >450 msec) and associated risk factors (i.e., hypokalemia, family history of long QT syndrome), syncope, cardiac conduction problems (e.g., clinically significant heart block), exercise-related cardiac events including syncope and pre-syncope, clinically significant bradycardia, or other serious cardiac problems.
(12) The subject has a known family history of sudden cardiac death or ventricular arrhythmia.
(13) The subject has any clinically significant abnormality on 12-lead ECG performed at screening or baseline such as serious arrhythmia, cardiac conduction problems, or other abnormalities deemed to be a potential safety issue.
(14) The subject has a concurrent chronic or acute illness, disability, or other condition (e.g., narcolepsy) that might confound the results of safety assessments conducted in the study.
(15) The subject has any medical condition that could interfere with the absorption of IN ketamine (e.g., nasal polyps, clinically significant deviated septum [corrected or persistent], or other physical abnormalities of the nose).
(16) The subject has symptomatic or uncontrolled hyper- or hypothyroidism or has had changes to their treatment for hyper or hypothyroidism within 90 days prior to Day 1.
(17) The subject meets the DSM-5 criteria for moderate or severe substance use disorder in the 6 months prior to screening, OR in the investigator's opinion, is at risk of withdrawal from substance use (e.g., opiate or alcohol dependent), OR has a lifetime history of ketamine, phencyclidine, lysergic acid diethylamide, or 4-methylenedioxy-methamphetamine hallucinogen-related use disorders. Nicotine use disorder is permitted.
(18) The subject has a positive urine test for phencyclidine (PCP), cocaine, or amphetamines (inclusive of amphetamine, methamphetamine [mAMP], and 3, 4 methylenedioxy-methamphetamine [MDMA]) at screening.
(19) The subject has positive hepatitis B, hepatitis C, or HIV results at screening.
(20) The subject has any history of using ketamine or esketamine for any psychiatric treatment.
(21) The subject has known or suspected intolerance or hypersensitivity to the investigational product(s), closely related compounds, or any ingredients.
(22) In the investigator's opinion, the subject has any clinically significant laboratory abnormality including an abnormality that indicates clinically significant hematologic, hepatobiliary, or renal disease.
(23) The subject has received an investigational product, including vaccines, within 30 days prior to the first dose of study drug.
(24) The subject was previously enrolled in the current study. Subjects previously screened for, but not randomized in, the current study may be rescreened with approval from the study medical monitor.
(25) The subject does not meet or is not willing to comply with the requirements listed in the protocol related to prohibited and restricted medications and therapies, as well as required washout periods prior to participation. Prohibited medications and therapies include, but are not limited to, monoamine oxidase inhibitors (MAOIs), opioids or drugs with activity at the opioid receptor, psychostimulants, lamotrigine, N-methyl-D-aspartate (NMDA) receptor modulators, magnesium, ECT therapy, transcranial magnetic stimulation (TMS) or any medication that might confound the results of safety assessments conducted in the study. Subjects who have received any of these prohibited medications within 2 weeks of screening are excluded from the study. Potent CYP 3A4 inhibitors are not permitted within 1 week of first dose and until at least 1 day after the last dose. Potent CYP 3A4 inducers, including nefazodone and fluvoxamine, are not permitted 1 week of first dose and until at least 1 day after the last dose. Potent CYP 3A4 inducers, including St. John's wort, are not permitted within 30 days of first dose and until at least 1 day after the last dose. Subjects who have recently discontinued lithium or calcium channel blockers within 3 months prior to screening are also excluded.
(26) The subject is an employee of the sponsor, clinical research organization, or research site personnel directly affiliated with this study or their immediate family member defined as a spouse, parent, child, or sibling, whether biological or legally adopted.
(27) The subject has pending legal charges or is on probation.
(28) The subject, in the investigator's opinion, is considered unsuitable or unlikely to comply with the study protocol for any reason.
(29) The subject is legally incapacitated, has been involuntarily hospitalized in the past year, or has another significant mental health issue, physical issue, or life circumstance that could interfere with the conduct or interpretation of study evaluations.

### Concomitant Treatment

SOC is required for all subjects during the study which should include evidence-/empirically based antidepressant options at the investigator's discretion. In addition, subjects are permitted to be participating in SOC psychotherapy, including behavioral therapy. Benzodiazepine (dose equivalent to ≤6 mg/day of lorazepam) medications may be taken on a limited basis as needed, as defined in the protocol restricted medications section, but may never be taken within 24 hours prior to study medication dosing and assessments. Short acting non-benzodiazepine hypnotics (e.g. zolpidem, zaleplon) are permitted.

### Criteria for Evaluation:

### Primary Efficacy Endpoint:

The primary efficacy endpoint is the change from baseline in the MADRS Total at 24 hours following the initial dose.

### Secondary Efficacy Endpoints:

The secondary endpoints are the change from baseline at 24 hours, as well as at Day 16, in the following:
(1) PGIS-SI/B (Day 16 only)
(2) S-STS total score (Day 16 only)
(3) CGI of Change in SI/B (CGIC-SI/B)
(4) PGI of Change in SI/B (PGIC-SI/B)
(5) S-STS Clinician-Rated Global Severity of Suicidal Impulses, Thoughts, and Behaviors
(6) S-STS Clinician judgment of subject's risk of a suicide attempt or death by suicide at this time
(7) S-STS Clinician judgment of subject's likelihood (risk) of making a suicide attempt or of dying by suicide in the next 7 days
(8) S-STS Subject-rated global severity of suicidal impulses, thoughts, and behavior
(9) S-STS Subject-rated treatment needed score
(10) MADRS item 10 response rate (response≤3 units)
(11) MADRS response (total score ≥50% reduction from baseline)
(12) MADRS remission (total score ≤12)

### Exploratory Endpoint:

The exploratory endpoint is the analysis of the validity, reliability, and ability to detect change by the STS-CMCM.

### Safety Endpoints:

The safety and tolerability of IN racemic ketamine were assessed by:
(1) The frequency and severity of treatment-emergent adverse events (TEAEs)
(2) Frequencies of new or worsening clinically significant laboratory, vital signs, ECGs, or physical examination abnormalities
(3) Clinician-Administered Dissociative States Scale (CADSS)
(4) Modified Observer's Assessment of Alertness/Sedation (MOAA/S) Scale
(5) Columbia Suicide Severity Rating Scale (C-SSRS)

### Statistical Methods

Separate summaries will be prepared for the Part 1 and Part 2 results. Descriptive statistics will be used to summarize the results of Part 1. For the analysis of the Part 2 data, the primary efficacy endpoint, the change in the MADRS Total from baseline at 24 hours following the initial dose, will be analyzed using an ANCOVA model including baseline MADRS Total as a covariate, treatment and class of SOC antidepressant as fixed effects. A sensitivity analysis will be done using MADRS item 10 as a covariate. The least square (LS) means, standard error (SE) and 95% confidence interval of the treatment difference will be estimated. A sensitivity analysis will be performed by a mixed-effects model using repeated measures (MMRM) based on the observed data. The model will include baseline MADRS total score as a covariate, visit, treatment, class of SOC antidepressant, and interaction of day-by treatment as fixed effects, and a random subject effect. The within subject variance between visits will be estimated via an unstructured variance-covariance matrix. The LS means, SE, and 95% confidence intervals will be estimated for the change from baseline to each time point in MADRS total score for each treatment group. Point estimates and 95% confidence intervals for the treatment differences will be provided for each time point as well. A sensitivity analysis using ANCOVA model will be conducted similar to the primary analysis by including MADRS item 10 as a covariate. A hierarchical testing procedure will be implemented to control the type 1 error rate at 5%. The primary and key secondary endpoints will be tested at the 5% level of significance. Further details of the statistical analyses, including key secondary efficacy endpoints analyses, sensitivity analyses, subgroup analyses, secondary efficacy endpoints analyses, and safety analyses will be described in the protocol and/or the Statistical Analysis Plan (SAP) or psychometric validation SAP.

### Sample Size Determination

In Part 1, the sample size planned is 16 subjects and is viewed to be sufficient to provide meaningful assessments of variability of the efficacy endpoints which can be used in planning of future trials

In Part 2, the sample size planned is calculated assuming an effect size of 0.50, a 2-sided significance level of 0.10, and assuming approximately a 15% dropout rate. Based on these assumptions, 60 subjects will need to be randomized to each treatment group to achieve 80% power. The treatment difference and standard deviation used in this calculation were based on results of previous ketamine studies and on clinical judgment.

### Study and Treatment Duration

The sequence and maximum duration of the study periods was as follows:
a 1- to 2-day screening phase
a 16-day treatment phase
a 2-week safety follow-up phase

Therefore, the maximum study duration for each subject is approximately 5 weeks.

All subjects were referred for follow up post study; this includes subjects who do not qualify for study, subjects who discontinue participation, and those subjects who complete the study.

TABLE B
Schedule of Events - Inpatient Days (Visit 1; Screening to Day 6)

| | Inpatient Period: Visit 1 | | | | | | | |
|---|---|---|---|---|---|---|---|---|
| | | Baseline | | | | | | Extended |
| Visit Number | Screening | 1 | | | 4 | | 6 | Inpatient^{a} |
| Study Day | −2 to −1 | Dosing | 2 | 3 | Dosing | 5 | Discharge | |
| Assessments & Procedures^{b} | | | | | | | | |
| Informed consent | X | | | | | | | |
| Inclusion/exclusion criteria | X | X | | | | | | |
| Medical and psychiatric | X | | | | | | | |
| history^{c} | | | | | | | | |
| Demographics | X | | | | | | | |
| Full physical examination^{d} | X | | | | | | X | X |
| Nasal cavity examination^{e} | X | X | X | | X | X | | |
| C-SSRS | X | Xⁿ | X^{r} | Xⁿ | Xⁿ | Xⁿ | Xⁿ | X^{o} |
| Look back period | Lifetime & past | SLA | SLA | SLA | SLA | SLA | SLA | SLA |
| | month | | | | | | | |
| 12-lead ECG^{f} | X | X^{g} | | | X^{h} | | Xⁱ | |
| Vital signs^{j} | X | X^{g} | X^{k} | | X^{l} | | Xⁱ | Xⁱ |
| Urine drug screen | X | | | | | | | |
| Serum pregnancy test | X | | | | | | | |
| Clinical laboratory tests | X | X | | | | | X | |
| Viral serology tests | X | | | | | | | |
| MINI 7.02 for Suicidality | X | | | | | | | |
| Disorders | | | | | | | | |
| ATRQ | X | | | | | | | |
| MOAA/S | X | X^{s} | | | X^{s} | | | |
| CADSS | X | X^{t} | | | X^{t} | | | |
| MADRS | X | X^{m} | X^{o} | | | X^{o} | X^{o} | X^{o} |
| Look back period | Last wk | Last | SLA | | | SLA | SLA | SLA |
| | | 24 hrs/SLA | | | | | | |
| S-STS CMCM | X | X^{m} | X^{o, p} | | | X^{o, p} | X^{o, p} | X^{o, p} |
| Look back period | Last wk incl today | Last | SLA | | | SLA | SLA | SLA |
| | | 24 hrs/SLAP | | | | | | |
| CGIS-SI/B and PGIS-SI/B | X | X^{m} | X | | | X | X | X |
| Look back period | Last wk incl today | Last | SLA | | | SLA | SLA | SLA |
| | | 24 hrs/SLA | | | | | | |
| CGIC-SI/B and PGIC-SI/B | | X^{q} | X | | | X | X | X |
| Look back period | | SBL | SBL | | | SBL | SBL | SBL |
| Prior and concomitant meds | X | X | X | X | X | X | X | X |
| AE monitoring | X | X | X | X | X | X | X | X |
| Randomization | | X^{u} | | | | | | |
| Review instructions on | | X^{w} | | | | | | |
| proper use of IN device | | | | | | | | |
| Study drug administration^{v} | | X | | | X | | | |
| Readiness for discharge | | | | X | | X | X | X |
| evaluation^{x} | | | | | | | | |

Abbreviations:
AE = adverse event;
ATRQ = antidepressant treatment response questionnaire;
BP = blood pressure;
C = change;
CADSS = Clinician-Administered Dissociative States Scale;
CGIC-SI/B = Clinical Global Impression of Change in Suicidal Ideation and Behavior;
CGIS-SI/B = Clinical Global Impression of Severity of Suicidal Ideation and Behavior;
C-SSRS = Columbia Suicide Severity Rating Scale;
ECG = electrocardiogram;
IN = intranasal;
MADRS = Montgomery Åsberg Depression Rating Scale;
MINI = Mini International Psychiatric Interview Version 7.02 for Suicidality Disorders;
MOAA/S = Modified Observer's Assessment of Alertness/Sedation (Scale);
PGIC-SI/B = Patient Global Impression of Change in Suicidal Ideation and Behavior;
PGIS-SI/B = Patient Global Impression of Severity of Suicidal Ideation and Behavior;
S = severity;
SBL = since baseline;
SLA = since last assessment;
S-STS CMCM = Sheehan-Suicidality Tracking Scale - Clinically Meaningful Change Measure;
wk = week;
hrs = hours
NOTE:
If needed, time points with multiple safety assessments may use a ±15-minute window. However, the C-SSRS assessment (also a safety measure) window remains at ±30 mins for each time point due to the time required for completion.
^{a}If the inpatient period is extended past Day 6, assessments included in this column must be completed on day of discharge (which can occur between Day 7 and Day 11), if not already required for the study day in Table 3.
^{b}The assessments and procedures are listed in the preferred order of completion (top to bottom).
^{c}Medical history will consist of a complete medical history including all hospitalizations, procedures, and psychiatric history.
^{d}Measure height and weight at the time of physical examination. Height to be measured only at screening.
^{e}Nasal cavity examination will be performed before dosing of study drug and at approximately 2 hours and 24 hours postdose.
^{f}On Day 1, predose, 3 serial, resting, supine 12-lead ECGs are to be conducted within 10 minutes total time. All other ECGs are single, resting, supine 12-lead recordings. When ECGs are to be collected at the same time point as a blood collection, ECGs should be collected first.
^{g}Perform predose (×3 for ECG only) (±10 min), and 1 (±10 min), 2 (±10 min), 4 (±15 min), and 6 (±10 min) hours postdose.
^{h}Perform predose, 1, and 4 hours postdose (all ±10 min).
ⁱPerform assessment anytime.
^{j}Vital signs include temperature, BP, respiration rate, and heart rate.
^{k}Perform 24 hours after Day 1 dose (±15 min).
^{l}Perform predose, and 1, 2, 4 hours postdose (all ±10 min).
^{m}Perform predose with a 24-hour look back period and 4 (+60 min) hours postdose with an SLA look back period.
ⁿPerform predose on dosing days. On non-dosing days, perform at the approximate dosing-day time (±30 min).
^{o}Perform at the approximate dosing-day time (±30 min). On Day 2, perform 24 (+60 min) hours after
Day 1 dose.
^{p}Perform the S-STS CMCM on this day excluding pages 4-5 and page 8. (On Day 1, applies ONLY to 4-hour assessment.)
^{q}Perform only at 4 (+60 min) hours postdose. For change scales, baseline is defined as the result of the severity assessment predose on Day 1.
^{r}Perform 24 hours after the Day 1 dose (±30 min).
^{s}Perform predose, 15, 30, 45, 60, 75, and 90 (all ±5) minutes postdose, and 2 (±10 min) hours postdose; however, if scores are ≤4, perform hourly until resolution.
^{t}Perform predose, 40 (±5) minutes postdose, and 1 (±10 min) and 2 (±10 min) hours postdose; however, if total scores are >4, perform hourly until resolution.
^{u}In Part 2, randomization will occur after subject is confirmed to have met inclusion/exclusion criteria.
^{v}On Day 1, dosing is suggested to occur at 10 am (±30 min). Dosing on subsequent days is suggested to occur at approximately the same time of day (±1 hour) as the Day 1 dose. The Nasal Spray Evaluation Form is to be completed following study drug administration.
^{w}Perform predose.
^{x}Investigator determines readiness for discharge and the need to extend inpatient period. If subject is not safe to discharge by Day 11/Visit 5 (as defined by a score of 5 or higher on the S-STS Clinician Judgment of Patient Risk of Suicide Attempt or Death at this time), or needs to discontinue for any reason, the subject should be discontinued and follow Visit 8/Endpoint assessments.

### Results

Data from the first eight days (of 16 total) in two subjects is described below and in FIGS. 13A-24.

Both subjects entered the trial with severe major depressive disorder (MDD) based on pre-dosing MADRS scores of 35 units and 38 units for Subjects 1 and 2, respectively. Scores of 35 units and up are considered severe, and scores of 18-34 units indicate moderate MDD.

The MADRS Total of Subject 1 decreased from the baseline score of 35 units to 8 units at 24 hours post-administration of intranasal racemic ketamine. Similarly, the MADRS Total of Subject 2 decreased from the baseline score of 38 units to 15 units at 24 hours post-administration of intranasal racemic ketamine. Thus, both subjects demonstrated an extremely rapid, clinically significant (>50% reduction) response, going from severe MDD to remission in only 24 hours based on the MADRS Total. Intranasal racemic ketamine also delivers a sustained response through the next dosing, and after receiving the second dose on Day 4, symptoms were further reduced, with Subject 2 having a MADRS Total of 16 units on Day 8, just above a remission score. See FIGS. 13A-13B.

The MADRS item 10 (suicidality)(range 0-6 units) score of Subject 1 decreased from the baseline score of 5 units to 0 units at 24 hours post-administration of intranasal racemic ketamine. The MADRS item 10 score also decreased from the baseline score of 5 units pre-dose to 0 units at 24 hours post-administration of intranasal racemic ketamine. Thus, both subjects also demonstrated a rapid, clinically significant response (meets the definition of MADRS item 10 response; score <3 units) that was sustained through the next dosing. See FIGS. 19A-19B.

The CGIS-SI/B (range 1-5 units) score of Subject 1 decreased from a baseline of 4 units to 1 unit (“not at all suicidal”) 24 hours post-administration of intranasal racemic ketamine, and remained at 1 unit thereafter. The CGIS-SI/B score of Subject 2 decreased from a baseline of 4 units down to 2 units (“mildly suicidal”) at 24 hours post-administration of intranasal racemic ketamine, and further decreased to 1 unit on Day 3. Thus, both subjects demonstrated remarkable clinical improvement. See FIGS. 14A-14B.

The STS-CMCM score (range 0-52 units) of Subject 1 decreased from 18 units to zero units at 24 hours post-administration of intranasal racemic ketamine. The STS-CMCM score of Subject 2 was similarly reduced from 22 units to 3 units at 24 hours post-administration of intranasal racemic ketamine. Consistent with the previous results, both subjects demonstrated rapid and clinically meaningful improvement. See FIGS. 15A-15B.

The PGIS-SI/B score (range 1-5 units) of Subject 1 decreased from a baseline of 3 units to 1 unit (“not present”) and the PGIS-SI/B score of Subject 2 decreased from 4 units to 1 unit, at 24 hours post-administration of intranasal racemic ketamine, and both subjects remained at a PGIS-SI/B score of 1 unit thereafter. This demonstrates rapid clinical improvement. See FIGS. 16A-16B.

The CGIC-SI/B score (range 1-7 units) of Subject 1 was 1 unit at 4 hours post-administration of intranasal racemic ketamine and the CGIC-SI/B score of Subject 2 was 1 unit at 24 hours post-administration of intranasal racemic ketamine, and both subjects remained at a CGIC-SI/B score of 1 unit thereafter. See FIG. 17.

The PGIC-SI/B score (range 1-5 units) of Subject 1 was reduced from 2 units to 1 unit at 24 hours post-administration of intranasal racemic ketamine, and remained at a PGIC-SI/B score of 1 unit thereafter. The PGIC-SI/B score of Subject 2 was reduced from 3 units to 2 units at 24 hours post-administration of intranasal racemic ketamine, and further decreased to 1 unit at Day 3. See FIG. 18.

The STS-CMCM score (range 0-9) of Subject 1 decreased from a baseline of 7 units to 3 units at 24 hours post-administration of intranasal racemic ketamine. The STS-CMCM score of Subject 2 decreased from 7 units to 3 units at 24 hours post-administration of intranasal racemic ketamine. These score decreased to 2 units at Day 8; Subject 1 remained at 2 units on Day 9, while Subject 2's score was zero units at Day 9. Consistent with the previous results, both subjects demonstrated rapid and clinically meaningful improvement. See FIGS. 20A-20B.

The STS-CMCM “Risk of Suicide at Within the Next 7 Days” score (range 1-10 units) of Subject 1 decreased from 5 units to 2 units at 24 hours post-administration of intranasal racemic ketamine. The STS-CMCM “Risk of Suicide at Within the Next 7 Days” score of Subject 2 decreased from a baseline of 7 units to 1 unit at 24 hours post-administration of intranasal racemic ketamine. The scores remained at 1 unit on Day 4 and further decreased to 0 units on Day 8. Consistent with the previous results, both subjects demonstrated rapid and clinically meaningful improvement. See FIGS. 21A-21B.

The C-SSRS score (“yes” or “no”) for suicidal ideation of Subject 1 improved from a baseline of yes to no at 24 hours post-administration of intranasal racemic ketamine, and the C-SSRS score for suicidal ideation of Subject 2 improved from a baseline of yes to no at 24 hours post-administration of intranasal racemic ketamine. The C-SSRS scores for suicidal ideation and suicidal behavior for both subjects remained a “no” throughout the remainder of the study. See FIG. 24.

The CADSS scale measures dissociation, a well-known adverse event with ketamine. Indeed, the Spravato® (intranasal (S)-ketamine) label carries a Black Box warning for risk of dissociation, in which patients must be monitored for at least 2 hours after dosing. The label also indicates that some 61%-69% of patients administered (S)-ketamine developed dissociative changes on the CADSS assessment (at 56 mg or 84 mg doses).

In contrast, intranasal racemic ketamine unexpectedly provides less dissociation, as shown in FIG. 23. Subject 1 never scored over zero units on the CADSS assessment (no dissociation) and Subject 2 scored 2 units at 40 minutes post-administration of intranasal racemic ketamine, which does not meet the clinical threshold for dissociation (a score >4 units). Subject 2 scored a zero units on the CADSS assessment at 1 hour post-administration. Thus, neither subject exhibited clinically relevant dissociation.

The MOAA/S scale is a measure of sedation in a patient. Intranasal (S)-ketamine also carries a Black Box warning for risk of sedation (patients must be monitored for at least 2 hours after dosing) due to 48-61% of subjects developed sedation at a 56 mg or 84 mg dose. Pre-dose, Subject 1 had a MOAA/S score of 5 units, which decreased to 4 units at 15 minutes post-administration of intranasal racemic ketamine, but was 5 units at 30 minutes. Subject 2 remained at a MOAA/S score of 5 units throughout the trial. Thus, in contrast to intranasal (S)-ketamine, subjects administered intranasal racemic ketamine unexpectedly exhibit little to no sedation at doses sufficient to provide rapid and clinically significant treatment. See FIG. 22.

Similarly, data for the entire patient cohort to the Day 29/30 endpoint indicates that intranasal racemic ketamine provide rapid, significant, and sustained improvements in clinical relevant measures of depression and suicidality, with no clinically meaningful sedation or dissociation. There is no anticipated loss of effect at day 16, (i.e., persistent effect), nor any change to the safety and tolerability of administration of intranasal racemic ketamine.

Based on the preliminary results obtained for 7 subjects who completed the 29/30-day follow-up, the MADRS Total (primary endpoint) and CGIS, S-STS, and PGIS (key secondary endpoints) data show rapid reductions in depressive symptoms and suicidality respectively. Additionally, a durable response was observed through the final Day 29/30 measure for MADRS Total and MADRS Item 10, through the Day 26 measure of S-STS, and through the Day 16 measure of CGIS and PGIS. See FIGS. 25A-25B.

The data show that intranasal racemic ketamine as a standalone treatment (with SOC) appears to provide a rapid decrease in suicidality scores as measured by MADRS Item 10, as well as a sustained response out to 15 days after the final administration of intranasal racemic ketamine.

Summary through Day 29/30

### MADRS

76.5% response rate on Day 1, 92.9% response rate on Day 16 and 100% response rate on Day 29; 14 days after the last dose on Day 15
   Remission rates (a score of less than 12) were 23.5% on Day 1 and 78.6% on Day 16
   Baseline=39.4, Day 1 mean=14.5, Day 16 mean=7.4, and Day 29 mean=6.3

### CGIS-SI/B

94% patients had >1 point within-patient change from their baseline on Day 1 and 100% of patients on Day 16 had 2, 3 or 4 point within-patient change from their baseline
   Baseline=3.8, Day 1 mean=1.8, and Day 16 mean=1.1

### STS

94% of patients had >14 points within-patient change from their baseline and 1 patient (6%)=4 points within-patient change on Day 1
   Baseline=21.9, Day 1 mean=1.7, and Day 16 and Day 29 mean values were 0.0 for all patients

### PGIS-SI/B

100% of patients had >1 point within-patient change from their baseline on Day 1 and 100% of patients on Day 16 had 2, 3 or 4 points within-patient change from their baseline:
   Baseline=3.5, Day 1 mean=1.6, and Day 16 mean=1.0

### MADRS Suicide Item-10:

94% response rate on Day 1 and 100% response rate on Day 16
   Baseline=5.2, Day 1 mean=0.8; Day 16 mean=0.1; Day 29 mean=0.0

TABLE 3
Summary of baseline and Clinical Endpoints
Mean Scores

| | Baseline | Day 1 | Day 16 |
|---|---|---|---|
| MADRS | 39.4 | 14.5 | 7.4 |
| CGIS-SI/B | 3.8 | 1.7 | 1.1 |
| STS | 21.9 | 1.7 | 0.0 |
| PGIS-SI/B | 3.5 | 1.6 | 1.0 |
| MADRS Item 10 | 5.2 | 0.8 | 0.0 |

In addition, no serious adverse events were observed, all adverse events were mild or moderate, transient in nature, and with no new or unique safety signals identified. Indeed, no subject exhibited clinically meaningful adverse events, as noted below. Potential dissociation was also evaluated using CADSS (total CADSS score >4 units=dissociation) at pre-dose, 40 minutes, and 1 hour, 2 hours, 4 hours, 6 hours, and 24 hours post dose. Dissociation, if observed, was noted at 40 minutes post dose on Day 1 and generally rapidly resolved (i.e., in less than 2 hours). On Days 1 and 4, four of the 7 subjects had no dissociation, and after Day 4, only 1 subject reported dissociation (on Day 11 at 40 minutes). These results demonstrate a very acceptable safety and tolerability profile.

### Clinician Administered Dissociative States Scale (CADSS)

Change from baseline (at 40 minutes)=3.8 after first dose, 2.3 on Day 4 (after second dose) and 0.4 on Day 8 (after third dose).

### Hemodynamic Effects

Systolic BP: Baseline=121; 1 hour (post first dose)=125.8; 2 hours (post first dose)=121.3; Diastolic BP: Baseline=79.0; 1 hour (post first dose)=80.9; 2 hours (post first dose)=78.5.

Possible sedation was assessed using the MOAA/S at pre-dose and 15 minutes, 30 minutes, 45 minutes, 60 minutes, 75 minutes, and at 90 minutes, as well as at 2 hours, 4 hours, and 6 hours post dose. On Day 1, 5 of 7 subjects had MOAA/S score of 5 units (“responds readily to name spoken in normal tone”) at all time-points, suggesting no sedation, and no one had a score of <4 units (4 units=“lethargic response to name spoken in normal tone”). On subsequent dosing days, there were no scores <5 units, except for 1 subject who had a score of 4 units at the 6-hour time-point on Day 4. Specifically, all but two patients were unchanged from baseline values 15 minutes after their first dose. One patient improved from 4 to 5 and one patient declined from 5 to 4. The latter patient's score returned to 5 when measured 15 minutes later (30 minutes after the first dose). All other patients maintained their score from the 15-minute measurement to the 30-minute measurement.

### Example 5. A Randomized, Double-blind, Phase 2a Study to Assess the Safety, Tolerability, and Efficacy of Racemic Ketamine Intranasally Administered to Adults with Post-Traumatic Stress Disorder at Imminent Risk of Suicide

This example describes a Phase 2a, multicenter double blind placebo controlled study to assess the safety, tolerability and efficacy, of intranasal racemic ketamine in adult subjects with post-traumatic stress disorder at imminent risk of suicide.

### Study Overview

This Phase 2a, multi-center, randomized, double-blind, placebo-controlled study in adult subjects diagnosed with post-traumatic stress disorder (PTSD) at imminent risk of suicide is designed to evaluate the safety, tolerability, and efficacy of intranasally administered racemic ketamine.

Sixty-four (64) subjects are randomized 2:1 to either intranasal racemic ketamine (75 mg) or matching placebo. Each subject's participation includes a 2-7 day screening phase, a 4-week treatment phase during which study medication is given 2 times per week, and a 2-week safety follow-up phase, for a total of up to 7 weeks of study participation. Subjects are treated as inpatients for a minimum of 8 days, and assuming the subject meets readiness for discharge criteria, are discharged on Day 6 to continue the trial as outpatients. Subjects return to the clinic to receive study medication and undergo study assessments 2 times per week until the end of treatment visit. An in-person safety follow-up visit is conducted 7 days after the last dose; a phone safety follow-up visit is conducted 14 days after the last dose.

### Objectives:

The primary objective of this study is to evaluate the safety and tolerability of intranasal racemic ketamine in adults with PTSD who are at imminent risk of suicide.

The secondary objectives are:
(1) To explore the efficacy of intranasal racemic ketamine on symptoms of suicidality in adults with PTSD; and
(2) To explore the psychometric properties of various outcome assessment tools focusing on suicidality and/or PTSD.

### Number of Subjects

Approximately 64 subjects will be randomized.

### Criteria for Inclusion:

Subjects must fulfill all of the following requirements to enter the study:
(1) The subject is able to speak, read, and understand English and/or the language of the investigative staff to sufficiently understand the nature of the study, to provide written informed consent, and to allow the completion of all study assessments;
(2) Subject is 20-55 years at the time of informed consent;
(3) Male subjects who are sexually active must agree to abstain from sexual activity or be willing to use a double-barrier method of birth control if they become sexually active from time of consent/assent and for 14 days after the last dose of study drug has been taken;
(4) Female subjects of child-bearing potential must have a negative serum pregnancy test at the screening visit, a negative urine pregnancy test at the baseline visit, and must not be breastfeeding. Females must be willing to abstain from sexual activity or use a medically accepted form of birth control if they are sexually active from time of consent/assent and for 14 days after the last dose of study drug has been taken;
(5) The subject meets Diagnostic and Statistical Manual of Mental Disorders, Fifth Edition (DSM-5) criteria for a diagnosis of current PTSD based on psychiatric intake and confirmed by the Mini International Psychiatric Interview Version 7.02 for Suicidality Disorders (MINI);
(6) In the investigator's opinion and confirmed by the MINI, the subject meets criteria for current PTSD Suicidality Disorder;
(7) If the subject's primary index trauma occurred during childhood and/or more than 15 years prior to Screening, current symptoms of PTSD are the result of a more recent trauma which, in the investigator's opinion, reactivated an earlier traumatic response to the original event;
(8) In the investigator's opinion, the subject requires psychiatric hospitalization due to significant risk of suicide and has a score of 8 (inclusive) or less on the STS-CMCM Clinician Judgement of Patient Risk of Suicide Attempt or Death;
(9) In the investigator's opinion, the subject has current suicidal ideation with intent, which is confirmed by the MINI Suicidality Module at screening and baseline, specifically a positive response related to present symptoms on Question B3, as well as a positive response related to symptoms within the past 24 hours on Question B10 or B11;
(10) The subject's diagnoses of PTSD and suicidality are considered primary (i.e., the primary sources of current symptoms and impairment;
(11) In the investigator's opinion and based on reported psychiatric medical history, the subject has history of chronic (>3 months) intermittent non-impulsive suicidality;
(12) The subject agrees to be hospitalized voluntarily for a minimum of 8 days (screening to study Day 6, and fully understands that the duration of hospitalization may be longer if clinically indicated (i.e., he/she is not safe to be discharged on Day 6);
(13) The subject is willing and able to maintain existing treatments and refrain from specific therapies contraindicated by the protocol; and
(14) The subject is able to complete intranasal administration of study medication.

### Exclusion Criteria:

The presence of any of the following criteria excludes a subject from participating in the study:
(1) The subject currently meets criteria for Impulse Attack Suicidality Disorder and/or Homicidal Suicidality Disorder, as confirmed by the MINI Version 7.02 for Suicidality Disorders;
(2) The subject has a score of 9 or 10 on the STS-CMCM Clinician Judgement of Patient Risk of Suicide Attempt or Death;
(3) The subject has a lifetime diagnosis of bipolar disorder, any mood disorder with psychotic features, schizophrenia or other psychotic disorder, obsessive-compulsive disorder, feeding and/or eating disorder, borderline personality disorder, or antisocial personality disorder, as confirmed by the MINI;
(4) The subject is currently diagnosed with moderate or severe alcohol or substance use disorder. Nicotine use disorder is permitted;
(5) The subject is diagnosed with intellectual disability, a neurocognitive disorder including dementia, or has a history of moderate or severe traumatic brain injury. Mild traumatic brain injuries are not necessarily exclusionary provided the investigator believes current symptoms would not interfere with conduct or interpretation of safety and/or efficacy assessments;
(6) The subject has a history or current finding of any clinically significant, hematological, hepatic, respiratory, renal, neurological, known positive HIV infection, gastrointestinal disorder, or other disease that might confound the results of safety assessments conducted in the study;
(7) The subject has a history of seizures (other than childhood febrile seizures);
(8) The subject has a body mass index (BMI) above 35 at screening or baseline;
(9) The subject has known, uncontrolled hypertension or blood pressure above 140/90 mmHg (BP may be repeated as per the site's SOPs) at the screening or baseline visit;
(10) The subject has a known history or current finding of cardiovascular disease, advanced arteriosclerosis, structural cardiac abnormality, cardiomyopathy, serious heart rhythm abnormalities, coronary artery disease, congenital heart disease, ischemic heart disease, cardiac insufficiency, supraventricular and ventricular heart rhythm disorder, prolonged QT syndrome (i.e., QTcF>450 msec) and associated risk factors (i.e., hypokalemia, family history of long QT syndrome), syncope, cardiac conduction problems (e.g., clinically significant heart block), exercise-related cardiac events including syncope and pre-syncope, clinically significant bradycardia, or other serious cardiac problems;
(11) The subject has a known family history of sudden cardiac death or ventricular arrhythmia;
(12) The subject has any clinically significant abnormality on 12-lead ECG performed at the screening visit (Visit 1) or the baseline visit (Visit 2) such as serious arrhythmia, cardiac conduction problems, or other abnormalities deemed to be a potential safety issue;
(13) The subject has a concurrent chronic or acute illness, disability, or other condition (e.g., sleep apnea or narcolepsy) that might confound the results of safety assessments conducted in the study;
(14) The subject has any medical condition that could interfere with the absorption of intranasal ketamine (e.g., nasal polyps, clinically significant deviated septum [corrected or persistent], or other physical abnormalities of the nose);
(15) The subject has a positive urine drug test at screening. Subjects testing positive for cannabis may be entered at the investigator's discretion, provided they are able to meet all other protocol requirements;
(16) The subject is has a positive urine pregnancy test at screening, or is lactating or breastfeeding;
(17) The subject has positive hepatitis B, hepatitis C, or human immunodeficiency virus (HIV) results at screening (Visit 1);
(18) The subject has any history of ketamine or PCP use disorder/abuse;
(19) The subject has any history of using ketamine or esketamine for any psychiatric treatment;
(20) The subject has a history of electroconvulsive therapy (ECT) for any reason;
(21) The subject has known or suspected intolerance or hypersensitivity to the investigational product(s), closely-related compounds, or any ingredients;
(22) The subject has any clinically significant laboratory abnormality including an abnormality that indicates clinically significant hematologic, hepatobiliary, or renal disease;
(23) The subject has used an investigational product, been enrolled in a clinical study including vaccines, or had any changes in eating habits, within 30 days prior to the first dose of investigational product;
(24) The subject was previously enrolled in this study. Subjects screened for the current study previously, but not randomized, may be rescreened with the approval from the study medical monitor;
(25) The subject does not meet or is not willing to comply with the related to prohibited and restricted medications, as well as required washout periods prior to participation. Prohibited medications include, but are not limited to, monoamine oxidase inhibitors, antipsychotics, opioids or drugs with activity at the opioid receptor, psychostimulants, NMDA receptor modulators, magnesium, lithium or any medication that might confound the results of safety assessments conducted in the study. Potent CYP 3A4 inhibitors are not permitted within 1 week of first dose and until at least 1 day after the last dose. Potent CYP 3A4 inducers are not permitted within 30 days of first dose and until at least 1 day after the last dose. Subjects who have recently discontinued lithium or calcium channel blockers within 3 month prior to screening are also excluded;
(26) The subject is an employee of the sponsor, clinical research organization or research site personnel directly affiliated with this study or their immediate family member defined as a spouse, parent, child or sibling, whether biological or legally adopted;
(27) The subject has pending legal charges or is on probation, or is in the process of litigating for compensation or currently receiving compensation for PTSD or other e psychiatric disorder;
(28) The subject, in the opinion of the investigator, is considered unsuitable or unlikely to comply with the study protocol for any reason; and
(29) The subject is legally incapacitated, has been involuntarily hospitalized in the past year, or has another significant mental health issue, physical issue, or life circumstance that could interfere with the conduct or interpretation of study evaluations.

### Concomitant Treatment

Subjects are permitted to be participating in standard of care psychotherapy, including behavioral therapy, if the therapy was initiated for ≥4 weeks prior to screening and has been reasonably stable, in the investigator's opinion. Selective serotonin reuptake inhibitors (SSRIs) or serotonin and norepinephrine reuptake inhibitors (SNRIs) are permitted if the patient has been taking the medication for >8 weeks at a therapeutic dose, as defined by the drug label, and the dose has not been changed within 4 weeks prior to screening. Benzodiazepines and non-hypnotic medications may be taken on a limited basis for sleep, as defined in the protocol restricted medications section, and may never be taken within 8 hours prior to study assessments. Initiation of any new psychotropic medication during the study is not allowed.

### Criteria for Evaluation:

### Efficacy Endpoints:

The effect of intranasal racemic ketamine on individual symptoms is based on changes detected with the following instruments:
(1) Sheehan Suicide Tracking Scale Clinically Meaningful Change Measure (STS-CMCM);
   a. STS-CMCM Clinician Judgement of Patient Risk of Suicide—At This Time,
   b. STS-CMCM Total Score,
   c. STS-CMCM Most Time Spent Per Day with Any Suicidal Impulses, Thoughts or Actions—past 24 hours,
   d. STS-CMCM Clinician Global Severity Rating, and
   e. STS-CMCM Patient Clinically Meaningful Change Measures;
(2) PTSD Checklist for DSM-5 (PCL-5);
(3) CAPS-5; and
(4) Montgomery Asberg Depression Rating Scale (MADRS).

The key efficacy endpoint is the STS-CMCM Clinician Judgement of Patient Risk of Suicide—Current, measured at 24 hours following the Day 0 dose.

### Safety Endpoints:

The safety and tolerability of intranasal racemic ketamine is assessed by:
(1) The frequency and severity of treatment-emergent adverse events (TEAEs);
(2) Clinical laboratory tests;
(3) Vital signs;
(4) ECG;
(5) Physical Examination;
(6) Clinician Administered Dissociative States Scale (CADSS);
(7) Brief Psychotic Rating Scale-Positive Symptoms Subscale (BPRS-+);
(8) Sternberg Short Term Memory Task (SSTM Task) and/or Choice Reaction Time Test (CRT);
(9) Modified Observer's Alertness/Sedation Scale MOAA/S; and
(10) Sheehan Suicide Tracking Scale Clinically Meaningful Change Measure (STS-CMCM).

### Statistical Methods

### Safety Analyses:

Adverse events are coded using the most recent version of the Medical Dictionary for Regulatory Activities. Treatment-emergent AEs are summarized by system organ class (SOC) and preferred term, with number and proportion of subjects reporting each event. Similar summaries are produced for post-treatment AEs, serious adverse events (SAEs), AEs leading to termination, and AEs with at least a possible relationship to the investigational product. The intensity of AEs and the relationship to investigational product are also summarized for each SOC and preferred term. Vital signs (systolic and diastolic blood pressure, pulse, and respiratory rate) and ECG results are summarized by visit using appropriate descriptive statistics. Safety scale outcomes (CADSS, MOAA/S, BPRS+, cognitive scales) are summarized using descriptive statistics.

### Efficacy Analyses:

Efficacy scale outcomes are summarized using descriptive statistics. Statistical testing are undertaken as described in the protocol, in an exploratory fashion and without a priori hypotheses. Continuous variables are summarized by N, the mean and standard deviation, and categorical variables by the number and percent of subjects in each category. Where appropriate, estimates are presented with 95% confidence intervals.

The key efficacy endpoint is the STS-CMCM Clinician Judgement of Patient Risk of Suicide-Current, measured at 24 hours following the Day 0 dose.

Additionally, exploratory MMRM analyses of change from baseline to endpoint on relevant STS-CMCM items are provided. Details are provided in the Statistical Analysis Plan.

### Sample Size Determination:

The sample size is considered adequate for this Phase 2a study in order to examine safety and tolerability of intranasal racemic ketamine in the described population, and allows exploratory evaluation of efficacy so that dose level and frequency is determined for future later phase, fully powered efficacy trials.

### Study and Treatment Duration:

The sequence and maximum duration of the study periods are as follows:
a 1-7 day screening phase,
a 4-week treatment phase,
a 2-week safety follow-up phase, and
The maximum study duration for each subject is approximately 7 weeks.

TABLE 4
Schedule of Events

| | | 2ⁱ/ | | | | | | |
|---|---|---|---|---|---|---|---|---|
| | | Baseline | | | 5 | | | 8/ |
| Visit Number | 1 | 0 | 3 | 4 | 3 | 6 | 7 | Discharge |
| Study Day | −7 to −1 | Dosing | 1 | 2 | Dosing | 4 | 5 | 6 |
| Informed consent | X | | | | | | | |
| Inclusion/Exclusion Criteria | X | X | | | | | | |
| Randomization | | X^{a} | | | | | | |
| Medical and psychiatric history^{b} | X | | | | | | | |
| Demographics | X | | | | | | | |
| Physical examination | X | | | | | | | X |
| Urine drug screen | X | | | | | | | |
| Serum pregnancy test | X | | | | | | | |
| Urine pregnancy test^{c} | | X^{c} | | | | | | |
| Clinical laboratory tests | X | X^{c} | | | | | | X |
| 12-lead ECG^{d} | X | X | X | | X | | | X |
| Vital signs^{e} | X | X | X | | X | | | X |
| MINI 7.02 for Suicidality | X | | | | | | | |
| Disorders | | | | | | | | |
| CAPS-5 | X | | | | | | | |
| STS-CMCM ^{f} | X | X | X | X | X | X | X | X |
| PCL-5 ^{g} | X | X | X | | X | | | X |
| MADRS ^{h} | X | X | X | | | | | X |
| Prior and concomitant meds ⁱ | X | X | X | | | | | X |
| AE monitoring ⁱ | X | X | X | | X | | | X |
| Safety Scales: BPRS+, CADSS, | X | X | X | | X | | | X |
| MOAA/S^{j} | | | | | | | | |
| Cognitive Scales: SSTM and | X | X | X | | X | | | |
| CRT ^{k} | | | | | | | | |
| Study Medication | | X | | | X | | | |
| Administration ^{l} | | | | | | | | |
| Inpatient period | | X | | | | | | |
| Readiness for Discharge | | | | | | | | X |
| evaluation^{m} | | | | | | | | |
| Need for Hospitalization | | | | | | | | X |
| Assessment/Certification | | | | | | | | |

^{a}Randomization will occur after subject is confirmed to have met inclusion exclusion criteria.
^{b}Medical history will consist of a complete medical history including all hospitalizations, procedures and psychiatric history.
^{c}On Day 0, urine pregnancy and clinical laboratory tests to be performed prior to the first dose.
^{d}On Day 0, ECG to be performed pre-dose, and 1, 2, 4, and 6 hours post-dose (all +/−10 min). On Day 1, ECG to be performed 24 hours after Day 0 dose (+/−10 min). At all relevant subsequent visits, if that visit is a dosing day, ECG to be performed pre-dose.
^{e}On Day 0, vital signs to be performed pre-dose, and 1, 2, 4, and 6 hours post-dose (all +/−10 min). On Day 1, vital signs to be performed 24 hours after Day 0 dose (+/−10 min). At all relevant subsequent visits, if that visit is a dosing day, vital signs to be performed pre-dose.
^{f} Full STS-CMCM is to be performed at Screening, Day 0 (pre-dose, 4 hours post-dose), Day 1 (24 hour post-dose), Day 27 and Day 34. All other STS-CMCM subscale assessments are to be conducted according to the protocol On Day 0, STS-CMCM clinician- and patient-rated scales to be performed pre-dose, 4, hours post first dose (all +/−10 min), and in addition, the patient-rated scales to be performed 12 hours post first dose (+−10 min). On Day 1, the STS-CMCM to be performed 24 hours after Day 0 dose (+/−10 min). The STS-CMCM should be done daily until discharge, approximately 24 hours (+/−30 min) after the previous day's assessment and prior to dosing with study medication- see Table 2.
^{g} PCL-5 to be performed pre-dose on Day 0. On Day 1, the PCL-5 to be performed 24 hours after the Day 0 dose (+/−10 min). At all relevant subsequent visits, if that visit is a dosing day, PCL-5 to be performed pre-dose.
^{h} On Day 0, MADRS to be performed pre-dose, 4 hours post first dose on Day 0. On Day 1, the MADRS to be performed 24 hours after the Day 0 dose (+/−10 min). At all relevant subsequent visits, if that visit is a dosing day, MADRS to be performed pre-dose.
ⁱ AEs and concomitant medications to be recorded every day during the inpatient period.
^{j}On Day 0, safety scales (BPRS+, CADSS, MOAA/S) to be administered pre-dose, and 0.5, 1, 2, 4, and 6 hours post-dose (all +/−10 min). On Day 1, safety scales to be performed 24 hours after Day 0 dose (+/−10 min). At all relevant subsequent visits, if that visit is a dosing day, safety scales to be performed pre-dose and 2 hours post-dose. At 2 hours post-dose, if any safety scale results demonstrate clinically meaningful impairment in the judgement of the investigator, the specific scale(s) should be repeated again in clinic, at 60 minute intervals, until the clinically meaningful impairment resolves.
^{k} On Day 0, cognitive scales (SSTM and CRT) to be administered pre-dose, and 1, 2, and 4 hours post-dose (all +/−10 min). On Day 1, cognitive scales to be performed 24 hours after Day 0 dose (+/−10 min). At all relevant subsequent visits, if that visit is a dosing day, cognitive scales to be performed pre-dose.
^{l} On Day 0, dosing will occur at 10am (+/−30 min). On Day 1, all 24 hour assessments must occur within 24 hours +/−10 min after the Day 0 dose. Doses on subsequent day must occur at approximately the same time of day (+/−1 hour) as the Day 0 dose.
^{m}Investigator determines the need to extend inpatient period until Day XX. If subject is not safe to discharge by study day 10/Visit11 (as defined by a score of 5 or higher on the STS-CMCM Clinician Judgement of Patient Risk of Suicide Attempt or Death), or needs to discontinue for any reason, the subject should be discontinued and follow Visit 21/Endpoint assessments.

### Example 6. A Randomized, Double-Blind, Placebo-Controlled, Phase 2 Study of the Efficacy and Safety of Intranasal Racemic Ketamine in Participants with Complex Regional Pain Syndrome

This example describes a Phase 2 randomized, double blind, placebo controlled study of the efficacy and safety of intranasal racemic ketamine in participants with complex regional pain syndrome (CRPS).

### Study Overview

This Phase 2, randomized, double-blind, placebo-controlled study of the efficacy and safety of intranasally administered racemic ketamine, is designed to include participants with CRPS. All participants will be between the ages of 18 and 65.

After signing the informed consent, participants have the following assessments: assessment of eligibility (inclusion/exclusion criteria), medical history, concomitant medications collection, physical examination, vital sign measurements, laboratory testing, 12 lead electrocardiogram (ECG), pregnancy testing (if applicable), and testing for drugs of abuse and alcohol. Participants also complete questionnaires according to the schedule of events. During the screening period, participants maintain a pain diary to record their average, least, and worst pain intensity each day using the 11-point Numerical Pain Rating Scale (NPRS). To qualify for randomization, the participant has an average daily pain intensity score of >6 on the NPRS over the 7 days prior to randomization.

Participants who meet all of the inclusion criteria and none of the exclusion criteria are eligible for study participation.

Participants continue to use the NPRS to rate their least and worst pain daily throughout the study.

Prior to the initial dose of study medication, on the last treatment day, and weekly during the course of the study, participants complete the following:
(1) PROMIS-29;
(2) Short-form McGill Pain Questionnaire;
(3) Pain Catastrophizing Scale; and
(4) Pain Self-Efficacy Questionnaire.

Participants complete a 14-day course of outpatient administration of intranasally administered racemic ketamine (HCl intranasal spray) daily. At the discretion of the investigator, participants on a stable dose may complete at-home dosing. Prior to the initial dose of study medication and then daily after dosing, participants complete the following:
(1) MOAA/S;
(2) CADSS; and
(3) C-SSRS.

On Day 1, participants receive intranasally administered racemic ketamine (HCl intranasal spray) 60-75 mg. Beginning on Day 2 through Day 7, the intranasal racemic ketamine dosage will be titrated up gradually as tolerated to a maximum of 180 mg daily, administered as individual intranasal spray doses every 2 hours, unless contraindicated due to tolerability issues. The target total daily dose on Days 8-14 is 180 mg (or maximum tolerated dose) administered in divided doses over 4 hours.

After the last dose of the study drug, participants complete follow-up visits on Day 21, Day 28, Day 42, and Day 84.

### Objectives:

The primary objective of this study is to evaluate change from baseline in average pain intensity using an 11-point numerical rating scale where 0=“no pain” and 10=“worst possible pain” over the last 7 days reported on the PROMIS-29 Profile at day 14.

The secondary objectives are to determine:
(1) Proportion of Participants With at Least 30 Percent Reduction in the Average Pain Intensity Score;
(2) Proportion of Participants With at Least 50 Percent Reduction in the Average Pain Intensity Score;
(3) Change from Baseline in the Worst Pain Intensity Score;
(4) Change from Baseline in the Least Pain Intensity Score;
(5) Change in the PROMIS-29 Profile Total Score;
(6) Patient Global Impression of Change (PGIC);
(7) Change from Baseline in Clinician CRPS Severity Score (CSS);
(8) Change from Baseline in the Short-form McGill Pain Questionnaire;
(9) Change from Baseline in the Pain Catastrophizing Scale;
(10) Change in the Pain Self-Efficacy Questionnaire;
(11) Allodynia;
(12) Safety and tolerability; and
(13) Change from Baseline in adherence to exercise/rehabilitation program.

### Number of Subjects

A sufficient number of subjects are screened and randomized to the treatment phase in order to ensure evaluable data for each arm.

### Criteria for Inclusion:

Subjects must fulfill all of the following criteria to be enter the study:
(1) The subject is able to speak, read, and understand English and/or the language of the investigate staff to sufficiently understand the nature of the study, to provide written informed consent, and to allow the completion of all study assessments;
(2) Subject is 18 to 65 years of age at the time of informed consent;
(3) Male subjects who are sexually active must agree to abstain from sexual activity or be willing to use medically acceptable contraception if they become sexually active from time of consent and for 3 months after the last dose of study drug;
(4) Female subjects must have negative serum pregnancy test at screening and must not be breastfeeding or lactating. Females must be willing to abstain from sexual activity or use medically accepted contraception if they are sexually active from time of consent and for 1 month after the last dose of study drug;
(5) Diagnosis of CRPS according to the International Association for the Study of Pain (IASP) version II (Budapest) criteria;
(6) Reports an average pain score over the last 7 days of >6 on the 11-point Numerical Pain Rating Scale at baseline;
(7) BMI within the range of 18.0-40.0 kg/m² (inclusive); and
(8) Willing and able to arrange for transportation to and from the clinic for the entire treatment period and a minder/sitter to stay with the participant for the first few nights after treatment.

### Exclusion Criteria

The presence of any of the following criteria excludes a subject from participating in the study:
(1) Subjects who have ongoing sequelae from prior COVID illness, or subjects who have either documented COVID infection or symptoms suggestive of recent COVID infection within 1 month of screening;
(2) History of allergy, intolerance, or contraindication (as judged by the investigator) to ketamine;
(3) Peripheral neuropathy, or any other chronic pain condition that would significantly affect a participant's ability to report CRPS-related pain;
(4) The subject is diagnosed with intellectual disability, a neurocognitive disorder including dementia, or has a history of moderate or severe traumatic brain injury. Mild traumatic brain injuries are not necessarily exclusionary provided the investigator believes current symptoms would not interfere with conduct or interpretation of safety and/or efficacy assessments;
(5) The subject has history or current finding of any clinically significant, hematological, hepatic, respiratory, renal, neurological, known positive human immunodeficiency virus (HIV) infection, gastrointestinal disorder, or other disease that might confound the results of safety assessments conducted in the study;
(6) The subject has a history of seizures (other than childhood febrile seizures);
(7) The subject has known, uncontrolled hypertension or blood pressure (BP) that, in the investigator's judgement, should exclude the subject at screening, or baseline (BP may be repeated as per the site's standard operating procedures [SOPs]);
(8) The subject has a known history or current finding of cardiovascular disease, advanced arteriosclerosis, structural cardiac abnormality, cardiomyopathy, serious heart rhythm abnormalities, coronary artery disease, congenital heart disease, ischemic heart disease, cardiac insufficiency, supraventricular and ventricular heart rhythm disorder, prolonged QT syndrome (i.e., QTcF >450 msec) and associated risk factors (i.e., hypokalemia, family history of long QT syndrome), syncope, cardiac conduction problems (e.g., clinically significant heart block), exercise-related cardiac events including syncope and pre-syncope, clinically significant bradycardia, or other serious cardiac problems;
(9) The subject has a known family history of sudden cardiac death or ventricular arrhythmia;
(10) The subject has any clinically significant abnormality on 12-lead ECG performed at screening or baseline such as serious arrhythmia, cardiac conduction problems, or other abnormalities deemed to be a potential safety issue;
(11) The subject has concurrent chronic or acute illness, disability, or other condition (e.g., narcolepsy) that might confound the results of safety assessments conducted in the study;
(12) The subject has any medical condition that could interfere with the absorption of SL ketamine (e.g., Current inflammatory or ulcerative disease of the oral cavity that could alter the absorption of sublingual medication);
(13) The subject has symptomatic or uncontrolled hyper- or hypothyroidism or has had changes to their treatment for hyper- or hypothyroidism within 90 days prior to Day 1;
(14) The subject meets the DSM-5 criteria for moderate or severe substance use disorder in the 6 months prior to screening, or in the investigator's opinion, is at risk of withdrawal from substance use (e.g., opiate or alcohol dependent), or has a lifetime history of ketamine, phencyclidine, lysergic acid diethylamide, or 4-methylenedioxy-methamphetamine hallucinogen-related use disorders. Nicotine use disorder is permitted;
(15) The subject has a positive urine test for ketamine, phencyclidine (PCP), cocaine, or amphetamines (inclusive of amphetamine, methamphetamine [mAMP], and 3, 4-methylenedioxy-methamphetamine [MDMA]) at screening;
(16) The subject has positive hepatitis B, hepatitis C, or HIV results at screening;
(17) The subject has any history of using ketamine or esketamine for any pain management or psychiatric treatment;
(18) The subject has known or suspected intolerance or hypersensitivity to the investigational product(s), closely related compounds, or any ingredients;
(19) In the investigator's opinion, the subject has any clinically significant laboratory abnormality including an abnormality that indicates clinically significant hematologic, hepatobiliary, or renal disease.
(20) The subject has received an investigational product, including vaccines, within 30 days prior to the first dose of study drug;
(21) The subject was previously enrolled in the current study. Subjects previously screened for, but not randomized in, the current study may be rescreened with approval from the study medical monitor;
(22) The subject does not meet or is not willing to comply with the requirements related to prohibited and restricted medications and therapies, as well as required washout periods prior to participation. Prohibited medications and therapies include, but are not limited to, monoamine oxidase inhibitors (MAOIs), opioids or drugs with activity at the opioid receptor, psychostimulants, lamotrigine, N-methyl-D-aspartate (NMDA) receptor modulators, magnesium, or any medication/therapy that might confound the results of safety assessments conducted in the study. Subjects who have received any of these prohibited medications within 2 weeks of screening are excluded from the study. Potent CYP 3A4 inhibitors, including nefazodone and fluvoxamine, are not permitted within 1 week of first dose and until at least 1 day after the last dose. Potent CYP 3A4 inducers, including St. John's wort, are not permitted within 30 days of first dose and until at least 1 day after the last dose. Subjects who have recently discontinued lithium or calcium channel blockers within 3 months prior to screening are also excluded;
(23) The subject is an employee of the sponsor, clinical research organization, or research site personnel directly affiliated with this study or their immediate family member defined as a spouse, parent, child, or sibling, whether biological or legally adopted.
(24) The subject has pending legal charges or is on probation;
(25) The subject, in the investigator's opinion, is considered unsuitable or unlikely to comply with the study protocol for any reason; and
(26) The subject is legally incapacitated, has been involuntarily hospitalized in the past year, or has another significant mental health issue, physical issue, or life circumstance that could interfere with the conduct or interpretation of study evaluations.

### Criteria for Evaluation:

### Primary Efficacy Endpoints

The primary efficacy endpoint is the change from baseline in the pain intensity score in the past 7 days reported on the PROMIS-29 Profile at day 14.

### Secondary Efficacy Endpoints

The secondary endpoints (in rank order) are:
(1) Proportion of Participants With at Least 30 Percent Reduction in the Average Pain Intensity Score based on the 11 point VAS PROMIS-29 Profile,
(2) Proportion of Participants With at Least 50 Percent Reduction in the Average Pain Intensity Score based on the 11 point VAS PROMIS-29 Profile,
(3) Change from Baseline in the Worst Pain Intensity Score,
(4) Change from Baseline in the Least Pain Intensity Score,
(5) Change from Baseline in the PROMIS-29 Profile Total Score,
(6) Patient Global Impression of Change (PGIC),
(7) Change from Baseline in Clinician CRPS Severity Score (CSS),
(8) Change from Baseline in the Short-form McGill Pain Questionnaire Total Score,
(9) Change from Baseline in the Pain Catastrophizing Scale Total Score,
(10) Change in the Pain Self-Efficacy Questionnaire,
(11) Allodynia,
(12) Safety and tolerability, and
(13) Change from Baseline in adherence to exercise/rehabilitation program.

### Safety Endpoints:

The safety and tolerability of intranasal racemic ketamine are assessed by:
(1) The frequency and severity of treatment-emergent adverse events (TEAEs);
(2) Frequencies of new or worsening clinically significant laboratory, vital signs, ECGs, or physical examination abnormalities;
(3) Clinician-Administered Dissociative States Scale (CADSS);
(4) Modified Observer's Assessment of Alertness/Sedation (MOAA/S) Scale; and
(5) Columbia Suicide Severity Rating Scale (C-SSRS).

### Study and Treatment Duration

The sequence and duration of the study periods are as follows:
A 1 to 28-day screening phase,
A 14-day treatment phase, and
A 10-week safety follow-up phase.

Therefore, the study duration for each subject is approximately 12 weeks.

All subjects are referred for follow up post study; this includes subjects who do not qualify for the study, subjects who discontinue participation, and those subjects who complete the study.

TABLE 5
Schedule of Events

| | | | | | | | Follow-up Visits | | | | |
|---|---|---|---|---|---|---|---|---|---|---|---|
| | Screening | Treatment Visits | | | | | Day 21 | Day 28 | Day 42 | Day 84 | |
| | Visit | Day | Days | Day | Days | Day | (+/−1 | (+/−1 | (+/−3 | (+/−3 | ET |
| Assessments | (−28 days) | 1 | 2-6 | 7 | 8-13 | 14 | day) | day) | days) | days) | Visit |
| Informed consent | X | | | | | | | | | | |
| Demographics | X | | | | | | | | | | |
| Inclusion/Exclusion | X | | | | | | | | | | |
| Medical history | X | | | | | | | | | | |
| Physical exam¹ | X | X | | X | | X | X | | X | X | X |
| Vital signs² | X | X | X | X | X | X | X | | X | X | X |
| Height, weight, BMI | X | | | | | X | | | X | | |
| | | | | | | (wt) | | | (wt) | | |
| Pregnancy test³ | X | X | | | | | | | | X | X |
| 12- lead ECG⁶ | X | X | | X | | X | | | | X | X |
| Clinical Laboratory | X | X | | X | | X | | | X | X | X |
| Testing⁴ | | | | | | | | | | | |
| Urinalysis⁵ | X | X | | X | | X | X | X | X | X | X |
| Urine test for drugs | X | X | | | | | | | | | |
| of abuse⁶ | | | | | | | | | | | |
| Alcohol breath test | X | X | | | | | | | | | |
| IP administration⁸ | | X | X | X | X | X | | | | | |
| Nasal assessment⁹ | X | X | X | X | X | X | | | | | X |
| Average pain over | X | X | X | X | X | X | X | | X | X | X |
| last 24 hrs¹⁰ | | | | | | | | | | | |
| Least pain over last | X | X | X | X | X | X | X | | X | X | X |
| 24 hrs¹⁰ | | | | | | | | | | | |
| Worst pain over last | X | X | X | X | X | X | X | | X | X | X |
| 24 hrs¹⁰ | | | | | | | | | | | |
| Richmond | | X | X | X | X | X | | | | | X |
| Agitation-Sedation | | | | | | | | | | | |
| Scale (RASS)¹¹ | | | | | | | | | | | |
| Clinical CRPS | X | X | | X | | X | X | | X | X | X |
| Severity Score | | | | | | | | | | | |
| (CSS)¹² | | | | | | | | | | | |
| PROMIS-29¹² | X | X | | X | | X | X | | X | X | X |
| Short Form McGill | X | X | | X | | X | X | X | X | X | X |
| Pain Questionnaire¹² | | | | | | | | | | | |
| Pain Self Efficacy | | X | | X | | X | X | X | X | X | X |
| Questionnaire | | | | | | | | | | | |
| (PSEQ)¹² | | | | | | | | | | | |
| Pain | | X | | X | | X | X | X | X | | X |
| Catastrophizing | | | | | | | | | | | |
| Scale (PCS)¹² | | | | | | | | | | | |
| Clinician | | X | X | X | X | X | | | | | |
| Administered | | | | | | | | | | | |
| Dissociative Scale | | | | | | | | | | | |
| (CADSS-6)¹³ | | | | | | | | | | | |
| Patient Global | | | | X | | X | | X | X | | X |
| Impression of | | | | | | | | | | | |
| Change¹⁴ | | | | | | | | | | | |
| Pelvic Pain and | X | X | | X | | X | X | X | X | X | X |
| Urinary/Frequency | | | | | | | | | | | |
| (PUF) Patient | | | | | | | | | | | |
| Symptom Scale¹⁵ | | | | | | | | | | | |
| Discharge home¹⁶ | | X | X | X | X | X | | | | | |
| Concomitant | X | | | | | | | | | | X |
| medications | | | | | | | | | | | |
| Collect adverse | | | | | | | | | | | |
| events | | | | | | | | | | | X |

¹Full physical exam, including oral cavity, sublingual area assessment, airway and respiratory history, required at screening. Otherwise, brief physical exams required as indicated on schedule.
²Vital signs include blood pressure, heart rate, respiratory rate, body temperature and oxygen saturation check. At each treatment visit conducted in the outpatient clinic, vital signs are required prior to commencing treatment, every 30 minutes (+/−5 minutes) X 2, then hourly (+/−15 minutes) thereafter until discharge. Participants will have continuous oxygen saturation monitoring during all treatment visits begin prior to commencement of treatment and continuing until discharge.
³Females of childbearing potential will have a serum pregnancy test at screening, followed by a urine pregnancy tests at other visits as indicated on schedule.
⁴Safety laboratory testing will include hematology and serum chemistry, including thyroid and liver panels. Serology testing for Hep B, C, and HIV will occur at screening only.
⁵Dipstick urinalysis testing will be performed for blood, protein, leukocytes, and nitrite. Microscopic examination will be performed if any dipstick tests are positive.
⁶The standard facility urine test for drugs of abuse will be used.
⁸On Treatment Day 1, participants will receive intranasal racemic ketamine 60-75 mg. On Treatment Days 2-7, participants will receive increasing doses of intranasal racemic ketamine gradually as tolerated, up to a maximum of a total daily dose of 180 mg administered as individual doses every 2 hours. The target total daily dose on Days 8-14 is 180 mg in divided doses over 4 hours.
⁹Abnormal findings on sublingual assessments will be recorded as adverse events.
¹⁰Prior to treatment on each treatment day, and at times indicated on the schedule, participants will rate their average, least and worst pain intensities over the last 24 hours using an 11-point NPRS, where 0 = no pain and 10 = worst possible pain.
¹¹Research staff will utilize the RASS to document sedation score prior to initiation of treatment and hourly during each treatment visit. For a RASS score greater than −3, the next dose of intranasal racemic ketamine may be delayed until sedation lessens.
¹²On treatment days, completion of the PROMIS-29, Short Form McGill Pain Questionnaire, PSEQ, PCS, and Modified LUTS will occur prior to the initiation of treatment.
¹³On Treatment Days indicated in the schedule, administer the CADSS-6 prior to initiation and at the conclusion of treatment. Recommend each day of dosing
¹⁴The Patient Global Impression of Change will be completed at the conclusion of the treatment visits indicated on the schedule (or at early termination, if applicable).
¹⁵At screening, participants will complete a Modified Lower Urinary Tract Symptom (LUTS) Questionnaire. Investigators will use the participant's answers when determining whether participant has a clinically significant bladder or urinary tract condition. At screening, participants will complete the questionnaire based on the last month. After screening, participants will base their answers based on time since last completion of questionnaire. Symptoms beginning or worsening after baseline will be captured as adverse events and followed until resolution or clinical stability is determined by the investigator.
¹⁶Participants will be discharged to home when the following criteria are met: 1. It has been a minimum of 1 hour since the last dose of IP. 2. Vital signs are stable. 3. Psychomimetic adverse events are stable and tolerable to participant. 4. A minder is present and agrees to stay with the participant for the first night after treatment.

### Example 7. A Double Blind, Placebo-Controlled Phase 2 Study to Assess the Efficacy, Safety, and Tolerability of Intranasal Racemic Ketamine Administered to Adults with Post-Traumatic Stress Disorder

This example describes a Phase 2 double blind, placebo controlled study to assess the efficacy, safety, and tolerability of intranasal racemic ketamine in adult subjects with post traumatic stress disorder (PTSD).

### Study Overview

This randomized, double-blind, placebo-controlled multicenter, study in adult subjects diagnosed with PTSD evaluates the efficacy, safety, and tolerability of intranasally administered racemic ketamine. Approximately 240 subjects are randomized 1:1 to receive either intranasal racemic ketamine (90 mg) or matching placebo.

Each subject participates in a 2- to 7-day screening phase, a 12 week treatment phase including standard of care (e.g., SSRIs) as judged necessary per the investigator during which study drug will be administered 2 times per week, the standard of care therapy (SOC) is continued throughout, and a 4-week follow-up phase for a total of 17 weeks of study participation. Ideally, SOC is not changed after baseline during the treatment phase and is optimized during the 4-week follow-up phase, unless necessary to address adverse events related to the SOC medication. Subjects are treated in the clinic as outpatients and return to the clinic to receive study drug and undergo study assessments 2 times per week. Subjects are evaluated for efficacy using multiple psychometric scales and for safety using clinical laboratory assessments, electrocardiograms (ECGs), vital signs, and physical examinations. After the last dose of study drug, subjects are monitored for 4 weeks, including 4 in-person safety follow-up visits at Week 13, 14, 15 & 16).

### Objectives:

The primary objectives of this study is to evaluate the efficacy of intranasal racemic ketamine on symptoms of PTSD in adults

The secondary objectives of this study is to evaluate the safety and tolerability of intranasal racemicketamine in adults with PTSD

### Number of Subjects

Approximately 240 subjects and, are randomized.

### Criteria for Inclusion:

Subjects must fulfill all of the following requirements to enter the study:
(1) The subject is able to speak, read, and understand English and/or the language of the investigative staff to sufficiently understand the nature of the study, to provide written informed consent, and to allow the completion of all study assessments;
(2) Subject is 18 to 65 years of age at the time of informed consent;
(3) Male subjects who are sexually active must agree to abstain from sexual activity or be willing to use medically acceptable contraception (as specified in the protocol if they become sexually active from time of consent and for 3 months after the last dose of study drug);
(4) Female subjects of childbearing potential must have a negative serum pregnancy test at screening and must not be breastfeeding or lactating. Females must be willing to abstain from sexual activity or use medically accepted contraception (as specified in the protocol) if they are sexually active from time of consent and for 1 month after the last dose of study drug;
(5) The subject meets Diagnostic and Statistical Manual of Mental Disorders, Fifth Edition (DSM-5) criteria for a diagnosis of current PTSD (i.e. past 6 months).
(6) Subject has CAPS-5 baseline severity indicative of PTSD;
(7) If the subject's primary index trauma occurred during childhood and/or more than 15 years prior to Screening, current symptoms of PTSD are the result of a more recent trauma which, in the investigator's opinion, reactivated an earlier traumatic response to the original event;
(8) If the subject is taking psychotropic medications, they must be willing to be maintained on a stable dose for the duration of the study;
(9) The subject is willing to avoid the use of alcohol, CBD oil, and recreational drugs, as well as specific therapies prohibited by the protocol, from screening through the last study visit; and
(10) The subject is able to complete intranasal administration of study drug.

### Exclusion Criteria:

The presence of any of the following criteria excludes a subject from participating in the study:
(1) Subjects who have ongoing sequelae from prior COVID illness, or subjects who have either documented COVID infection or symptoms suggestive of recent COVID infection within 1 month of screening;
(2) Participants meeting DSM-5 criteria for a history of or current psychotic or bipolar affective disorders. Those participants should be referred clinically to ensure they have appropriate level of clinical care;
(3) In the investigator's opinion, the subject has a current diagnosis of borderline personality disorder or, if the subject has not met full diagnostic criteria for borderline personality disorder within the last 5 years, the subject has a history of recurrent non-suicidal self-injury, or self-mutilating behavior;
(4) Suicide attempt within the last 12 months, on the C-SSRS, and a score of 2, 3, or 4 for Actual Lethality/Medical Damage;
(5) The subject is diagnosed with intellectual disability, a neurocognitive disorder including dementia, or has a history of moderate or severe traumatic brain injury. Mild traumatic brain injuries are not necessarily exclusionary provided the investigator believes current symptoms would not interfere with conduct or interpretation of safety and/or efficacy assessments;
(6) The subject has a history or current finding of any clinically significant, hematological, hepatic, respiratory, renal, neurological, known positive human immunodeficiency virus (HIV) infection, gastrointestinal disorder, or other disease that might confound the results of safety assessments conducted in the study;
(7) The subject has a history of seizures (other than childhood febrile seizures);
(8) The subject has a body mass index (BMI)>40 or <18 at screening;
(9) The subject has known, uncontrolled hypertension or blood pressure (BP) that, in the investigator's judgment, should exclude the subject at screening or baseline (BP may be repeated as per the site's standard operating procedures [SOPs]);
(10) The subject has a known history or current finding of cardiovascular disease, advanced arteriosclerosis, structural cardiac abnormality, cardiomyopathy, serious heart rhythm abnormalities, coronary artery disease, congenital heart disease, ischemic heart disease, cardiac insufficiency, supraventricular and ventricular heart rhythm disorder, prolonged QT syndrome (i.e., QTcF >450 msec) and associated risk factors (i.e., hypokalemia, family history of long QT syndrome), syncope, cardiac conduction problems (e.g., clinically significant heart block), exercise-related cardiac events including syncope and pre-syncope, clinically significant bradycardia, or other serious cardiac problems;
(11) The subject has a known family history of sudden cardiac death or ventricular arrhythmia;
(12) The subject has any clinically significant abnormality on 12-lead ECG performed at screening or baseline such as serious arrhythmia, cardiac conduction problems, or other abnormalities deemed to be a potential safety issue;
(13) The subject has a concurrent chronic or acute illness, disability, or other condition (e.g., narcolepsy) that might confound the results of safety assessments conducted in the study.
(14) The subject has any medical condition that could interfere with the absorption of intranasal ketamine (e.g., nasal polyps, clinically significant deviated septum [corrected or persistent], or other physical abnormalities of the nose);
(15) The subject has symptomatic or uncontrolled hyper- or hypothyroidism or has had changes to their treatment for hyper- or hypothyroidism within 90 days prior to Day 1;
(16) The subject meets the DSM-5 criteria for moderate or severe substance use disorder in the 6 months prior to screening, or in the investigator's opinion, is at risk of withdrawal from substance use (e.g., opiate or alcohol dependent), or has a lifetime history of ketamine, phencyclidine, lysergic acid diethylamide, or 4-methylenedioxy-methamphetamine hallucinogen-related use disorders. Nicotine use disorder is permitted;
(17) The subject has a positive urine test for phencyclidine (PCP), cocaine, or amphetamines (inclusive of amphetamine, methamphetamine [mAMP], and 3, 4-methylenedioxy-methamphetamine [MDMA]) at screening;
(18) The subject requires daily benzodiazepine use of >2 mg of lorazepam or dose equivalent;
(19) The subject has any history of using ketamine or esketamine for any psychiatric treatment;
(20) The subject has known or suspected intolerance or hypersensitivity to the investigational product(s), closely related compounds, or any ingredients;
(21) In the investigator's opinion, the subject has any clinically significant laboratory abnormality including an abnormality that indicates clinically significant hematologic, hepatobiliary, or renal disease. Note, any screening abnormality must be discussed with the Medical Monitor for approval prior to randomization;
(22) The subject has received an investigational product, including investigational vaccines, within 6 months prior to the first dose of study drug;
(23) The subject was previously enrolled in the current study. Subjects previously screened for, but not randomized in, the current study may be rescreened with approval from the study medical monitor;
(24) The subject does not meet or is not willing to comply with the requirements listed in protocol related to prohibited and restricted medications and therapies, as well as required washout periods prior to participation. Prohibited medications and therapies include, but are not limited to, monoamine oxidase inhibitors (MAOIs), opioids or drugs with activity at the opioid receptor, psychostimulants, lamotrigine, N-methyl-D-aspartate (NMDA) receptor modulators, magnesium, electroconvulsive therapy (ECT), transcranial magnetic stimulation (TMS) or any medication/therapy that might confound the results of safety assessments conducted in the study. Subjects who have received any of these prohibited medications within 2 weeks of screening are excluded from the study. Potent CYP 3A4 inhibitors, including nefazodone and fluvoxamine, are not permitted within 1 week of first dose and until at least 1 day after the last dose. Potent CYP 3A4 inducers, including St. John's wort, are not permitted within 30 days of first dose and until at least 1 day after the last dose. Subjects who have recently discontinued lithium or calcium channel blockers within 3 months prior to screening are also excluded;
(25) The subject is an employee of the sponsor, clinical research organization, or research site personnel directly affiliated with this study or their immediate family member defined as a spouse, parent, child, or sibling, whether biological or legally adopted;
(26) The subject has pending legal charges or is on probation or is on disability for PTSD.
(27) The subject, in the investigator's opinion, is considered unsuitable or unlikely to comply with the study protocol for any reason; and
(28) The subject is legally incapacitated, has been involuntarily hospitalized in the past year, or has another significant mental health issue, physical issue, or life circumstance that could interfere with the conduct or interpretation of study evaluations.

### Concomitant Treatment

Evidence-/empirically based SSRI treatment is given to subjects as necessary prior to study start. SOC should remain stable during the 12-week treatment phase. In addition, Subjects are permitted to be participating in SOC psychotherapy, including behavioral therapy. Benzodiazepine (dose equivalent to ≤2 mg/day of lorazepam) medications may be taken on a limited basis as needed, as defined in the protocol restricted medications section, but should not be taken within 24 hours prior to study medication dosing and assessments. Short acting non-benzodiazepine hypnotics (e.g. zolpidem, zaleplon) are permitted, but may never be taken within 10 hours prior to study medication dosing.

### Criteria for Evaluation:

### Primary Efficacy Endpoints:

The primary efficacy endpoint is the change from baseline in the Clinician Administered PTSD Scale DSM-5 (CAPS-5) at 12 weeks.

### Secondary Efficacy Endpoints:

The additional secondary endpoints are the change from baseline in the following:
(1) Number and % of CAPS-5 responders (total score reduction of 12 points from baseline) at each efficacy timepoint,
(2) Number and % of persistent CAPS-5 responders from onset at each timepoint and maintained through Week 12,
(3) Number and % of persistent CAPS-5 responders from onset at each timepoint and maintained through Week 16,
(4) CGIS at week 12,
(5) PGIS at week 12,
(6) Clinical Global Impression Change (CGIC) at week 16,
(7) Patient Global Impression Change (PGIC) at week 16,
(8) Sheehan Disability Scale (SDS) at week 16, and
(9) Posttreatment Phase—time to relapse for responders in the treatment phase, who subsequently relapse during the 4-week safety follow-up. (Relapse is defined as a CAPS-5 increase of ≥12 points for two consecutive weeks).

### Safety Endpoints:

The safety and tolerability of intranasal racemic ketamine are assessed by:
(1) The frequency and severity of treatment-emergent adverse events (TEAEs);
(2) Frequencies of new or worsening clinically significant laboratory, vital signs, ECGs, or physical examination abnormalities;
(3) Clinician-Administered Dissociative States Scale (CADSS);
(4) Modified Observer's Assessment of Alertness/Sedation (MOAA/S) Scale; and
(5) Columbia Suicide Severity Rating Scale (C-SSRS).

### Statistical Methods

For the analysis of the primary efficacy endpoint, the change in the CAPS-5 total score from baseline at 24 hours following the initial dose, is analyzed using an ANCOVA model including baseline CAPS-5 total score as a covariate, treatment as fixed effect and a random subject effect. A hierarchical testing procedure is used to maintain the experiment-wise type 1 error rate at 5%. Endpoints, starting with the primary endpoint, are tested in the pre-specified order of importance. Once a p-value greater than 0.05 is obtained, all subsequent hypothesis testing are stopped. Patients are stratified based on SSRI or SNRI use as a sensitivity analysis. Further details of the statistical analyses, including secondary efficacy, safety, and sensitivity analyses, are described in the protocol and/or the Statistical Analysis Plan (SAP).

### Sample Size Determination

The sample size is calculated assuming an effect size of 0.45, a 2-sided significance level of 0.05, and assuming approximately a 15% dropout rate. Based on these assumptions, 120 subjects are randomized to each treatment group to achieve 90% power. The effect size used in this calculation was based on results of previous ketamine studies and on clinical judgment.

### Study and Treatment Duration

The sequence and maximum duration of the study periods are as follows:
a 2- to 7-day screening phase,
a 12-week treatment phase of investigational study drug during which SOC is maintained, and
a 4-week safety follow up during which SOC is continued and optimized as clinically warranted.

Therefore, the maximum study duration for each subject is approximately 17 weeks.

All subjects are referred for follow up post study; this includes subjects who do not qualify for study, subjects who discontinue participation, and those subjects who complete the study.

TABLE 7
Schedule of Events

| | | | | | Phone | | |
|---|---|---|---|---|---|---|---|
| | | | | Dosing | Follow up | | Final |
| | 2 - | | | Every 4 | 24 hrs | Final | Followup |
| 1 - | Baseline | Follow- | Phone | days ^{s} | after dose | Dose | 24 hours |

| Visit Name | Screen | Dosing | up | Dosing | Followup | Week 2 - Week 12 | | Day 85 | after final |
|---|---|---|---|---|---|---|---|---|---|
| Study Day | −7 to −2 | 1 | 2 | 5 | 6 | (Days 9-81) | | (±1) | dose |
| Assessments & Procedures^{a} | | | | | | | | | |
| Informed consent | X | | | | | | | | |
| Inclusion/exclusion criteria | X | X | | | | | | | |
| Medical and psychiatric history^{b} | X | | | | | | | | |
| Demographics | X | | | | | | | | |
| Physical examination^{c} | X | | | | | | | | X |
| Nasal cavity examination^{d} | X | X | X | X | | X | | X | |
| C-SSRS^{e} | X | X | X | X | X | X | X | X | X |
| Look back period | Life&Last | SLA | SLA | SLA | SLA | SLA | SLA | SLA | SLA |
| | mo | | | | | | | | |
| 12-lead ECG^{f} | X | X^{g} | | X^{h} | | X^{h} | | X^{h} | |
| Vital signsⁱ | X | X^{g} | X^{j} | X^{k} | | X^{k} | | X^{k} | X |
| Urine drug screen | X | X | X | X | | X | | X | |
| Serum pregnancy test | X | | | | | | | | X |
| Clinical laboratory tests | X | X | | | | | | | X |
| Viral serology tests | X | | | | | | | | |
| MOAA/S | X | X^{l} | | X^{l} | | X^{l} | | X^{l} | |
| CADSS | X | X^{m} | | X^{m} | | X^{m} | | X^{m} | |
| Sheehan Disability Scale | | X | | | | | | | X |
| CAPS-5 | X | X^{r} | X^{j} | | X^{j} | | X^{j} | | X^{j} |
| CGIS and PGIS | X | Xⁿ | X^{j} | | X^{j} | | X^{j} | | X^{j} |
| Look back period | Last | SLA | SLA | | SLA | | SLA | | SLA |
| | wk | | | | | | | | |
| CGIC and PGIC^{o} | | | X^{j} | | X^{j} | | X^{j} | | X^{j} |
| Look back period | | | SBL | | SBL | | SBL | | SBL |
| Prior and concomitant meds | X | X | X | X | X | X | X | X | X |
| AE monitoring | X | X | X | X | X | X | X | X | X |
| Randomization | | X^{p} | | | | | | | |
| Study drug administration^{q} | | X | | X | | X | | X | |

| | |
|---|---|
| Review instructions on proper use | X^{r} |
| of IN device | |

Abbreviations:
AE = adverse event;
ATRQ = antidepressant treatment response questionnaire;
BP = blood pressure;
C = change;
CADSS = Clinician-Administered Dissociative States Scale;
CGIC-SI/B = Clinical Global Impression of Change in Suicidal Ideation and Behavior;
CGIS-SI/B = Clinical Global Impression of Severity of Suicidal Ideation and Behavior;
C-SSRS = Columbia Suicide Severity Rating Scale;
CAPS-5 = Clinician Administered PTSD Scale for DSM-5;
ECG = electrocardiogram;
IN = intranasal;
MOAA/S = Modified Observer's Assessment of Alertness/Sedation (Scale);
mos = months;
PGIC-SI/B = Patient Global Impression of Change in Suicidal Ideation and Behavior;
PGIS-SI/B = Patient Global Impression of Severity of Suicidal Ideation and Behavior;
S = severity;
SBL = since baseline;
SLA = since last assessment;
wk = week
If needed, time points with multiple safety assessments may use a ±15-minute window. However, the C-SSRS assessment (also a safety measure) window remains at ±30 mins for each time point due to the time required for completion.
^{a}The assessments and procedures are listed in the preferred order of completion (top to bottom).
^{b}Medical history will consist of a complete medical history including all hospitalizations, procedures, and psychiatric history.
^{c}Measure height and weight at the time of physical examination. Height to be measured only at screening.
^{d}Nasal cavity examination will be performed before dosing of study drug and at approximately 2 hours and 24 hours post dose (first dose only).
^{e}Perform predose on dosing days and 24 hours post dose (±30 min).
^{f}On Day 1, predose, 3 serial, resting, supine 12-lead ECGs are to be conducted within 10 minutes total time. All other ECGs are single, resting, supine 12-lead recordings. When ECGs are to be collected at the same time point as a blood collection, ECGs should be collected first.
^{g}Perform predose (×3 for ECG only), and 1, 2, and 4 hours post dose (all ±10 min).
^{h}Perform predose and 1-hour post dose (all ±10 min).
ⁱVital signs include temperature, BP, respiration rate, and heart rate.
^{j}Perform approximately 24 (±30 min) hours post dose.
^{k}Perform predose, 1 and 2 hours post dose (all ±10 min).
^{l}Perform predose, 15, 30, 45, 60, 75, and 90 (all ±5) minutes post dose, and 2 (±10 min) hours post dose; however, if scores are ≤4, perform hourly until resolution.
^{m}Perform predose, 40 (±5) minutes post dose, and 1 (±10 min) and 2 (±10 min) hours post dose; however, if total scores are >4, perform hourly until resolution.
ⁿPerform predose with a SLA look back period.
^{o}For change scales, baseline is defined as the result of the severity assessment predose on Day 1.
^{p}Randomization will occur after subject is confirmed to have met inclusion/exclusion criteria.
^{q}On Day 1, dosing is suggested to occur in the morning hours. Doses on subsequent days should occur at approximately the same time of day (±1 hour) as the Day 1 dose.
^{r}Perform predose.
^{s}Dosing should occur every 4 days with dosing occurring on the following days (±/−1 day): Days 9, 13, 17, 21, 25, 29, 33, 37, 41, 45, 49, 53, 5, 61, 65, 69, 73, 77, 81 & 85.

TABLE 8
Schedule of Events

| | Visit Name | | | | |
|---|---|---|---|---|---|
| | | Safety | Safety | Safety | Safety |
| | Early | Follow | Follow | Follow | Follow |
| | Term | Up 1 | Up 2 | Up 3 | Up 4 |
| | Study Day | | | | |
| | | Week 13 | Week 14 | Week 15 | Week 16 |
| Assessments & | | (91 +/− | (98 +/− | (105 +/− | (112 +/− |
| Procedures^{a} | N/A | 1 day) | 1 day) | 1 day) | 1 day) |
| Physical examination^{b} | X (brief) | | | | X |
| Nasal cavity exam^{c} | X | | | | |
| C-SSRS^{d} | X | X | X | X | X |
| 12-lead ECG^{e} | X^{g} | | | | X |
| Vital signs^{h} | X^{g} | X^{g} | X^{g} | X^{g} | X^{g} |
| Urine drug screen | X | | | | X |
| Serum pregnancy test | X | | | | |
| Clinical laboratory tests | X | | | | X |
| Sheehan Disability Scale | X | | | | X |
| CAPS-5 | X | X | X | X | X |
| CGIS and PGIS | X | X | X | X | X |
| Look back period | SLA | SLA | SLA | SLA | SLA |
| CGIC and PGIC | X | X | X | X | X |
| Look back period^{l} | SBL | SBL | SBL | SBL | SBL |
| Prior and concomitant meds | X | X | X | X | X |
| AE monitoring | X | X | X | X | X |

Abbreviations:
AE = adverse event;
BP = blood pressure;
C = change;
CADSS = Clinician-Administered Dissociative States Scale;
CGIC-SI/B = Clinical Global Impression of Change in Suicidal Ideation and Behavior;
CGIS-SI/B = Clinical Global Impression of Severity of Suicidal Ideation and Behavior;
C-SSRS = Columbia Suicide Severity Rating Scale;
ECG = electrocardiogram;
MOAA/S = Modified Observer's Assessment of Alertness/Sedation (Scale);
mos = months;
PGIC-SI/B = Patient Global Impression of Change in Suicidal Ideation and Behavior;
PGIS-SI/B = Patient Global Impression of Severity of Suicidal Ideation and Behavior,
S = severity;
SBL = since baseline;
SLA = since last assessment;
wk = week
All efforts should be made to maintain the outlined study schedule; however, if dosing visit on Day 15 cannot be performed on that day, they may be performed (±1 day) as herein.
^{a}The assessments and procedures are listed in the preferred order of completion (top to bottom).
^{b}Measure weight at the time of physical examination.
^{c}Nasal cavity examination will be performed before dosing of study drug and approximately 2 hours post dose. The exam will also be performed on Day 16 (or early termination).
^{d}Perform predose on dosing day; for non-dosing days, perform at the approximate dosing-day time (±30 min).
^{e}ECGs are single, resting, supine 12-lead recordings. When ECGs are to be collected at the same time point as a blood collection, ECGs should be collected first.
^{f} Perform predose and 1-hour post dose (all ±10 min).
^{g}Perform assessment at any time during the visit.
^{h}Vital signs include temperature, BP, respiration rate, and heart rate.
ⁱ Perform predose, 1 and 2 hours post dose (all ±10 min).
^{j} Perform MOAA/S predose, 15, 30, 45, 60, 75, and 90 (±5) minutes post dose, and 2 hours post dose (all ±10 min); however, if scores are ≤4, perform hourly until resolution.
^{k} Perform CADSS predose, 40 (±5) minutes post dose, and 1, and 2 hours post dose (all ±10 min); however, if total scores are >4, perform hourly until resolution.
^{l}For change scales, baseline is defined as the result of the severity assessment predose on Day 1.
^{m} Dosing is suggested to occur at approximately the same time of day (±1 hour) as the Day 1 dose.

### Example 8. A Double Blind, Placebo-Controlled Phase 3 Study to Assess the Efficacy, Safety, and Tolerability of Intranasal Racemic Ketamine Administered to Adults with Bipolar I or II Disorder with a Moderate or Severe Major Depressive Episode (Bipolar Depression)

This example describes a Phase 3, multicenter double blind placebo controlled study to assess the efficacy, safety, and tolerability of intranasal ketamine in adult subjects with with Bipolar I or II Disorder with a moderate or severe depressive episode (Bipolar Depression).

### Study Overview

This Phase 3, randomized, double-blind, placebo-controlled multicenter, study in adult subjects diagnosed with Bipolar Depression evaluates the efficacy, safety, and tolerability of intranasally administered racemic ketamine. Approximately 220 subjects are randomized 1:1 to receive either intranasal racemic ketamine (90 mg) or matching placebo.

The study is designed to assess acute rapid response therapy in Bipolar Depression when co-administered with standard antidepressant treatment. Standard antidepressant treatment includes co-initiation of SSRI or SNRI, or optimization of current antidepressant regimen.

Each subject participates in a 1- to 2-day screening phase, a 16-day treatment phase including antidepressant standard of care during which study drug is administered 2 times per week, the standard antidepressant regimen is continued throughout, and a 4-week follow-up phase for a total of approximately 6 weeks of study participation. Subjects are treated in the clinic as outpatients, and return to the clinic to receive study drug and undergo study assessments 2 times per week until Day 16. Subjects are evaluated for efficacy using multiple psychometric scales and for safety using clinical laboratory assessments, electrocardiograms (ECGs), vital signs, and physical examinations. After the last dose of study drug, subjects are monitored for 4 weeks, including 4 in-person safety follow-up visits on Days 23, 30, 37, and 44 (all ±1).

### Objectives:

The primary objective of this study is to evaluate the efficacy of intranasal racemic ketamine on symptoms of depression in adults with moderate or severe depressive symptoms in patients with Bipolar I or II Disorder.

The secondary objectives are:
(1) To evaluate the timing of onset and persistence of response of intranasal racemic ketamine in adults with Bipolar I or II Disorder; and
(2) To evaluate the safety and tolerability of intranasal racemic ketamine in adults with Bipolar I or II Disorder.

### Number of Subjects

Approximately 220 randomized subjects including 110 subjects per arm.

### Criteria for Inclusion:

Subjects must fulfill all of the following requirements to enter the study:
(1) The subject is able to speak, read, and understand English and/or the language of the investigative staff to sufficiently understand the nature of the study, to provide written informed consent, and to allow the completion of all study assessments;
(2) Subject is 18 to 65 years of age at the time of informed consent;
(3) Male subjects who are sexually active must agree to abstain from sexual activity or be willing to use medically acceptable contraception if they become sexually active from time of consent and for 3 months after the last dose of study drug;
(4) Female subjects must have a negative serum pregnancy test at screening, and must not be breastfeeding or lactating. Females must be willing to abstain from sexual activity or use medically accepted contraception if they are sexually active from time of consent and for 1 month after the last dose of study drug;
(5) The subject meets Diagnostic and Statistical Manual of Mental Disorders, Fifth Edition (DSM-5) criteria for a diagnosis of bipolar disorder I or II, current major depressive episode (without psychotic features and rapid cycling disease course, ie no less than 4 episodes of mood disturbance in the 12 months prior to screening), with symptoms present for at least 4 weeks, based on psychiatric intake and confirmed by the Mini International Psychiatric Interview Version 7.02 (MINI);
(6) The subject has a Montgomery Åsberg Depression Rating Scale (MADRS) total score of ≥28 predose on Day 1;
(7) The subject is willing and able to take prescribed non-investigational antidepressant therapy for at least the duration of the study;
(8) The subject is willing to avoid the use of alcohol, CBD oil, and recreational drugs, as well as specific therapies prohibited by the protocol, from screening through the last study visit (Day 44); and
(9) The subject is able to complete intranasal administration of study drug.

### Exclusion Criteria:

The presence of any of the following criteria excludes a subject from participating in the study:
(1) Subjects who have ongoing sequelae from prior COVID illness, or subjects who have either documented COVID infection or symptoms suggestive of recent COVID infection within 1 month of screening;
(2) The subject has a lifetime diagnosis of any mood disorder with psychotic features, schizophrenia or other psychotic disorder, obsessive-compulsive disorder, or antisocial personality disorder, as confirmed by the MINI. (Note subjects who have post-traumatic stress disorder and generalized anxiety disorder (GAD)/panic disorder are not necessarily excluded as long as bipolar I or II is the most prominent diagnosis.);
(3) In the investigator's opinion, the subject has chronic, refractory treatment-resistant depression from >4 adequate therapeutic trials of antidepressants (with or without adjuvants and/or electroconvulsive therapy [ECT]) as confirmed by ATRQ;
(4) In the investigator's opinion, the subject has a current diagnosis of borderline personality disorder or, if the subject has not met full diagnostic criteria for borderline personality disorder within the last 5 years, the subject has a history of recurrent non-suicidal self-injury, or self-mutilating behavior;
(5) In the investigator's opinion, the subject is judged to be an acute suicide risk, scoring ≥4 on MADRS item 10 in the past month, or has a history of severe near-fatal attempts requiring hospitalization;
(6) The subject is diagnosed with intellectual disability, a neurocognitive disorder including dementia, or has a history of moderate or severe traumatic brain injury. Mild traumatic brain injuries are not necessarily exclusionary provided the investigator believes current symptoms would not interfere with conduct or interpretation of safety and/or efficacy assessments;
(7) The subject has a history or current finding of any clinically significant, hematological, hepatic, respiratory, renal, neurological, known positive human immunodeficiency virus (HIV) infection, gastrointestinal disorder, or other disease that might confound the results of safety assessments conducted in the study;
(8) The subject has a history of seizures (other than childhood febrile seizures);
(9) The subject has a body mass index (BMI)>40 or <18 at screening;
(10) The subject has known, uncontrolled hypertension or blood pressure (BP) that, in the investigator's judgment, should exclude the subject at screening or baseline (BP may be repeated as per the site's standard operating procedures [SOPs]);
(11) The subject has a known history or current finding of cardiovascular disease, advanced arteriosclerosis, structural cardiac abnormality, cardiomyopathy, serious heart rhythm abnormalities, coronary artery disease, congenital heart disease, ischemic heart disease, cardiac insufficiency, supraventricular and ventricular heart rhythm disorder, prolonged QT syndrome (i.e., QTcF >450 msec) and associated risk factors (i.e., hypokalemia, family history of long QT syndrome), syncope, cardiac conduction problems (e.g., clinically significant heart block), exercise-related cardiac events including syncope and pre-syncope, clinically significant bradycardia, or other serious cardiac problems,
(12) The subject has a known family history of sudden cardiac death or ventricular arrhythmia;
(13) The subject has any clinically significant abnormality on 12-lead ECG performed at screening or baseline such as serious arrhythmia, cardiac conduction problems, or other abnormalities deemed to be a potential safety issue;
(14) The subject has a concurrent chronic or acute illness, disability, or other condition (e.g., narcolepsy) that might confound the results of safety assessments conducted in the study;
(15) The subject has any medical condition that could interfere with the absorption of intranasal ketamine (e.g., nasal polyps, clinically significant deviated septum [corrected or persistent], or other physical abnormalities of the nose);
(16) The subject has symptomatic or uncontrolled hyper- or hypothyroidism or has had changes to their treatment for hyper- or hypothyroidism within 90 days prior to Day 1;
(17) The subject meets the DSM-5 criteria for moderate or severe substance use disorder in the 6 months prior to screening, or in the investigator's opinion, is at risk of withdrawal from substance use (e.g., opiate or alcohol dependent), or has a lifetime history of ketamine, phencyclidine, lysergic acid diethylamide, or 4-methylenedioxy-methamphetamine hallucinogen-related use disorders. Nicotine use disorder is permitted;
(18) The subject requires daily benzodiazepine use of >2 mg of lorazepam or dose equivalent;
(19) The subject has a positive urine test for phencyclidine (PCP), cocaine, or amphetamines (inclusive of amphetamine, methamphetamine [mAMP], and 3, 4-methylenedioxy-methamphetamine [MDMA]) at screening;
(20) The subject has positive hepatitis B, hepatitis C, or HIV results at screening;
(21) The subject has any history of using ketamine or esketamine for any psychiatric treatment;
(22) The subject has known or suspected intolerance or hypersensitivity to the investigational product(s), closely related compounds, or any ingredients.;
(23) In the investigator's opinion, the subject has any clinically significant laboratory abnormality including an abnormality that indicates clinically significant hematologic, hepatobiliary, or renal disease. Note, any screening abnormality must be discussed with the Medical Monitor for approval prior to randomization;
(24) The subject has received an investigational product, including investigational vaccines, within 6 months prior to the first dose of study drug;
(25) The subject was previously enrolled in the current study. Subjects previously screened for, but not randomized in, the current study may be rescreened with approval from the study medical monitor;
(26) The subject does not meet or is not willing to comply with the requirements related to prohibited and restricted medications and therapies, as well as required washout periods prior to participation. Prohibited medications and therapies include, but are not limited to, monoamine oxidase inhibitors (MAOIs), opioids or drugs with activity at the opioid receptor, psychostimulants, lamotrigine, N-methyl-D-aspartate (NMDA) receptor modulators, magnesium, electroconvulsive therapy (ECT), transcranial magnetic stimulation (TMS) or any medication/therapy that might confound the results of safety assessments conducted in the study. Subjects who have received any of these prohibited medications within 2 weeks of screening are excluded from the study. Potent CYP 3A4 inhibitors, including nefazodone and fluvoxamine, are not permitted within 1 week of first dose and until at least 1 day after the last dose. Potent CYP 3A4 inducers, including St. John's wort, are not permitted within 30 days of first dose and until at least 1 day after the last dose;
(27) The subject is an employee of the sponsor, clinical research organization, or research site personnel directly affiliated with this study or their immediate family member defined as a spouse, parent, child, or sibling, whether biological or legally adopted;
(28) The subject has pending legal charges or is on probation;
(29) The subject, in the investigator's opinion, is considered unsuitable or unlikely to comply with the study protocol for any reason; and
(30) The subject is legally incapacitated, has been involuntarily hospitalized in the past year, or has another significant mental health issue, physical issue, or life circumstance that could interfere with the conduct or interpretation of study evaluations.

### Concomitant Treatment

Optimization of current antidepressant treatment or initiation of a new SSRI or SNRI antidepressant as well as a mood stabilizer is required for all subjects during the study, at study start. In addition, subjects are permitted to be participating in standard of care psychotherapy, including behavioral therapy. Benzodiazepine (dose equivalent to ≤2 mg/day of lorazepam) medications may be taken on a limited basis as needed, as defined in the protocol restricted medications section, but should not be taken within 24 hours prior to study medication dosing and assessments. Short acting non-benzodiazepine hypnotics (e.g., zolpidem, zaleplon) are permitted, but may never be taken within 10 hours prior to study medication dosing.

### Criteria for Evaluation:

### Primary Efficacy Endpoints:

The primary efficacy endpoint is the change from baseline in the MADRS total score at 24 hours following the initial dose.

### Key Secondary Endpoints:

The key secondary endpoints (in rank order) is the change from baseline in:
(1) MADRS total score at Day 16,
(2) Clinical Global Impression Severity (CGIS) at 24 hours,
(3) Patient Global Impression Severity (PGIS) at 24 hours,
(4) CGIS at Day 16, and
(5) PGIS at Day 16.

### Additional Secondary Endpoints:

The additional secondary endpoints are the change from baseline at 24 hours, as well as at Day 16, and end of week 6 in the following:
(1) MADRS response (total score ≥50% reduction from baseline),
(2) Onset of clinical response (MADRS total score ≥50% reduction from baseline by 24 hrs and maintained to Day 16),
(3) MADRS remission (total score ≤12),
(4) MADRS Total Score at end of week 6,
(5) Clinical Global Impression Change (CGIC) at end of week 6,
(6) Patient Global Impression Change (PGIC) at end of week 6,
(7) Sheehan Disability Scale (not assessed at 24 hr),
(8) Young Mania Rating Scale,
(9) Posttreatment Phase—time to relapse for patients who were in remission in the treatment phase, who subsequently relapse during the 4 week safety follow-up, and
(10) Posttreatment Phase—time to relapse for patients who were responders in the treatment phase, who subsequently relapse during the 4 week safety follow-up (Relapse is defined as MADRS total score ≥22 for two consecutive weeks).

### Safety Endpoints:

The safety and tolerability of intranasal racemic ketamine are assessed by:
(1) The frequency and severity of treatment-emergent adverse events (TEAEs);
(2) Frequencies of new or worsening clinically significant laboratory, vital signs, ECGs, or physical examination abnormalities;
(3) Clinician-Administered Dissociative States Scale (CADSS);
(4) Modified Observer's Assessment of Alertness/Sedation (MOAA/S) Scale;
(5) Columbia Suicide Severity Rating Scale (C-SSRS); and
(6) Physician Withdrawal Checklist 20-item (PWC-20).

### Statistical Methods

For the analysis of the primary efficacy endpoint, the change in the MADRS total score from baseline at 24 hours following the initial dose, is analyzed using an ANCOVA model including baseline MADRS total score as a covariate, treatment as fixed effect and a random subject effect. A hierarchical testing procedure is used to maintain the experiment-wise type 1 error rate at 5%. Endpoints, starting with the primary endpoint, are tested in the pre-specified order of importance. Once a p-value greater than 0.05 is obtained, all subsequent hypothesis testing are stopped. Further details of the statistical analyses, including secondary efficacy, safety, and sensitivity analyses, are described in the protocol and/or the Statistical Analysis Plan (SAP).

### Sample Size Determination

The sample size is calculated assuming an effect size of 0.45, a 2-sided significance level of 0.05, and assuming approximately a 15% dropout rate. Based on these assumptions, 110 subjects are randomized to each treatment group to achieve 90% power. The effect size used in this calculation was based on results of previous ketamine studies and on clinical judgment.

### Study and Treatment Duration

The sequence and maximum duration of the study periods are as follows:
a 2- to 7-day screening phase,
a 16-day treatment phase of investigational study drug which is co-administered with a standard of care antidepressant regimen, and
a 4-week safety follow up.

Therefore, the maximum study duration for each subject is approximately 6 weeks.

All subjects are referred for follow up post study; this includes subjects who do not qualify for study, subjects who discontinue participation, and those subjects who complete the study.

### Example 9. A Double Blind, Placebo-Controlled Phase 3 Study to Assess the Efficacy, Safety, and Tolerability of Intranasal Racemic Ketamine Administered to Adults with Moderate or Severe Major Depressive Disorder

This example describes a Phase 3 double blind, placebo controlled study to assess the efficacy, safety, and tolerability of intranasal racemic ketamine in adult subjects with moderate or severe major depressive disorder (MDD) plus standard of care (SOC).

### Study Overview

This Phase 3 randomized, double-blind, placebo-controlled multicenter, study in adult subjects diagnosed with MDD evaluates the efficacy, safety, and tolerability of intranasally administered racemic ketamine plus SOC. Approximately 240 subjects are randomized 1:1 to receive either intranasal racemic ketamine (90 mg) or matching placebo.

The study is designed to assess acute therapy in MDD when co-administered with standard antidepressant treatment. Standard antidepressant treatment includes co-initiation of SSRI or SNRI, or optimization of current antidepressant regimen.

Each subject participates in a 2- to 7-day screening phase, a 16-day treatment phase including antidepressant SOC during which study drug is administered 2 times per week, the standard antidepressant regimen is continued throughout, and a 4-week follow-up phase for a total of approximately 6 weeks of study participation. SOC is not changed after baseline during the 16-day treatment phase and is optimized if necessary during the 4-week follow-up phase. Subjects are treated in the clinic as outpatients, and return to the clinic to receive study drug and undergo study assessments 2 times per week until Day 16. Subjects are evaluated for efficacy using multiple psychometric scales and for safety using clinical laboratory assessments, electrocardiograms (ECGs), vital signs, and physical examinations. After the last dose of study drug, subjects are monitored for 4 weeks, including 4 in-person safety follow-up visits on Days 23, 30, 37, and 44 (all ±1).

### Objectives:

The primary objective of this study is to evaluate the efficacy of intranasal racemic ketamine plus SOC on symptoms of depression in adults with moderate or severe major depressive disorder (MDD).

The secondary objectives are:
(1) To evaluate the timing of onset of response and, for responders, the persistence of benefit of intranasal racemic ketamine plus SOC in adults with MDD; and
(2) To evaluate the safety and tolerability of intranasal racemic ketamine plus SOC in adults with MDD.

### Number of Subjects

Approximately 240 randomized subjects including 120 subjects per arm.

### Criteria for Inclusion:

Subjects must fulfill all of the following requirements to enter the study:
(1) The subject is able to speak, read, and understand English and/or the language of the investigative staff to sufficiently understand the nature of the study, to provide written informed consent, and to allow the completion of all study assessments;
(2) Subject is 18 to 65 years of age at the time of informed consent;
(3) Male subjects who are sexually active must agree to abstain from sexual activity or be willing to use medically acceptable contraception if they become sexually active from time of consent and for 3 months after the last dose of study drug;
(4) Female subjects of childbearing potential must have a negative serum pregnancy test at screening, and must not be breastfeeding or lactating. Females must be willing to abstain from sexual activity or use medically accepted contraception if they are sexually active from time of consent and for 1 month after the last dose of study drug;
(5) The subject meets Diagnostic and Statistical Manual of Mental Disorders, Fifth Edition (DSM-5) criteria for a diagnosis of current MDD (unipolar without psychotic features), with symptoms present for at least 4 weeks, based on psychiatric intake and confirmed by the Mini International Psychiatric Interview Version 7.02 (MINI);
(6) The subject has a Montgomery Åsberg Depression Rating Scale (MADRS) total score of ≥28 predose on Day 1;
(7) The subject is willing and able to take prescribed non-investigational antidepressant therapy for at least the duration of the study;
(8) The subject is willing to avoid the use of alcohol, CBD oil, and recreational drugs, as well as specific therapies prohibited by the protocol, from screening through the last study visit (Day 44); and
(9) The subject is able to complete intranasal administration of study drug.

### Exclusion Criteria:

The presence of any of the following criteria excludes a subject from participating in the study:
(1) Subjects who have ongoing sequelae from prior COVID illness, or subjects who have either documented COVID infection or symptoms suggestive of recent COVID infection within 1 month of screening;
(2) The subject has a lifetime diagnosis of bipolar disorder, any mood disorder with psychotic features, schizophrenia or other psychotic disorder, obsessive-compulsive disorder, or antisocial personality disorder, as confirmed by the MINI (Note subjects who have post-traumatic stress disorder and generalized anxiety disorder (GAD)/panic disorder are not necessarily excluded as long as MDD is the most prominent diagnosis.);
(3) In the investigator's opinion, the subject has chronic, refractory treatment-resistant depression from >4 adequate therapeutic trials of antidepressants (with or without adjuvants and/or electroconvulsive therapy [ECT]) as confirmed by ATRQ;
(4) In the investigator's opinion, the subject has a current diagnosis of borderline personality disorder or, if the subject has not met full diagnostic criteria for borderline personality disorder within the last 5 years, the subject has a history of recurrent non-suicidal self-injury, or self-mutilating behavior;
(5) In the investigator's opinion, the subject is judged to be an acute suicide risk, scoring ≥4 on MADRS item 10 in the past month;
(6) Suicide attempt within the last 12 months, on the C-SSRS, and a score of 2, 3, or 4 for Actual Lethality/Medical Damage;
(7) The subject is diagnosed with intellectual disability, a neurocognitive disorder including dementia, or has a history of moderate or severe traumatic brain injury. Mild traumatic brain injuries are not necessarily exclusionary provided the investigator believes current symptoms would not interfere with conduct or interpretation of safety and/or efficacy assessments;
(8) The subject has a history or current finding of any clinically significant, hematological, hepatic, respiratory, renal, neurological, known positive human immunodeficiency virus (HIV) infection, gastrointestinal disorder, or other disease that might confound the results of safety assessments conducted in the study;
(9) The subject has a history of seizures (other than childhood febrile seizures);
(10) The subject has a body mass index (BMI)>40 or <18 at screening;
(11) The subject has known, uncontrolled hypertension or blood pressure (BP) that, in the investigator's judgment, should exclude the subject at screening or baseline (BP may be repeated as per the site's standard operating procedures [SOPs]);
(12) The subject has a known history or current finding of cardiovascular disease, advanced arteriosclerosis, structural cardiac abnormality, cardiomyopathy, serious heart rhythm abnormalities, coronary artery disease, congenital heart disease, ischemic heart disease, cardiac insufficiency, supraventricular and ventricular heart rhythm disorder, prolonged QT syndrome (i.e., QTcF >450 msec) and associated risk factors (i.e., hypokalemia, family history of long QT syndrome), syncope, cardiac conduction problems (e.g., clinically significant heart block), exercise-related cardiac events including syncope and pre-syncope, clinically significant bradycardia, or other serious cardiac problems;
(13) The subject has a known family history of sudden cardiac death or ventricular arrhythmia;
(14) The subject has any clinically significant abnormality on 12-lead ECG performed at screening or baseline such as serious arrhythmia, cardiac conduction problems, or other abnormalities deemed to be a potential safety issue;
(15) The subject has a concurrent chronic or acute illness, disability, or other condition (e.g., narcolepsy) that might confound the results of safety assessments conducted in the study.
(16) The subject has any medical condition that could interfere with the absorption of intranasal ketamine (e.g., nasal polyps, clinically significant deviated septum [corrected or persistent], or other physical abnormalities of the nose);
(17) The subject has symptomatic or uncontrolled hyper- or hypothyroidism or has had changes to their treatment for hyper- or hypothyroidism within 90 days prior to Day 1;
(18) The subject meets the DSM-5 criteria for moderate or severe substance use disorder in the 6 months prior to screening, or in the investigator's opinion, is at risk of withdrawal from substance use (e.g., opiate or alcohol dependent), or has a lifetime history of ketamine, phencyclidine, lysergic acid diethylamide, or 4-methylenedioxy-methamphetamine hallucinogen-related use disorders. Nicotine use disorder is permitted;
(19) The subject requires daily benzodiazepine use of >2 mg of lorazepam or dose equivalent
(20) The subject has a positive urine test for phencyclidine (PCP), cocaine, or amphetamines (inclusive of amphetamine, methamphetamine [mAMP], and 3, 4-methylenedioxy-methamphetamine [MDMA]) at screening;
(21) The subject has positive hepatitis B, hepatitis C, or HIV results at screening;
(22) The subject has any history of using ketamine or esketamine for any psychiatric treatment;
(23) The subject has known or suspected intolerance or hypersensitivity to the investigational product(s), closely related compounds, or any ingredients;
(24) In the investigator's opinion, the subject has any clinically significant laboratory abnormality including an abnormality that indicates clinically significant hematologic, hepatobiliary, or renal disease. Note, any screening abnormality must be discussed with the Medical Monitor for approval prior to randomization;
(25) The subject has received an investigational product, including investigational vaccines, within 6 months prior to the first dose of study drug;
(26) The subject was previously enrolled in the current study. Subjects previously screened for, but not randomized in, the current study may be rescreened with approval from the study medical monitor;
(27) The subject does not meet or is not willing to comply with the requirements related to prohibited and restricted medications and therapies, as well as required washout periods prior to participation. Prohibited medications and therapies include, but are not limited to, monoamine oxidase inhibitors (MAOIs), opioids or drugs with activity at the opioid receptor, psychostimulants, lamotrigine, N-methyl-D-aspartate (NMDA) receptor modulators, magnesium, electroconvulsive therapy (ECT), transcranial magnetic stimulation (TMS) or any medication/therapy that might confound the results of safety assessments conducted in the study. Subjects who have received any of these prohibited medications within 2 weeks of screening are excluded from the study. Potent CYP 3A4 inhibitors, including nefazodone and fluvoxamine, are not permitted within 1 week of first dose and until at least 1 day after the last dose. Potent CYP 3A4 inducers, including St. John's wort, are not permitted within 30 days of first dose and until at least 1 day after the last dose. Subjects who have recently discontinued lithium or calcium channel blockers within 3 months prior to screening are also excluded;
(28) The subject is an employee of the sponsor, clinical research organization, or research site personnel directly affiliated with this study or their immediate family member defined as a spouse, parent, child, or sibling, whether biological or legally adopted;
(29) The subject has pending legal charges or is on probation;
(30) The subject, in the investigator's opinion, is considered unsuitable or unlikely to comply with the study protocol for any reason; and
(31) The subject is legally incapacitated, has been involuntarily hospitalized in the past year, or has another significant mental health issue, physical issue, or life circumstance that could interfere with the conduct or interpretation of study evaluations.

### Concomitant Treatment

Evidence-/empirically based optimization of current antidepressant treatment or initiation of a new SSRI or SNRI antidepressant is required for all subjects during the study, at study start. SOC should remain stable during the 16-day treatment phase. In addition, subjects are permitted to be participating in SOC psychotherapy, including behavioral therapy. Benzodiazepine (dose equivalent to ≤2 mg/day of lorazepam) medications may be taken on a limited basis as needed, as defined in the protocol restricted medications section, but should not be taken within 24 hours prior to study medication dosing and assessments. Short acting non-benzodiazepine hypnotics (e.g. zolpidem, zaleplon) are permitted, but may never be taken within 10 hours prior to study medication dosing.

### Criteria for Evaluation:

### Primary Efficacy Endpoints:

The primary efficacy endpoint is the change from baseline in the MADRS total score at 24 hours following the initial dose.

### Key Secondary Endpoints:

The key secondary endpoints (in rank order) is the change from baseline in:
(1) MADRS total score at Day 16,
(2) Clinical Global Impression Severity (CGIS) at 24 hours,
(3) Patient Global Impression Severity (PGIS) at 24 hours,
(4) CGIS at Day 16, and
(5) PGIS at Day 16.

### Additional Secondary Endpoints:

The additional secondary endpoints are the change from baseline in the following:
(1) Number and % of MADRS responders (total score ≥50% reduction from baseline) at each efficacy timepoint,
(2) Number and % of persistent MADRS responders from onset at each timepoint and maintained through Day 16,
(3) Number and % of persistent MADRS responders from onset at each timepoint and maintained through Week 6,
(4) MADRS remission (total score ≤12) at each efficacy timepoint,
(5) MADRS Total Score at week 6,
(6) CGIS at week 6,
(7) PGIS at week 6,
(8) Clinical Global Impression Change (CGIC) at Day 16 and week 6,
(9) Patient Global Impression Change (PGIC) at Day 16 and week 6,
(10) Sheehan Disability Scale at Day 16 and week 6, and
(11) Posttreatment Phase—time to relapse for responders in the treatment phase, who subsequently relapse during the 4 week safety follow-up (Relapse is defined as MADRS total score ≥22 for two consecutive weeks).

### Safety Endpoints:

The safety and tolerability of intranasal racemic ketamine are assessed by:
(1) The frequency and severity of treatment-emergent adverse events (TEAEs);
(2) Frequencies of new or worsening clinically significant laboratory, vital signs, ECGs, or physical examination abnormalities;
(3) Clinician-Administered Dissociative States Scale (CADSS);
(4) Modified Observer's Assessment of Alertness/Sedation (MOAA/S) Scale;
(5) Columbia Suicide Severity Rating Scale (C-SSRS); and
(6) Physician Withdrawal Checklist 20-item (PWC-20).

### Statistical Methods

For the analysis of the primary efficacy endpoint, the change in the MADRS total score from baseline at 24 hours following the initial dose, is analyzed using an ANCOVA model including baseline MADRS total score as a covariate, treatment and class of antidepressant as fixed effect, and subject within class of antidepressant as random effect. A hierarchical testing procedure is used to maintain the experiment-wise type 1 error rate at 5%. Endpoints, starting with the primary endpoint, are tested in the pre-specified order of importance. Once a p-value greater than 0.05 is obtained, all subsequent hypothesis testing are stopped. Further details of the statistical analyses, including secondary efficacy, safety, and sensitivity analyses, are described in the protocol and/or the Statistical Analysis Plan (SAP).

### Sample Size Determination

The sample size is calculated assuming an effect size of 0.45, a 2-sided significance level of 0.05, and assuming approximately a 15% dropout rate. Based on these assumptions, 120 subjects are randomized to each treatment group to achieve 90% power. The effect size used in this calculation was based on results of previous ketamine studies and on clinical judgment.

### Study and Treatment Duration

The sequence and maximum duration of the study periods are as follows:
a 2- to 7-day screening phase,
a 16-day treatment phase of investigational study drug which is co-administered with a SOC antidepressant regimen (this regimen should remain stable during this phase), and
a 4-week safety follow up during which SOC is continued and optimized as clinically warranted.

Therefore, the maximum study duration for each subject is approximately 7 weeks.

All subjects are referred for follow up post study; this includes subjects who do not qualify for study, subjects who discontinue participation, and those subjects who complete the study.

TABLE 9
Schedule of Events

| | | 2 - | | | 5 | | 7 | | 9 |
|---|---|---|---|---|---|---|---|---|---|
| | | Baseline | | 4 | 5 | 6 | 9 | 8 | 12 |
| Visit Number | 1 - Screen | 1 | 3 | 4 | Phone | 8 | Phone | 11 | Phone |
| Study Day | −7 to −2 | Dosing | 2 | Dosing | Followup | Dosing | Followup | Dosing | Follow up |
| Assessments & | | | | | | | | | |
| Procedures^{a} | | | | | | | | | |
| Informed consent | X | | | | | | | | |
| Inclusion/exclusion | X | X | | | | | | | |
| criteria | | | | | | | | | |
| Medical and | X | | | | | | | | |
| psychiatric history^{b} | | | | | | | | | |
| Demographics | X | | | | | | | | |
| Physical examination^{c} | X | | | | | | | | |
| Nasal cavity | X | X | X | X | | X | | X | |
| examination^{d} | | | | | | | | | |
| C-SSRS^{e} | X | X | X | X | X | X | X | X | X |
| Look back period | Lifetime | SLA | SLA | SLA | SLA | SLA | SLA | SLA | SLA |
| | & past | | | | | | | | |
| | month | | | | | | | | |
| 12-lead ECG^{f} | X | X^{g} | | X^{h} | | X^{h} | | X^{h} | |
| Vital signsⁱ | X | X^{g} | X^{j} | X^{k} | | X^{k} | | X^{k} | |
| Urine drug screen | X | X | X | X | | X | | X | |
| Serum pregnancy test | X | | | | | | | | |
| Clinical laboratory | X | X | | | | | | | |
| tests | | | | | | | | | |
| Viral serology tests | X | | | | | | | | |
| MINI 7.02 | X | | | | | | | | |
| ATRQ | X | | | | | | | | |
| MOAA/S | X | X^{l} | | X^{l} | | X^{l} | | X^{l} | |
| CADSS | X | X^{m} | | X^{m} | | X^{m} | | X^{m} | |
| Sheehan Disability | | X | | | | | | | |
| Scale | | SLA | | | | | | | |
| MADRS | X | Xⁿ | X^{j} | | X^{j} | | X^{j} | | X^{j} |
| Look back period | Last wk | SLA | SLA | | SLA | | SLA | | SLA |
| CGIS and PGIS | X | Xⁿ | X^{j} | | X^{j} | | X^{j} | | X^{j} |
| Look back period | Last wk | SLA | SLA | | SLA | | SLA | | SLA |
| CGIC and PGIC^{o} | | | X^{j} | | X^{j} | | X^{j} | | X^{j} |
| Look back period | | | SBL | | SBL | | SBL | | SBL |
| Prior and concomitant | X | X | X | X | X | X | X | X | X |
| meds | | | | | | | | | |
| AE monitoring | X | X | X | X | X | X | X | X | X |
| Randomization | | X^{p} | | | | | | | |
| Study drug | | X | | X | | X | | X | |
| administration^{q} | | | | | | | | | |
| Review instructions | | X^{r} | | | | | | | |
| on proper use of IN | | | | | | | | | |
| device | | | | | | | | | |

Abbreviations:
AE = adverse event;
ATRQ = antidepressant treatment response questionnaire;
BP = blood pressure;
C = change;
CADSS = Clinician-Administered Dissociative States Scale;
CGIC-SI/B = Clinical Global Impression of Change in Suicidal Ideation and Behavior;
CGIS-SI/B = Clinical Global Impression of Severity of Suicidal Ideation and Behavior;
C-SSRS = Columbia Suicide Severity Rating Scale;
ECG = electrocardiogram;
IN = intranasal;
MADRS = Montgomery Åsberg Depression Rating Scale;
MINI = Mini International Psychiatric Interview Version 7.02;
MOAA/S = Modified Observer's Assessment of Alertness/Sedation (Scale);
mos = months;
PGIC-SI/B = Patient Global Impression of Change in Suicidal Ideation and Behavior;
PGIS-SI/B = Patient Global Impression of Severity of Suicidal Ideation and Behavior;
S = severity;
SBL = since baseline;
SLA = since last assessment;
wk = week
If needed, time points with multiple safety assessments may use a ±15-minute window. However, the C-SSRS assessment (also a safety measure) window remains at ±30 mins for each time point due to the time required for completion.
All efforts should be made to maintain the outlined study schedule; however, if dosing visits on Days 4, 8, and 11 cannot be performed on that day, they may be performed (±1 day) as described herein.
^{a}The assessments and procedures are listed in the preferred order of completion (top to bottom).
^{b}Medical history will consist of a complete medical history including all hospitalizations, procedures, and psychiatric history.
^{c}Measure height and weight at the time of physical examination. Height to be measured only at screening.
^{d}Nasal cavity examination will be performed before dosing of study drug and at approximately 2 hours and 24 hours postdose (first dose only).
^{e}Perform predose on dosing days and 24 hours postdose (±30 min).
^{f}On Day 1, predose, 3 serial, resting, supine 12-lead ECGs are to be conducted within 10 minutes total time. All other ECGs are single, resting, supine 12-lead recordings. When ECGs are to be collected at the same time point as a blood collection, ECGs should be collected first.
^{g}Perform predose (×3 for ECG only), and 1, 2, and 4 hours postdose (all ±10 min).
^{h}Perform predose and 1 hour postdose (all ±10 min).
ⁱVital signs include temperature, BP, respiration rate, and heart rate.
^{j}Perform approximately 24 (±30 min) hours postdose.
^{k}Perform predose, 1 and 2 hours postdose (all ±10 min).
^{l}Perform predose, 15, 30, 45, 60, 75, and 90 (all ±5) minutes postdose, and 2 (±10 min) hours postdose; however, if scores are ≤4, perform hourly until resolution.
^{m}Perform predose, 40 (±5) minutes postdose, and 1 (±10 min) and 2 (±10 min) hours postdose; however, if total scores are >4, perform hourly until resolution.
ⁿPerform predose with a SLA look back period.
^{o}For change scales, baseline is defined as the result of the severity assessment predose on Day 1.
^{p}Randomization will occur after subject is confirmed to have met inclusion/exclusion criteria.
^{q}On Day 1, dosing is suggested to occur at 10 am (±30 min). Doses on subsequent days must occur at approximately the same time of day (±1 hour) as the Day 1 dose.
^{r}Perform predose.

TABLE 10
Schedule of Events

| | | 11 | 12 | 13 | 14 | 15 |
|---|---|---|---|---|---|---|
| | | Endpt/ | Safety | Safety | Safety | Safety |
| | 10 | Early | Follow | Follow | Follow | Follow |
| Visit Number | 15 | Term | Up 1 | Up 2 | Up 3 | Up 4 |
| Study Day | Dosing | 16 + 1 | 23 ± 1 | 30 ± 1 | 37 ± 1 | 44 ± 1 |
| Assessments & Procedures^{a} | | | | | | |
| Physical examination^{b} | | X (brief) | | | | X |
| Nasal cavity exam^{c} | X | X | | | | |
| C-SSRS^{d} | X | X | X | X | X | X |
| 12-lead ECG^{e} | X^{f} | X^{g} | | | | X |
| Vital signs^{h} | Xⁱ | X^{g} | X^{g} | X^{g} | X^{g} | X^{g} |
| Urine drug screen | X | X | | | | X |
| Serum pregnancy test | | X | | | | |
| Clinical laboratory tests | | X | | | | |
| MOAA/S | X^{j} | | | | | |
| CADSS | X^{k} | | | | | |
| PWC-20 | X | X | X | X | | |
| Sheehan Disability Scale | | X | | X | | X |
| | | SLA | | SLA | | SLA |
| MADRS | | X | X | X | X | X |
| Look back period | | SLA | SLA | SLA | SLA | SLA |
| CGIS and PGIS | | X | X | X | X | X |
| Look back period | | SLA | SLA | SLA | SLA | SLA |
| CGIC and PGIC | | X | X | X | X | X |
| Look back period^{l} | | SBL | SBL | SBL | SBL | SBL |
| Prior and concomitant meds | X | X | X | X | X | X |
| AE monitoring | X | X | X | X | X | X |
| Study drug administration | X^{m} | | | | | |

Abbreviations:
AE = adverse event;
BP = blood pressure;
C = change; CADSS = Clinician-Administered Dissociative States Scale;
CGIC-SI/B = Clinical Global Impression of Change in Suicidal Ideation and Behavior;
CGIS-SI/B = Clinical Global Impression of Severity of Suicidal Ideation and Behavior;
C-SSRS = Columbia Suicide Severity Rating Scale;
ECG = electrocardiogram;
MADRS = Montgomery Åsberg Depression Rating Scale;
MINI = Mini International Psychiatric Interview Version 7.02;
MOAA/S = Modified Observer's Assessment of Alertness/Sedation (Scale);
mos = months;
PGIC-SI/B = Patient Global Impression of Change in Suicidal Ideation and Behavior;
PGIS-SI/B = Patient Global Impression of Severity of Suicidal Ideation and Behavior;
Physician Withdrawal Checklist = PWC;
S = severity;
SBL = since baseline;
SLA = since last assessment;
wk = week
All efforts should be made to maintain the outlined study schedule; however, if dosing visit on Day 15 cannot be performed on that day, they may be performed (±1 day) as described herein.
^{a}The assessments and procedures are listed in the preferred order of completion (top to bottom).
^{b}Measure weight at the time of physical examination.
^{c}Nasal cavity examination will be performed before dosing of study drug and approximately 2 hours postdose. The exam will also be performed on Day 16 (or early termination).
^{d}Perform predose on dosing day; for non-dosing days, perform at the approximate dosing-day time (±30 min).
^{e}ECGs are single, resting, supine 12-lead recordings. When ECGs are to be collected at the same time point as a blood collection, ECGs should be collected first.
^{f}Perform predose and 1 hour postdose (all ±10 min).
^{g}Perform assessment at any time during the visit.
^{h}Vital signs include temperature, BP, respiration rate, and heart rate.
ⁱPerform predose, 1 and 2 hours postdose (all ±10 min).
^{j}Perform MOAA/S predose, 15, 30, 45, 60, 75, and 90 (±5) minutes postdose, and 2 hours postdose (all ±10 min); however, if scores are ≤4, perform hourly until resolution.
^{k}Perform CADSS predose, 40 (±5) minutes postdose, and 1, and 2 hours postdose (all ±10 min); however, if total scores are >4, perform hourly until resolution.
^{l}For change scales, baseline is defined as the result of the severity assessment predose on Day 1.
^{m}Dosing is suggested to occur at approximately the same time of day (±1 hour) as the Day 1 dose.

The contents of each of the references cited in the present disclosure are hereby incorporated by reference in their entirety.

A number of embodiments of the present disclosure have been described.

Nevertheless, it will be understood that various modifications may be made without departing from the spirit and scope of the disclosure. Accordingly, other embodiments are within the scope of the following claims.

### Embodiments

Embodiment 1: A method for treating MDD in a subject in need thereof, the method comprising intranasally administering a therapeutically effective amount of racemic ketamine, or a pharmaceutically acceptable salt thereof, to the subject.

Embodiment 2: A method for treating TRD in a subject in need thereof, the method comprising intranasally administering a therapeutically effective amount of racemic ketamine, or a pharmaceutically acceptable salt thereof, to the subject.

Embodiment 3: A method for treating PTSD in a subject in need thereof, the method comprising intranasally administering a therapeutically effective amount of racemic ketamine, or a pharmaceutically acceptable salt thereof, to the subject.

Embodiment 4: A method for treating bipolar depression in a subject in need thereof, comprising intranasally administering to the subject a therapeutically effective amount of racemic ketamine, or a pharmaceutically acceptable salt thereof.

Embodiment 5: A method for treating post-partum depression in a subject in need thereof, comprising intranasally administering to the subject a therapeutically effective amount of racemic ketamine, or a pharmaceutically acceptable salt thereof.

Embodiment 6: A method for treating chronic pain in a subject in need thereof, comprising intranasally administering to the subject a therapeutically effective amount of racemic ketamine, or a pharmaceutically acceptable salt thereof.

Embodiment 7: A method for treating neuropathic pain in a subject in need thereof, comprising intranasally administering to the subject a therapeutically effective amount of racemic ketamine, or a pharmaceutically acceptable salt thereof.

Embodiment 8: A method for treating Rett syndrome in a subject in need thereof, comprising intranasally administering to the subject a therapeutically effective amount of racemic ketamine, or a pharmaceutically acceptable salt thereof.

Embodiment 9: A method for treating epilepsy in a subject in need thereof, comprising intranasally administering to the subject a therapeutically effective amount of racemic ketamine, or a pharmaceutically acceptable salt thereof.

Embodiment 10: A method for treating agitation associated with dementia in a subject in need thereof, comprising intranasally administering to the subject a therapeutically effective amount of racemic ketamine, or a pharmaceutically acceptable salt thereof.

Embodiment 11: A method for treating agitation associated with schizophrenia in a subject in need thereof, comprising intranasally administering to the subject a therapeutically effective amount of racemic ketamine, or a pharmaceutically acceptable salt thereof.

Embodiment 12: A method for treating agitation associated with bipolar disorder in a subject in need thereof, comprising intranasally administering to the subject a therapeutically effective amount of racemic ketamine, or a pharmaceutically acceptable salt thereof.

Embodiment 13: The method of any one of Embodiments 1-12, further comprising administering one or more additional therapies consisting of typical antipsychotics, atypical antipsychotics, antidepressants, electroconvulsive therapy, transcranial magnetic stimulation, and mood stabilizers.

Embodiment 14: The method of any one of Embodiments 1-13, wherein the subject has previously been administered one or more additional therapies consisting of typical antipsychotics, atypical antipsychotics, antidepressants, electroconvulsive therapy, transcranial magnetic stimulation, benzodiazepines, mood stabilizers, and pramipexole; wherein the subject was not responsive to the previous one or more therapies.

Embodiment 15: The method of Embodiment 13 or 14, wherein the typical antipsychotic is chlorpromazine, chlorprothixene, levomepromazine, mesoridazine, periciazine, promazine, loxapine, molindone, perphenazine, thiothixene, droperidol, flupentixol, fluphenazine, haloperidol, pimozide, prochlorperazine, thioproperazine, trifluoperazine, or zuclopenthixol.

Embodiment 16: The method of Embodiment 13 or 14, wherein the atypical antipsychotic is aripiprazole, risperidone, olanzapine, quetiapine, asenapine, paliperidone, ziprasidone, or lurasidone.

Embodiment 17: The method of Embodiment 12 or 14 or 5, wherein the antidepressant consists of atypical antidepressants, selective serotonin reuptake inhibitors, selective serotonin and norepinephrine reuptake inhibitors, monoamine oxidase inhibitors, and selective norepinephrine reuptake inhibitors.

Embodiment 18: The method of Embodiment 17, wherein the atypical antidepressant is mirtazapine, mianserin, bupropion, trazodone, nefazodone, tianeptine, opipramol, agomelatine, vilazodone, or vortioxetine.

Embodiment 19: The method of Embodiment 17, wherein the selective serotonin reuptake inhibitor is citalopram, escitalopram, fluoxetine, fluvoxamine, paroxetine, or sertraline.

Embodiment 20: The method of Embodiment 17, wherein the selective serotonin and norepinephrine reuptake inhibitor is atomoxetine, desvenlafaxine, duloxetine, levomilnacipran, milnacipran, sibutramine, or venlafaxine.

Embodiment 21: The method of Embodiment 17, wherein the monoamine oxidase inhibitor is moclobemide, rasagiline, selegiline, or safinamide.

Embodiment 22: The method of Embodiment 17, wherein the selective norepinephrine reuptake inhibitor is reboxetine.

Embodiment 23: The method of Embodiment 13 or 14, wherein the benzodiazepine is alprazolam, bromazepam, chlordiazepoxide, clonazepam, clorazepate, diazepam, flurazepam, lorazepam, oxazepam, temazepam, or triazolam.

Embodiment 24: The method of Embodiment 13 or 14, wherein the mood stabilizer is lithium, valproic acid, lamotrigine, or carbamazepine.

Embodiment 25: The method of Embodiment 13 or 14, wherein the one or more therapies are electroconvulsive therapy or transcranial magnetic stimulation.

Embodiment 26: The method of any one of Embodiments 1-25, further comprising decreasing the duration of hospitalization of the subject relative to administration of an equivalent dose of intravenous racemic ketamine, or a pharmaceutically acceptable salt thereof.

Embodiment 27: The method of any one of Embodiments 1 or 13-26, wherein the MDD of the subject resolves faster relative to administration of an equivalent dose of intravenous racemic ketamine, or a pharmaceutically acceptable salt thereof; or relative to an equivalent dose of intranasal (S)-ketamine, or a pharmaceutically acceptable salt thereof.

Embodiment 28: The method of any one of Embodiments 2 or 13-26, wherein the TRD of the subject resolves faster relative to administration of an equivalent dose of intravenous racemic ketamine, or a pharmaceutically acceptable salt thereof; or relative to an equivalent dose of intranasal (S)-ketamine, or a pharmaceutically acceptable salt thereof.

Embodiment 29: The method of any one of Embodiments 3 or 13-26, wherein the PTSD of the subject resolves faster relative to administration of an equivalent dose of intravenous racemic ketamine, or a pharmaceutically acceptable salt thereof; or relative to an equivalent dose of intranasal (S)-ketamine, or a pharmaceutically acceptable salt thereof.

Embodiment 30: The method of any one of Embodiments 4 or 13-26, wherein the bipolar depression of the subject resolves faster relative to administration of an equivalent dose of intravenous racemic ketamine, or a pharmaceutically acceptable salt thereof; or relative to an equivalent dose of intranasal (S)-ketamine, or a pharmaceutically acceptable salt thereof.

Embodiment 31: The method of any one of Embodiments 5 or 13-26, wherein the post-partum depression of the subject resolves faster relative to administration of an equivalent dose of intravenous racemic ketamine, or a pharmaceutically acceptable salt thereof; or relative to an equivalent dose of intranasal (S)-ketamine, or a pharmaceutically acceptable salt thereof.

Embodiment 32: The method of any one of Embodiments 6 or 13-26, wherein the chronic pain of the subject resolves faster relative to administration of an equivalent dose of intravenous racemic ketamine, or a pharmaceutically acceptable salt thereof; or relative to an equivalent dose of intranasal (S)-ketamine, or a pharmaceutically acceptable salt thereof.

Embodiment 33: The method of any one of Embodiments 7 or 13-26, wherein the neuropathic pain of the subject resolves faster relative to administration of an equivalent dose of intravenous racemic ketamine, or a pharmaceutically acceptable salt thereof; or relative to an equivalent dose of intranasal (S)-ketamine, or a pharmaceutically acceptable salt thereof.

Embodiment 34: The method of any one of Embodiments 8 or 13-26, wherein the Rett syndrome of the subject resolves faster relative to administration of an equivalent dose of intravenous racemic ketamine, or a pharmaceutically acceptable salt thereof; or relative to an equivalent dose of intranasal (S)-ketamine, or a pharmaceutically acceptable salt thereof.

Embodiment 35: The method of any one of Embodiments 9 or 13-26, wherein the epilepsy of the subject resolves faster relative to administration of an equivalent dose of intravenous racemic ketamine, or a pharmaceutically acceptable salt thereof; or relative to an equivalent dose of intranasal (S)-ketamine, or a pharmaceutically acceptable salt thereof.

Embodiment 36: The method of any one of Embodiments 10 or 13-26, wherein the agitation associated with dementia of the subject resolves faster relative to administration of an equivalent dose of intravenous racemic ketamine, or a pharmaceutically acceptable salt thereof, or relative to an equivalent dose of intranasal (S)-ketamine, or a pharmaceutically acceptable salt thereof.

Embodiment 37: The method of any one of Embodiments 11 or 13-26, wherein the agitation associated with schizophrenia of the subject resolves faster relative to administration of an equivalent dose of intravenous racemic ketamine, or a pharmaceutically acceptable salt thereof, or relative to an equivalent dose of intranasal (S)-ketamine, or a pharmaceutically acceptable salt thereof.

Embodiment 38: The method of any one of Embodiments 12-26, wherein the agitation associated with bipolar disorder of the subject resolves faster relative to administration of an equivalent dose of intravenous racemic ketamine, or a pharmaceutically acceptable salt thereof; or relative to an equivalent dose of intranasal (S)-ketamine, or a pharmaceutically acceptable salt thereof.

Embodiment 39: The method of any one of Embodiments 1-38, wherein the dose of the one or more therapies administered in combination with the intranasal racemic ketamine, or a pharmaceutically acceptable salt thereof, is reduced relative to the dose of the one or more therapies administered to the subject prior to treatment with the intranasal racemic ketamine, or a pharmaceutically acceptable salt thereof.

Embodiment 40: The method of any one of Embodiments 13-39, wherein the one or more therapies is a selective serotonin reuptake inhibitor, a selective serotonin and norepinephrine reuptake inhibitor, or a selective norepinephrine reuptake inhibitor.

Embodiment 41: The method of any one of Embodiments 13-40, wherein the one or more therapies is sertraline.

Embodiment 42: The method of any one of Embodiments 13-41, wherein the one or more additional therapies is venlafaxine.

Embodiment 43: The method of any one of Embodiments 1-42, wherein the subject does not experience a clinically significant weight gain relative to the administration of the one or more additional therapies in the absence of intranasal administration of racemic ketamine, or a pharmaceutically acceptable salt thereof.

Embodiment 44: The method of any one of Embodiments 1-43, wherein the magnitude of one or more side effects of the intranasal racemic ketamine, or a pharmaceutically acceptable salt thereof, are reduced relative to an equivalent dose of intravenous racemic ketamine, or a pharmaceutically acceptable salt thereof.

Embodiment 45: The method of any one of Embodiments 1-44, wherein the subject exhibits reduction in one or more side effects as compared to an equivalent dose of intravenous racemic ketamine, or a pharmaceutically acceptable salt thereof.

Embodiment 46: The method of any one of Embodiments 1-45, wherein one or more side effects of the one or more additional therapies are reduced relative to an equivalent dose of the one or more additional therapies in the absence of intranasal administration of racemic ketamine, or a pharmaceutically acceptable salt thereof.

Embodiment 47: The method of any one of Embodiments 1-46, further comprising reducing one or more side effects of ketamine in the subject.

Embodiment 48: The method of any one of Embodiments 1-47, wherein the one or more side effects are reduced relative to the one or more side effects observed after intranasal administration of an equivalent dose of (S)-ketamine, or a pharmaceutically acceptable salt thereof.

Embodiment 49: The method of Embodiment 47, wherein the one or more side effects are reduced relative to the one or more side effects observed after administration of an equivalent dose of intravenous racemic ketamine, or a pharmaceutically acceptable salt thereof.

Embodiment 50: The method of any one of Embodiments 47-49 wherein the one or more side effects comprise cognitive impairment, motor impairment, vertigo, nausea, vomiting, sweating, increased blood pressure, ulcerative cystitis, or interstitial cystitis.

Embodiment 51: The method of Embodiment 50, wherein the cognitive impairment comprises one or more of psychotomimetic effects, dizziness, dysgeusia, sedation, dissociation, euphoria, changes in hearing, changes in vision, and hallucinations.

Embodiment 52: The method of Embodiment 51, wherein the cognitive impairment comprises sedation.

Embodiment 53: The method of Embodiment 51 or 52, wherein the cognitive impairment is sedation.

Embodiment 54: The method of any one of Embodiments 50-53, wherein the motor impairment comprises tremors, issues with balance, or dystonic movements.

Embodiment 55: The method of any one of Embodiments 1-54, wherein the Columbia-Suicide Severity Rating Scale (C-SSRS) score is greater than 1, 2, or 3 units prior to intranasal administration of the racemic ketamine, or a pharmaceutically acceptable salt thereof.

Embodiment 56: The method of any one of Embodiments 1-55, wherein the C-SSRS score is 2 unit prior to intranasal administration of the racemic ketamine, or a pharmaceutically acceptable salt thereof.

Embodiment 57: The method of any one of Embodiments 1-55, wherein the C-SSRS score is 3 units prior to intranasal administration of the racemic ketamine, or a pharmaceutically acceptable salt thereof.

Embodiment 58: The method of any one of Embodiments 1-54, wherein the Columbia-Suicide Severity Rating Scale (C-SSRS) score is 4, 5, or 6 units prior to intranasal administration of the racemic ketamine, or a pharmaceutically acceptable salt thereof.

Embodiment 59: The method of any one of Embodiments 1-54 or 58, wherein the C-SSRS score is 5 units prior to intranasal administration of the racemic ketamine, or a pharmaceutically acceptable salt thereof.

Embodiment 60: The method of any one of Embodiments 1-54 or 58, wherein the C-SSRS score is 6 unit prior to intranasal administration of the racemic ketamine, or a pharmaceutically acceptable salt thereof.

Embodiment 61: The method of any one of Embodiments 1-54, wherein the C-SSRS score is determined about 1 hour to about 24 hours prior to intranasal administration of the racemic ketamine, or a pharmaceutically acceptable salt thereof.

Embodiment 62: The method of any one of Embodiments 1-54, wherein the C-SSRS decreases by 1 unit after intranasal administration of the racemic ketamine, or a pharmaceutically acceptable salt thereof.

Embodiment 63: The method of any one of Embodiments 1-54, wherein the C-SSRS decreases by 2 units after intranasal administration of the racemic ketamine, or a pharmaceutically acceptable salt thereof.

Embodiment 64: The method of any one of Embodiments 1-54, wherein a first C-SSRS score is determined about 1 hour to about 12 hours prior to intranasal administration of the racemic ketamine, or a pharmaceutically acceptable salt thereof; and a second C-SSRS score is determined about 1 hour to about 12 hours after intranasal administration of the racemic ketamine, or a pharmaceutically acceptable salt thereof.

Embodiment 65: The method of Embodiment 64, wherein the first C-SSRS score from the subject is determined about 4 hours to about 8 hours prior to intranasal administration of the racemic ketamine, or a pharmaceutically acceptable salt thereof.

Embodiment 66: The method of any Embodiment 64 or 65, wherein the second C-SSRS score from the subject is determined about 4 hours to about 8 hours after intranasal administration of the racemic ketamine, or a pharmaceutically acceptable salt thereof.

Embodiment 67: The method of any Embodiment 64 or 65, wherein the second C-SSRS score from the subject is determined about 12 hours to about 24 hours after intranasal administration of the racemic ketamine, or a pharmaceutically acceptable salt thereof.

Embodiment 68: The method of Embodiment any one of Embodiments 1-67, wherein the Modified Observer's Assessment of Alertness and Sedation (MOAA/S) score of the subject is 4 or 5, prior to intranasal administration of the racemic ketamine, or a pharmaceutically acceptable salt thereof.

Embodiment 69: The method of Embodiment any one of Embodiments 1-68, wherein the MOAA/S score of the subject is 5, prior to intranasal administration of the racemic ketamine, or a pharmaceutically acceptable salt thereof.

Embodiment 70: The method of Embodiment any one of Embodiments 1-69, wherein the MOAA/S score of the subject is determined about 1 hour to about 24 hours prior to intranasal administration of the racemic ketamine, or a pharmaceutically acceptable salt thereof.

Embodiment 71: The method of Embodiment any one of Embodiments 1-70, wherein the MOAA/S score of the subject is 5, about 10 minutes to about 2 hours after intranasal administration of the racemic ketamine, or a pharmaceutically acceptable salt thereof.

Embodiment 72: The method of Embodiment 71, wherein the MOAA/S score of the subject is 1 to 4 units higher about 10 minutes to about 2 hours after intranasal administration of the racemic ketamine, or a pharmaceutically acceptable salt thereof, than a subject administered an equivalent dose of intravenous racemic ketamine, or a pharmaceutically acceptable salt thereof.

Embodiment 73: The method of Embodiment 71, wherein the MOAA/S score of the subject is 1 to 4 units higher after about 10 minutes to about 2 hours after intranasal administration of the racemic ketamine, or a pharmaceutically acceptable salt thereof, than a subject administered an equivalent dose of (S)-ketamine, or a pharmaceutically acceptable salt thereof.

Embodiment 74: The method of Embodiment 72, wherein the rate of increase in the MOAA/S score is greater than the rate of increase in the MOAA/S score in a subject administered an equivalent dose of intravenous racemic ketamine, or a pharmaceutically acceptable salt thereof.

Embodiment 75: The method of Embodiment 73, wherein the rate of increase in the MOAA/S score is greater than the rate of increase in the MOAA/S score in a subject administered an equivalent dose of (S)-ketamine, or a pharmaceutically acceptable salt thereof.

Embodiment 76: The method of Embodiment 71, wherein the MOAA/S score of the subject is 2 to 4 units higher about 10 minutes to about 2 hours after intranasal administration of the racemic ketamine, or a pharmaceutically acceptable salt thereof, than a subject administered an equivalent dose of (S)-ketamine, or a pharmaceutically acceptable salt thereof, and 2 to 4 units higher about 10 minutes to about 2 hours after intranasal administration of the racemic ketamine, or a pharmaceutically acceptable salt thereof, than a subject administered an equivalent dose of intravenous racemic ketamine, or a pharmaceutically acceptable salt thereof.

Embodiment 77: The method of any one of Embodiments 1-76, wherein the Bowdle Visual Analog Scale score is 0 to 50, about 45 minutes to about 24 hours prior to intranasal administration of the racemic ketamine, or a pharmaceutically acceptable salt thereof.

Embodiment 78: The method of any one of Embodiments 1-77, wherein the Bowdle Visual Analog Scale score is 0 to 50, about 45 minutes to about 24 hours after intranasal administration of the racemic ketamine, or a pharmaceutically acceptable salt thereof.

Embodiment 79: The method of any one of Embodiments 1-78, wherein the Bowdle Visual Analog Scale score is reduced by about 10 to about 1300, about 45 minutes to about 24 hours after intranasal administration of the racemic ketamine, or a pharmaceutically acceptable salt thereof.

Embodiment 80: The method of Embodiment 78 or 79, wherein the Bowdle Visual Analog Scale score is determined about 1 hour to about 4 hours after intranasal administration of the racemic ketamine, or a pharmaceutically acceptable salt thereof.

Embodiment 81: The method of any one of Embodiments 1-80, wherein the Clinician Administered Dissociative States Scale (CADDS) score is 0 to 10, about 45 minutes to about 24 hours after intranasal administration of the racemic ketamine, or a pharmaceutically acceptable salt thereof.

Embodiment 82: The method of Embodiment 81, wherein the CADDS score is reduced by about 1 to about 91, about 45 minutes to about 24 hours after intranasal administration of the racemic ketamine, or a pharmaceutically acceptable salt thereof.

The method of Embodiment 82, wherein the CADDS score is determined about 1 hour to about 4 hours after intranasal administration of the racemic ketamine, or a pharmaceutically acceptable salt thereof.

Embodiment 83: The method of any one of Embodiments 1-73, wherein the Profile of Mood States score of the subject is substantially the same from about 1 hour to about 24 hours after intranasal administration of the racemic ketamine, or a pharmaceutically acceptable salt thereof, as prior to the administration.

Embodiment 84: The method of any one of Embodiments 1-84, wherein the Profile of Mood States score of the subject is substantially the same about 1 hour to about 12 hours after intranasal administration of the racemic ketamine, or a pharmaceutically acceptable salt thereof, as prior to the administration.

Embodiment 85: The method of any one of Embodiments 1-85, wherein the Profile of Mood States score of the subject is substantially the same from about 1 hour to about 4 hours after intranasal administration of the racemic ketamine, or a pharmaceutically acceptable salt thereof, as prior to the administration.

Embodiment 86: The method of Embodiment any one of Embodiments 1-76, wherein the Choice Reaction Time Test score of the subject is substantially the same from about 1 hour to about 24 hours after intranasal administration of the racemic ketamine, or a pharmaceutically acceptable salt thereof, as prior to the administration.

Embodiment 87: The method of Embodiment any one of Embodiments 1-76, wherein the Choice Reaction Time Test score of the subject is substantially the same from about 1 hour to about 12 hours after intranasal administration of the racemic ketamine, or a pharmaceutically acceptable salt thereof, as prior to the administration.

Embodiment 88: The method of Embodiment any one of Embodiments 1-88, wherein the Choice Reaction Time Test score of the subject is substantially the same from about 1 hour to about 4 hours after intranasal administration of the racemic ketamine, or a pharmaceutically acceptable salt thereof, as prior to the administration.

Embodiment 89: The method of Embodiment any one of Embodiments 1-88, wherein the Sternberg Short-Term Memory score of the subject is substantially the same from about 1 hour to about 24 hours after intranasal administration of the racemic ketamine, or a pharmaceutically acceptable salt thereof, as prior to the administration.

Embodiment 90: The method of Embodiment any one of Embodiments 1-89, wherein the Sternberg Short-Term Memory score of the subject is substantially the same from about 4 hours to about 12 hours after intranasal administration of the racemic ketamine, or a pharmaceutically acceptable salt thereof, as prior to the administration.

Embodiment 91: The method of Embodiment any one of Embodiments 1-90, wherein the Sternberg Short-Term Memory score of the subject is substantially the same from about 1 hour to about 12 hours after intranasal administration of the racemic ketamine, or a pharmaceutically acceptable salt thereof, as prior to the administration.

Embodiment 92: The method of Embodiment any one of Embodiments 1-91, wherein the Sternberg Short-Term Memory score of the subject is substantially the same from about 1 hour to about 4 hours after intranasal administration of the racemic ketamine, or a pharmaceutically acceptable salt thereof, as prior to the administration.

Embodiment 93: The method of any one of Embodiments 1-92, wherein the subject has a score of 1 or 0 on the Subject-Rated Assessment of Intranasal Irritation after intranasal administration of the racemic ketamine, or a pharmaceutically acceptable salt thereof.

Embodiment 94: The method of any one of Embodiments 1-93, wherein no clinically meaningful sedation is observed in the subject within about 24 hours after intranasal administration of the racemic ketamine, or a pharmaceutically acceptable salt thereof.

Embodiment 95: The method of Embodiment 94, wherein no clinically meaningful sedation is observed in the subject at about 4 hours after intranasal administration of the racemic ketamine, or a pharmaceutically acceptable salt thereof.

Embodiment 96: The method of Embodiment 94, wherein no clinically meaningful sedation is observed in the subject at about 1 hour after intranasal administration of the racemic ketamine, or a pharmaceutically acceptable salt thereof.

Embodiment 97: The method of any one of Embodiments 1-96, wherein no clinically meaningful dissociation is observed in the subject within about 24 hours after intranasal administration of the racemic ketamine, or a pharmaceutically acceptable salt thereof.

Embodiment 98: The method of Embodiment 97, wherein no clinically meaningful dissociation is observed in the subject at about 4 hours after intranasal administration of the racemic ketamine, or a pharmaceutically acceptable salt thereof.

Embodiment 99: The method of Embodiment 97, wherein no clinically meaningful dissociation is observed in the subject at about 1 hour after intranasal administration of the racemic ketamine, or a pharmaceutically acceptable salt thereof.

Embodiment 100: The method of any one of Embodiments 1-99, wherein the subject has been previously diagnosed with one or more of: major depressive disorder, treatment-resistant depression, and post-traumatic stress disorder.

Embodiment 101: The method of any one of Embodiments 1-100, wherein the subject is currently suffering from one or more of: major depressive disorder, treatment-resistant depression, and post-traumatic stress disorder.

Embodiment 102: The method of Embodiment 100 or 101, wherein the treatment-resistant depression is Stage I to Stage IV.

Embodiment 103: The method of Embodiment 100 or 101, wherein the treatment resistant depression is Stage V.

Embodiment 104: The method of Embodiment 100 or 101, wherein the Massachusetts General Hospital Staging Method to Classify Treatment Resistant Depression score of the subject is from 2 and 10, prior to intranasal administration of the racemic ketamine, or a pharmaceutically acceptable salt thereof.

Embodiment 105: The method of Embodiment 100 or 101, wherein the Antidepressant Treatment History Form score of the subject is 1 to 4, prior to intranasal administration of the racemic ketamine, or a pharmaceutically acceptable salt thereof.

Embodiment 106: The method of Embodiment 100 or 101, wherein the subject exhibits one or more of the following characteristics: unwanted upsetting memories, nightmares, flashbacks, emotional distress after exposure to traumatic reminders, or physical reactivity after exposure to traumatic reminders; and one or more of trauma-related thoughts or feelings and trauma-related external reminders, prior to intranasal administration of the racemic ketamine, or a pharmaceutically acceptable salt thereof.

Embodiment 107: The method of Embodiment 106, wherein the subject exhibits two or more of the following characteristics: inability to recall key features of a traumatic event, overly negative thoughts and assumptions about oneself or the world, exaggerated blame of self or others for causing a traumatic event, negative affect, decreased interest in activities, feeling isolated, and difficulty experiencing positive affect.

Embodiment 108: The method of Embodiment 106 or 107, wherein the subject exhibits one or more of the following characteristics: irritability or aggression, risky or destructive behavior, hypervigilance, heightened startle reaction, difficulty concentrating, and difficulty sleeping.

Embodiment 109: The method of any one of Embodiments 106-108, wherein the characteristics are present for more than about 1 month, create distress and/or functional impairment in social or occupational situations, and are not due to medication or substance abuse.

Embodiment 110: The method of any one of Embodiments 1-109, wherein about 30 mg to about 90 mg of racemic ketamine, or a pharmaceutically acceptable salt thereof, is intranasally administered to the subject.

Embodiment 111: The method of any one of Embodiments 1-110, wherein about 30 mg to about 60 mg of racemic ketamine, or a pharmaceutically acceptable salt thereof, is intranasally administered to the subject.

Embodiment 112: The method of any one of Embodiments 1-111, wherein about 60 mg to about 90 mg of racemic ketamine, or a pharmaceutically acceptable salt thereof, is intranasally administered to the subject.

Embodiment 113: The method of any one of Embodiments 1-112, wherein about 30 mg, about 60 mg, about 75 mg, or about 90 mg of racemic ketamine, or a pharmaceutically acceptable salt thereof, is intranasally administered to the subject, preferably about 60 mg per dose.

Embodiment 114: The method of any one of Embodiments 1-113, wherein the t½ for ketamine is about 2 hours to about 9 hours following intranasal administration of racemic ketamine.

Embodiment 115: The method of any one of Embodiments 1-114, wherein the t½ for ketamine is about 4 hours to about 7 hours following intranasal administration of racemic ketamine.

Embodiment 116: The method of any one of Embodiments 1-115, wherein the t½ for 6-hydroxynorketamine is about 5.5 hours to about 21.5 hours following intranasal administration of racemic ketamine.

Embodiment 117: The method of any one of Embodiments 1-116, wherein the t½ for 6-hydroxynorketamine is about 10 hours to about 12 hours following intranasal administration of racemic ketamine.

Embodiment 118: The method of any one of Embodiments 1-117, wherein the t½ for norketamine is about 4.5 hours to about 12.5 hours following intranasal administration of racemic ketamine.

Embodiment 119: The method of any one of Embodiments 1-118, wherein the t½ for norketamine is about 7 hours to about 8 hours following intranasal administration of racemic ketamine.

Embodiment 120: The method of any one of Embodiments 1-119, wherein racemic ketamine, or a pharmaceutically acceptable salt thereof, is intranasally administered from about once per day to about once per month.

Embodiment 121: The method of any one of Embodiments 1-120, wherein racemic ketamine, or a pharmaceutically acceptable salt thereof, is intranasally administered from about once per day to about once every two weeks.

Embodiment 122: The method of any one of Embodiments 1-121, wherein racemic ketamine, or a pharmaceutically acceptable salt thereof, is intranasally administered from about once per day to about once per week.

Embodiment 123: The method of any one of Embodiments 1-122, wherein racemic ketamine, or a pharmaceutically acceptable salt thereof, is intranasally administered from about once per week to about twice per week.

Embodiment 124: The method of any one of Embodiments 1-123, wherein racemic ketamine, or a pharmaceutically acceptable salt thereof, is intranasally administered twice per week.

Embodiment 125: The method of any one of Embodiments 1-124, wherein racemic ketamine, or a pharmaceutically acceptable salt thereof, is intranasally administered once per day, once every other day, three times per week, twice per week, or once per week.

Embodiment 126: The method of any one of Embodiments 1-125, wherein the Tₘₐₓ of the ketamine is from about 20 minutes to about 120 minutes following intranasal administration of racemic ketamine.

Embodiment 127: The method of any one of Embodiments 1-126, wherein the Tₘₐₓ of the ketamine is from about 30 minutes to about 90 minutes following intranasal administration of racemic ketamine.

Embodiment 128: The method of any one of Embodiments 1-127, wherein the Tₘₐₓ of 6-hydroxynorketamine is from about 45 minutes to about 8 hours following intranasal administration of racemic ketamine.

Embodiment 129: The method of any one of Embodiments 1-128, wherein the Tₘₐₓ of norketamine is from about 45 minutes to about 360 minutes following intranasal administration of racemic ketamine.

Embodiment 130: The method of any one of Embodiments 1-129, wherein the Cₘₐₓ of ketamine is from about 15 ng/mL to about 225 ng/ml following intranasal administration of racemic ketamine.

Embodiment 131: The method of any one of Embodiments 1-130, wherein the Cₘₐₓ of ketamine is from about 70 ng/ml to about 205 ng/ml following intranasal administration of racemic ketamine.

Embodiment 132: The method of any one of Embodiments 1-131, wherein the Cₘₐₓ of 6-hydroxynorketamine is from about 15 ng/mL to about 275 ng/ml following intranasal administration of racemic ketamine.

Embodiment 133: The method of any one of Embodiments 1-132, wherein the Cₘₐₓ of 6-hydroxynorketamine is from about 80 ng/ml to about 265 ng/ml following intranasal administration of racemic ketamine.

Embodiment 134: The method of any one of Embodiments 1-133, wherein the Cₘₐₓ of norketamine is from about 40 ng/ml to about 375 ng/ml following intranasal administration of racemic ketamine.

Embodiment 135: The method of any one of Embodiments 1-134, wherein the Cₘₐₓ of norketamine is from about 160 ng/ml to about 195 ng/mL following intranasal administration of racemic ketamine.

Embodiment 136: The method of any one of Embodiments 1-135, wherein the AUC₀₋ₜ for 6-hydroxynorketamine is from about 300 ng*h/mL to about 3,100 ng*h/mL following intranasal administration of racemic ketamine.

Embodiment 137: The method of any one of Embodiments 1-136, wherein the AUC₀₋ₜ for 6-hydroxynorketamine is from about 850 ng*h/mL to about 950 ng*h/mL following intranasal administration of racemic ketamine.

Embodiment 138: The method of any one of Embodiments 1-137, wherein the AUC₀₋ₜ for norketamine is from about 250 ng*h/mL to about 2,200 ng*h/mL following intranasal administration of racemic ketamine.

Embodiment 139: The method of any one of Embodiments 1-138, wherein the AUC₀₋ₜ for norketamine is from about 900 ng*h/mL to about 1,550 ng*h/mL following intranasal administration of racemic ketamine.

Embodiment 140: The method of any one of Embodiments 1-139, wherein the AUC_{0-inf} for 6-hydroxynorketamine is from about 375 ng*h/mL to about 3,700 ng*h/mL following intranasal administration of racemic ketamine.

Embodiment 141: The method of any one of Embodiments 1-140, wherein the AUC_{0-inf} for 6-hydroxynorketamine is from about 1,200 ng*h/mL to about 1,400 ng*h/mL following intranasal administration of racemic ketamine.

Embodiment 142: The method of any one of Embodiments 1-141 wherein the AUC_{0-inf} for norketamine is from about 250 ng*h/mL to about 875 ng*h/mL following intranasal administration of racemic ketamine.

Embodiment 143: The method of any one of Embodiments 1-142, wherein the AUC_{0-inf} for norketamine is from about 450 ng*h/mL to about 675 ng*h/mL following intranasal administration of racemic ketamine.

Embodiment 144: The method of Embodiment 50, wherein the cognitive impairment comprises dissociation.

Embodiment 145: The method of Embodiment 50 or 51, wherein the cognitive impairment is dissociation.

Embodiment 146: The method of any one of Embodiments 1-145, wherein intranasal administration of the racemic ketamine exhibits one or more of:

an AUC₀₋ₜ of norketamine that is at least 1.5 times higher than the AUC₀₋ₜ of norketamine exhibited by intravenous administration of an equivalent dose of racemic ketamine;
an AUC_{0-inf} of norketamine that is at least 1.5 times higher than the AUC_{0-inf} of norketamine exhibited by intravenous administration of an equivalent dose of racemic ketamine; and
a Cₘₐₓ of norketamine that is at least 2 times higher than the Cₘₐₓ of norketamine exhibited by intravenous administration of an equivalent dose of racemic ketamine.

Embodiment 147: The method of any one of Embodiments 1-146, wherein intranasal administration of the racemic ketamine exhibits an AUC₀₋ₜ of norketamine that is about 1.7 to about 2.5 times higher than the AUC₀₋ₜ of norketamine exhibited by intravenous administration of an equivalent dose of racemic ketamine.

Embodiment 148: The method of any one of Embodiments 1-147, wherein intranasal administration of the racemic ketamine exhibits an AUC₀₋ₜ of norketamine that is about 1.9 to about 2.3 times higher than the AUC₀₋ₜ of norketamine exhibited by intravenous administration of an equivalent dose of racemic ketamine.

Embodiment 149: The method of any one of Embodiments 1-148, wherein intranasal administration of the racemic ketamine exhibits an AUC_{0-inf} of norketamine that is about 1.5 to about 2.5 times higher than the AUC_{0-inf} of norketamine exhibited by intravenous administration of an equivalent dose of racemic ketamine.

Embodiment 150: The method of any one of Embodiments 1-149, wherein intranasal administration of the racemic ketamine exhibits an AUC₀₋ₜ of norketamine that is about 1.8 to about 2.2 times higher than the AUC_{0-inf} of norketamine exhibited by intravenous administration of an equivalent dose of racemic ketamine.

Embodiment 151: The method of any one of Embodiments 1-150, wherein intranasal administration of the racemic ketamine exhibits a Cₘₐₓ of norketamine that is about 2.2 to about 3.5 times higher than the Cₘₐₓ of norketamine exhibited by intravenous administration of an equivalent dose of racemic ketamine.

Embodiment 152: The method of any one of Embodiments 1-151, wherein intranasal administration of the racemic ketamine exhibits a Cₘₐₓ of norketamine that is about 2.4 to about 3.2 times higher than the Cₘₐₓ of norketamine exhibited by intravenous administration of an equivalent dose of racemic ketamine.

Embodiment 153: The method of any one of Embodiments 1-152, wherein intranasal administration of the racemic ketamine exhibits a Tₘₐₓ of norketamine that is about 80% to about 125% of the Tₘₐₓ of norketamine exhibited by an intravenous administration of an equivalent dose of racemic ketamine.

Embodiment 154: The method of any one of Embodiments 1-153, wherein intranasal administration of the racemic ketamine exhibits a Tₘₐₓ of norketamine that is about 90% to about 110% of the Tₘₐₓ of norketamine exhibited by an intravenous administration of an equivalent dose of racemic ketamine.

Embodiment 155: The method of any one of Embodiments 1-154, wherein one or more of the AUC₀₋ₜ, AUC_{0-inf}, Cₘₐₓ, and Tₘₐₓ of norketamine is determined following one dose of racemic ketamine.

Embodiment 156: The method of any one of Embodiments 1-154, wherein one or more of the AUC₀₋ₜ, AUC_{0-inf}, Cₘₐₓ, and Tₘₐₓ of norketamine is determined following two doses of racemic ketamine.

Embodiment 157: The method of any one of Embodiments 1-154, wherein one or more of the AUC₀₋ₜ, AUC_{0-inf}, Cₘₐₓ, and Tₘₐₓ of norketamine is determined following three doses of racemic ketamine.

Embodiment 158: The method of any one of Embodiments 1-157, wherein intranasal administration of the racemic ketamine exhibits one or more of:

an AUC₀₋ₜ of hydroxynorketamine that is at least 1.5 times higher than the AUC₀₋ₜ of hydroxynorketamine exhibited by intravenous administration of an equivalent dose of racemic ketamine;
an AUC_{0-inf} of hydroxynorketamine that is at least 1.2 times higher than the AUC_{0-inf} of hydroxynorketamine exhibited by intravenous administration of an equivalent dose of racemic ketamine; and
a Cₘₐₓ of hydroxynorketamine that is at least 2 times higher than the Cₘₐₓ of norketamine exhibited by intravenous administration of an equivalent dose of racemic ketamine.

Embodiment 159: The method of any one of Embodiments 1-158, wherein intranasal administration of the racemic ketamine exhibits an AUC₀₋ₜ of hydroxynorketamine that is about 1.7 to about 2.5 times higher than the AUC₀₋ₜ of hydroxynorketamine exhibited by intravenous administration of an equivalent dose of racemic ketamine.

Embodiment 160: The method of any one of Embodiments 1-159, wherein intranasal administration of the racemic ketamine exhibits an AUC₀₋ₜ of hydroxynorketamine that is about 1.9 to about 2.3 times higher than the AUC₀₋ₜ of hydroxynorketamine exhibited by intravenous administration of an equivalent dose of racemic ketamine.

Embodiment 161: The method of any one of Embodiments 1-160, wherein intranasal administration of the racemic ketamine exhibits an AUC_{0-inf} of hydroxynorketamine that is about 1.5 to about 2.5 times higher than the AUC_{0-inf} of hydroxynorketamine exhibited by intravenous administration of an equivalent dose of racemic ketamine.

Embodiment 162: The method of any one of Embodiments 1-161, wherein intranasal administration of the racemic ketamine exhibits an AUC₀₋ₜ of hydroxynorketamine that is about 1.7 to about 2.1 times higher than the AUC_{0-inf} of hydroxynorketamine exhibited by intravenous administration of an equivalent dose of racemic ketamine.

Embodiment 163: The method of any one of Embodiments 1-162, wherein intranasal administration of the racemic ketamine exhibits a Cₘₐₓ of hydroxynorketamine that is about 2.2 to about 3.2 times higher than the Cₘₐₓ of hydroxynorketamine exhibited by intravenous administration of an equivalent dose of racemic ketamine.

Embodiment 164: The method of any one of Embodiments 1-163, wherein intranasal administration of the racemic ketamine exhibits a Cₘₐₓ of hydroxynorketamine that is about 2.4 to about 2.8 times higher than the Cₘₐₓ of hydroxynorketamine exhibited by intravenous administration of an equivalent dose of racemic ketamine.

Embodiment 165: The method of any one of Embodiments 1-164, wherein intranasal administration of the racemic ketamine exhibits a Tₘₐₓ of hydroxynorketamine that is about 80% to about 125% of the Tₘₐₓ of hydroxynorketamine exhibited by an intravenous administration of an equivalent dose of racemic ketamine.

Embodiment 166: The method of any one of Embodiments 1-165, wherein intranasal administration of the racemic ketamine exhibits a Tₘₐₓ of hydroxynorketamine that is about 90% to about 110% of the Tₘₐₓ of hydroxynorketamine exhibited by an intravenous administration of an equivalent dose of racemic ketamine.

Embodiment 167: The method of any one of Embodiments 1-166, wherein one or more of the AUC₀₋ₜ, AUC_{0-inf}, Cₘₐₓ, and Tₘₐₓ of hydroxynorketamine is determined following one dose of racemic ketamine.

Embodiment 168: The method of any one of Embodiments 1-167, wherein one or more of the AUC₀₋ₜ, AUC_{0-inf}, Cₘₐₓ, and Tₘₐₓ of hydroxynorketamine is determined following two doses of racemic ketamine.

Embodiment 169: The method of any one of Embodiments 1-167, wherein one or more of the AUC₀₋ₜ, AUC_{0-inf}, Cₘₐₓ, and Tₘₐₓ of hydroxynorketamine is determined following three doses of racemic ketamine.

Embodiment 170: The method of any one of Embodiments 1-169, wherein the Montgomery-Asberg Depression Rating Scale (MADRS) Total Score of the subject is from 20-60 units prior to intranasal administration of the racemic ketamine, or a pharmaceutically acceptable salt thereof.

Embodiment 171: The method of any one of Embodiments 1-170, wherein the MADRS Total Score of the subject is from 20-40 units prior to intranasal administration of the racemic ketamine, or a pharmaceutically acceptable salt thereof.

Embodiment 172: The method of any one of Embodiments 1-171, wherein the MADRS Total Score of the subject is reduced by at least 50% about 24 hours after intranasal administration of the racemic ketamine, or a pharmaceutically acceptable salt thereof.

Embodiment 173: The method of any one of Embodiments 1-172, wherein the MADRS Total Score of the subject is less than or equal to 15 units about 24 hours after intranasal administration of the racemic ketamine, or a pharmaceutically acceptable salt thereof.

Embodiment 174: The method of any one of Embodiments 1-173, wherein the MADRS Total Score of the subject is less than or equal to 12 units about 48 hours after intranasal administration of the racemic ketamine, or a pharmaceutically acceptable salt thereof.

Embodiment 175: The method of any one of Embodiments 1-174, wherein the MADRS Item 10 Score of the subject is 0, 1, 2, or 3 units prior to intranasal administration of the racemic ketamine, or a pharmaceutically acceptable salt thereof.

Embodiment 176: The method of any one of Embodiments 1-175, wherein the MADRS Item 10 Score of the subject is 1 or 2 units prior to intranasal administration of the racemic ketamine, or a pharmaceutically acceptable salt thereof.

Embodiment 177: The method of any one of Embodiments 1-176, wherein the MADRS Item 10 Score of the subject is 4, 5, or 6 units prior to intranasal administration of the racemic ketamine, or a pharmaceutically acceptable salt thereof.

Embodiment 178: The method of any one of Embodiments 1-177, wherein the MADRS Item 10 Score of the subject is reduced by at least 1 unit about 4 hours administration of the racemic ketamine, or a pharmaceutically acceptable salt thereof.

Embodiment 179: The method of any one of Embodiments 1-178, wherein the Clinical Global Impression of Severity for Suicide Ideation and Behavior (CGIS-SI/B) score of the subject is 0, 1, 2, or 3 units prior to intranasal administration of the racemic ketamine, or a pharmaceutically acceptable salt thereof.

Embodiment 180: The method of any one of Embodiments 1-179, wherein the Clinical Global Impression of Severity for Suicide Ideation and Behavior (CGIS-SI/B) score of the subject is 4, 5, 6, or 7 units prior to intranasal administration of the racemic ketamine, or a pharmaceutically acceptable salt thereof.

Embodiment 181: The method of any one of Embodiments 1-180, wherein the Clinical Global Impression of Severity for Suicide Ideation and Behavior (CGIS-SI/B) score of the subject is 3, 4, or units prior to intranasal administration of the racemic ketamine, or a pharmaceutically acceptable salt thereof.

Embodiment 182: The method of any one of Embodiments 1-181, wherein the CGIS-SI/B score of the subject is 0 or 1 unit about 24 hours after intranasal administration of the racemic ketamine, or a pharmaceutically acceptable salt thereof.

Embodiment 183: The method of any one of Embodiments 1-182, wherein the Sheehan-Suicidality Tracking Scale (STS) Clinically Meaningful Change Measure (CMCM) score of the subject is from 1-52 units prior to intranasal administration of the racemic ketamine, or a pharmaceutically acceptable salt thereof.

Embodiment 184: The method of any one of Embodiments 1-183, wherein the Sheehan-Suicidality Tracking Scale (STS) Clinically Meaningful Change Measure (CMCM) score of the subject is from 15-52 units prior to intranasal administration of the racemic ketamine, or a pharmaceutically acceptable salt thereof.

Embodiment 185: The method of any one of Embodiments 1-184, wherein the STS-CMCM score of the subject is from 20-52 units prior to intranasal administration of the racemic ketamine, or a pharmaceutically acceptable salt thereof.

Embodiment 186: The method of any one of Embodiments 1-185, wherein the STS-CMCM score of the subject is reduced by at least 50% about 24 hours after intranasal administration of the racemic ketamine, or a pharmaceutically acceptable salt thereof.

Embodiment 187: The method of any one of Embodiments 1-186, wherein the STS-CMCM score of the subject is from 1-3 units about 24 hours after intranasal administration of the racemic ketamine, or a pharmaceutically acceptable salt thereof.

Embodiment 188: The method of any one of Embodiments 1-187, wherein the Sheehan-Suicidality Tracking Scale (STS) Clinically Meaningful Change Measure (CMCM) Risk of Suicide at Within the Next 7 Days score of the subject is from 5 to 10 units prior to intranasal administration of the racemic ketamine, or a pharmaceutically acceptable salt thereof.

Embodiment 189: The method of any one of Embodiments 1-188, wherein the STS-CMCM Risk of Suicide at Within the Next 7 Days score of the subject is reduced by at least 50% about 24 hours after intranasal administration of the racemic ketamine, or a pharmaceutically acceptable salt thereof.

Embodiment 190: The method of any one of Embodiments 1-189, wherein the STS-CMCM Risk of Suicide at Within the Next 7 Days score of the subject is from 0-2 units about 24 hours after intranasal administration of the racemic ketamine, or a pharmaceutically acceptable salt thereof.

Embodiment 191: The method of any one of Embodiments 1-189, wherein the STS-CMCM Risk of Suicide at Within the Next 7 Days score of the subject is 0 or 1 unit about 96 hours after intranasal administration of the racemic ketamine, or a pharmaceutically acceptable salt thereof.

Embodiment 192: The method of any one of Embodiments 1-1191, wherein the Modified Observer's Assessment of Alertness/Sedation (MOAA/S) score of the subject is 5 units prior to intranasal administration of the racemic ketamine, or a pharmaceutically acceptable salt thereof.

Embodiment 193: The method of any one of Embodiments 1-192, wherein the MOAA/S score of the subject is 4 or 5 units from 15 minutes to 6 hours after intranasal administration of the racemic ketamine, or a pharmaceutically acceptable salt thereof.

Embodiment 194: The method of any one of Embodiments 1-193, wherein the CADSS score of the subject is zero units prior to intranasal administration of the racemic ketamine, or a pharmaceutically acceptable salt thereof.

Embodiment 195: The method of any one of Embodiments 1-194, wherein the CADSS score of the subject is zero units from about 1 hour to about 6 hours after intranasal administration of the racemic ketamine, or a pharmaceutically acceptable salt thereof.

Embodiment 196: The method of any one of Embodiments 1-195, wherein the C-SSRS score of the subject is from 2 units to 9 units prior to intranasal administration of the racemic ketamine, or a pharmaceutically acceptable salt thereof.

Embodiment 197: The method of any one of Embodiments 1-196, wherein the C-SSRS score of the subject is from 2 units to 5 units prior to intranasal administration of the racemic ketamine, or a pharmaceutically acceptable salt thereof.

Embodiment 198: The method of any one of Embodiments 1-197, wherein the CSSR-S score of the subject is zero units 24 hours after intranasal administration of the racemic ketamine, or a pharmaceutically acceptable salt thereof.

Embodiment 199: The method of any one of Embodiments 1-198, wherein the subject does not have suicidality.

Embodiment 200: The method of any one of Embodiments 1-199, wherein the subject has not been diagnosed with suicidality.

Embodiment 201: The method of any one of Embodiments 1-200, wherein the subject is not suffering from suicidality.

Embodiment 202: The method of any one of Embodiments 1-201, wherein the subject is not at imminent risk of suicide.

What is claimed is:

1. A method for treating MDD in a subject in need thereof, the method comprising intranasally administering a therapeutically effective amount of racemic ketamine, or a pharmaceutically acceptable salt thereof, to the subject.

2. A method for treating TRD in a subject in need thereof, the method comprising intranasally administering a therapeutically effective amount of racemic ketamine, or a pharmaceutically acceptable salt thereof, to the subject.

3. A method for treating PTSD in a subject in need thereof, the method comprising intranasally administering a therapeutically effective amount of racemic ketamine, or a pharmaceutically acceptable salt thereof, to the subject.

4. A method for treating bipolar depression in a subject in need thereof, comprising intranasally administering to the subject a therapeutically effective amount of racemic ketamine, or a pharmaceutically acceptable salt thereof.

5. A method for treating post-partum depression in a subject in need thereof, comprising intranasally administering to the subject a therapeutically effective amount of racemic ketamine, or a pharmaceutically acceptable salt thereof.

6. A method for treating chronic pain in a subject in need thereof, comprising intranasally administering to the subject a therapeutically effective amount of racemic ketamine, or a pharmaceutically acceptable salt thereof.

7. A method for treating neuropathic pain in a subject in need thereof, comprising intranasally administering to the subject a therapeutically effective amount of racemic ketamine, or a pharmaceutically acceptable salt thereof.

8. A method for treating Rett syndrome in a subject in need thereof, comprising intranasally administering to the subject a therapeutically effective amount of racemic ketamine, or a pharmaceutically acceptable salt thereof.

9. A method for treating epilepsy in a subject in need thereof, comprising intranasally administering to the subject a therapeutically effective amount of racemic ketamine, or a pharmaceutically acceptable salt thereof.

10. A method for treating agitation associated with dementia in a subject in need thereof, comprising intranasally administering to the subject a therapeutically effective amount of racemic ketamine, or a pharmaceutically acceptable salt thereof.

11. A method for treating agitation associated with schizophrenia in a subject in need thereof, comprising intranasally administering to the subject a therapeutically effective amount of racemic ketamine, or a pharmaceutically acceptable salt thereof.

12. A method for treating agitation associated with bipolar disorder in a subject in need thereof, comprising intranasally administering to the subject a therapeutically effective amount of racemic ketamine, or a pharmaceutically acceptable salt thereof.

13. The method of any one of claims 1-12, further comprising decreasing the duration of hospitalization of the subject relative to administration of an equivalent dose of intravenous racemic ketamine, or a pharmaceutically acceptable salt thereof; or relative to an equivalent dose of intranasal (S)-ketamine, or a pharmaceutically acceptable salt thereof.

14. The method of claim 1, wherein the MDD of the subject resolves faster relative to administration of an equivalent dose of intravenous racemic ketamine, or a pharmaceutically acceptable salt thereof; or relative to an equivalent dose of intranasal (S)-ketamine, or a pharmaceutically acceptable salt thereof.

15. The method of claim 2, wherein the TRD of the subject resolves faster relative to administration of an equivalent dose of intravenous racemic ketamine, or a pharmaceutically acceptable salt thereof; or relative to an equivalent dose of intranasal (S)-ketamine, or a pharmaceutically acceptable salt thereof.

16. The method of claim 3, wherein the PTSD of the subject resolves faster relative to administration of an equivalent dose of intravenous racemic ketamine, or a pharmaceutically acceptable salt thereof, or relative to an equivalent dose of intranasal (S)-ketamine, or a pharmaceutically acceptable salt thereof.

17. The method of claim 4, wherein the bipolar depression of the subject resolves faster relative to administration of an equivalent dose of intravenous racemic ketamine, or a pharmaceutically acceptable salt thereof; or relative to an equivalent dose of intranasal (S)-ketamine, or a pharmaceutically acceptable salt thereof.

18. The method of claim 5, wherein the post-partum depression of the subject resolves faster relative to administration of an equivalent dose of intravenous racemic ketamine, or a pharmaceutically acceptable salt thereof; or relative to an equivalent dose of intranasal (S)-ketamine, or a pharmaceutically acceptable salt thereof.

19. The method of claim 6, wherein the chronic pain of the subject resolves faster relative to administration of an equivalent dose of intravenous racemic ketamine, or a pharmaceutically acceptable salt thereof; or relative to an equivalent dose of intranasal (S)-ketamine, or a pharmaceutically acceptable salt thereof.

20. The method of claim 7, wherein the neuropathic pain of the subject resolves faster relative to administration of an equivalent dose of intravenous racemic ketamine, or a pharmaceutically acceptable salt thereof; or relative to an equivalent dose of intranasal (S)-ketamine, or a pharmaceutically acceptable salt thereof.

21. The method of claim 8, wherein the Rett syndrome of the subject resolves faster relative to administration of an equivalent dose of intravenous racemic ketamine, or a pharmaceutically acceptable salt thereof; or relative to an equivalent dose of intranasal (S)-ketamine, or a pharmaceutically acceptable salt thereof.

22. The method of claim 9, wherein the epilepsy of the subject resolves faster relative to administration of an equivalent dose of intravenous racemic ketamine, or a pharmaceutically acceptable salt thereof; or relative to an equivalent dose of intranasal (S)-ketamine, or a pharmaceutically acceptable salt thereof.

23. The method of claim 10, wherein the agitation associated with dementia of the subject resolves faster relative to administration of an equivalent dose of intravenous racemic ketamine, or a pharmaceutically acceptable salt thereof; or relative to an equivalent dose of intranasal (S)-ketamine, or a pharmaceutically acceptable salt thereof.

24. The method of claim 11, wherein the agitation associated with schizophrenia of the subject resolves faster relative to administration of an equivalent dose of intravenous racemic ketamine, or a pharmaceutically acceptable salt thereof; or relative to an equivalent dose of intranasal (S)-ketamine, or a pharmaceutically acceptable salt thereof.

25. The method of claim 12, wherein the agitation associated with bipolar disorder of the subject resolves faster relative to administration of an equivalent dose of intravenous racemic ketamine, or a pharmaceutically acceptable salt thereof; or relative to an equivalent dose of intranasal (S)-ketamine, or a pharmaceutically acceptable salt thereof.

26. The method of any one of claims 1-25, wherein the magnitude of one or more side effects of the intranasal racemic ketamine, or a pharmaceutically acceptable salt thereof, are reduced relative to an equivalent dose of intravenous racemic ketamine, or a pharmaceutically acceptable salt thereof.

27. The method of any one of claims 1-25, wherein the one or more side effects are reduced relative to the one or more side effects observed after intranasal administration of an equivalent dose of (S)-ketamine, or a pharmaceutically acceptable salt thereof.

28. The method of claim 26 or 27, wherein the one or more side effects comprise cognitive impairment, motor impairment, vertigo, nausea, vomiting, sweating, increased blood pressure, ulcerative cystitis, or interstitial cystitis.

29. The method of claim 28, wherein the cognitive impairment comprises one or more of psychotomimetic effects, dizziness, dysgeusia, sedation, dissociation, euphoria, changes in hearing, changes in vision, and hallucinations.

30. The method of claim 29, wherein the cognitive impairment comprises sedation.

31. The method of any one of claims 1-30, wherein no clinically meaningful sedation is observed in the subject within about 24 hours after intranasal administration of the racemic ketamine, or a pharmaceutically acceptable salt thereof.

32. The method of any one of claims 1-30, wherein no clinically meaningful sedation is observed in the subject at about 4 hours after intranasal administration of the racemic ketamine, or a pharmaceutically acceptable salt thereof.

33. The method of any one of claims 1-32, wherein no clinically meaningful sedation is observed in the subject at about 1 hour after intranasal administration of the racemic ketamine, or a pharmaceutically acceptable salt thereof.

34. The method of any one of claims 1-33, wherein no clinically meaningful dissociation is observed in the subject within about 24 hours after intranasal administration of the racemic ketamine, or a pharmaceutically acceptable salt thereof.

35. The method of any one of claims 1-34, wherein no clinically meaningful dissociation is observed in the subject at about 4 hours after intranasal administration of the racemic ketamine, or a pharmaceutically acceptable salt thereof.

36. The method of any one of claims 1-35, wherein no clinically meaningful dissociation is observed in the subject at about 1 hour after intranasal administration of the racemic ketamine, or a pharmaceutically acceptable salt thereof.
